(19) United States
(12) Patent Application Publication
(10) Pub. No.: US 2022/0231226 A1
(43) Pub. Date: Jul. 21, 2022

(54) ELECTRONIC DEVICE

(71) Applicant: Merck Patent GmbH, Darmstadt (DE)

(72) Inventors: Florian MAIER-FLAIG, Weinheim (DE); Frank VOGES, Bad Duerkheim (DE); Elvira MONTENEGRO, Weinheim (DE); Teresa MUJICA-FERNAUD, Tenerife (ES); Aurélie LUDEMANN, Frankfurt am Main (DE)

(21) Appl. No.: 17/608,495

(22) PCT Filed: Apr. 30, 2020
(86) PCT No.: PCT/EP2020/061982

§ 371 Date: Nov. 3, 2021

(30) Foreign Application Priority Data
May 3, 2019 (EP) 19172610.8

(51) Int. Cl. H01L 51/00; C07C 211/61; C09K 11/06; C07C 211/54

(52) U.S. Cl. CPC H01L 51/006; C07C 211/61; H01L 51/5056; C07C 211/54; C09K 11/06

(57) ABSTRACT

The application relates to an electronic device comprising an organic layer containing a mixture of at least two different compounds.

The present application relates to an electronic device comprising, in this sequence, an anode, a hole injection layer, a hole-transporting layer, an emitting layer, and a cathode. The hole-transporting layer contains a first compound selected from spirobifluoreneamine and fluoreneamine compounds, and a second compound which is different from the first compound and is selected from spirobifluoreneamine and fluoreneamine compounds.

Electronic devices in the context of this application are understood to mean what are called organic electronic devices, which contain organic semiconductor materials as functional materials. More particularly, these are understood to mean OLEDs (organic light-emitting diodes, organic electroluminescent devices). These are electronic devices which have one or more layers comprising organic compounds and emit light on application of electrical voltage. The construction and general principle of function of OLEDs are known to those skilled in the art.

A hole injection layer is understood to mean a layer which, in operation of the electronic device, supports the injection of holes from the anode into the hole-transporting layers of the OLED. The hole injection layer preferably directly adjoins the anode, and there are one or more hole-transporting layers on the cathode side that directly adjoin the hole injection layer.

A hole-transporting layer is understood to be a layer capable of transporting holes in operation of the electronic device. More particularly, it is a layer disposed between anode and the closest emitting layer to the anode in an OLED.

In electronic devices, especially OLEDs, there is great interest in an improvement in the performance data, especially lifetime, efficiency, operating voltage and colour purity. In these aspects, it has not yet been possible to find any entirely satisfactory solution.

Hole-transporting layers have a great influence on the abovementioned performance data of the electronic devices. They may occur as an individual hole-transporting layer between anode and emitting layer, or occur in the form of multiple hole-transporting layers, for example 2 or 3 hole-transporting layers, between anode and emitting layer.

Materials for hole-transporting layers that are known in the prior art are primarily amine compounds, especially triarylamine compounds. Examples of such triarylamine compounds are spirobifluoreneamines, fluoreneamines, indenofluoreneamines, phenanthreneamines, carbazoleamines, xantheneamines, spirodihydroacridineamines, biphenylamines and combinations of these structural elements having one or more amino groups, this being just a selection, and the person skilled in the art being aware of further structure classes.

It has now been found that an electronic device containing, in this sequence, anode, hole injection layer, hole-transporting layer, emitting layer, and cathode, wherein the hole-transporting layer contains a first compound selected from spirobifluoreneamine and fluoreneamine compounds and a second compound other than the first compound that is selected from spirobifluoreneamine and fluoreneamine compounds, has better performance data than an electronic device according to the prior art in which the hole-transporting layer is formed from a single compound. More particularly, the lifetime and/or efficiency of such a device is improved compared to the abovementioned device according to the prior art.

The present application thus provides an electronic device comprising
anode,
cathode,
emitting layer disposed between anode and cathode,
a hole injection layer disposed between anode and emitting layer;
a hole-transporting layer disposed between hole injection layer and emitting layer and directly adjoining the emitting layer on the anode side, and containing two different compounds conforming to identical or different formulae selected from formulae (I) and (II)

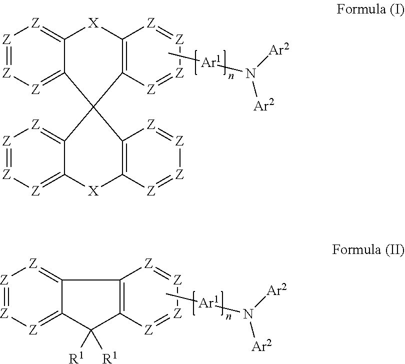

where
   Z is the same or different at each instance and is selected from CR¹ and N, where Z is C when a

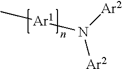

group is bonded thereto;
   X is the same or different at each instance and is selected from single bond, O, S, C(R¹)₂ and NR¹;
   Ar¹ and Ar² are the same or different at each instance and are selected from aromatic ring systems which have 6 to 40 aromatic ring atoms and are substituted by one or more R² radicals and heteroaromatic ring systems which have 5 to 40 aromatic ring atoms and are substituted by one or more R² radicals;
   R¹ and R² are the same or different at each instance and are selected from H, D, F, Cl, Br, I, C(═O)R³, CN, Si(R³)₃, N(R³)₂, P(═O)(R³)₂, OR³, S(═O)R³, S(═O)₂R³, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ or R² radicals may be joined to one another and may form a ring; where the alkyl, alkoxy, alkenyl and alkynyl groups mentioned and the aromatic ring systems and heteroaromatic ring systems mentioned are each substituted by R³ radicals; and where one or more CH₂ groups in the alkyl, alkoxy, alkenyl and alkynyl groups mentioned may be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
   R³ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R³ radicals may be joined to one another and may form a ring; and where the alkyl, alkoxy, alkenyl and alkynyl groups, aromatic ring systems and heteroaromatic ring systems mentioned may be substituted by one or more radicals selected from F and ON;
   n is 0, 1, 2, 3 or 4, where, when n=0, the Ar¹ group is absent and the nitrogen atom is bonded directly to the rest of the formula.

When n=2, two Ar¹ groups are bonded successfully in a row, as -Ar¹-Ar¹—. When n=3, three Ar¹ groups are bonded successfully in a row, as -Ar¹-Ar¹—Ar¹—. When n=4, four Ar¹ groups are bonded successfully in a row, as -Ar¹—Ar¹-Ar¹-Ar¹—.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatoms as aromatic ring atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more non-aromatic rings fused to at least one aryl group. These non-aromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more non-aromatic rings fused to at least one aryl or heteroaryl group. The non-aromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a non-aromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording should also be understood to mean that, if one of the two radicals is hydrogen, the second radical binds to the position to which the hydrogen atom was bonded, forming a ring.

The electronic device is preferably an organic electroluminescent device (OLED).

Preferred anodes of the electronic device are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes should be transparent or partly transparent in order to enable either the irradiation of the organic material (organic solar cell) or the emission of light (OLED, O-LASER). Preferred anode materials in this case are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

The emitting layer of the device may be a fluorescent or phosphorescent emitting layer. The emitting layer of the device is preferably a fluorescent emitting layer, especially preferably a blue-fluorescing emitting layer. In fluorescent emitting layers, the emitter is preferably a singlet emitter, i.e. a compound that emits light from an excited singlet state in the operation of the device. In phosphorescent emitting layers, the emitter is preferably a triplet emitter, i.e. a compound that emits light from an excited triplet state in the operation of the device or from a state having a higher spin quantum number, for example a quintet state.

In a preferred embodiment, fluorescent emitting layers used are blue-fluorescing layers.

In a preferred embodiment, phosphorescent emitting layers used are green- or red-phosphorescing emitting layers.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In general, all phosphorescent complexes as used for phosphorescent OLEDs according to the prior art and as known to those skilled in the art in the field of organic electroluminescent devices are suitable for use in the devices of the invention.

Preferred compounds for use as phosphorescent emitters are shown in the following table:

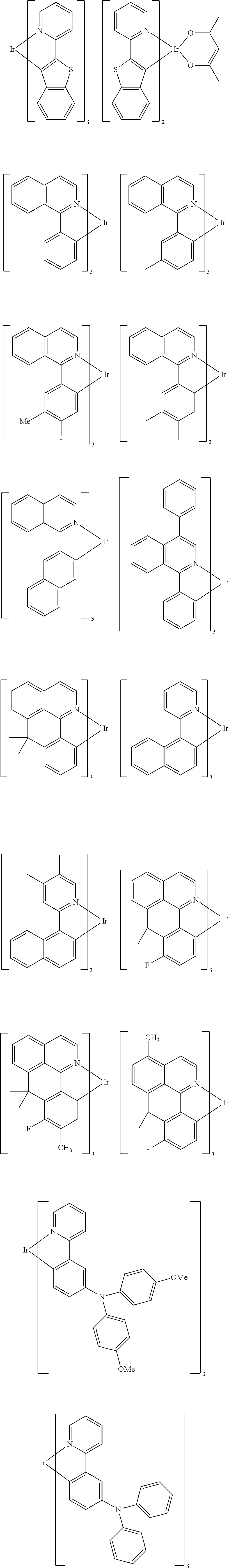

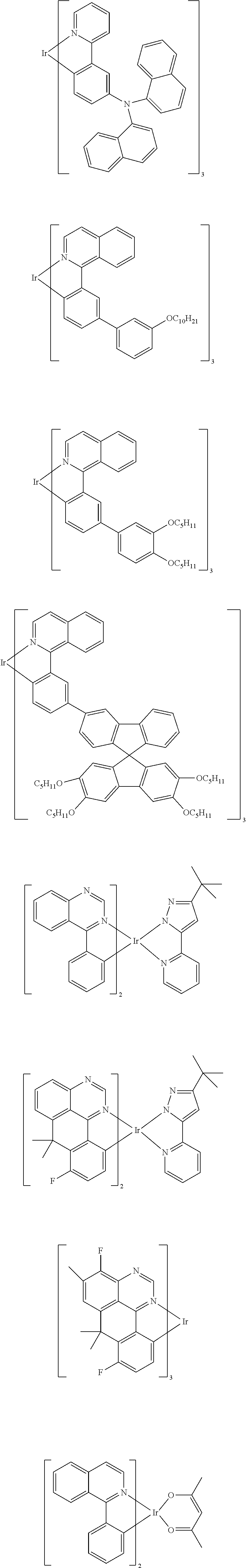

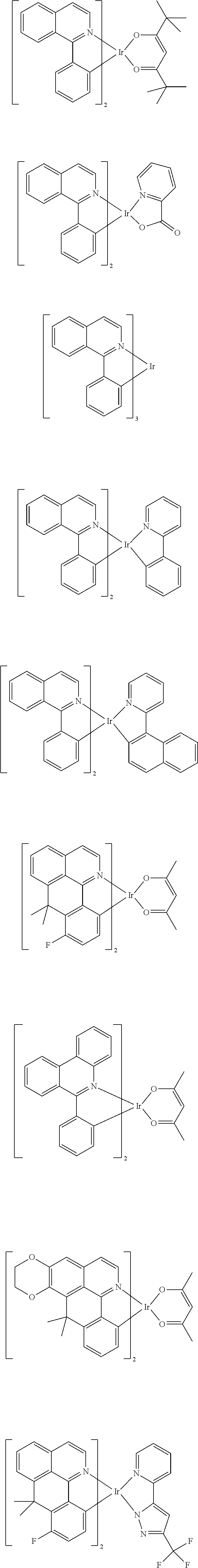

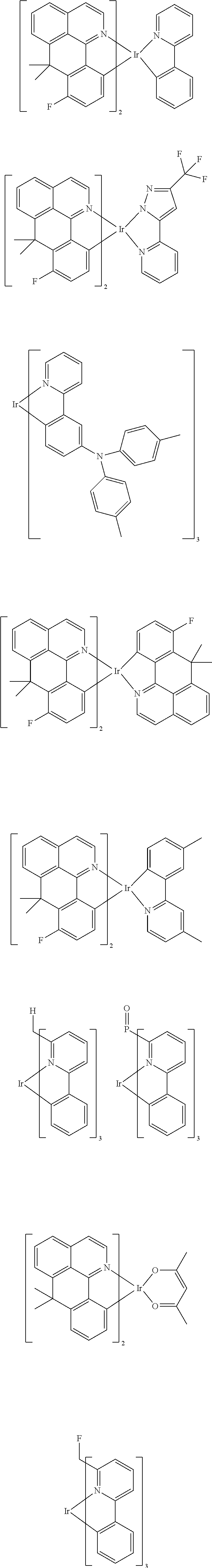

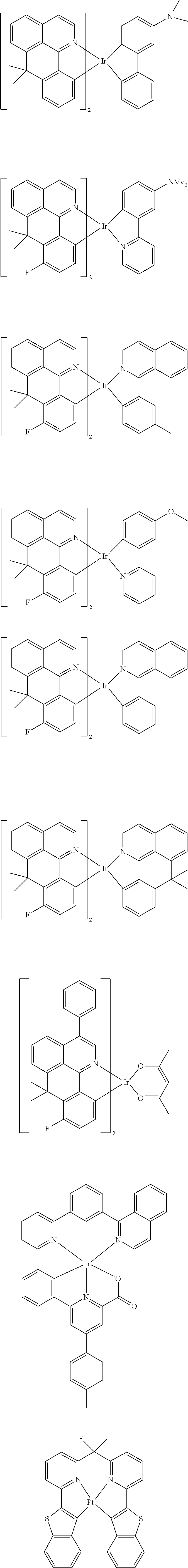

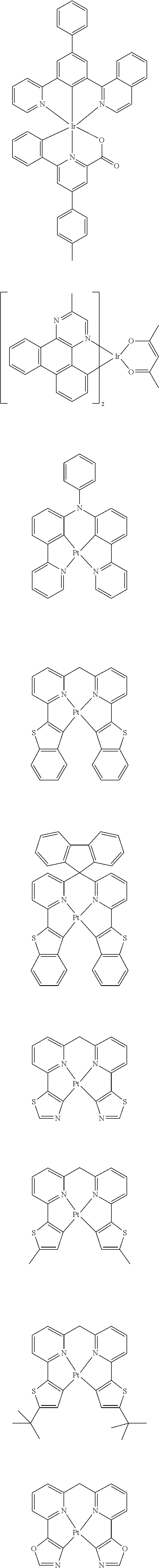

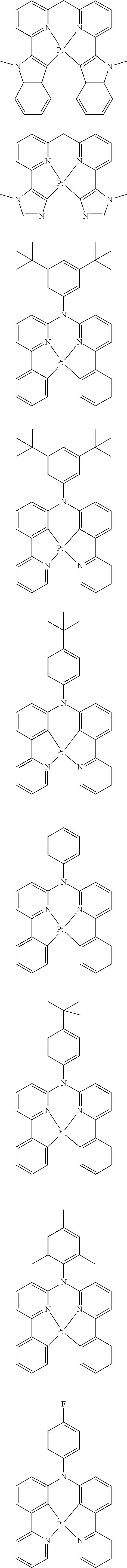

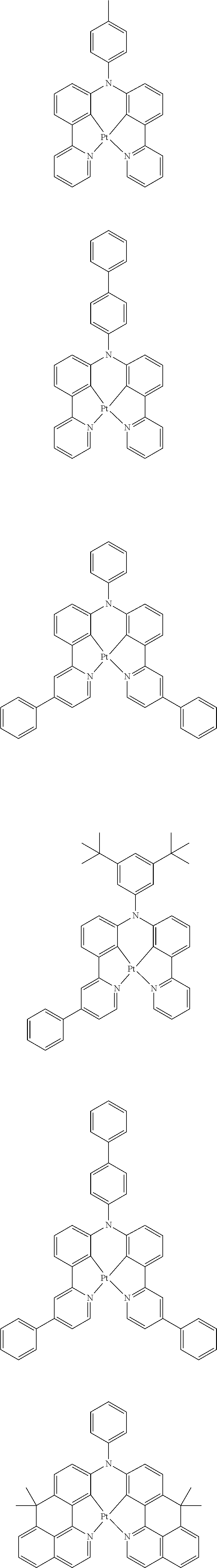

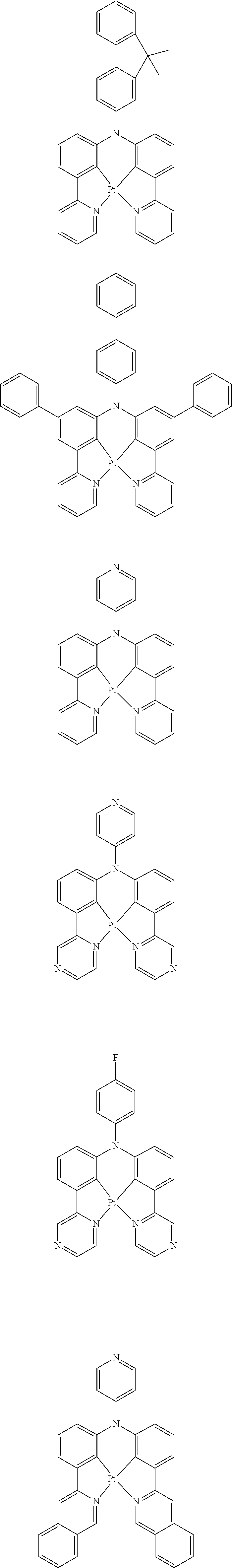

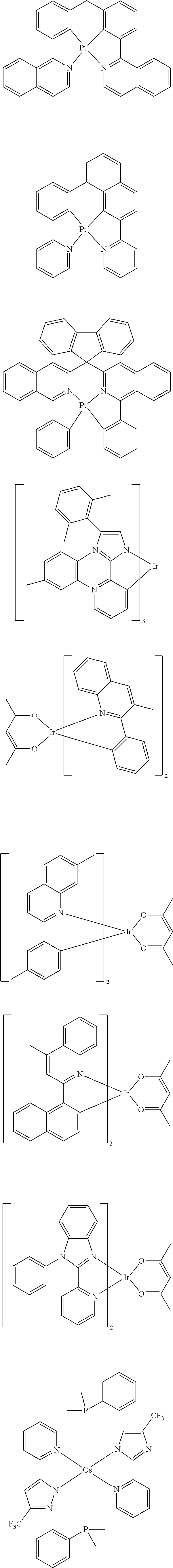

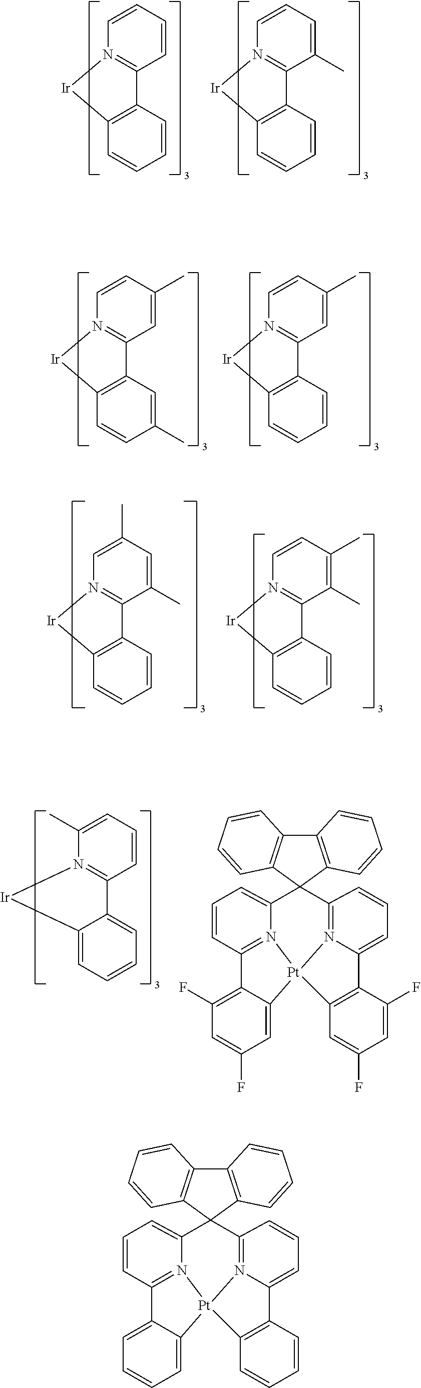

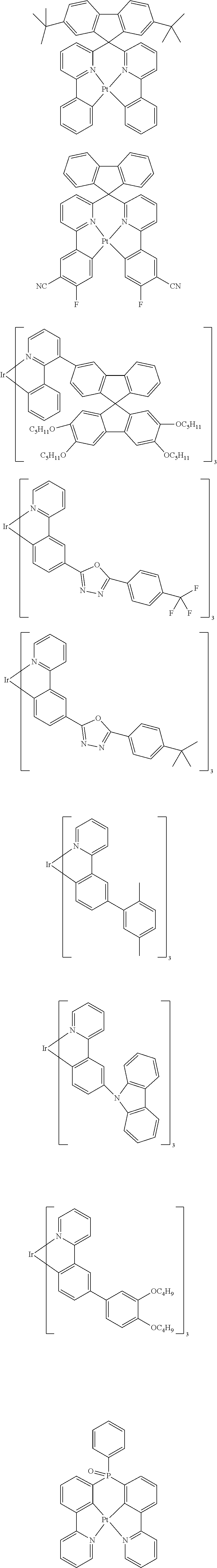

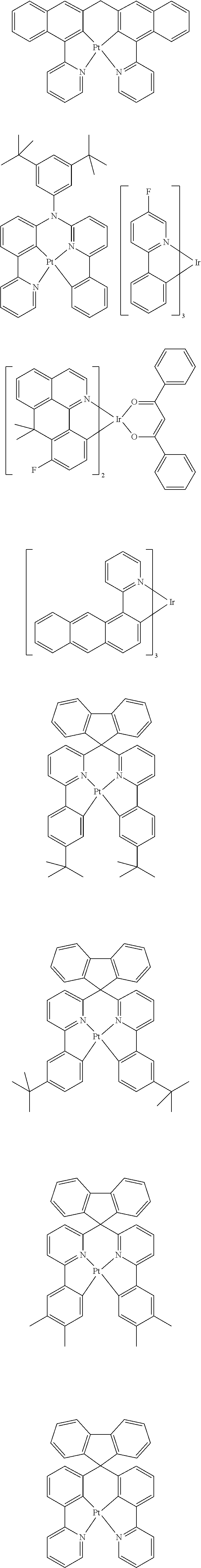

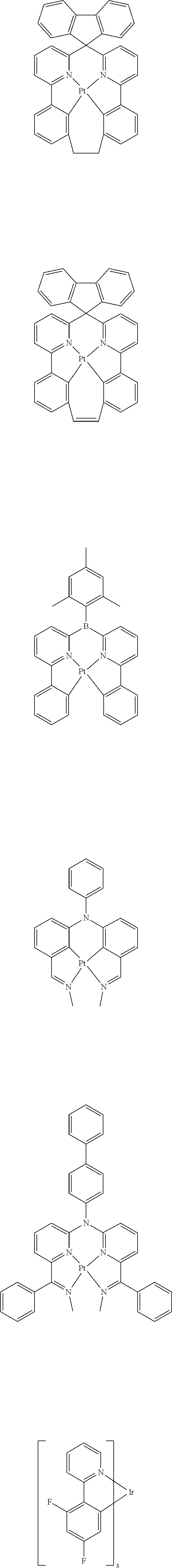

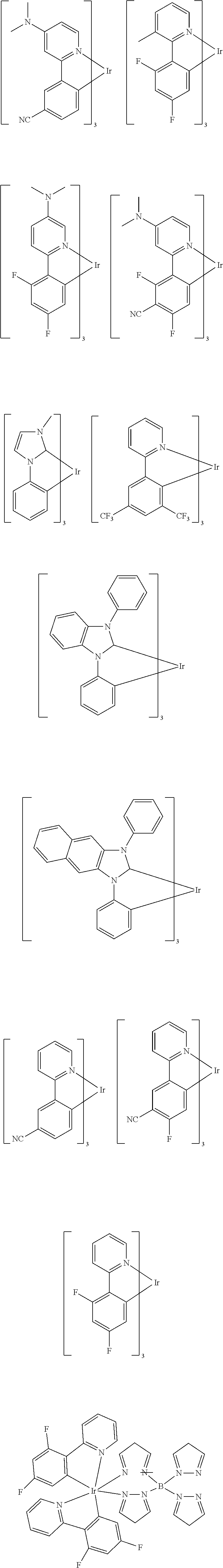

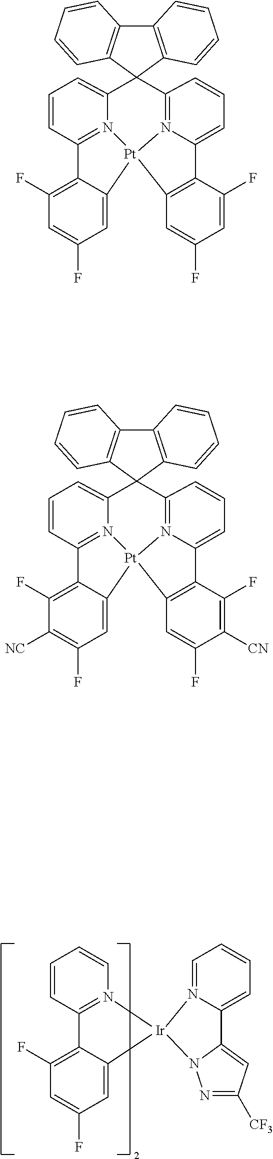

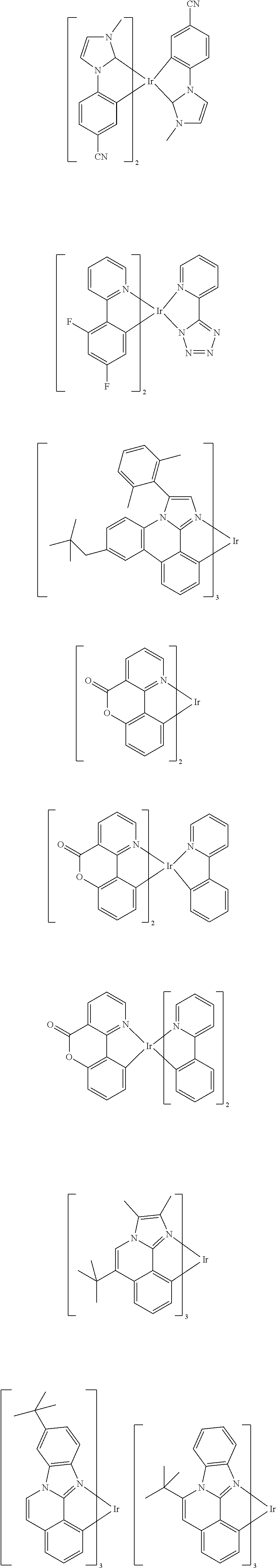

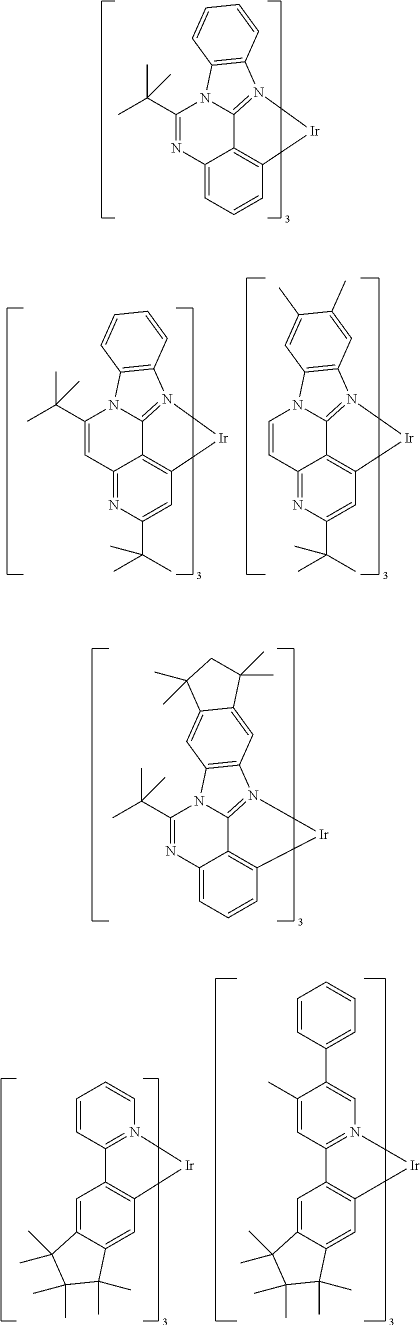

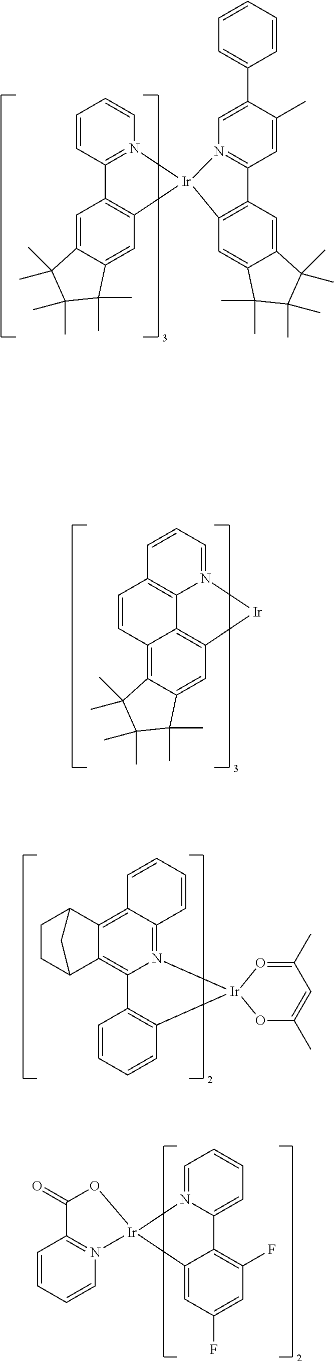

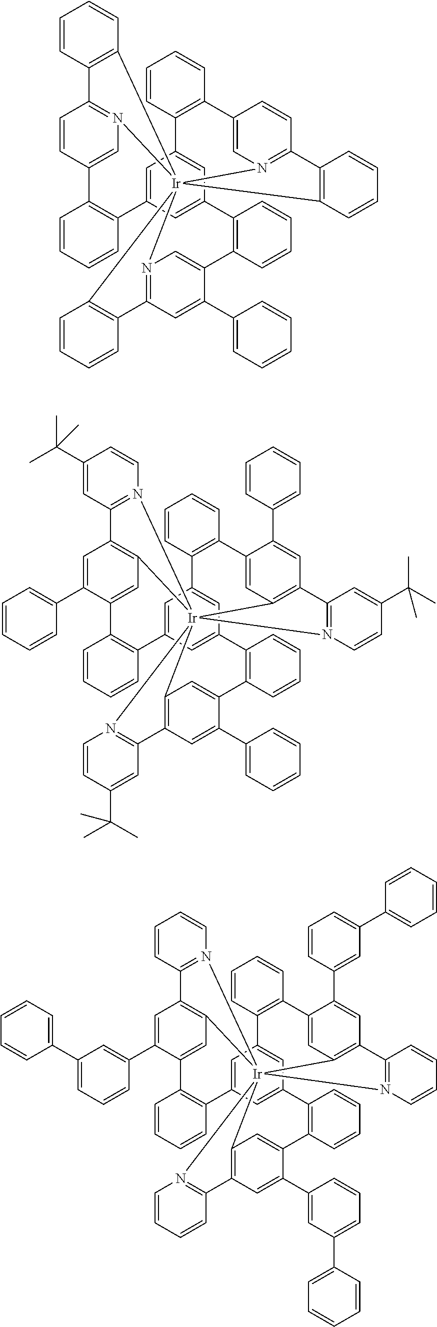

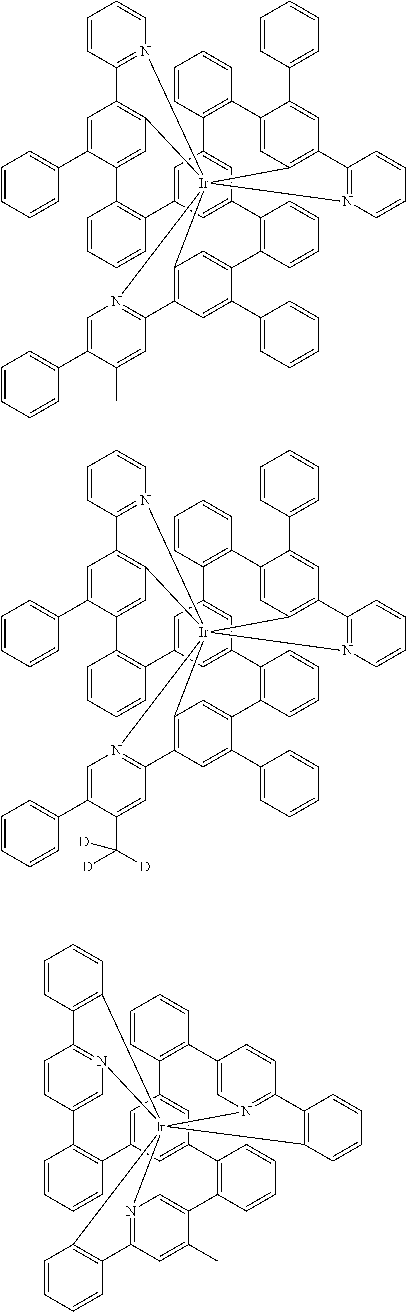

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Preferred compounds for use as fluorescent emitters are shown in the following table:

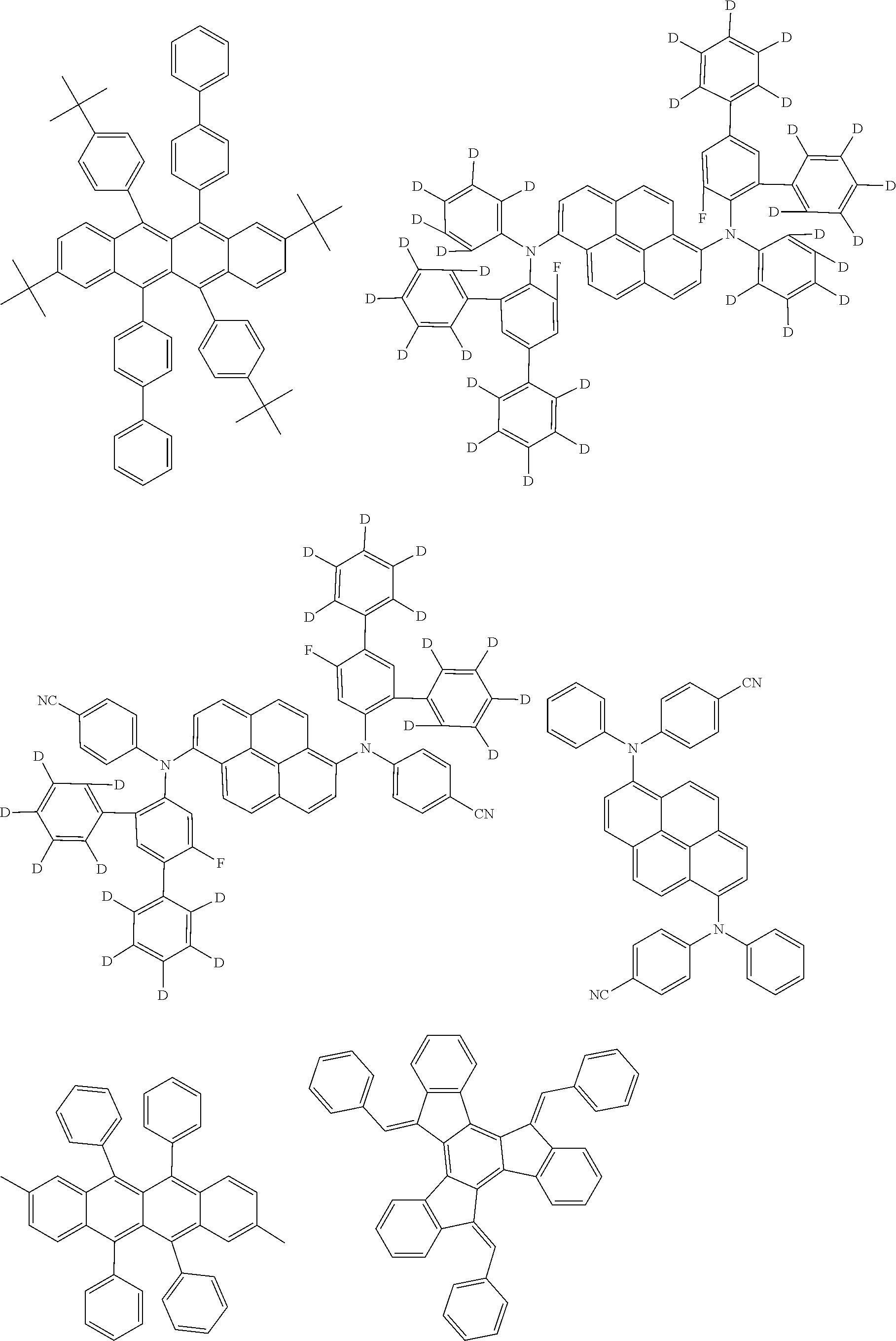

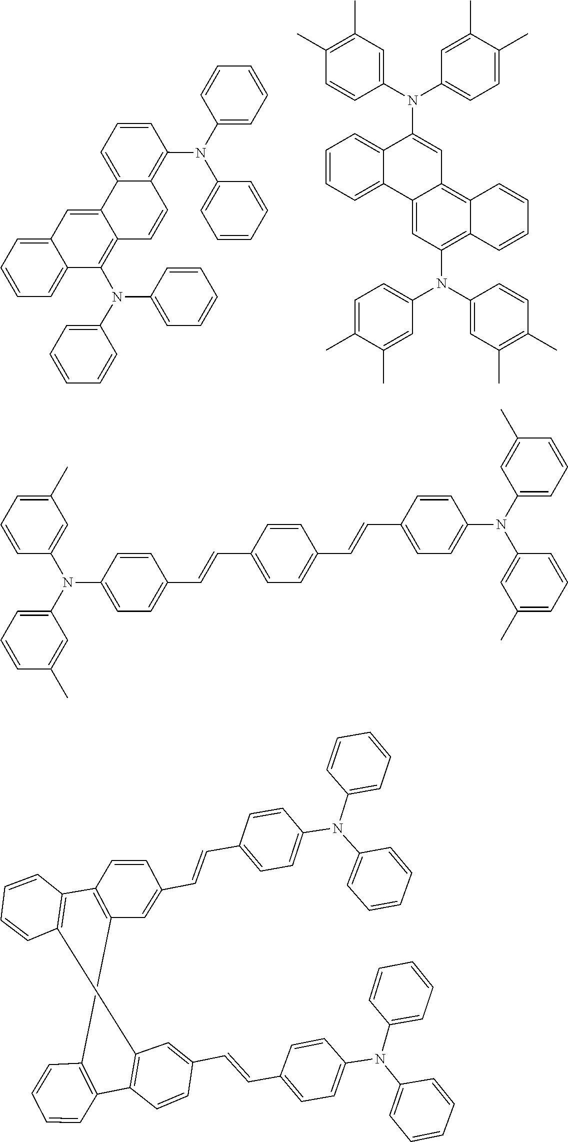

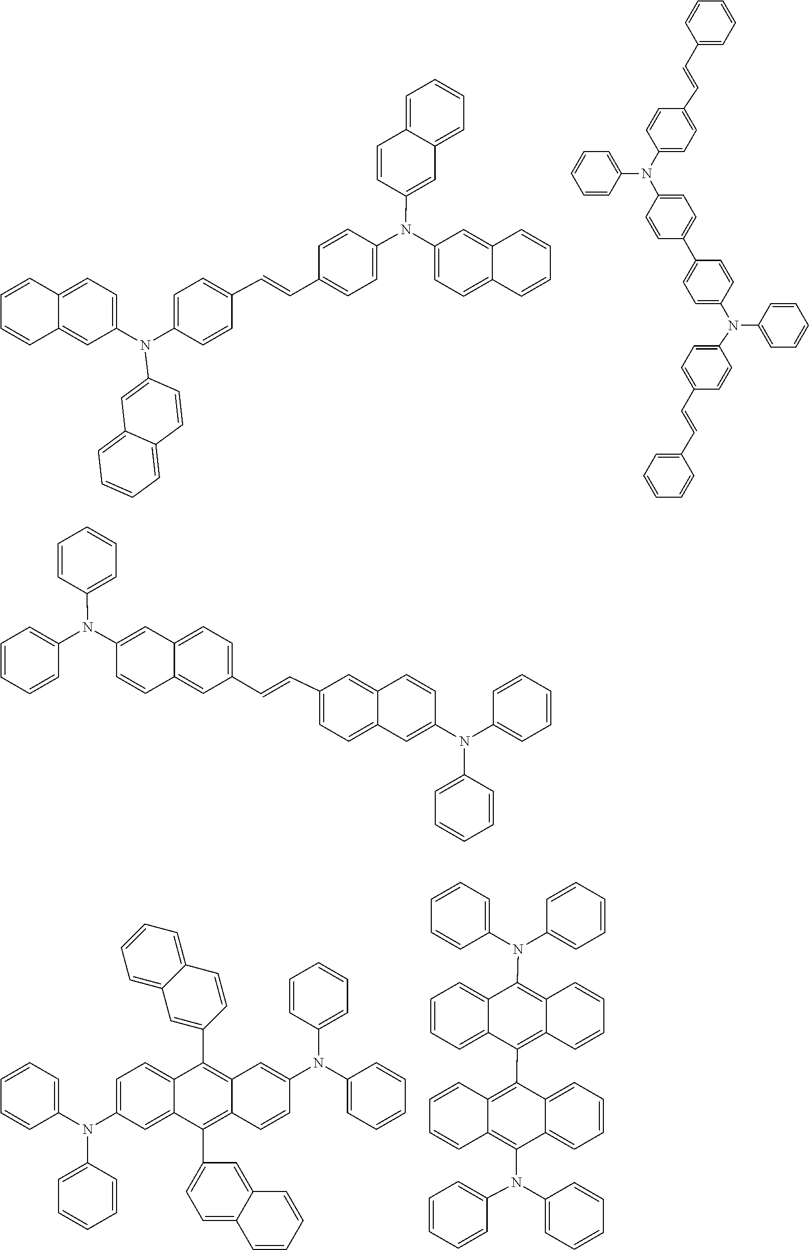

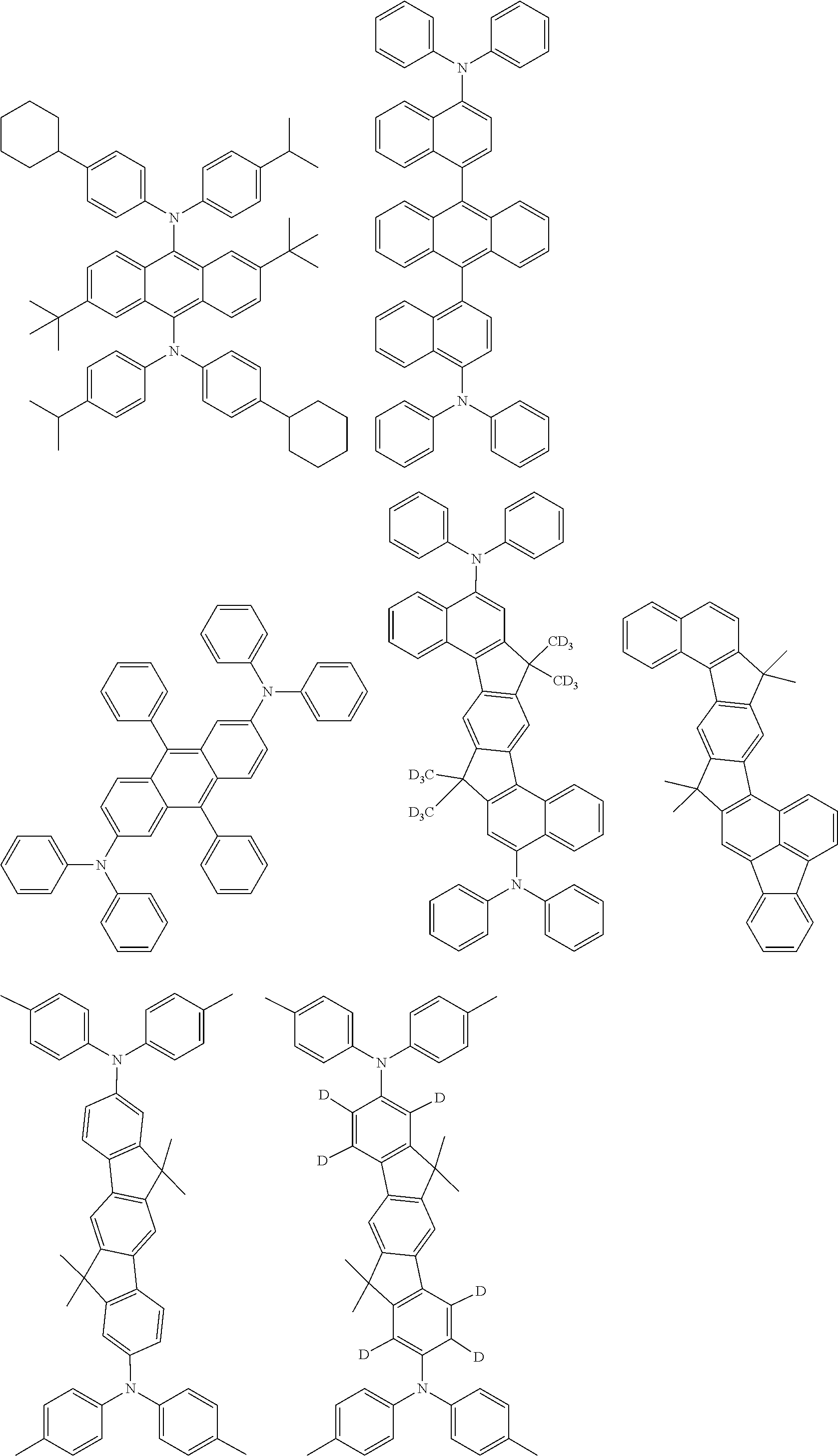

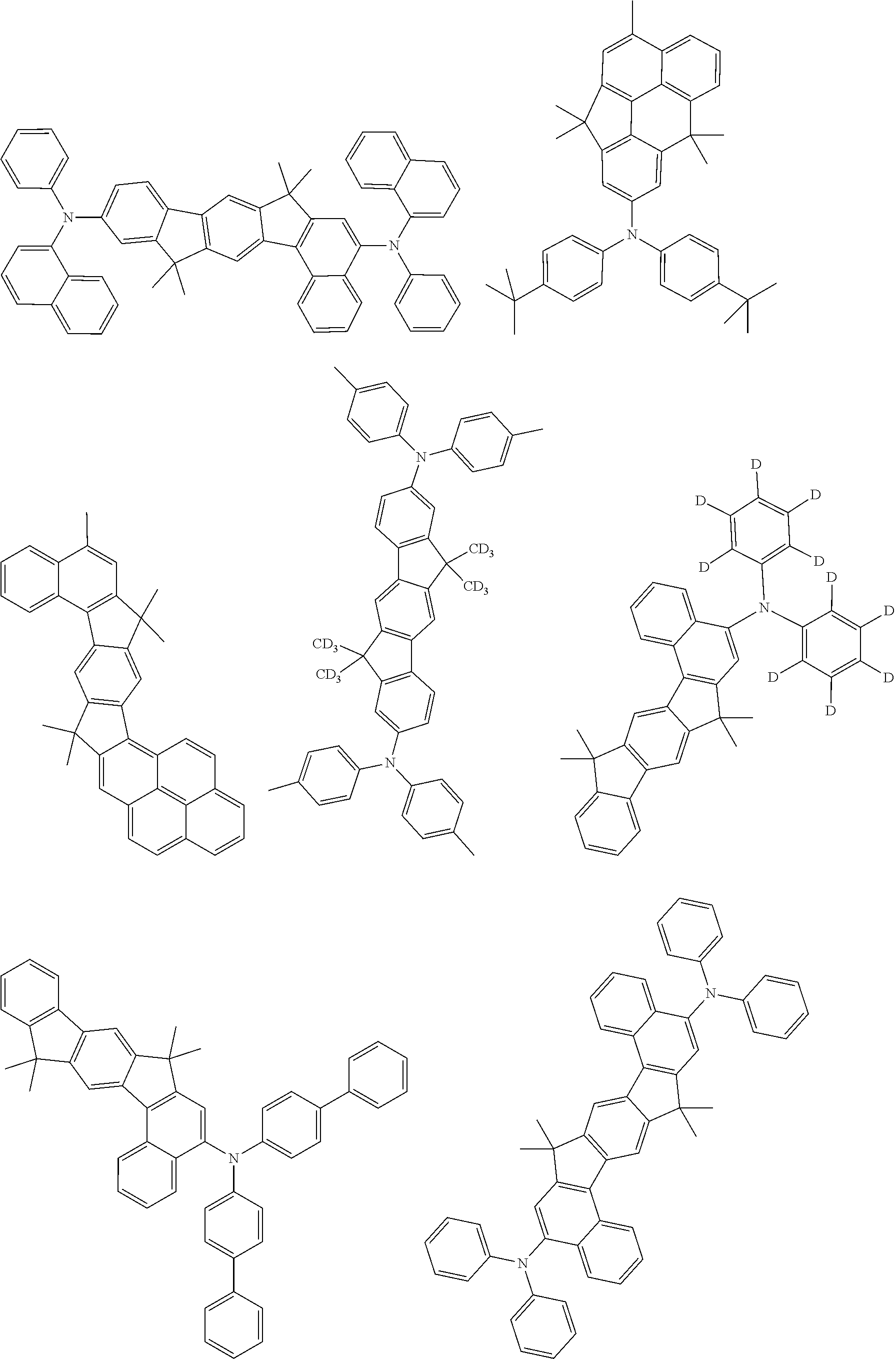

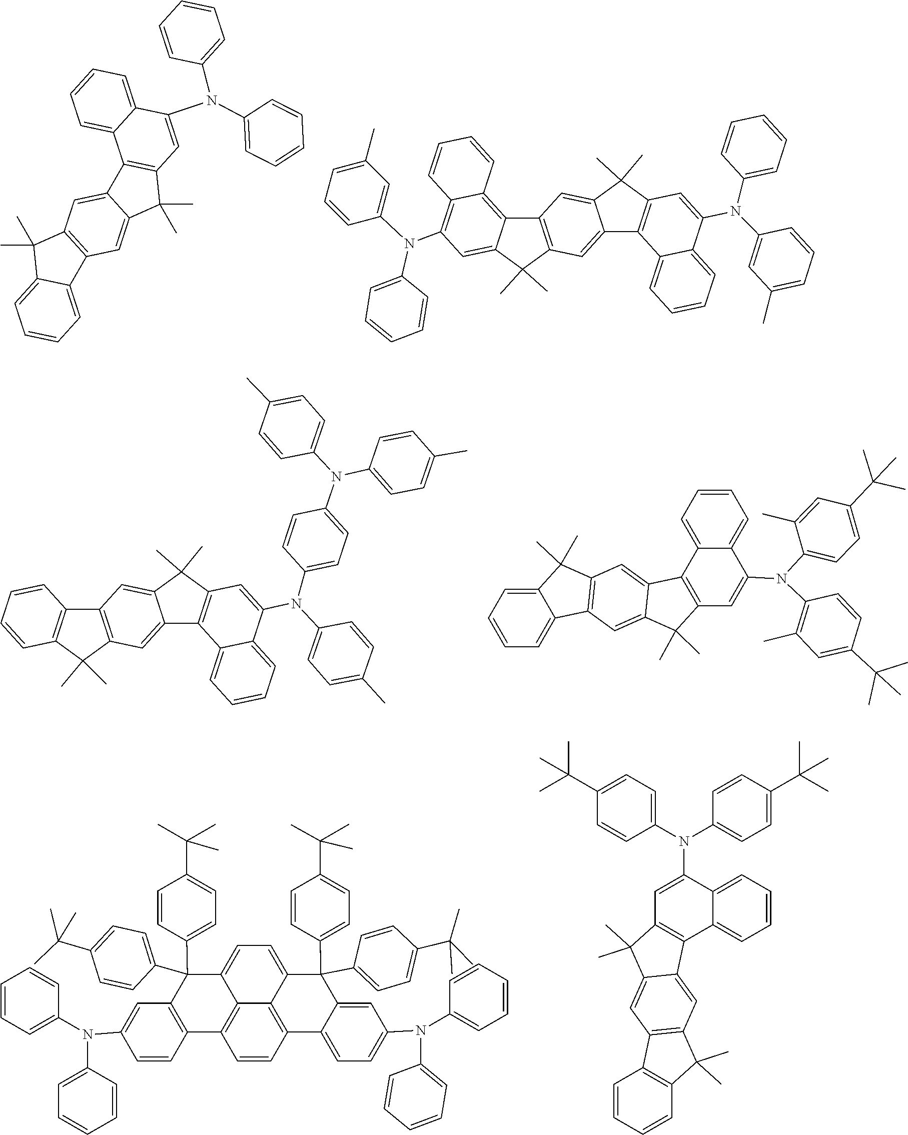

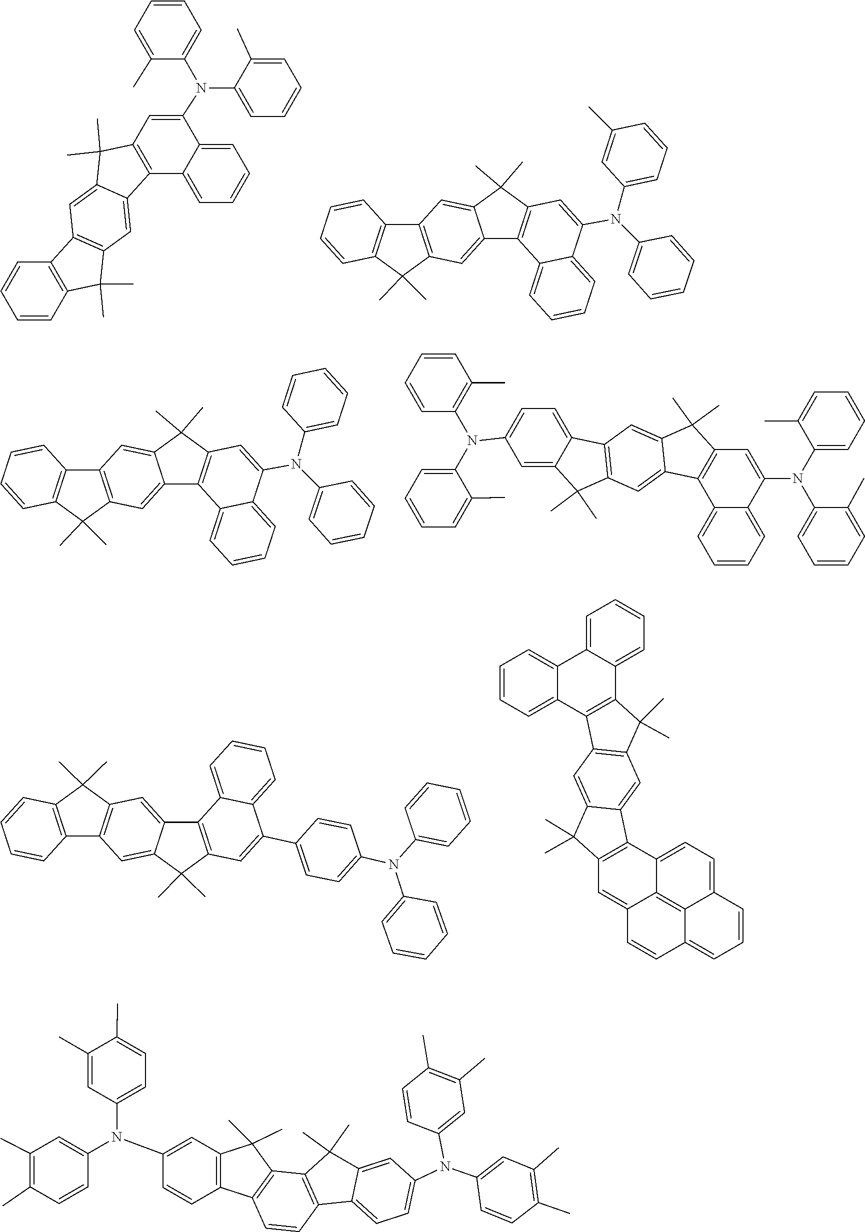

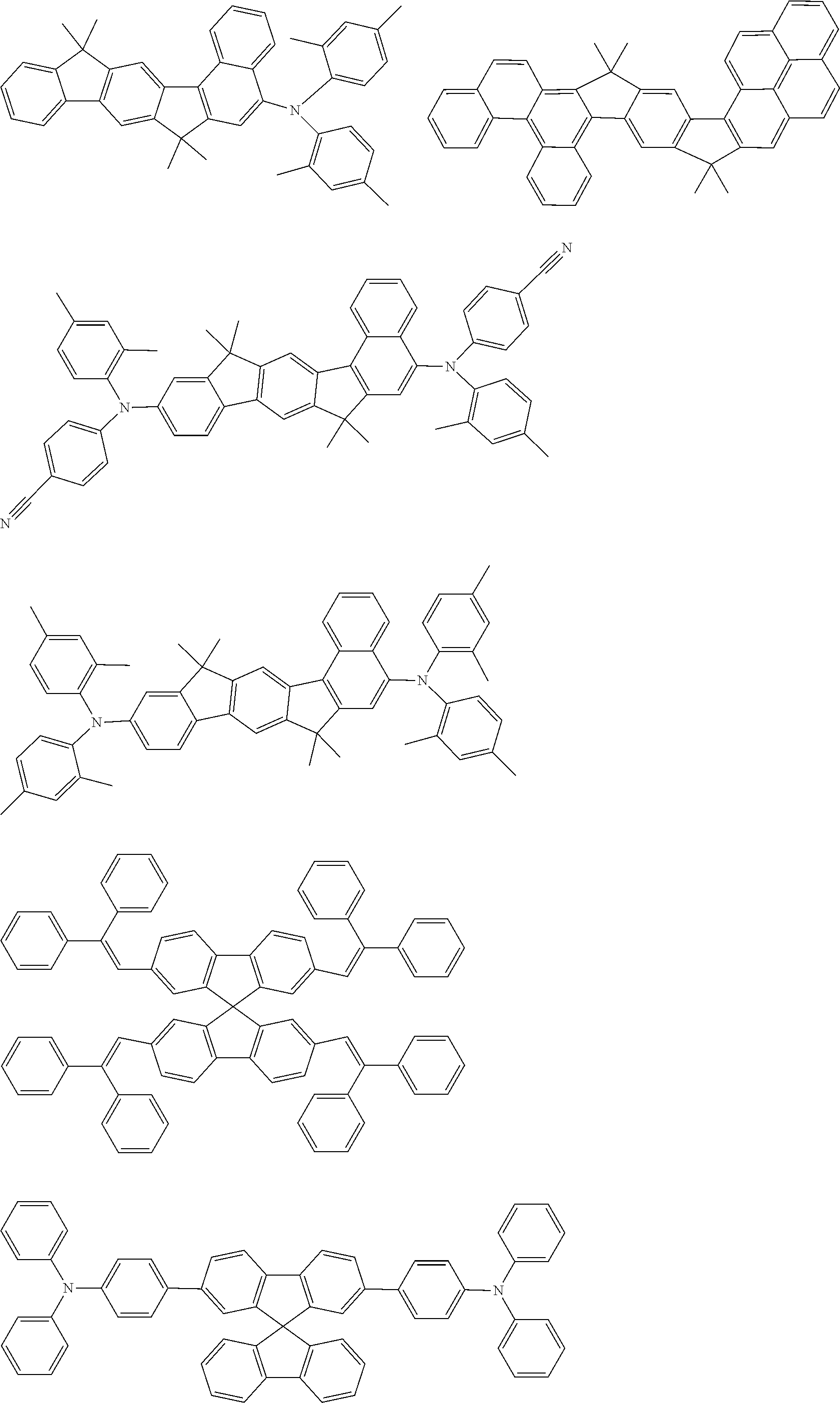

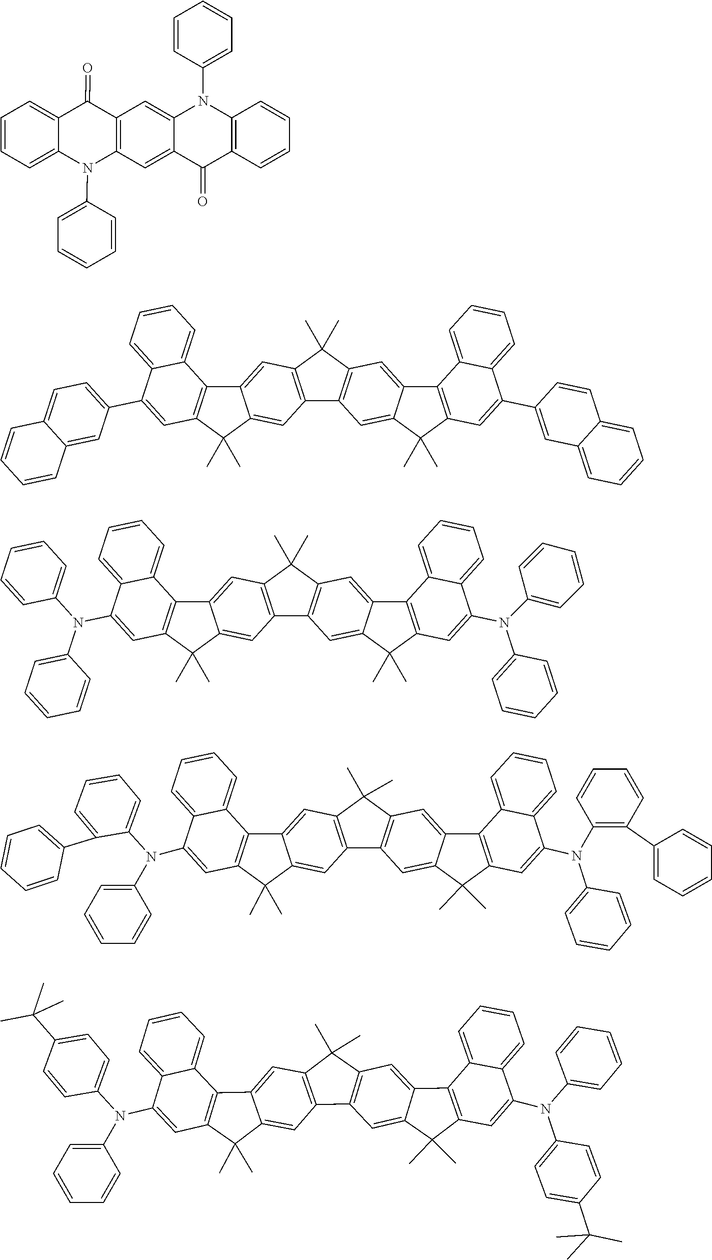

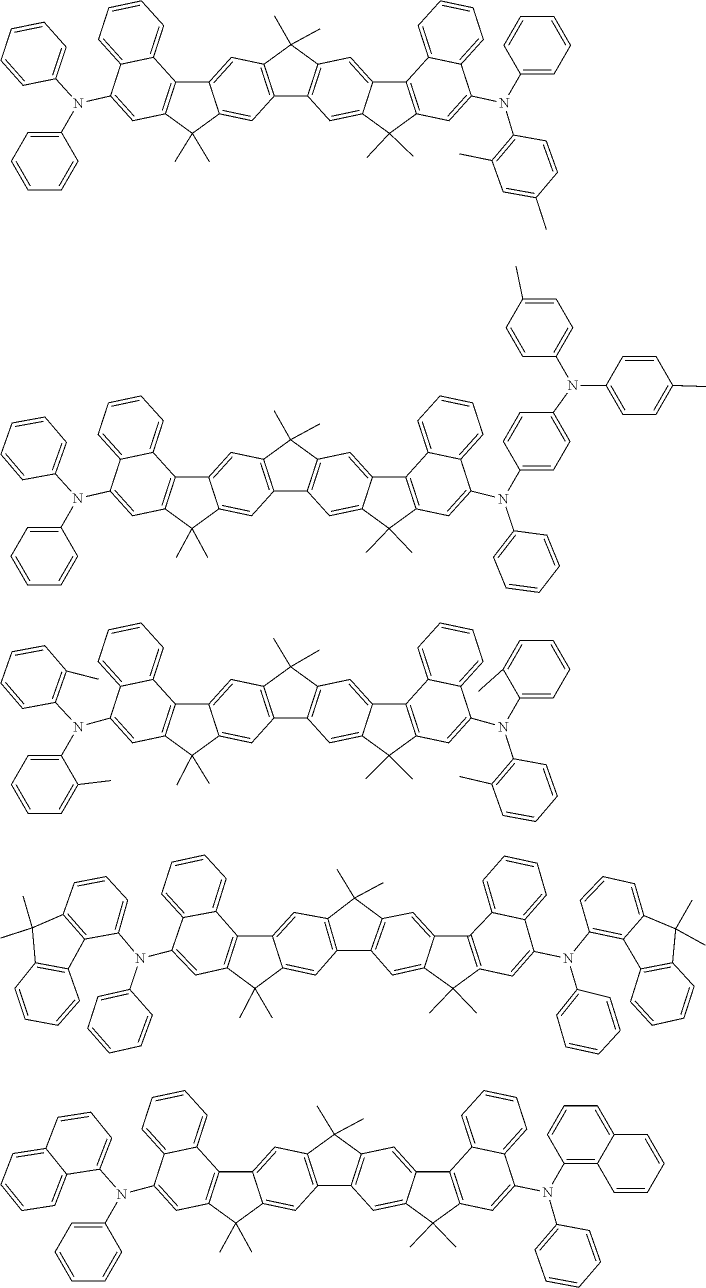

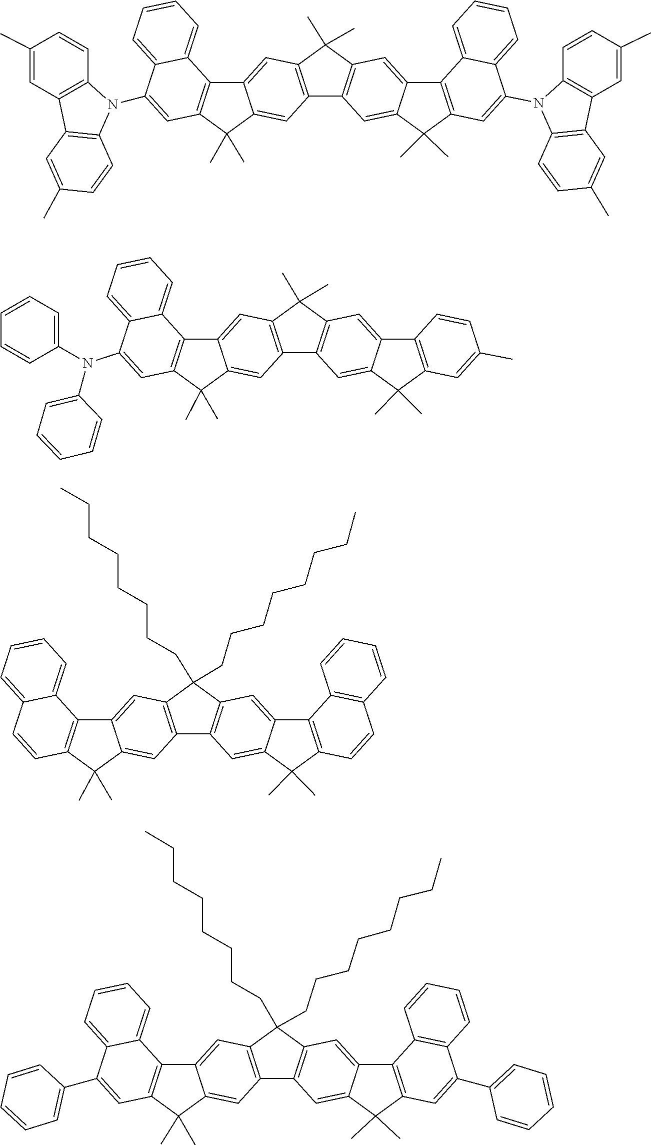

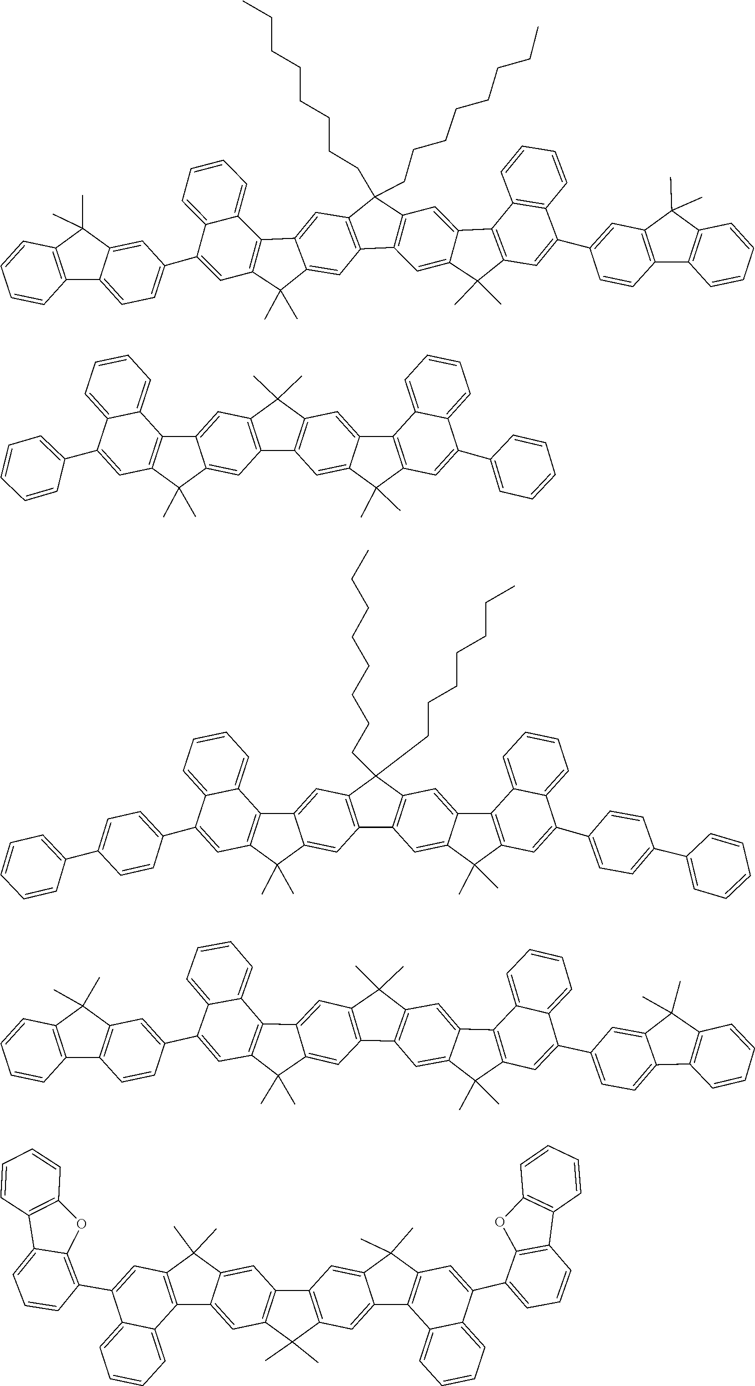

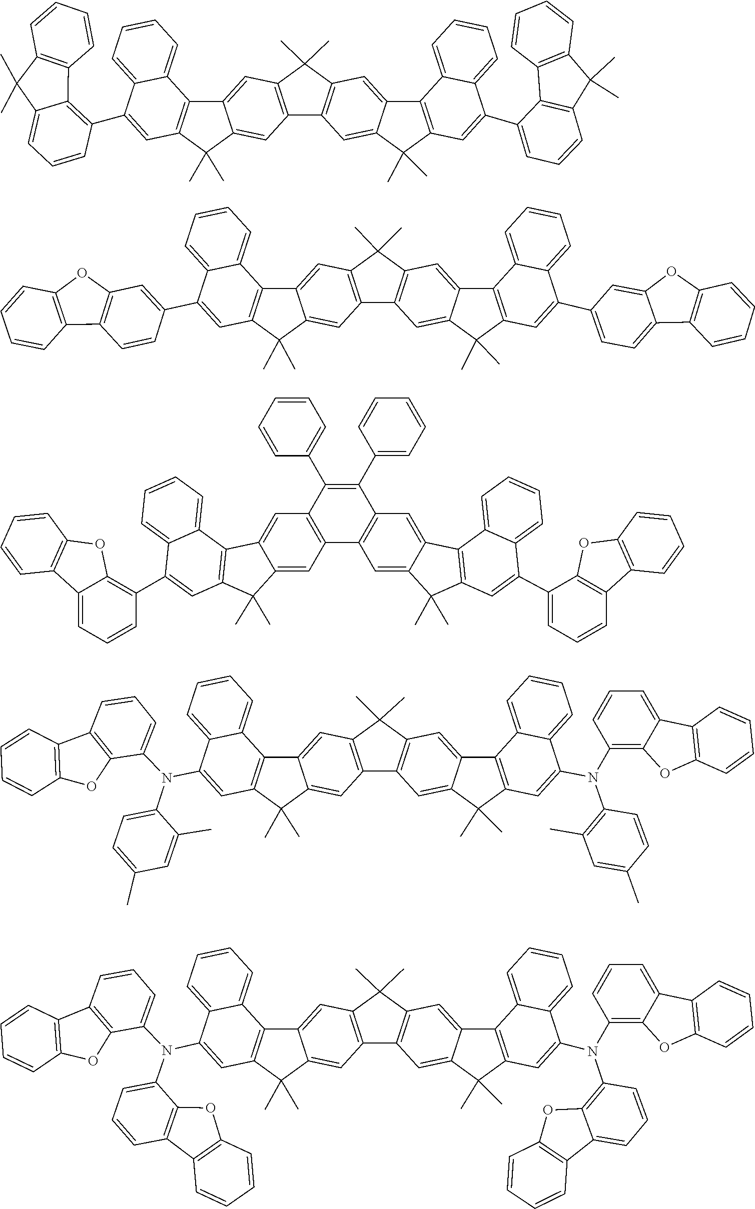

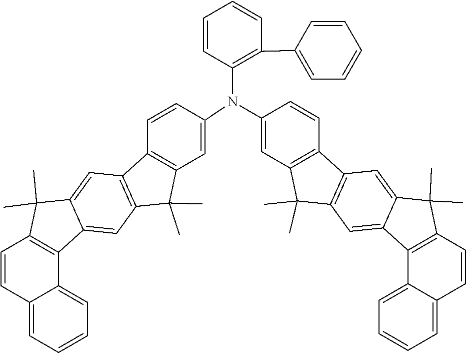 [SMILES: CC1(C)c2cc(N(c3ccc4c(c3)C(C)(C)c3cc5c(cc3-4)C(C)(C)c3ccc4ccccc4c3-5)c3ccccc3-c3ccccc3)ccc2-c2cc3c(cc21)-c1c(ccc2ccccc12)C3(C)C]

In a preferred embodiment, the emitting layer of the electronic device contains exactly one matrix compound. A matrix compound is understood to mean a compound that is not an emitting compound. This embodiment is especially preferred in the case of fluorescent emitting layers.

In an alternative preferred embodiment, the emitting layer of the electronic device contains exactly two or more, preferably exactly two, matrix compounds. This embodiment, which is also referred to as mixed matrix system, is especially preferred in the case of phosphorescent emitting layers.

The total proportion of all matrix materials in the case of a phosphorescent emitting layer is preferably between 50.0% and 99.9%, more preferably between 80.0% and 99.5% and most preferably between 85.0% and 97.0%.

The figure for the proportion in % is understood here to mean the proportion in % by volume in the case of layers that are applied from the gas phase, and the proportion in % by weight in the case of layers that are applied from solution.

Correspondingly, the proportion of the phosphorescent emitting compound is preferably between 0.1% and 50.0%, more preferably between 0.5% and 20.0%, and most preferably between 3.0% and 15.0%.

The total proportion of all matrix materials in the case of a fluorescent emitting layer is preferably between 50.0% and 99.9%, more preferably between 80.0% and 99.5% and most preferably between 90.0% and 99.0%.

Correspondingly, the proportion of the fluorescent emitting compound is between 0.1% and 50.0%, preferably between 0.5% and 20.0%, and more preferably between 1.0% and 10.0%.

Mixed matrix systems preferably comprise two or three different matrix materials, more preferably two different matrix materials. Preferably, in this case, one of the two materials is a material having properties including hole-transporting properties and the other material is a material having properties including electron-transporting properties. Further matrix materials that may be present in mixed matrix systems are compounds having a large energy difference between HOMO and LUMO (wide bandgap materials). The two different matrix materials may be present in a ratio of 1:50 to 1:1, preferably 1:20 to 1:1, more preferably 1:10 to 1:1 and most preferably 1:4 to 1:1. Preference is given to using mixed matrix systems in phosphorescent organic electroluminescent devices.

Preferred matrix materials for fluorescent emitting compounds are selected from the classes of the oligoarylenes (e.g. 2,2′,7,7-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives and the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Preferred matrix materials for fluorescent emitting compounds are shown in the following table:

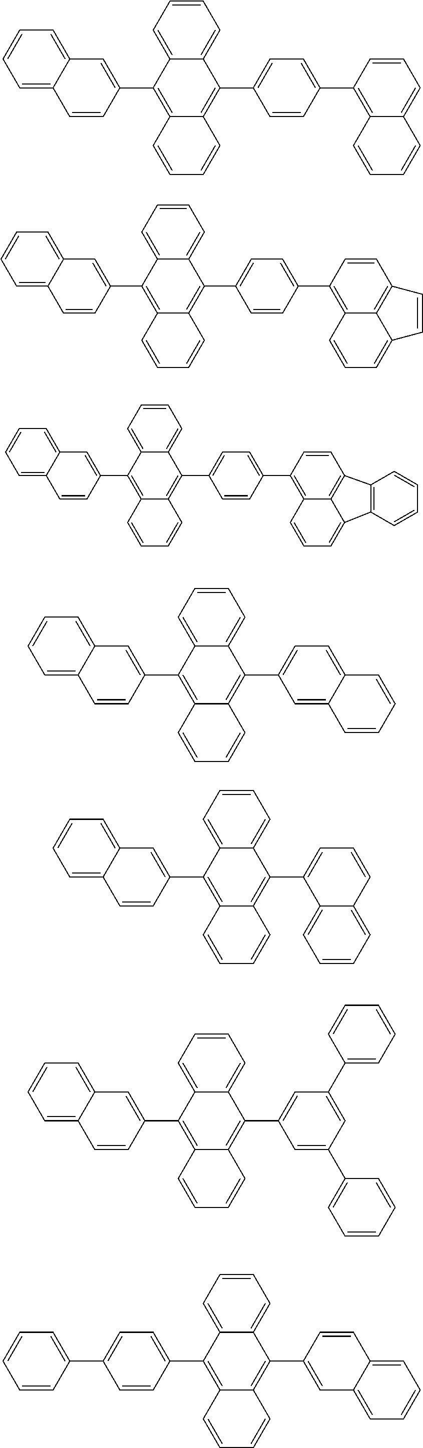

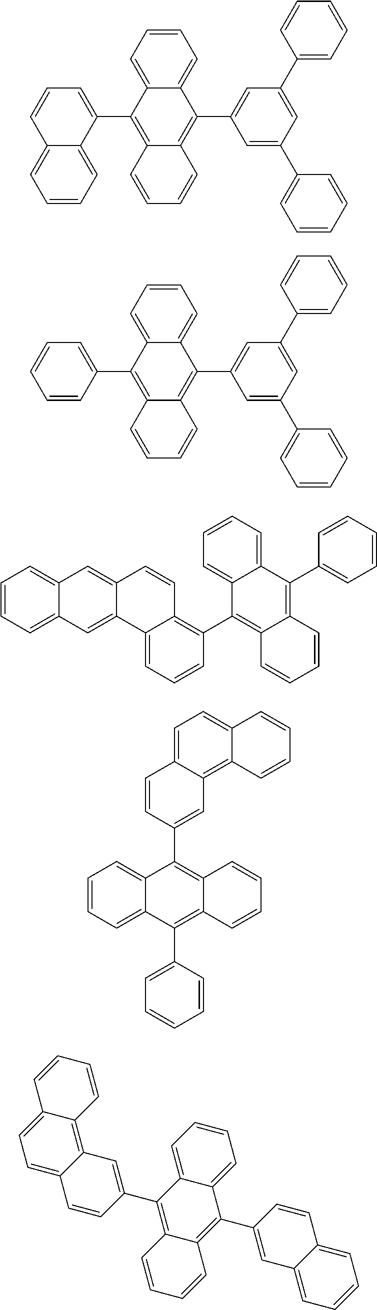

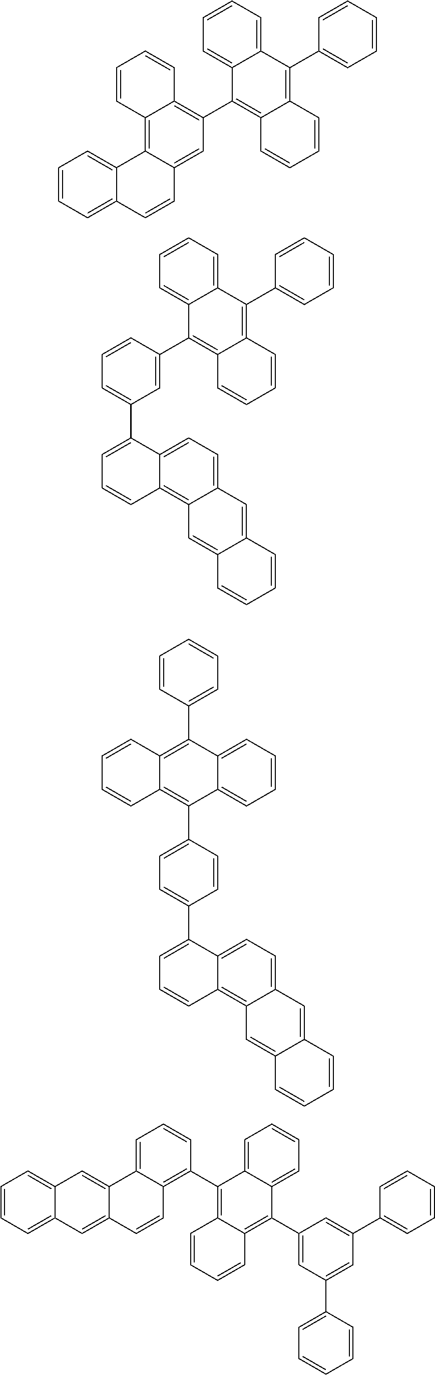

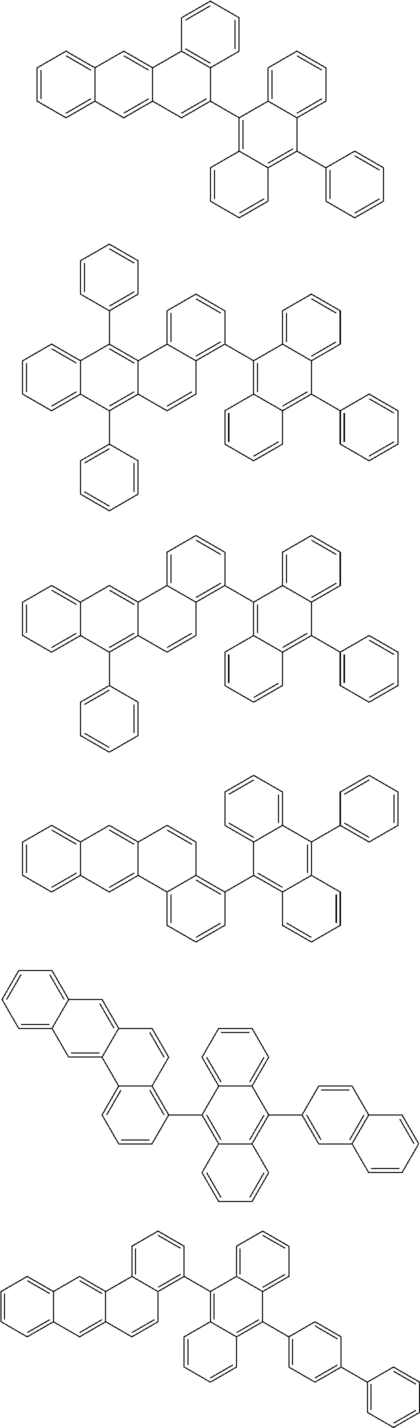

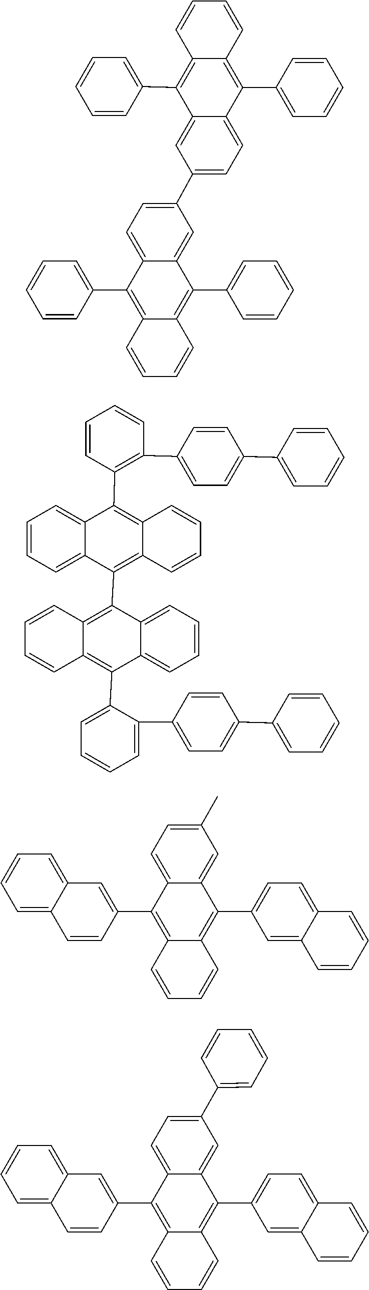

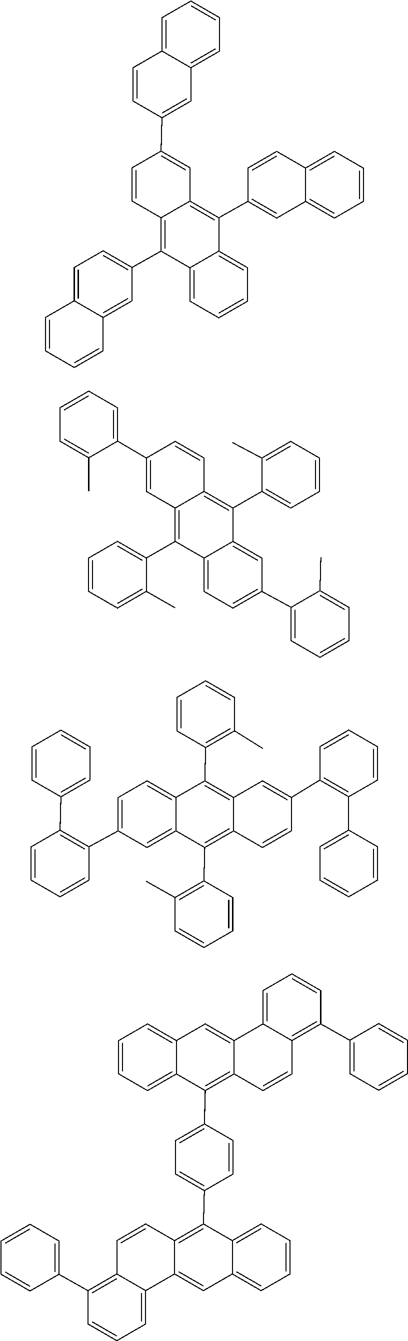

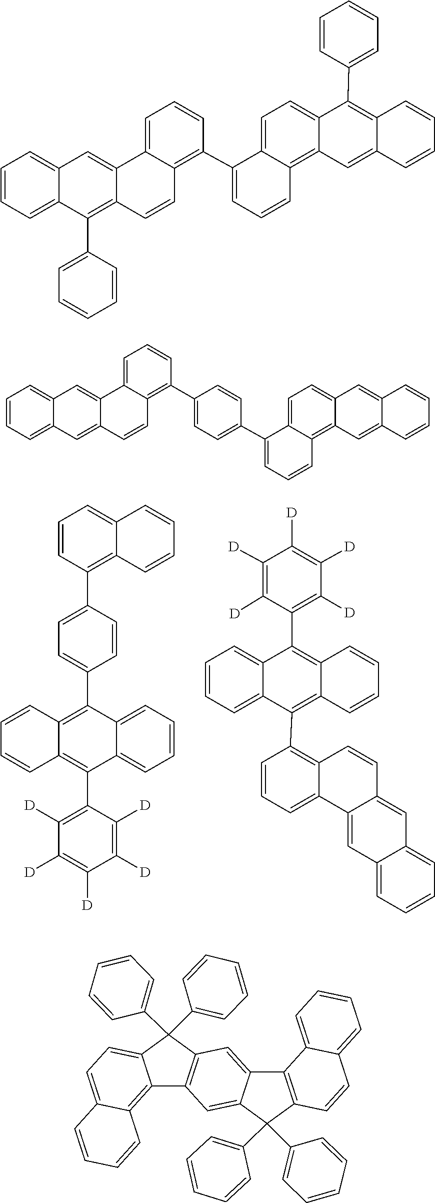

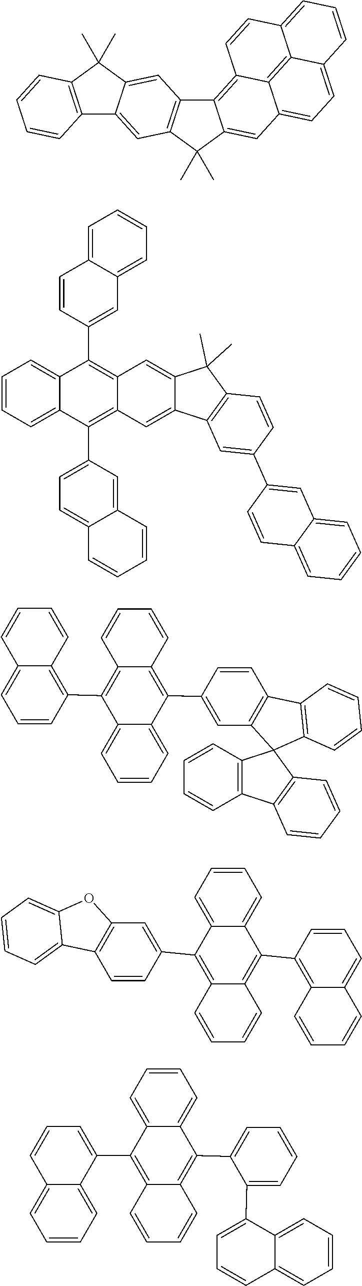

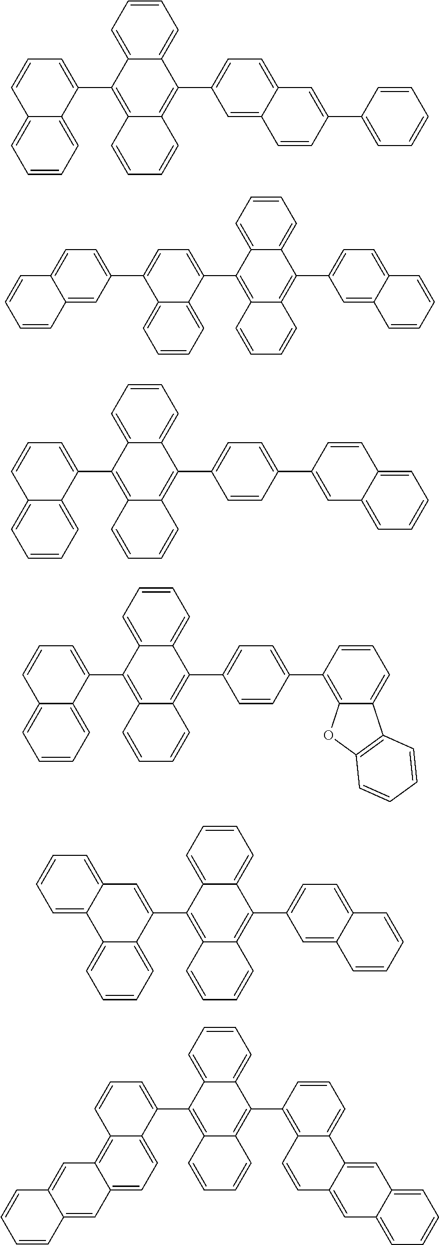

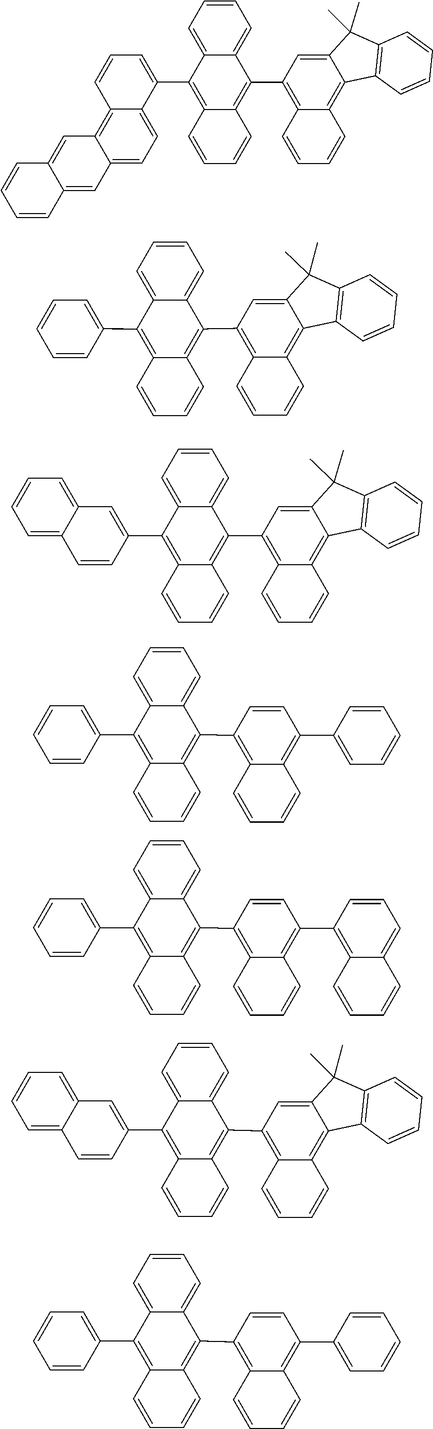

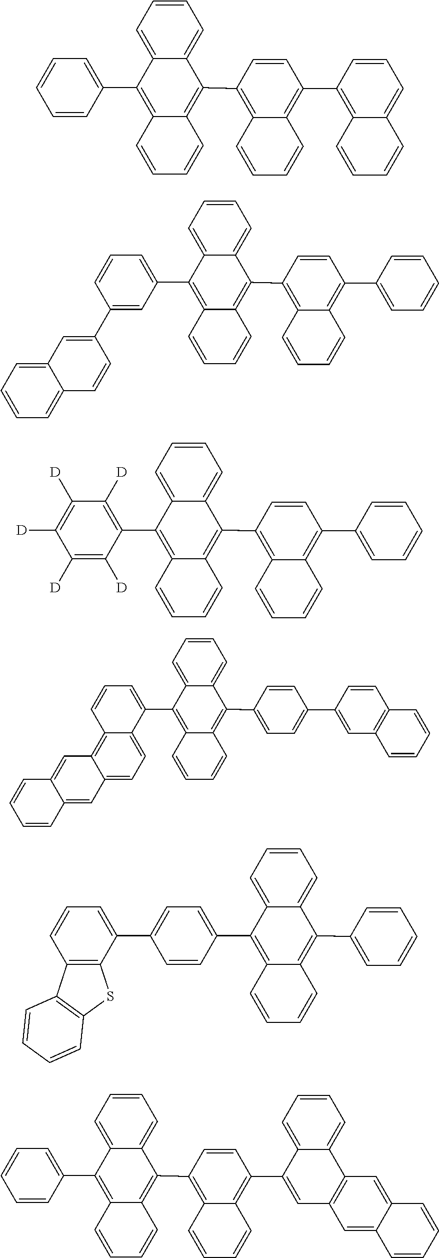

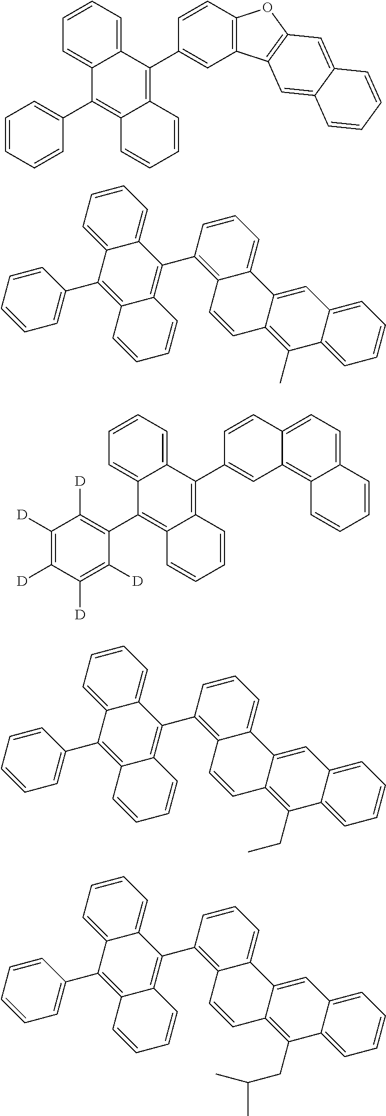

Preferred matrix materials for phosphorescent emitters are aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

In a preferred embodiment, the electronic device contains exactly one emitting layer.

In an alternative preferred embodiment, the electronic device contains multiple emitting layers, preferably 2, 3 or 4 emitting layers. This is especially preferable for white-emitting electronic devices.

More preferably, the emitting layers in this case have several emission maxima between 380 nm and 750 nm overall, such that the electronic device emits white light; in other words, various emitting compounds which can fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. For the production of white light, rather than a plurality of colour-emitting emitter compounds, it is also possible to use an individual emitter compound which emits over a broad wavelength range.

In a preferred embodiment of the invention, the electronic device comprises two or three, preferably three, identical or different layer sequences stacked one on top of another, where each of the layer sequences comprises the following layers: hole injection layer, hole-transporting layer, electron blocker layer, emitting layer, and electron transport layer, and where at least one, preferably all, of the layer sequences contain(s) the following layers:
a hole injection layer disposed between anode and emitting layer;
a hole-transporting layer disposed between hole injection layer and emitting layer and directly adjoining the emitting layer on the anode side, and containing two different compounds conforming to identical or different formulae selected from formulae (I) and (II).

A double layer composed of adjoining n-CGL and p-CGL is preferably arranged between the layer sequences in each case, where the n-CGL is disposed on the anode side and the p-CGL correspondingly on the cathode side. CGL here stands for charge generation layer. Materials for use in such layers are known to the person skilled in the art. Preference is given to using a p-doped amine in the p-CGL, more preferably a material selected from the preferred structure classes of hole transport materials that are mentioned below.

The hole-transporting layer preferably has a layer thickness of 20 nm to 300 nm, more preferably of 30 nm to 250 nm. It is further preferable that the hole-transporting layer has a layer thickness of not more than 250 nm.

Preferably, the hole-transporting layer contains exactly 2, 3 or 4, preferably exactly 2 or 3, most preferably exactly 2, different compounds conforming to identical or different formulae selected from formulae (I) and (II).

Preferably, the hole-transporting layer consists of compounds conforming to identical or different formulae selected from formulae (I) and (II). “Consist of” is understood here to mean that no further compounds are present in the layer, not counting minor impurities as typically occur in the production process for OLEDs as further compounds in the layer.

In an alternative preferred embodiment, the hole-transporting layer, in addition to the compounds conforming to identical or different formulae selected from formulae (I) and (II), contains a p-dopant.

p-Dopants used according to the present invention are preferably those organic electron acceptor compounds capable of oxidizing one or more of the other compounds in the mixture.

Particularly preferred p-dopants are quinodimethane compounds, azaindenofluorenediones, azaphenalenes, azatriphenylenes, I₂, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as bonding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (Ill) oxidation state, more particularly bismuth(III) complexes with electron-deficient ligands, more particularly carboxylate ligands.

The p-dopants are preferably in substantially homogeneous distribution in the p-doped layer. This can be achieved, for example, by co-evaporation of the p-dopant and the hole transport material matrix. The p-dopant is preferably present in a proportion of 1% to 10% in the p-doped layer.

Preferred p-dopants are especially the following compounds:

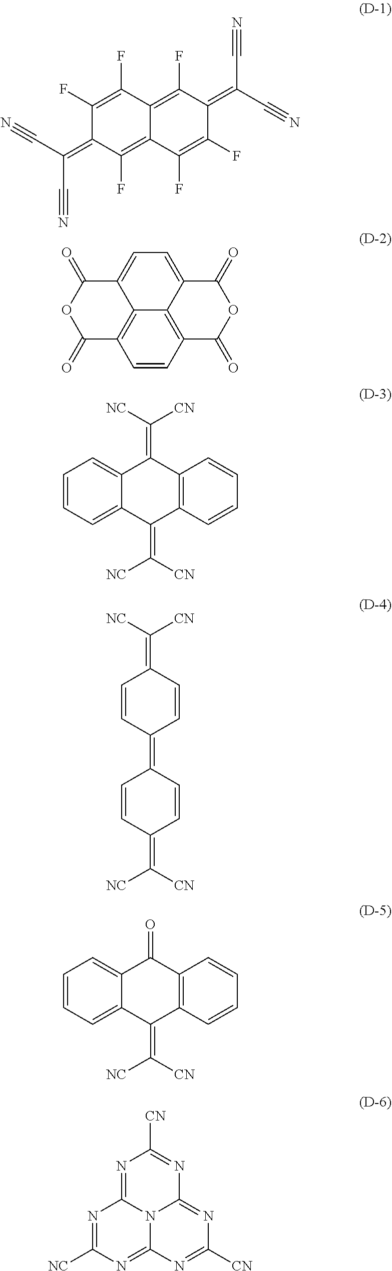

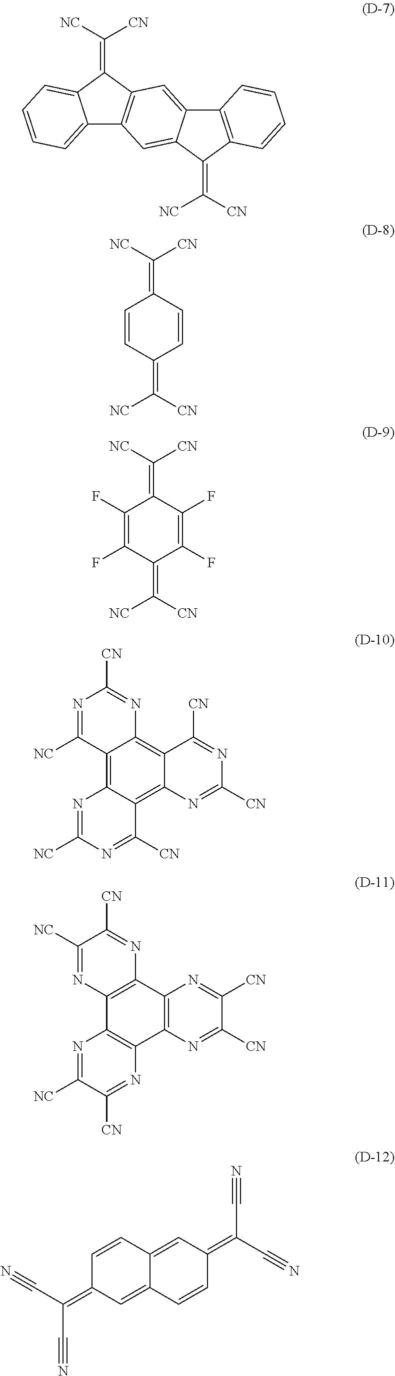

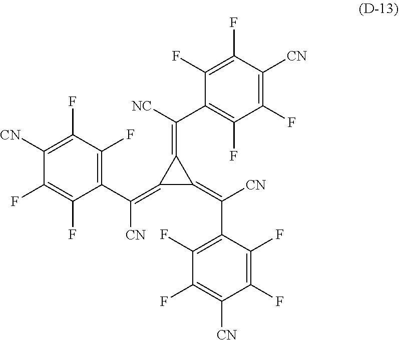

In a preferred embodiment of the invention, the hole-transporting layer contains two different compounds that conform to a formula (I).

The two different compounds conforming to identical or different formulae selected from formulae (I) and (II) are preferably each present in the hole-transporting layer in a proportion of at least 5%. They are more preferably present in a proportion of at least 10%. It is preferable that one of the compounds is present in a higher proportion than the other compound, more preferably in a proportion two to five times as high as the proportion of the other compound. This is the case especially when the hole-transporting layer contains exactly two compounds conforming to identical or different formulae selected from formulae (I) and (II). Preferably, the proportion in the layer is 15% to 35% for one of the compounds, and the proportion in the layer is 65% to 85% for the other of the two compounds.

Among the formulae (I) and (II), preference is given to formula (I).

Formulae (I) and/or (II) are subject to one or more, preferably all, preferences selected from the following preferences:

In a preferred embodiment, the compounds have a single amino group. An amino group is understood to mean a group having a nitrogen atom having three binding partners. This is preferably understood to mean a group in which three groups selected from aromatic and heteroaromatic groups bind to a nitrogen atom.

In an alternative preferred embodiment, the compounds have exactly two amino groups.

Z is preferably CR¹, where Z is C when a

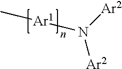

group is bonded thereto;

X is preferably a single bond;

Ar¹ is preferably the same or different at each instance and is selected from divalent groups derived from benzene, biphenyl, terphenyl, naphthalene, fluorene, indenofluorene, indenocarbazole, spirobifluorene, dibenzofuran, dibenzothiophene, and carbazole, each of which are substituted by one or more R² radicals. Most preferably, Ar¹ is the same or different at each instance and is a divalent group derived from benzene which is substituted in each case by one or more R² radicals. Ar¹ groups may be the same or different at each instance.

Index n is preferably 0, 1 or 2, more preferably 0 or 1, and most preferably 0.

Preferred -(Ar¹)ₙ- groups in the case that n=1 conform to the following formulae:

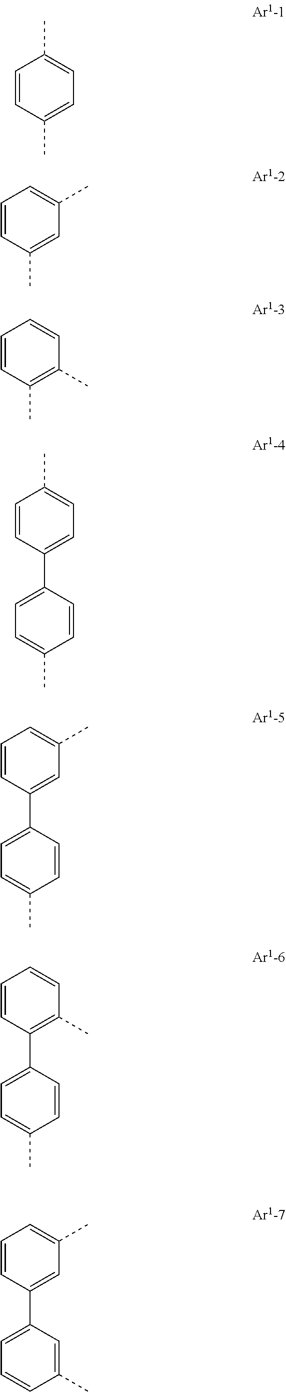

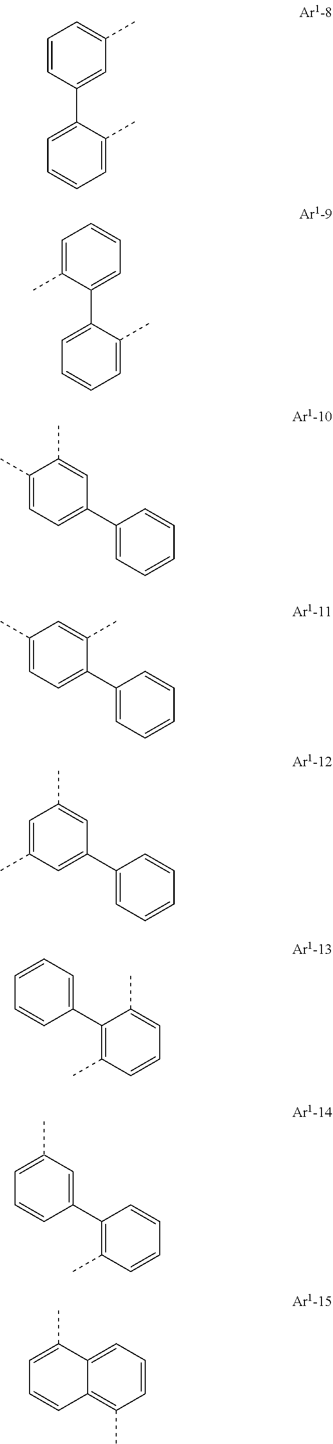

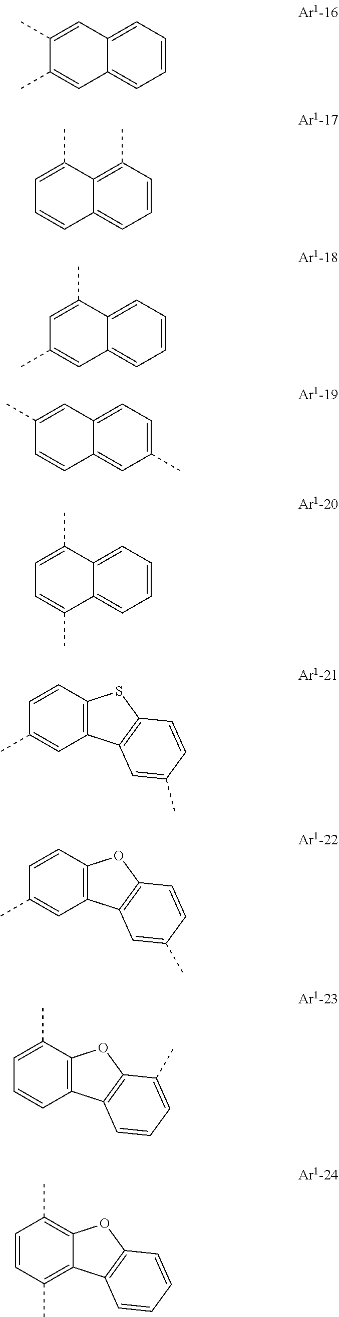

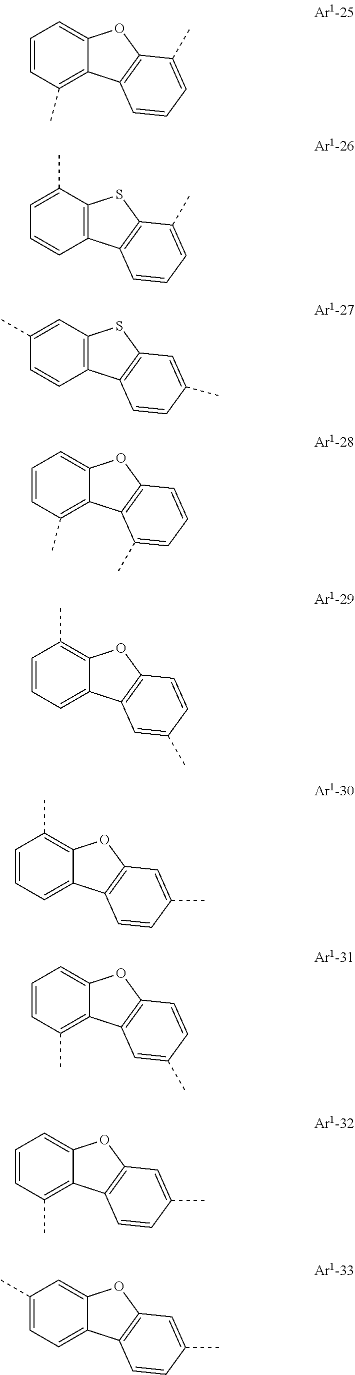

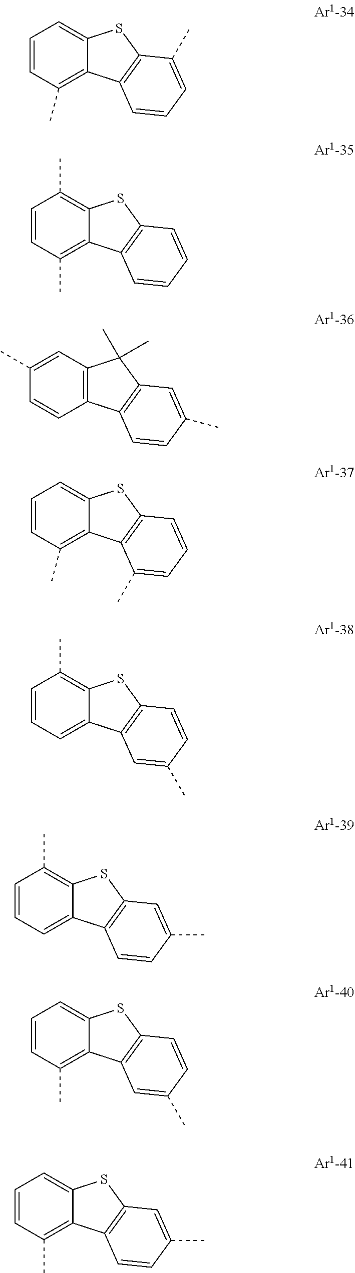

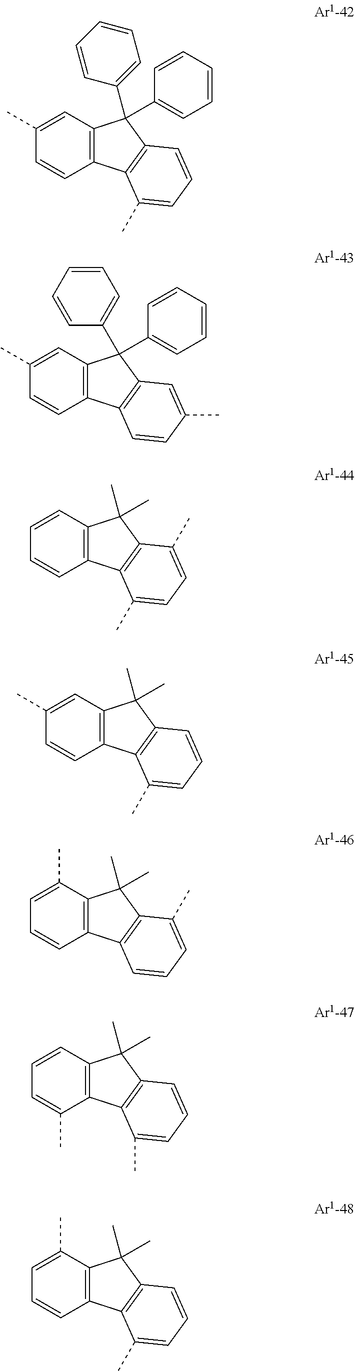

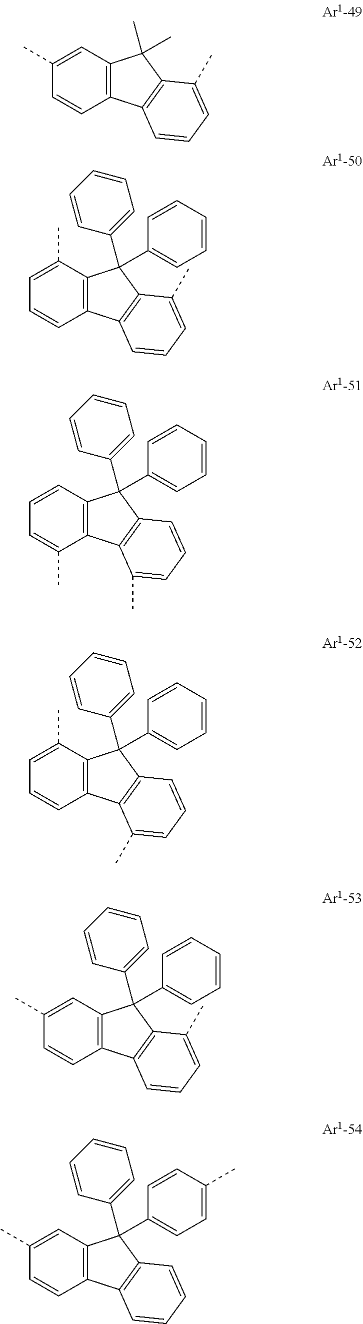

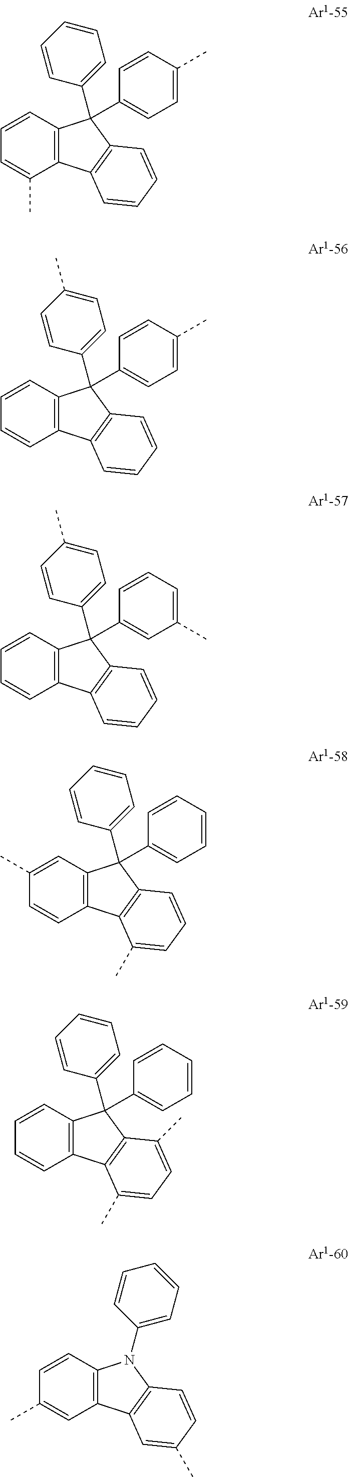

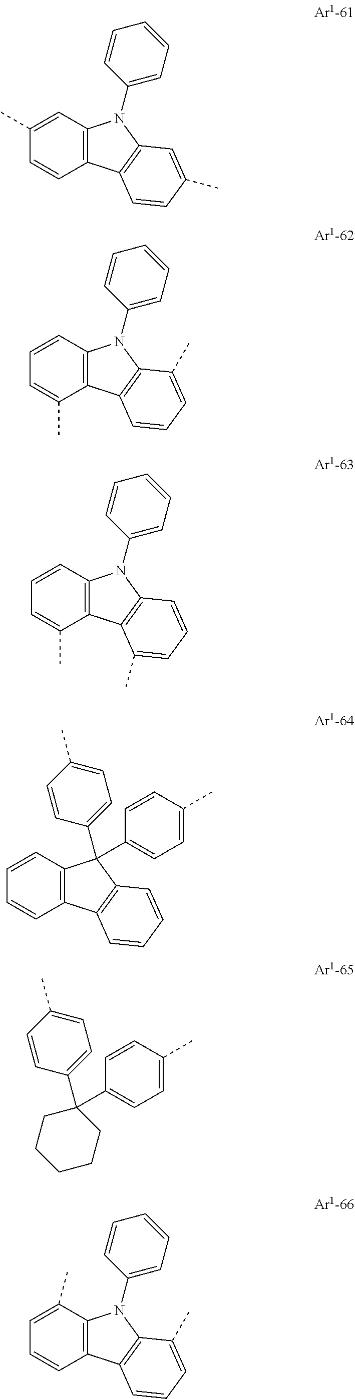

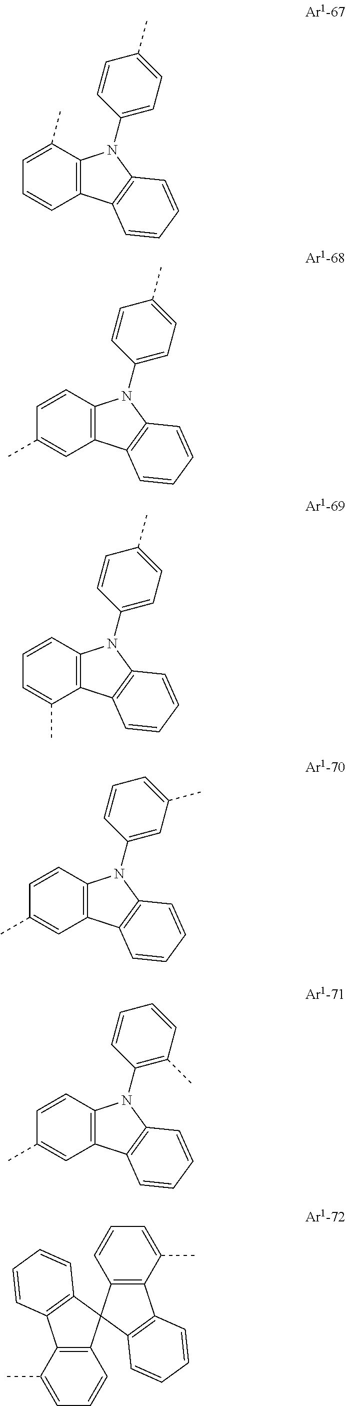

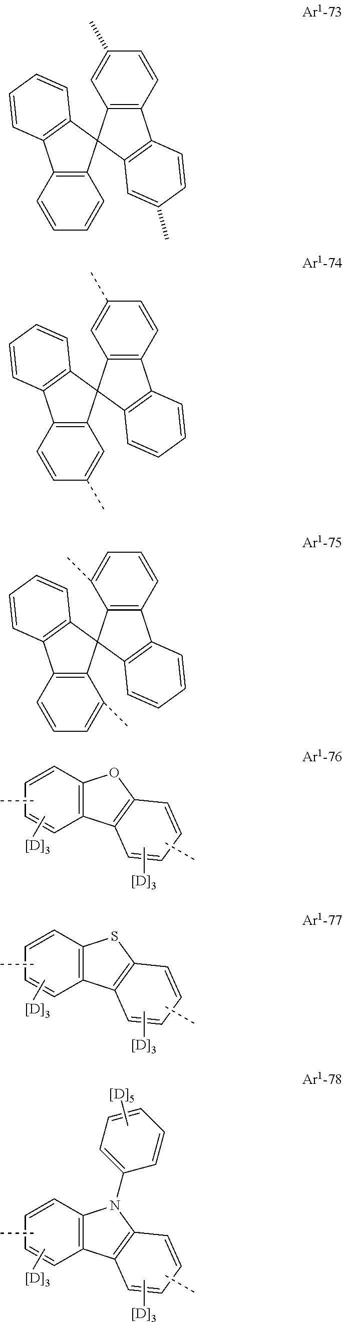

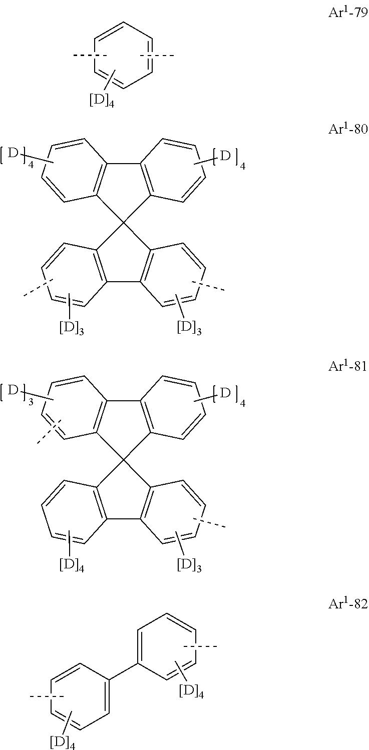

where the dotted lines represent the bonds to the rest of the formula, and where the groups at the positions shown as unsubstituted are each substituted by R² radicals, where the R² radicals in these positions are preferably H.

Ar² groups are preferably the same or different at each instance and are selected from monovalent groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where the monovalent groups are each substituted by one or more R² radicals. Alternatively, the Ar² groups are the same or different at each instance and may preferably be selected from combinations of groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, carbazole, benzofuran, benzothiophene, indole, quinoline, pyridine, pyrimidine, pyrazine, pyridazine and triazine, where the groups are each substituted by one or more R² radicals.

Particularly preferred Ar² groups are the same or different at each instance and are selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by one or more R² radicals.

Particularly preferred Ar² groups are the same or different and are selected from the following formulae:

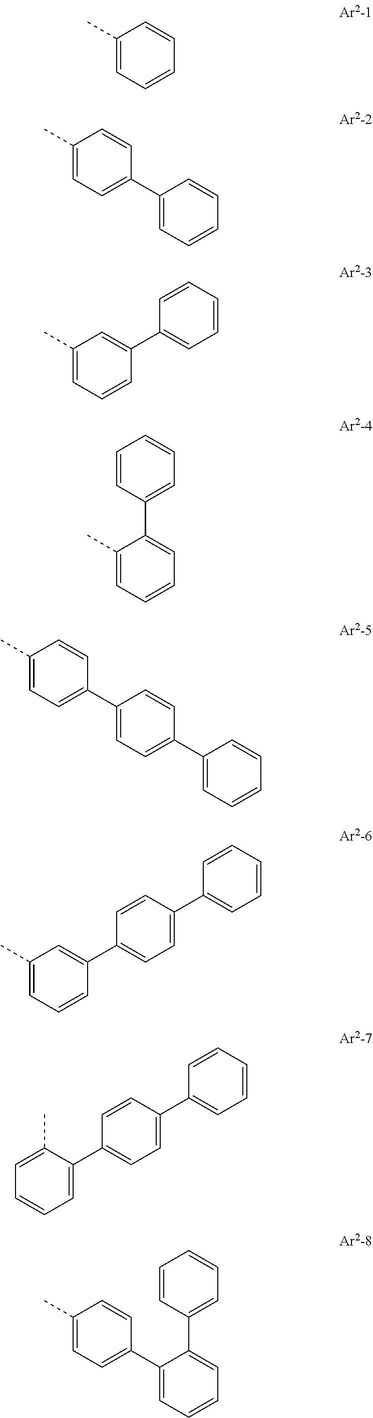

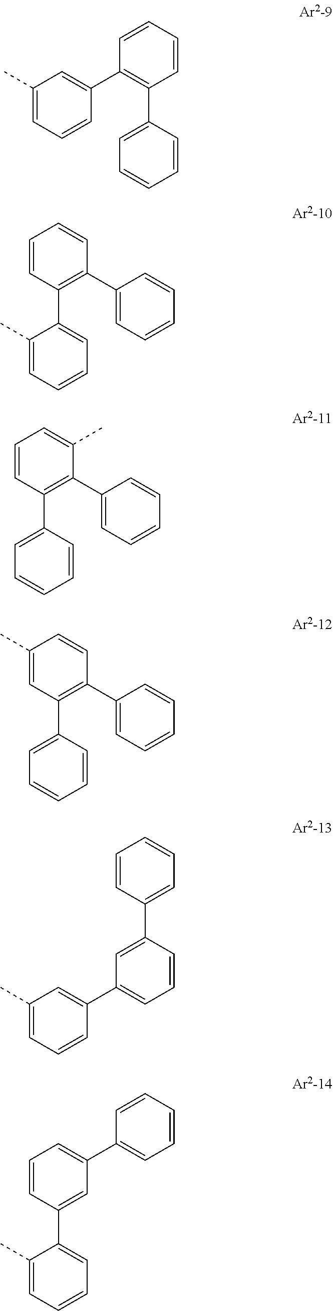

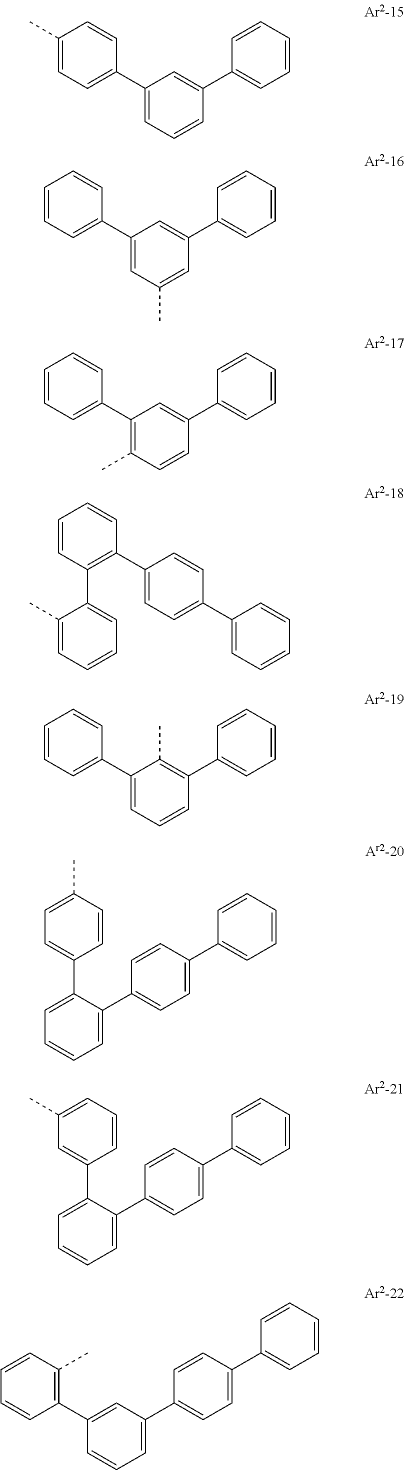

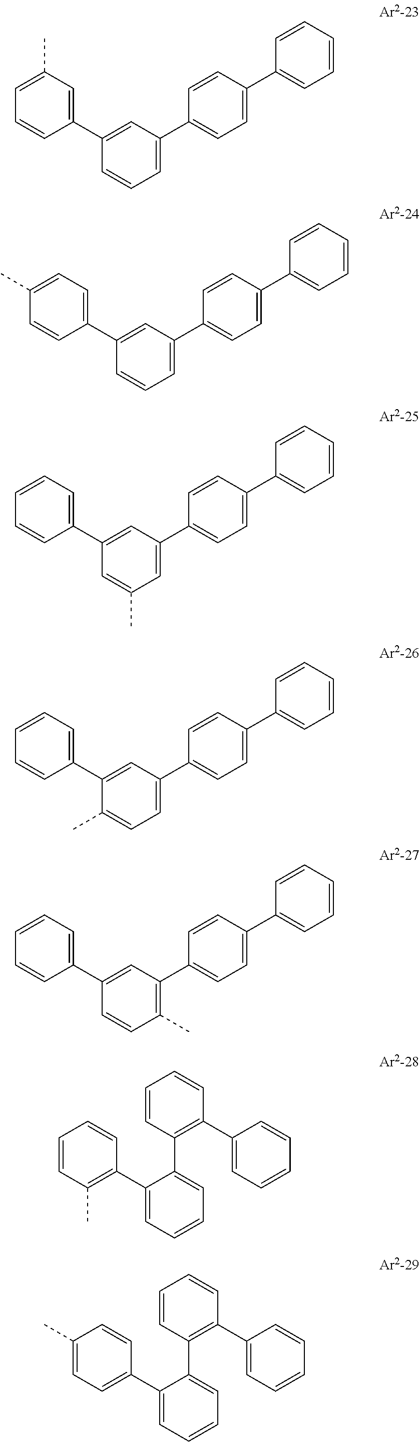

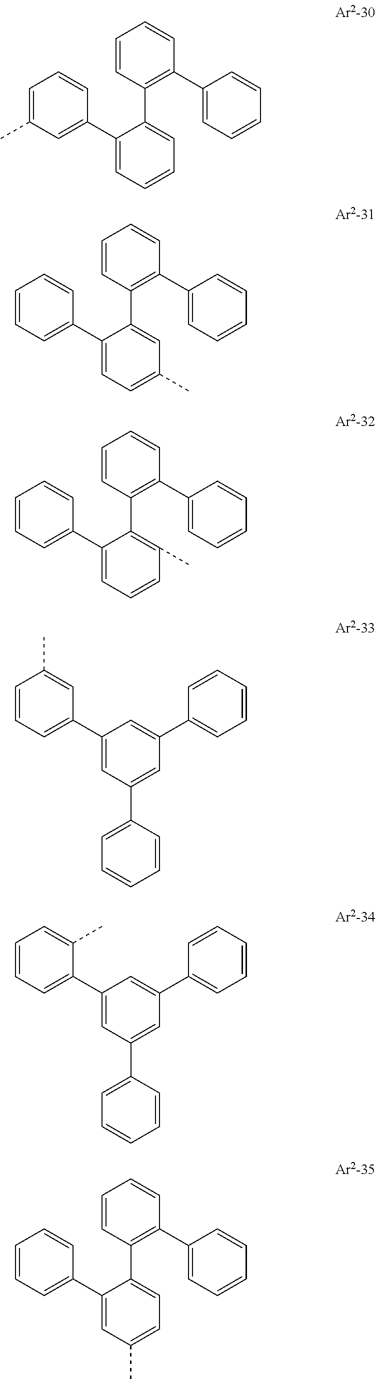

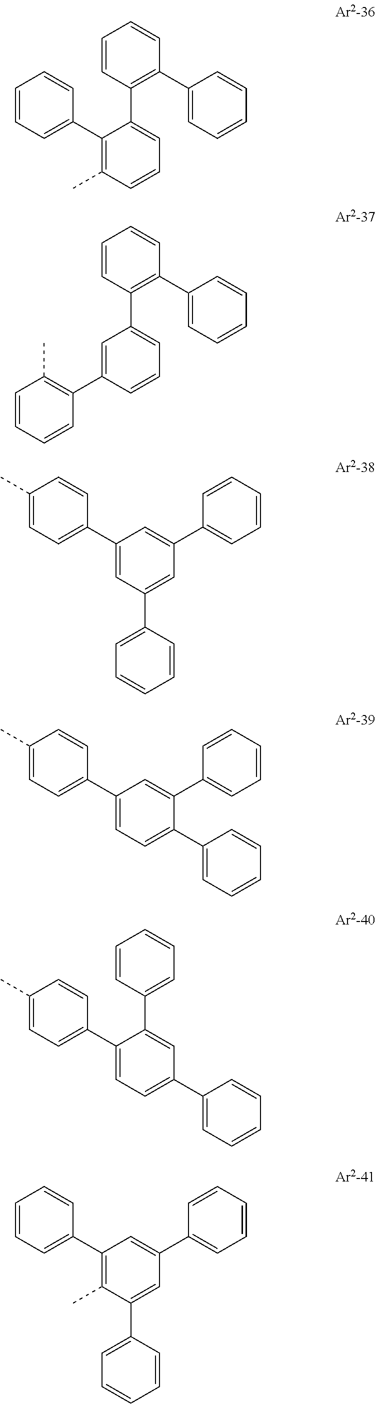

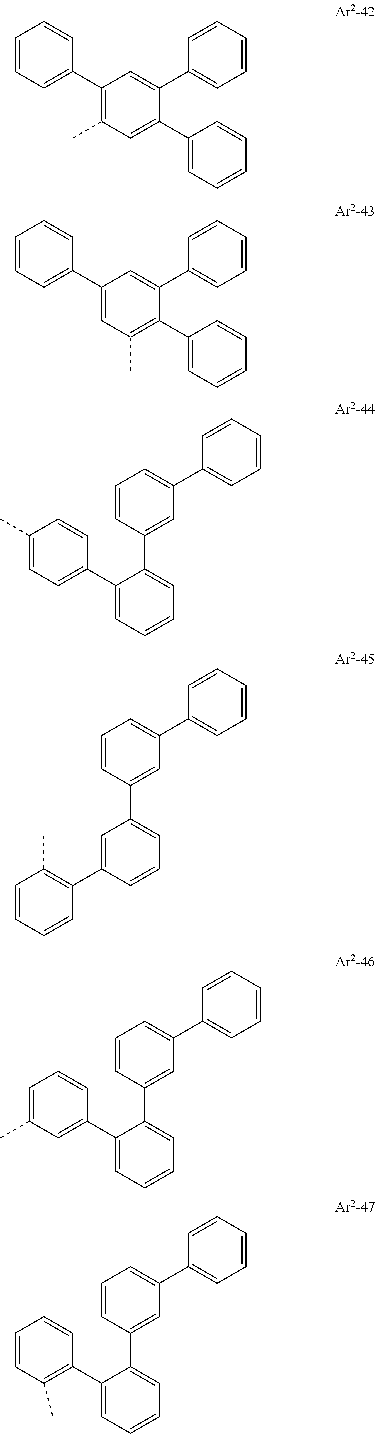

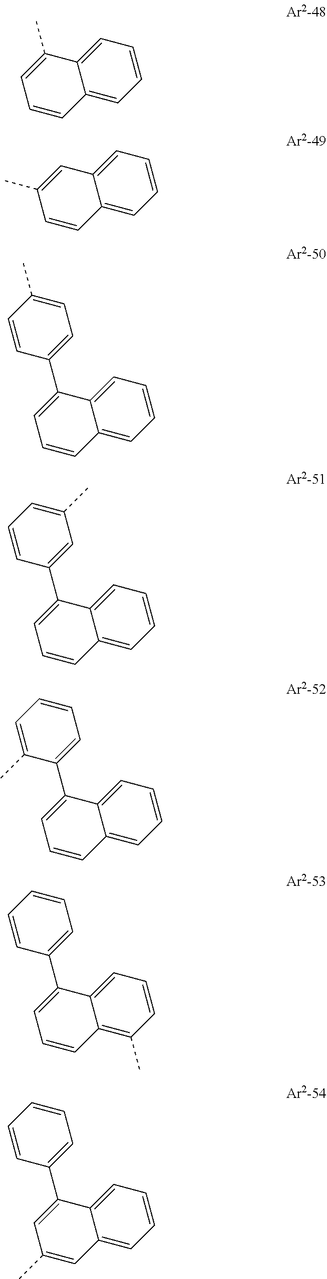

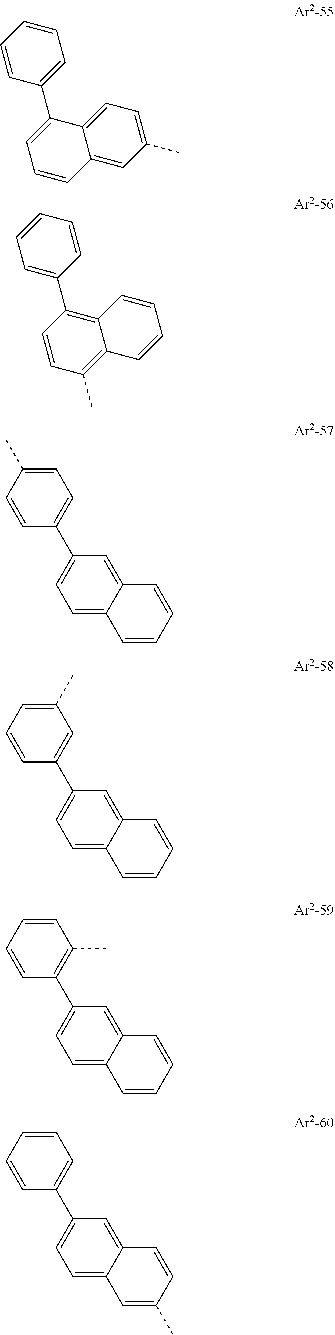

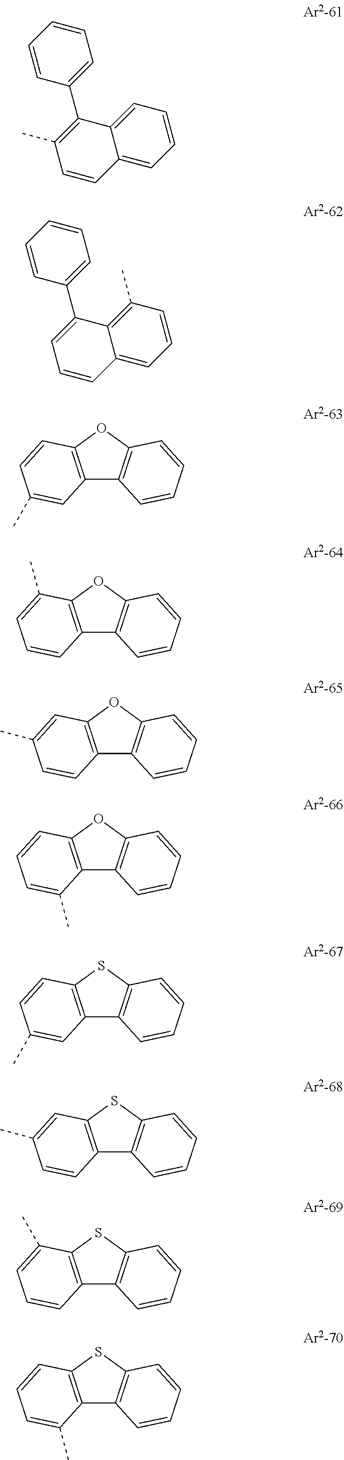

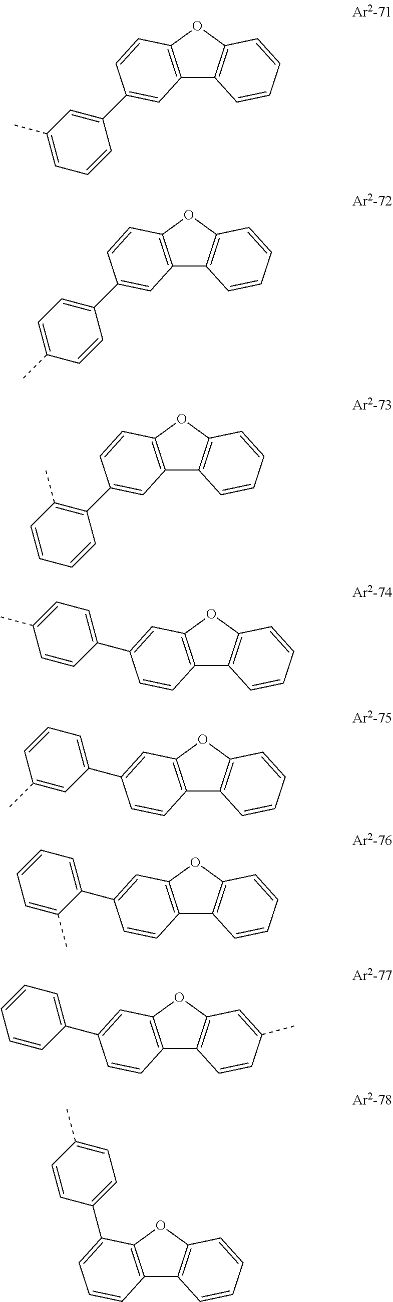

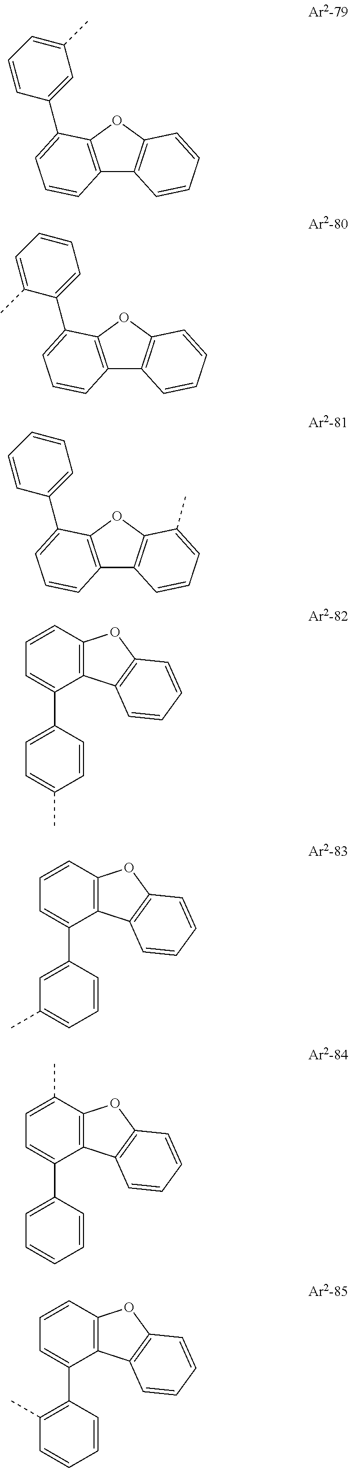

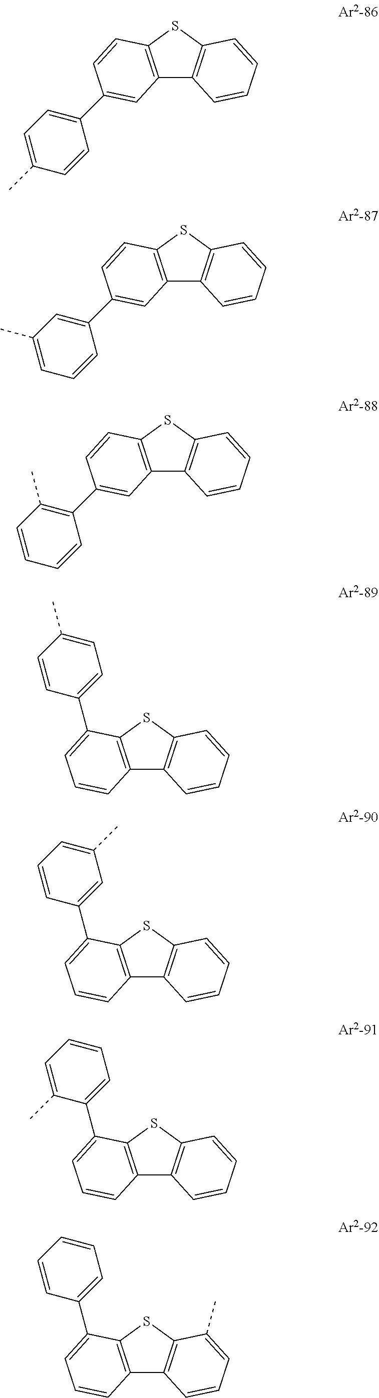

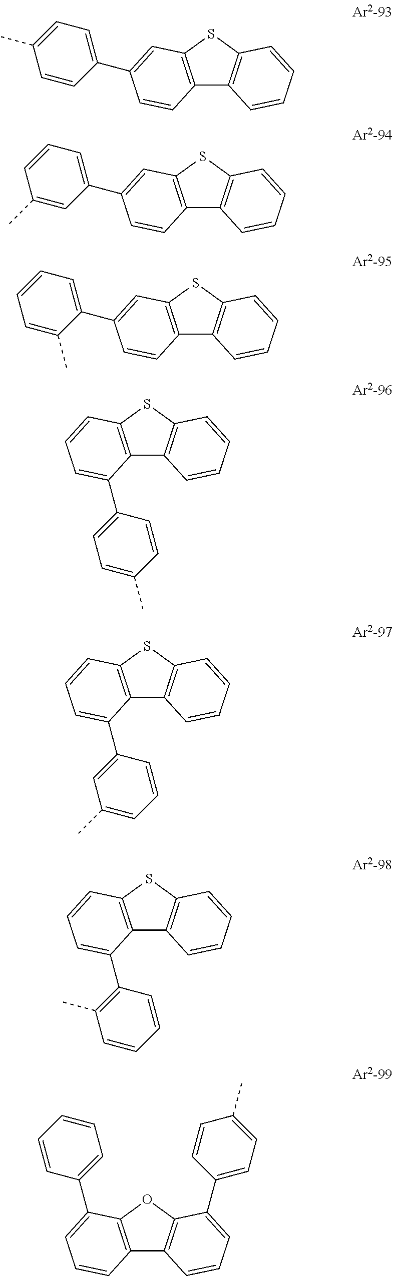

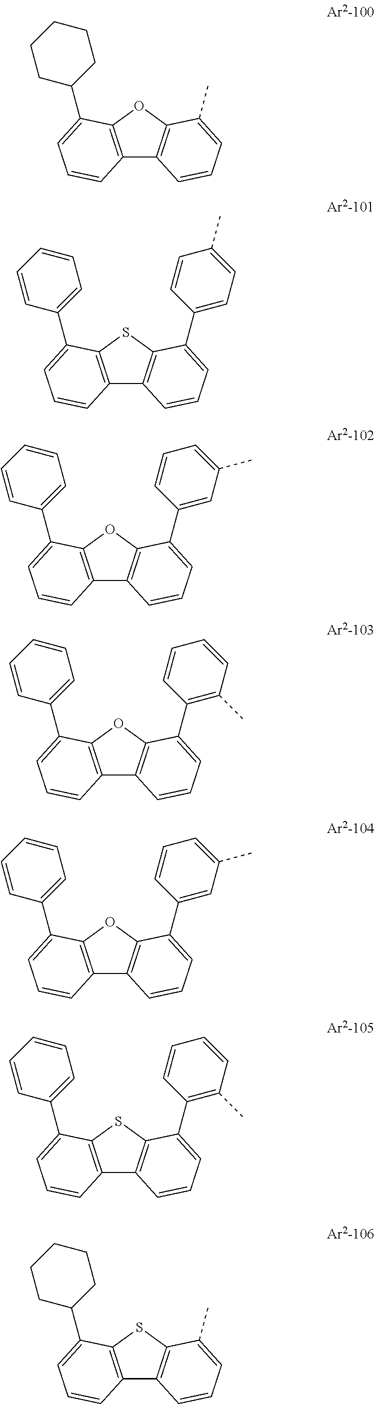

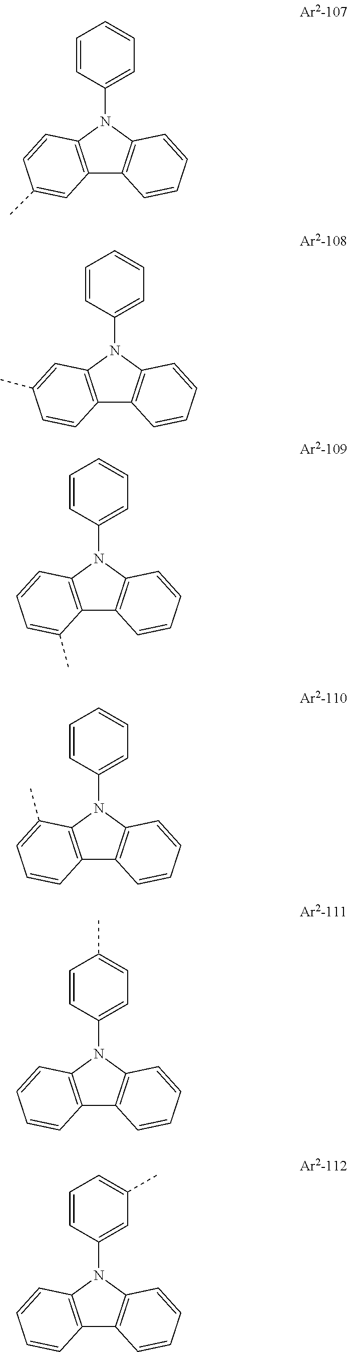

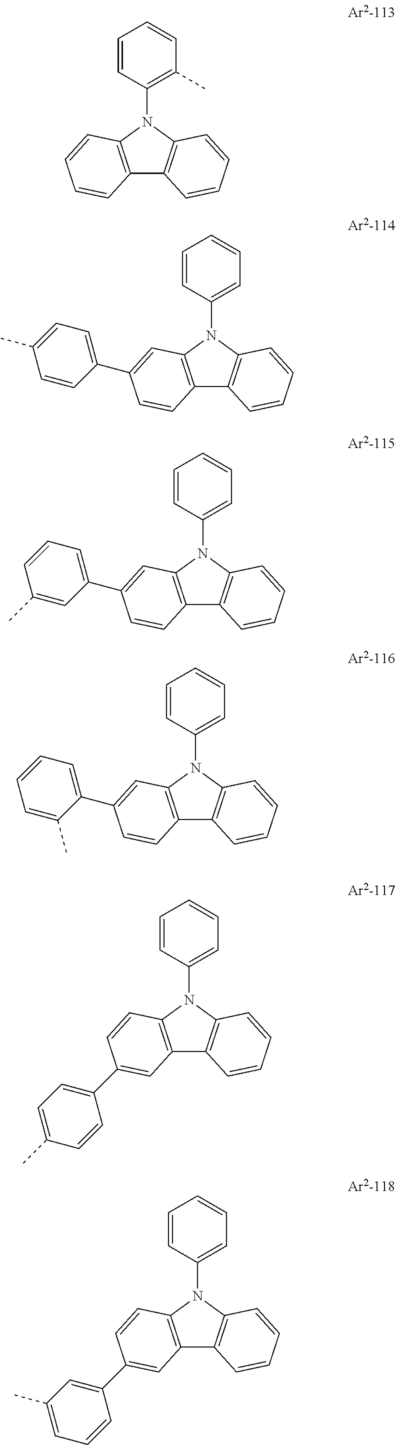

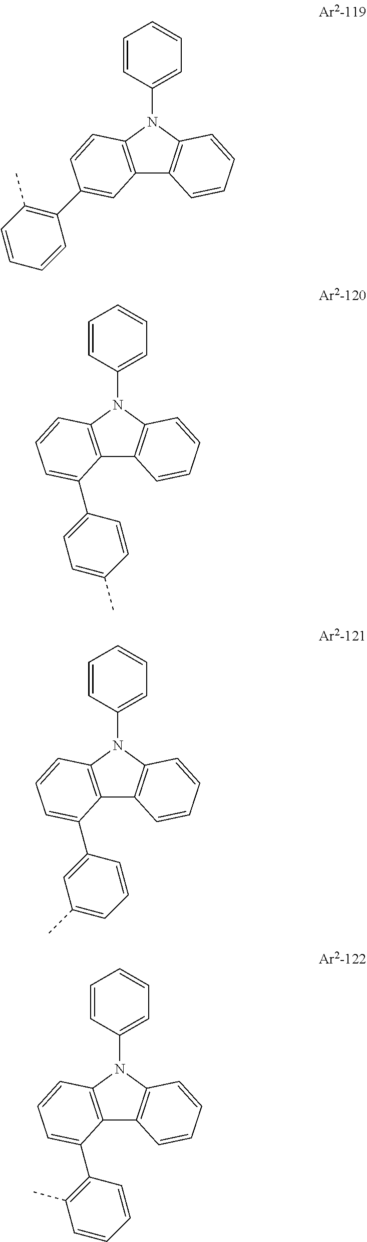

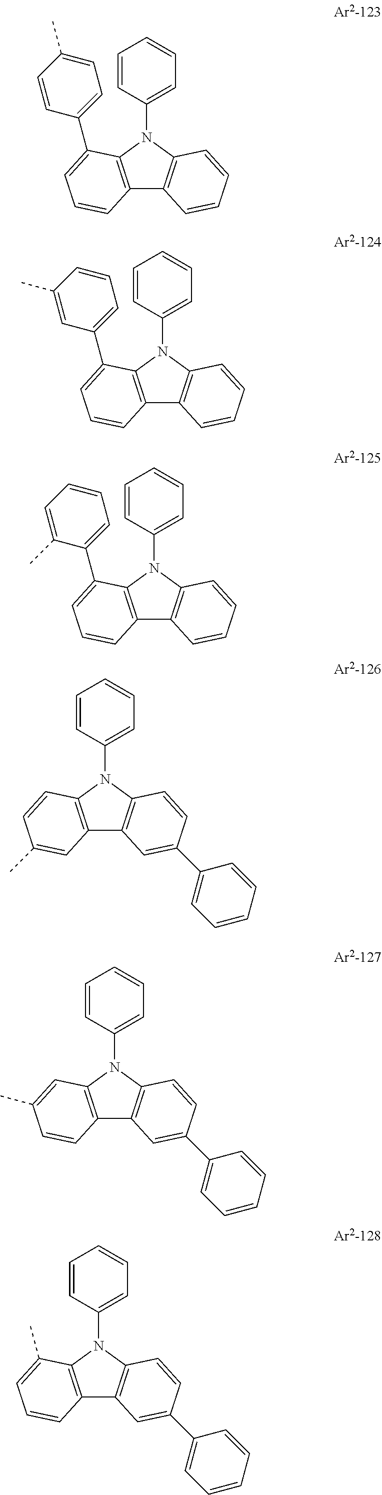

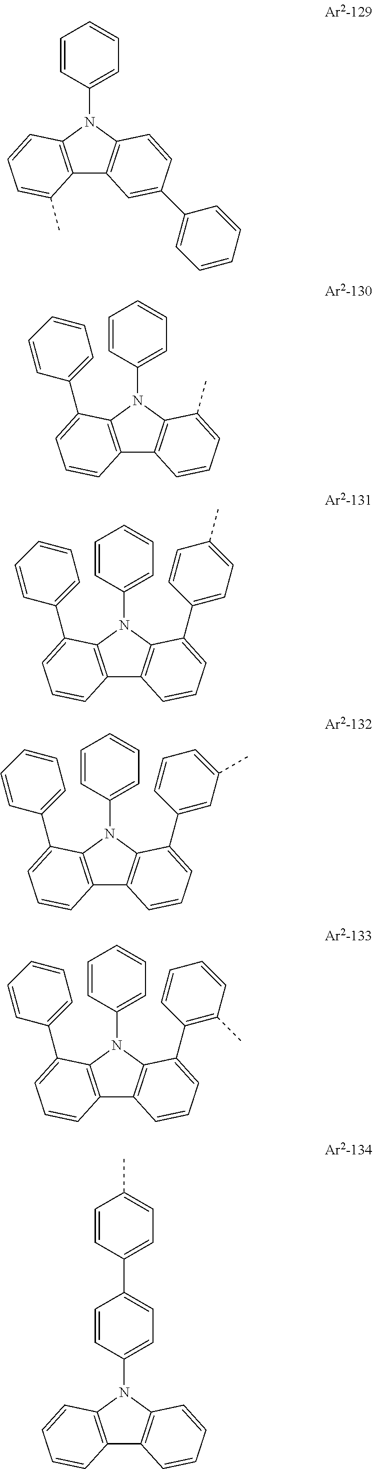

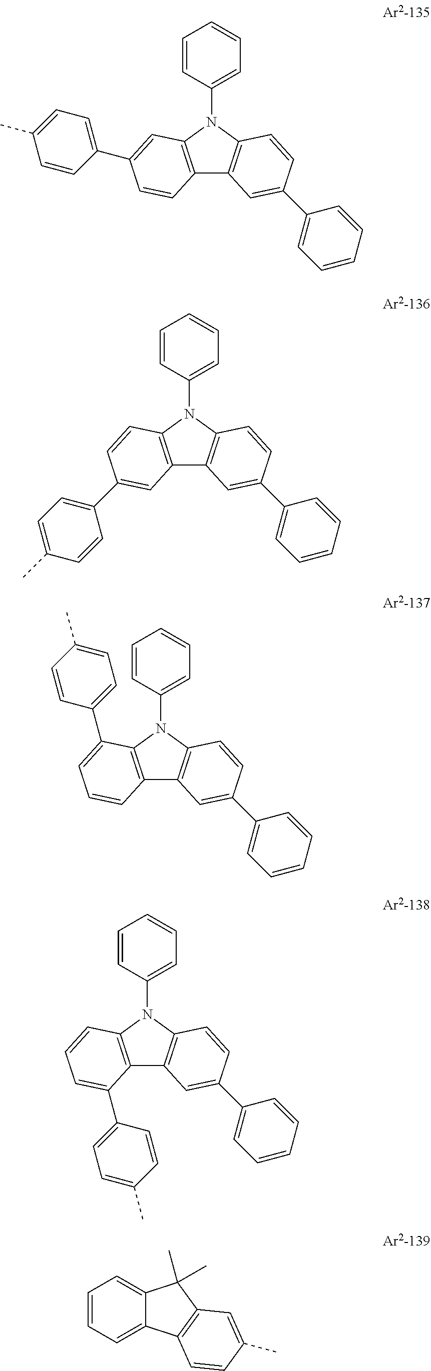

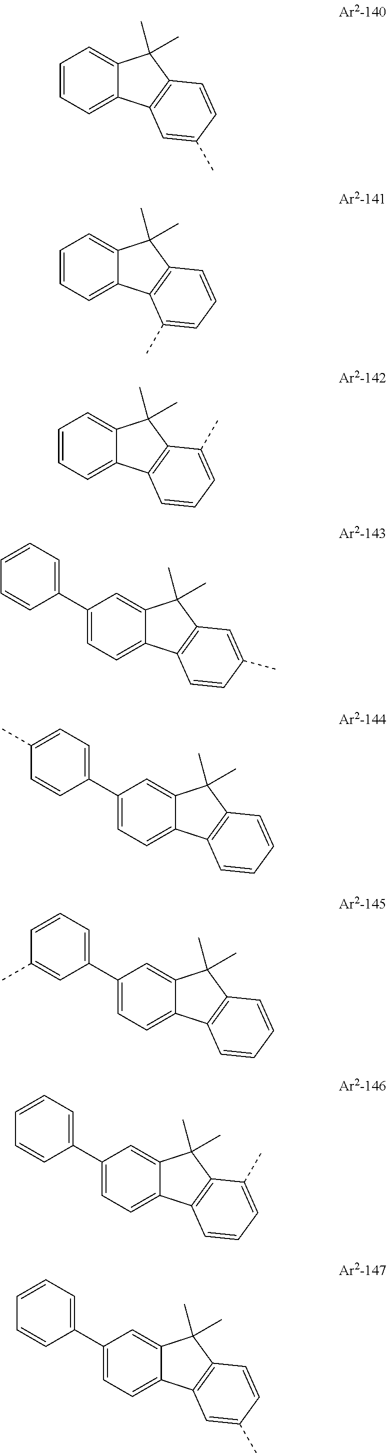

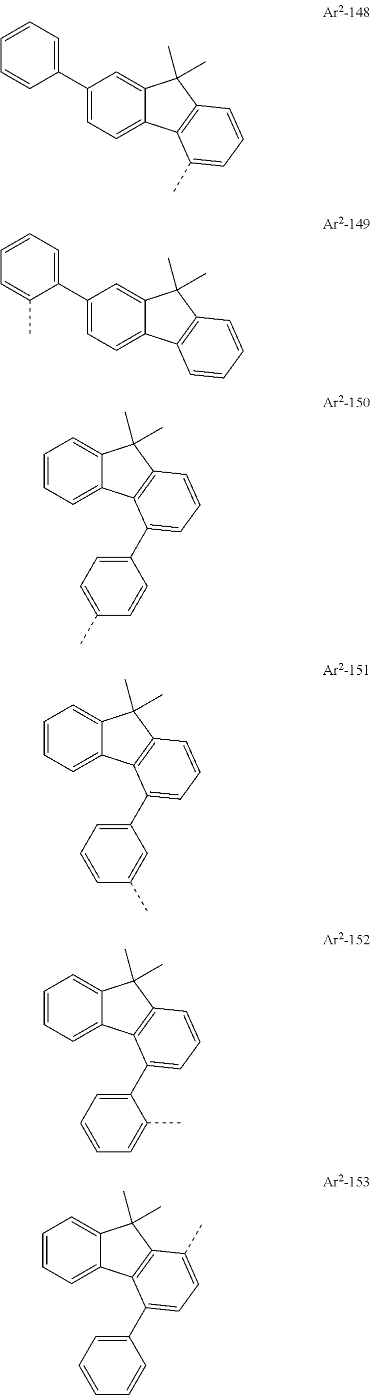

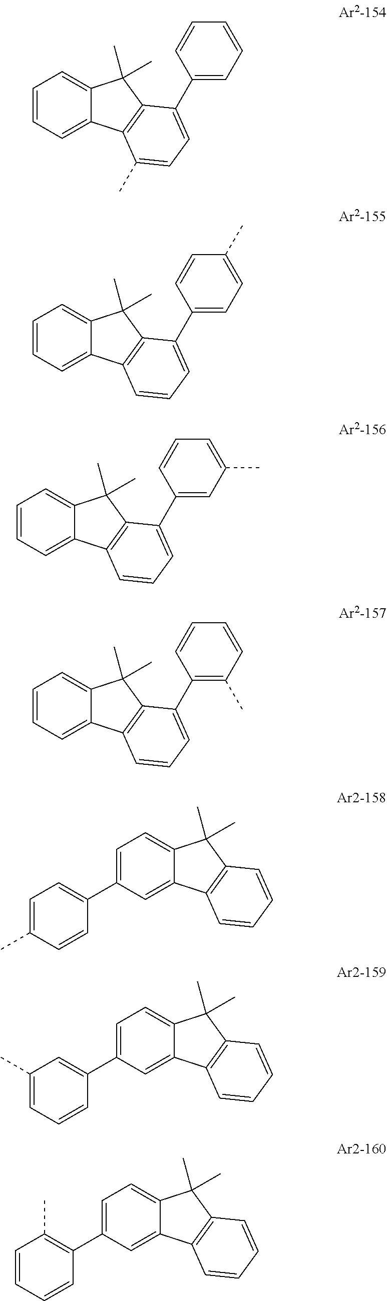

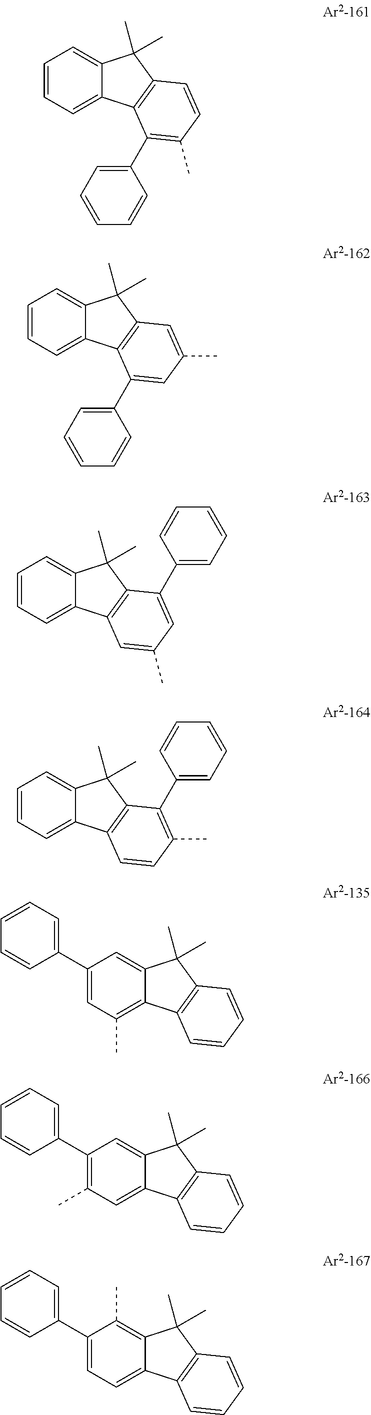

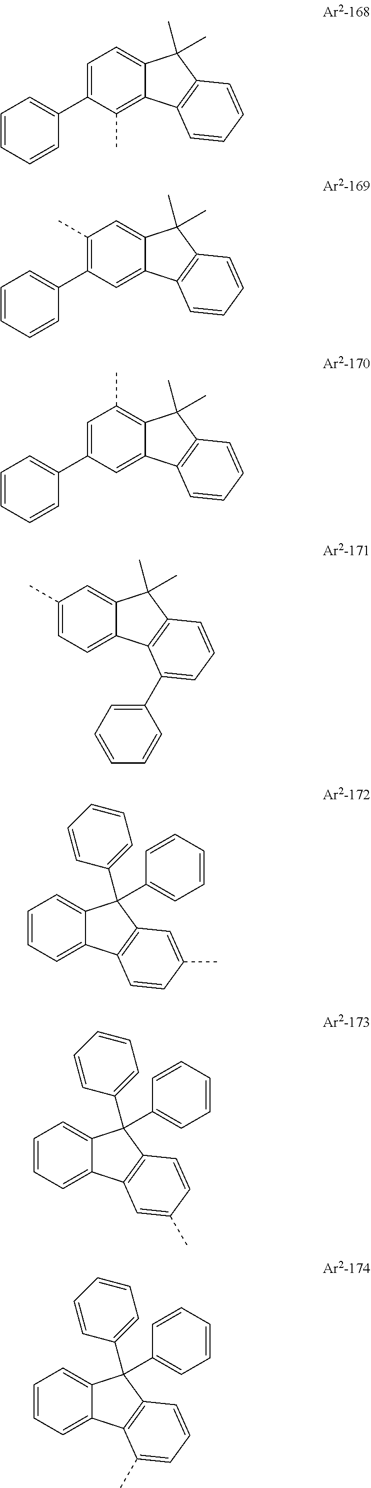

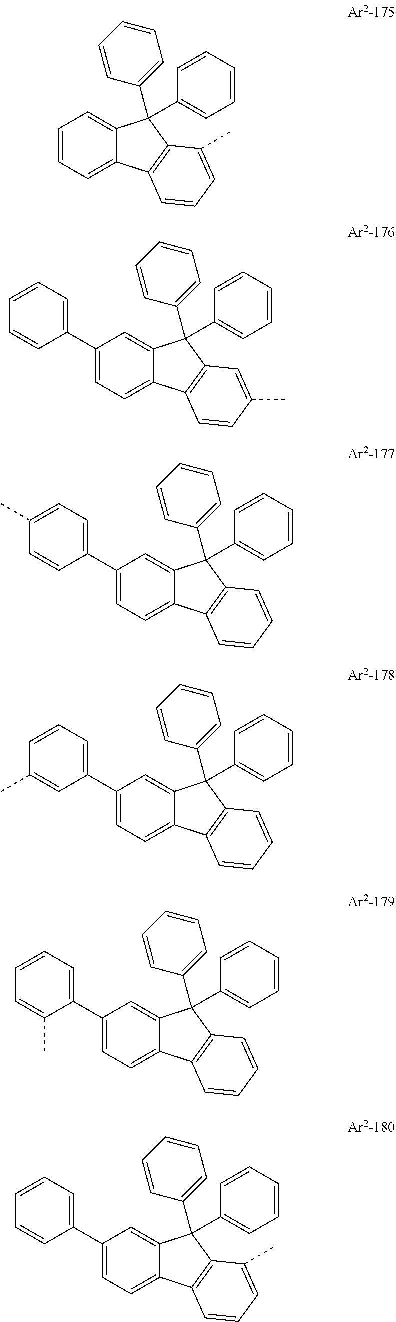

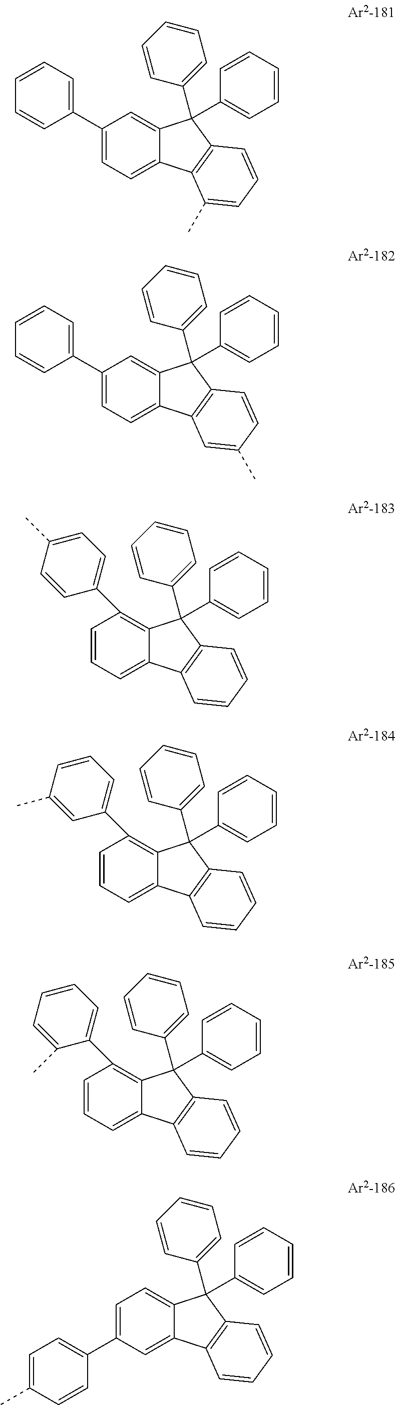

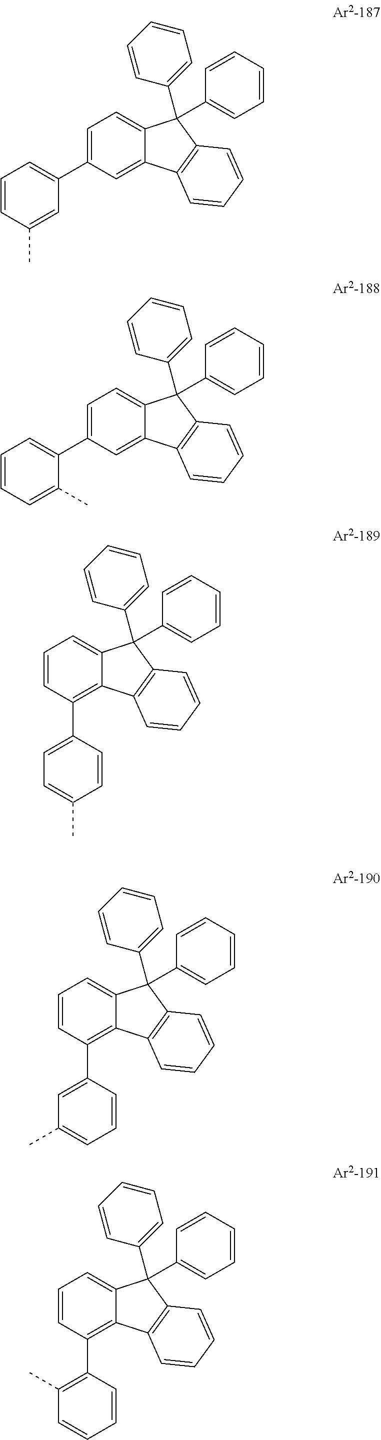

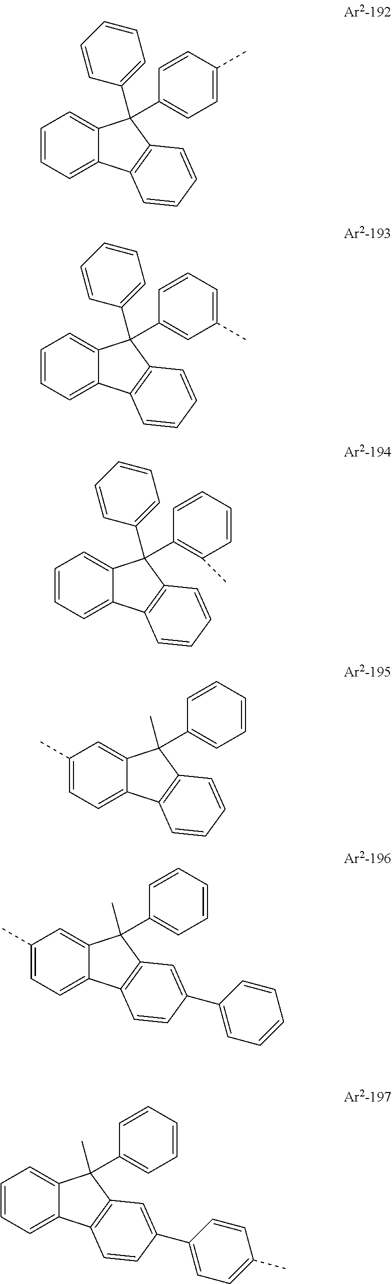

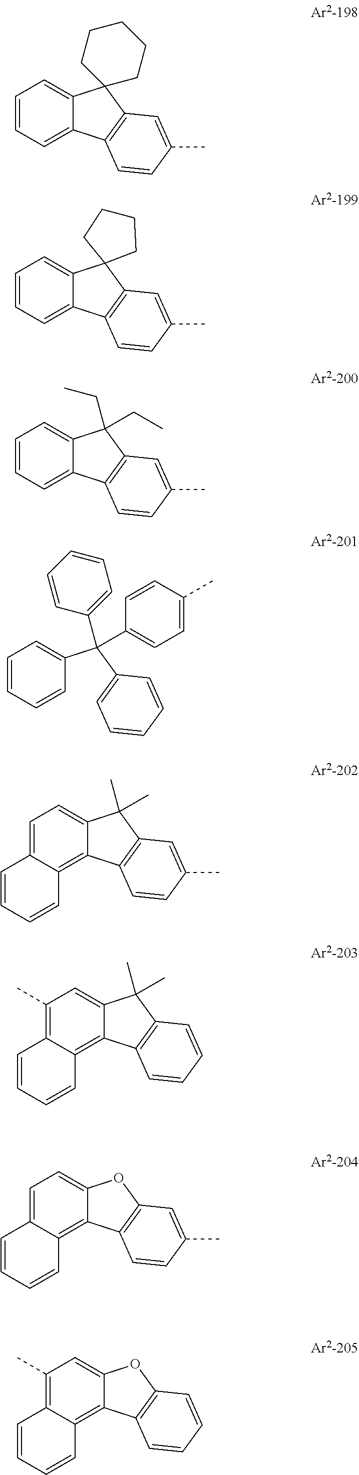

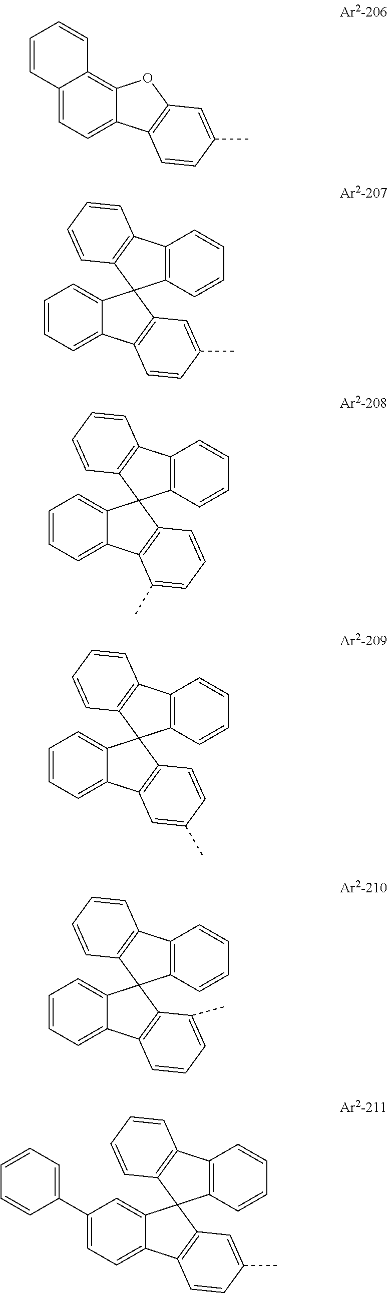

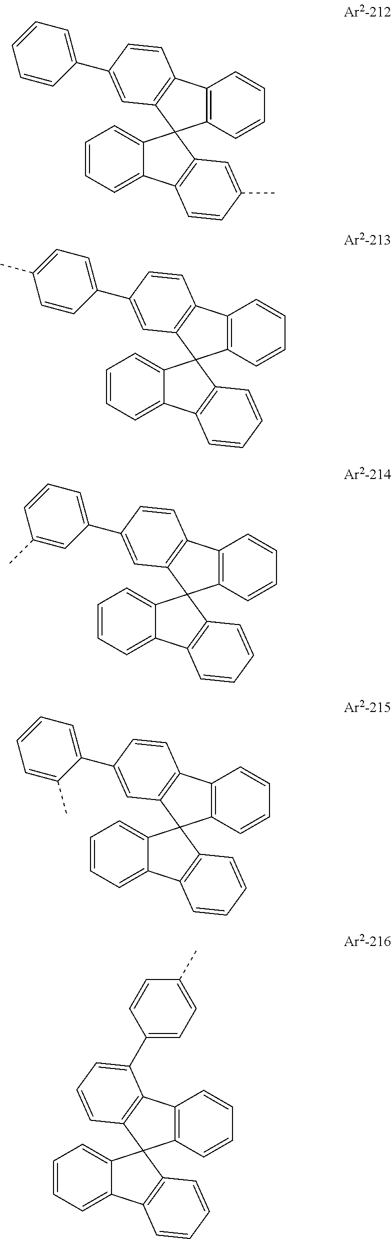

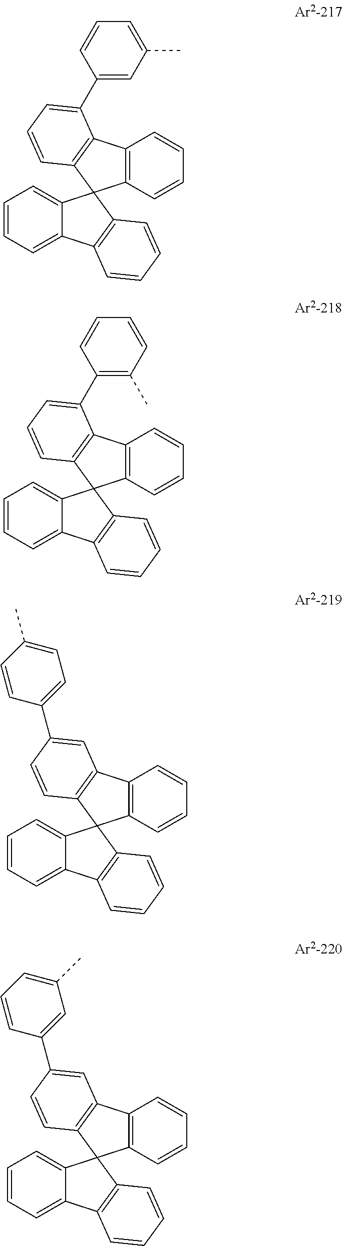

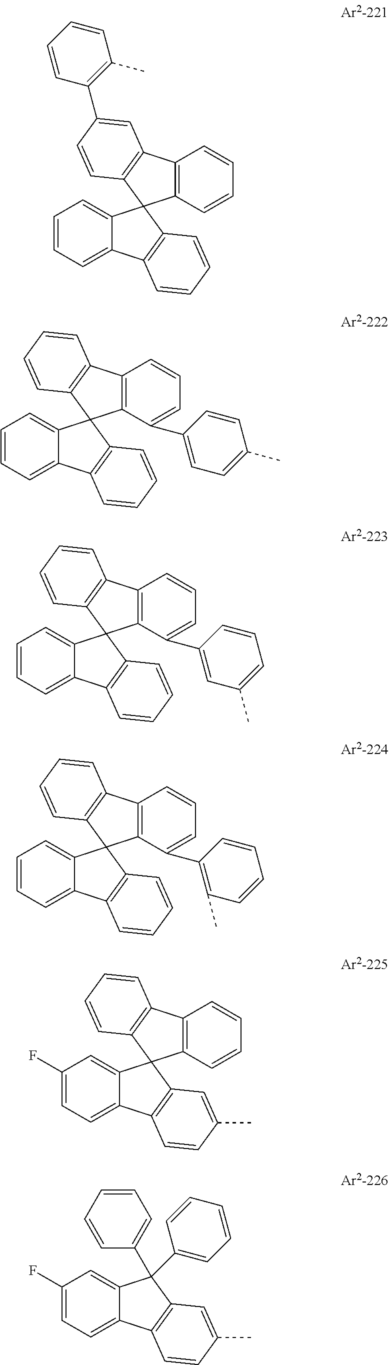

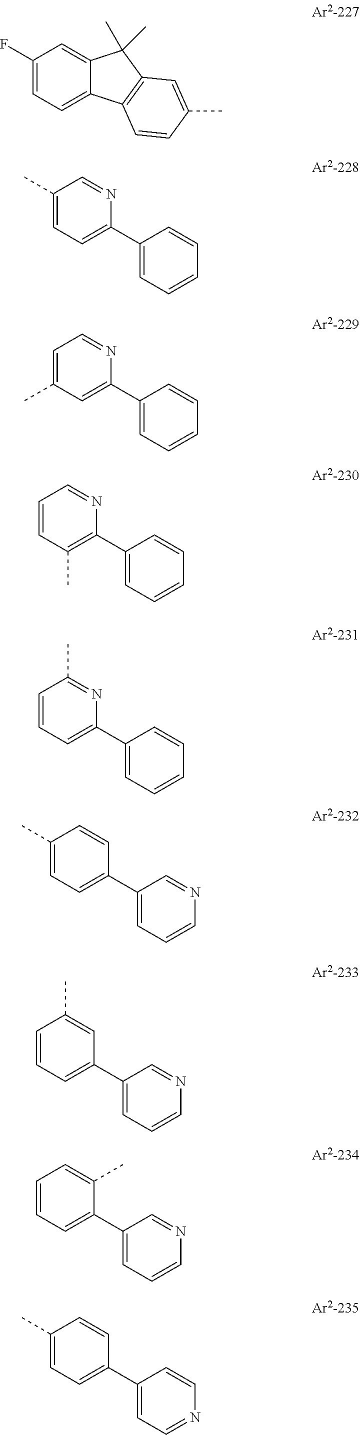

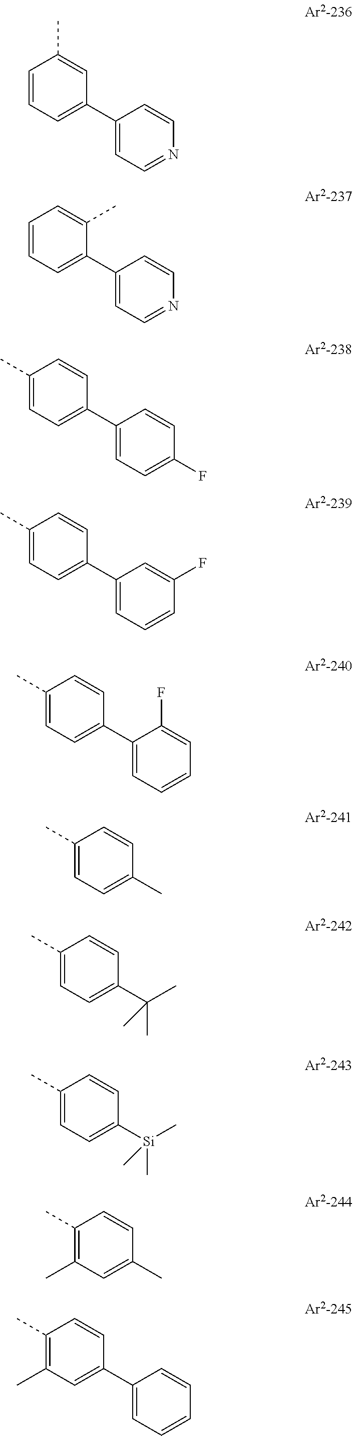

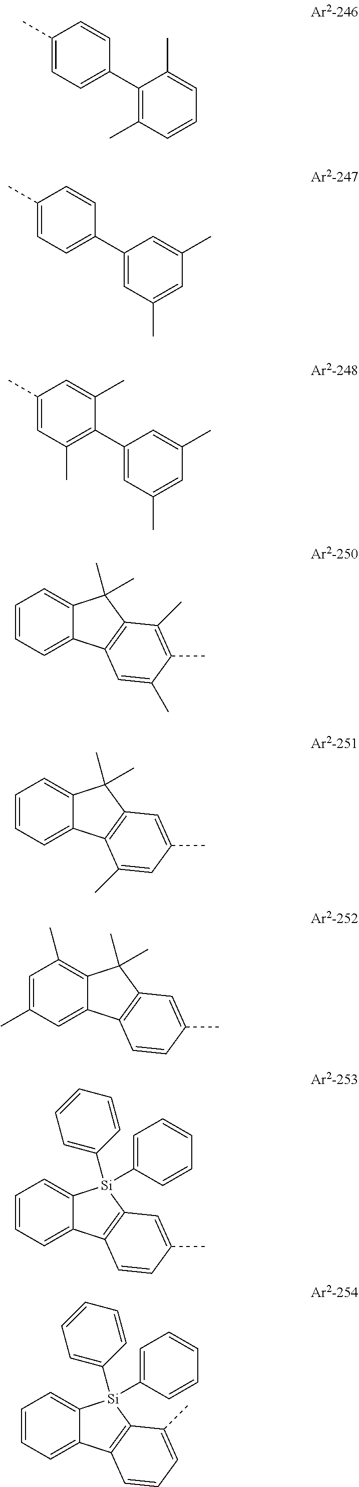

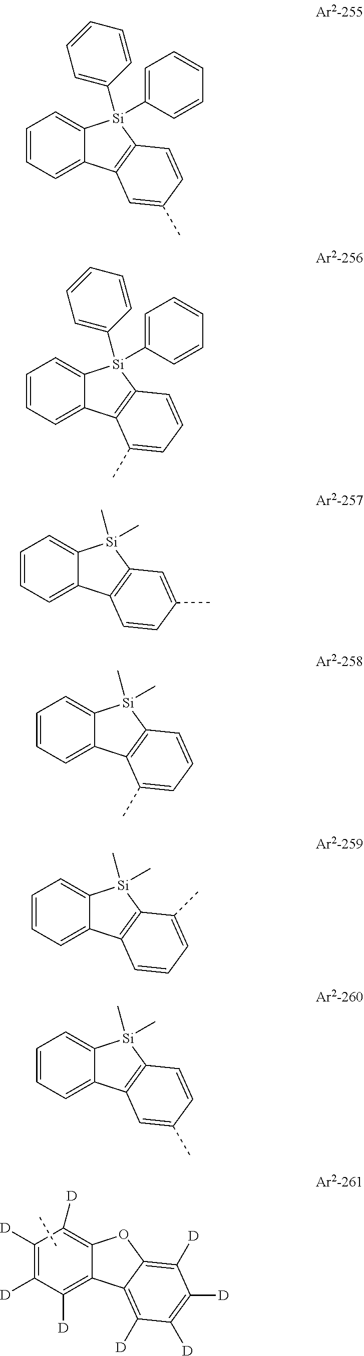

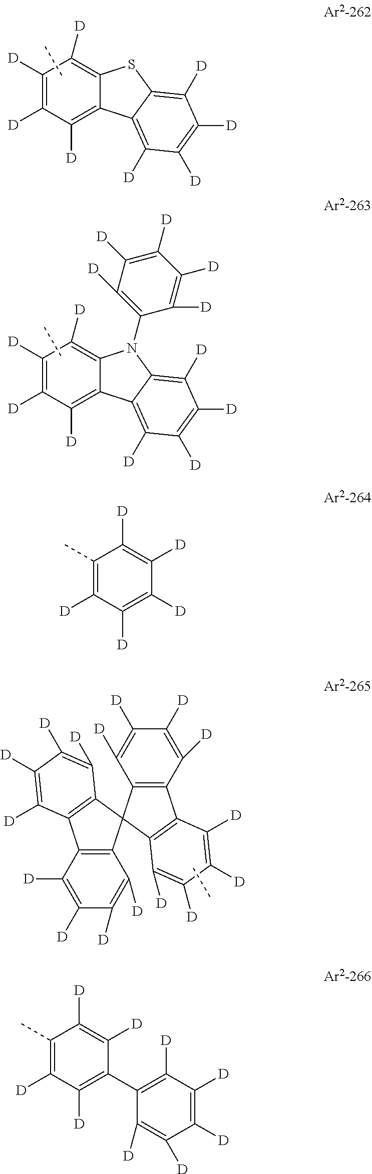

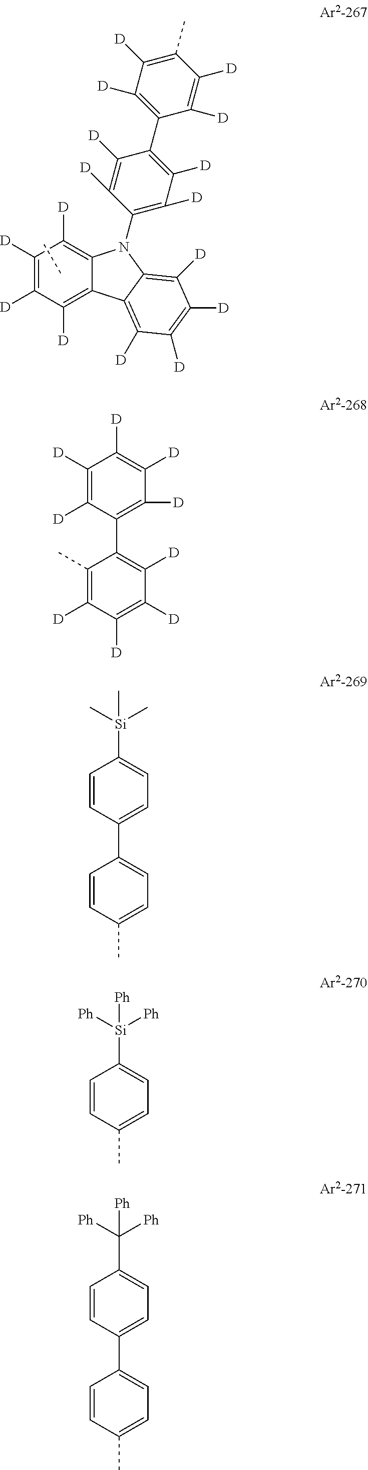

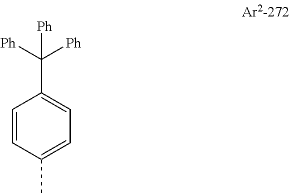

where the groups at the positions shown as unsubstituted are substituted by R² radicals, where R² in these positions is preferably H, and where the dotted bond is the bond to the amine nitrogen atom.

Preferably, R¹ and R² are the same or different at each instance and are selected from H, D, F, CN, Si(R³)₃, N(R³)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R³ radicals; and where one or more CH₂ groups in the alkyl or alkoxy groups mentioned may be replaced by —C≡C—, R³C═CR³—, Si(R³)₂, C═O, C═NR³, —NR³—, —O—, —S—, —C(═O)O— or —C(═O)NR³—.

More preferably, R¹ is the same or different at each instance and is selected from H, D, F, CN, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R³ radicals.

More preferably, R² is the same or different at each instance and is selected from H, D, F, CN, Si(R³)₄, straight-chain alkyl groups having 1 to carbon atoms, branched or cyclic alkyl groups having 3 to 20 carbon atoms, aromatic ring systems having 6 to 40 aromatic ring atoms and heteroaromatic ring systems having 5 to 40 aromatic ring atoms, where the alkyl groups mentioned, the aromatic ring systems mentioned and the heteroaromatic ring systems mentioned are each substituted by R³ radicals.

It is particularly preferable that:
Z is CR¹, where Z is C when a

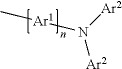

group is bonded thereto;
X is a single bond;
Ar¹ is the same or different at each instance and is a divalent group derived from benzene which is substituted in each case by one or more R² radicals;
index n is 0 or 1;
Ar² is the same or different at each instance and is selected from the abovementioned formulae Ar²-1 to Ar²-272;
R¹ is the same or different at each instance and is selected from H, D, F, CN, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R³ radicals;
R² is the same or different at each instance and is selected from H, D, F, CN, Si(R³)₄, straight-chain alkyl groups having 1 to 10 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms, where the alkyl groups mentioned, the aromatic ring systems mentioned and the heteroaromatic ring systems mentioned are each substituted by R³ radicals.

Formula (I) preferably conforms to a formula (I-1)

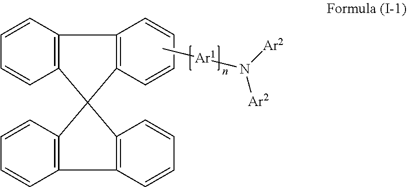

where the groups that occur are as defined above and are preferably defined according to their preferred embodiments, and where the unoccupied positions on the spirobifluorene are substituted by R¹ radicals.

Formula (II) preferably conforms to a formula (II-1)

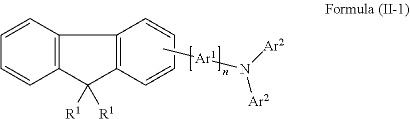

where the groups that occur are as defined above and are preferably defined according to their preferred embodiments, and where the unoccupied positions on the fluorene are substituted by R¹ radicals.

Preferred embodiments of compounds of the formula (I) are the compounds cited as example structures in WO2015/158411, WO2011/006574, WO2013/120577, WO2016/078738, WO2017/012687, WO2012/034627, WO2013/139431, WO2017/102063, WO2018/069167, WO2014/072017, WO2017/102064, WO2017/016632, WO2013/083216 and WO2017/133829.

Preferred embodiments of compounds of the formula (II) are the compounds cited as example structures in WO2014/015937, WO2014/015938, WO2014/015935 and WO2015/082056.

Hereinafter, one of the two different compounds in the hole-transporting layer that conform to identical or different formulae selected from formulae (I) and (II) is referred to as HTM-1, and the other of the two different compounds in the hole-transporting layer that conform to identical or different formulae selected from formulae (I) and (II) is referred to as HTM-2.

In a preferred embodiment, HTM-1 conforms to a formula selected from formulae (I-1-A) and (II-1-A)

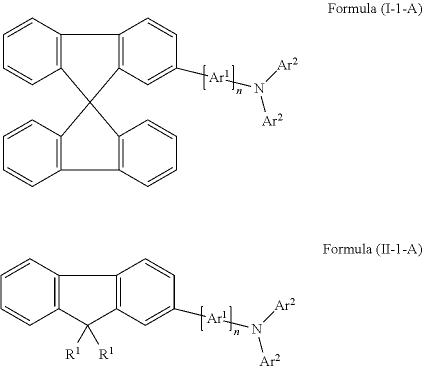

and
HTM-2 conforms to a formula selected from formulae (I-1-B), (I-1-C), (I-1-D), (II-1-B), (II-1-C), and (II-1-D)

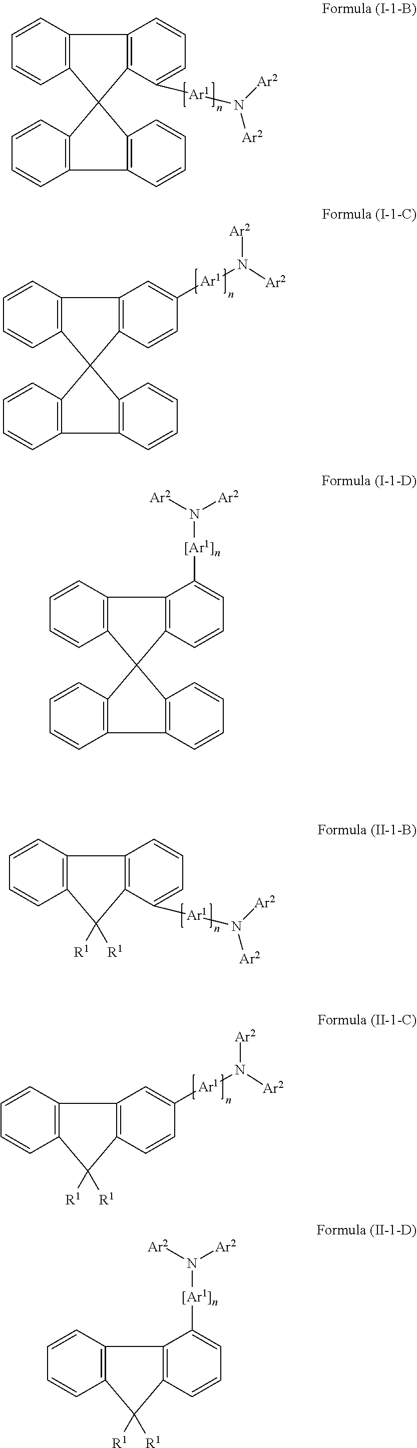

where the groups that occur in the formulae (I-1-A) to (I-1-D) and (II-1-B) to (II-1-D) are as defined above and are preferably defined according to their preferred embodiments, and where the unoccupied positions on the spirobifluorene and fluorene are each substituted by R¹ radicals. More preferably, HTM-2 conforms to a formula (I-1-B) or (I-1-D), most preferably to a formula (I-1-D). In an alternative preferred embodiment, HTM-2 conforms to a formula (II-1-B) or (II-1-D), most preferably to a formula (II-1-D).

Preferably, HTM-1 is present in the hole-transporting layer in a proportion five to two times as high as the proportion of HTM-2 in the layer.

Preferably, HTM-1 is present in the layer in a proportion of 50%-95%, more preferably in a proportion of 60%-90%, and most preferably in a proportion of 65%-85%.

Preferably, HTM-2 is present in the layer in a proportion of 5%-50%, more preferably in a proportion of 10%-40%, and most preferably in a proportion of 15%-35%.

Preferably, HTM-1 is present in the layer in a proportion of 65% to 85%, and HTM-2 is present in the layer in a proportion of 15% to 35%.

In a preferred embodiment, HTM-1 has a HOMO of −4.8 eV to −5.2 eV, and HTM-2 has a HOMO of −5.1 eV to −5.4 eV. More preferably, HTM-1 has a HOMO of −5.0 to −5.2 eV, and HTM-2 has a HOMO of −5.1 to −5.3 eV. It is further preferable that HTM-1 has a higher HOMO than HTM-2. More preferably, HTM-1 has a HOMO higher than HTM-2 by 0.02 to 0.3 eV. “Higher HOMO” is understood here to mean that the value in eV is less negative.

The HOMO energy level is determined by means of cyclic voltammetry (CV), by the method described at page 28 line 1 to page 29 line 21 of the published specification WO 2011/032624.

Preferred embodiments of compounds HTM-1 are shown in the following table:

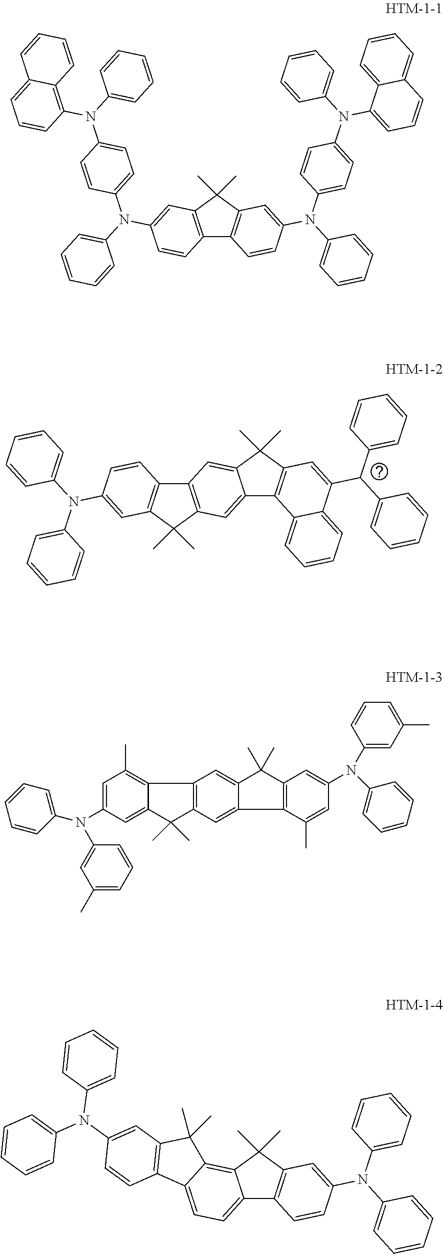 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21 ; CC1(C)c2cc(N(c3ccccc3)c3ccccc3)ccc2-c2cc3c(cc21)-c1c(cc(C(c2ccccc2)c2ccccc2)c2ccccc12)C3(C)C ; Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1 ; CC1(C)c2cc(N(c3ccccc3)c3ccccc3)ccc2-c2ccc3c(c21)C(C)(C)c1cc(N(c2ccccc2)c2ccccc2)ccc1-3]

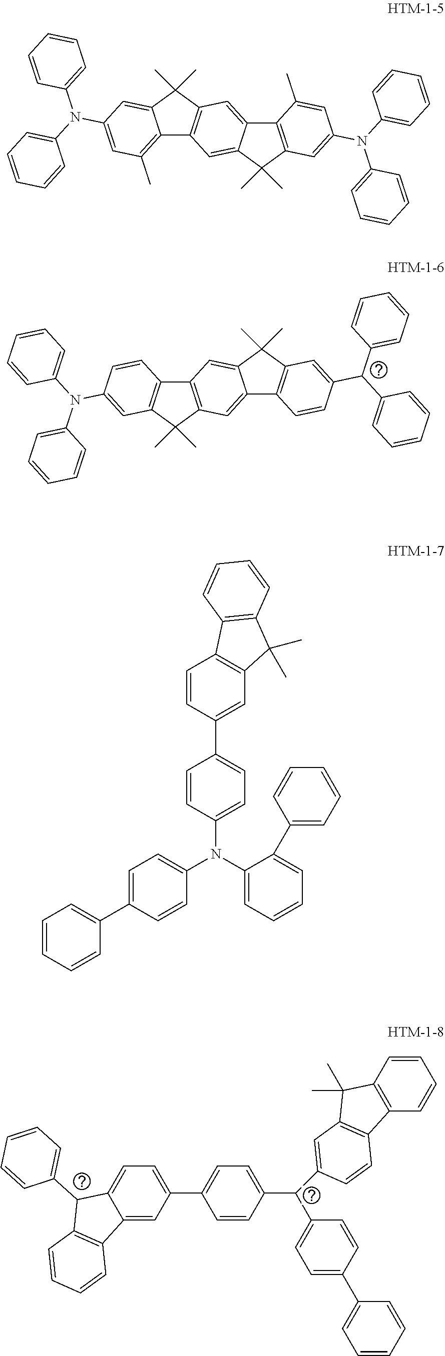

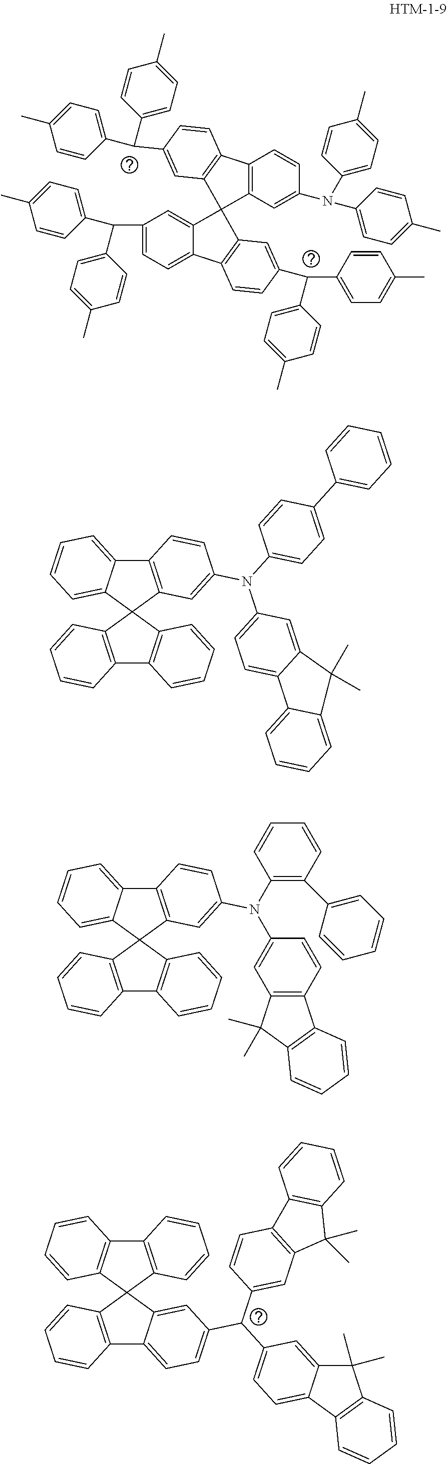

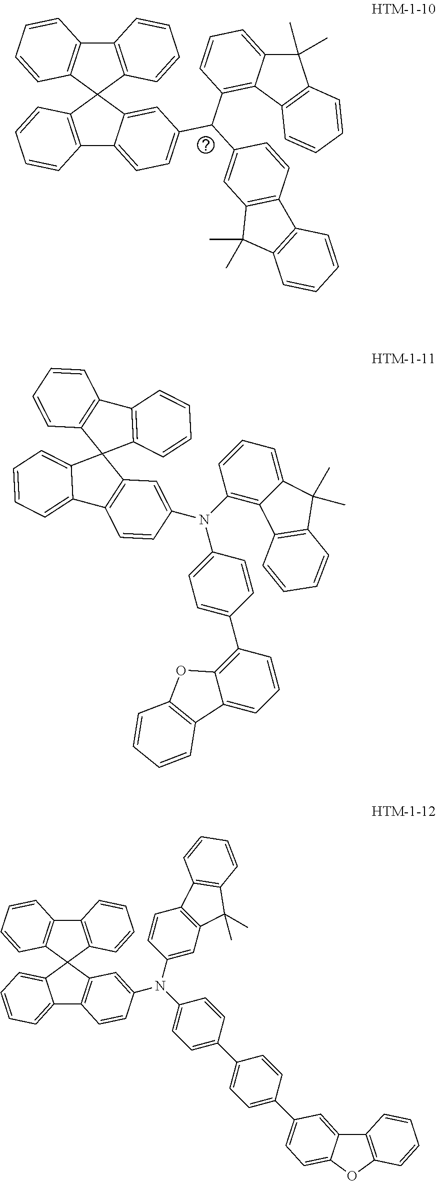

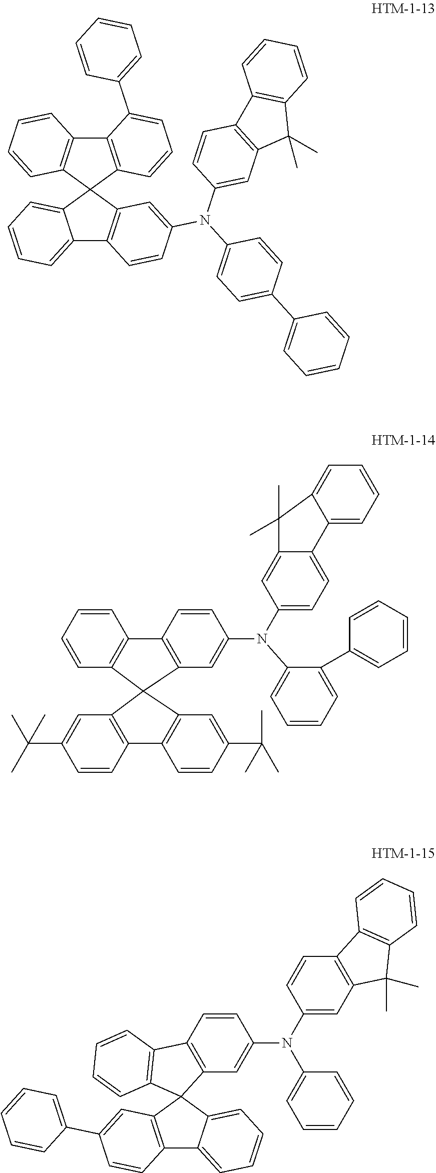 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)cccc43)cc21 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; CC1(C)c2ccccc2-c2ccc(N(c3ccccc3)c3ccc4c(c3)C3(c5ccccc5-c5ccc(-c6ccccc6)cc53)c3ccccc3-4)cc21]

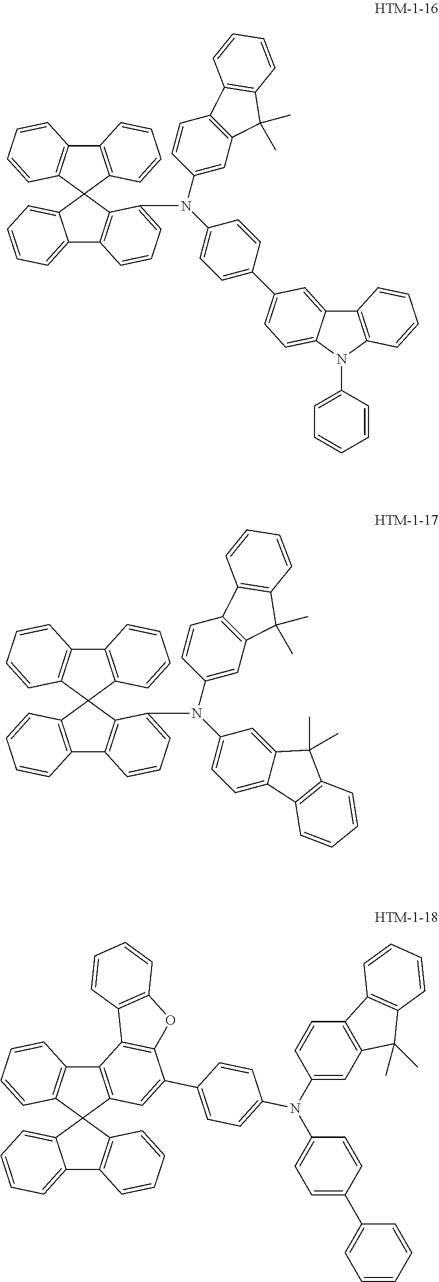 [SMILES: CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21 ; CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21 ; CC1(C)c2ccccc2-c2ccc(N(c3ccc(-c4ccccc4)cc3)c3ccc(-c4cc5c(c6c4oc4ccccc46)-c4ccccc4C54c5ccccc5-c5ccccc54)cc3)cc21]

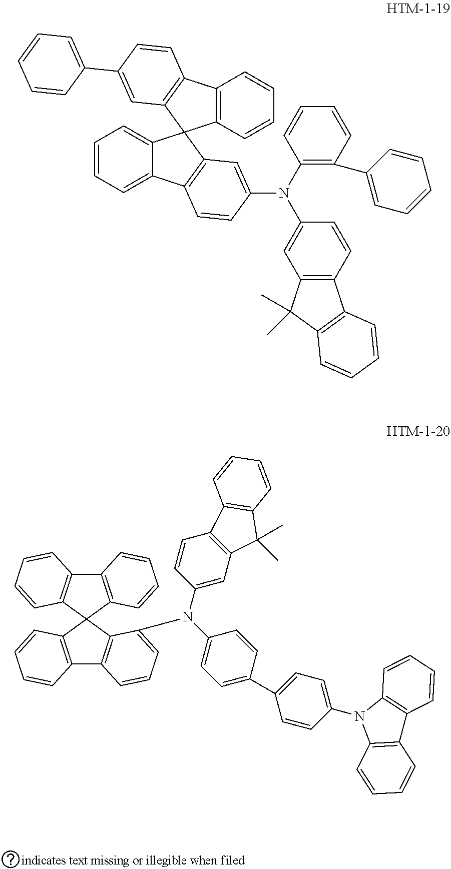 [SMILES: CC1(C)c2ccccc2-c2ccc(N(c3ccc4c(c3)C3(c5ccccc5-c5ccc(-c6ccccc6)cc53)c3ccccc3-4)c3ccccc3-c3ccccc3)cc21 ; CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]

Preferred embodiments of compounds HTM-2 are shown in the following table:

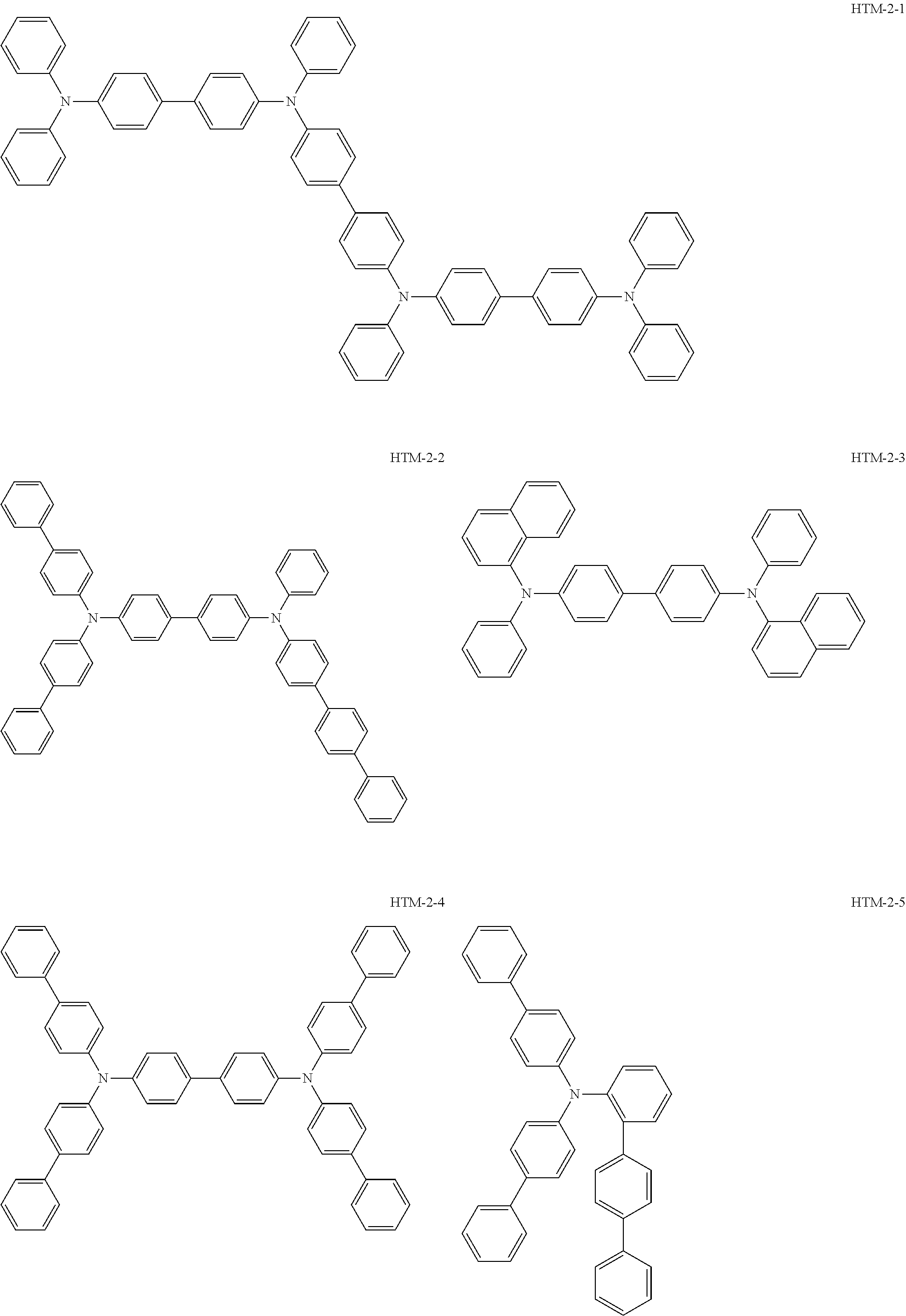 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(-c3ccccc3N(c3ccc(-c4ccccc4)cc3)c3ccc(-c4ccccc4)cc3)cc2)cc1]

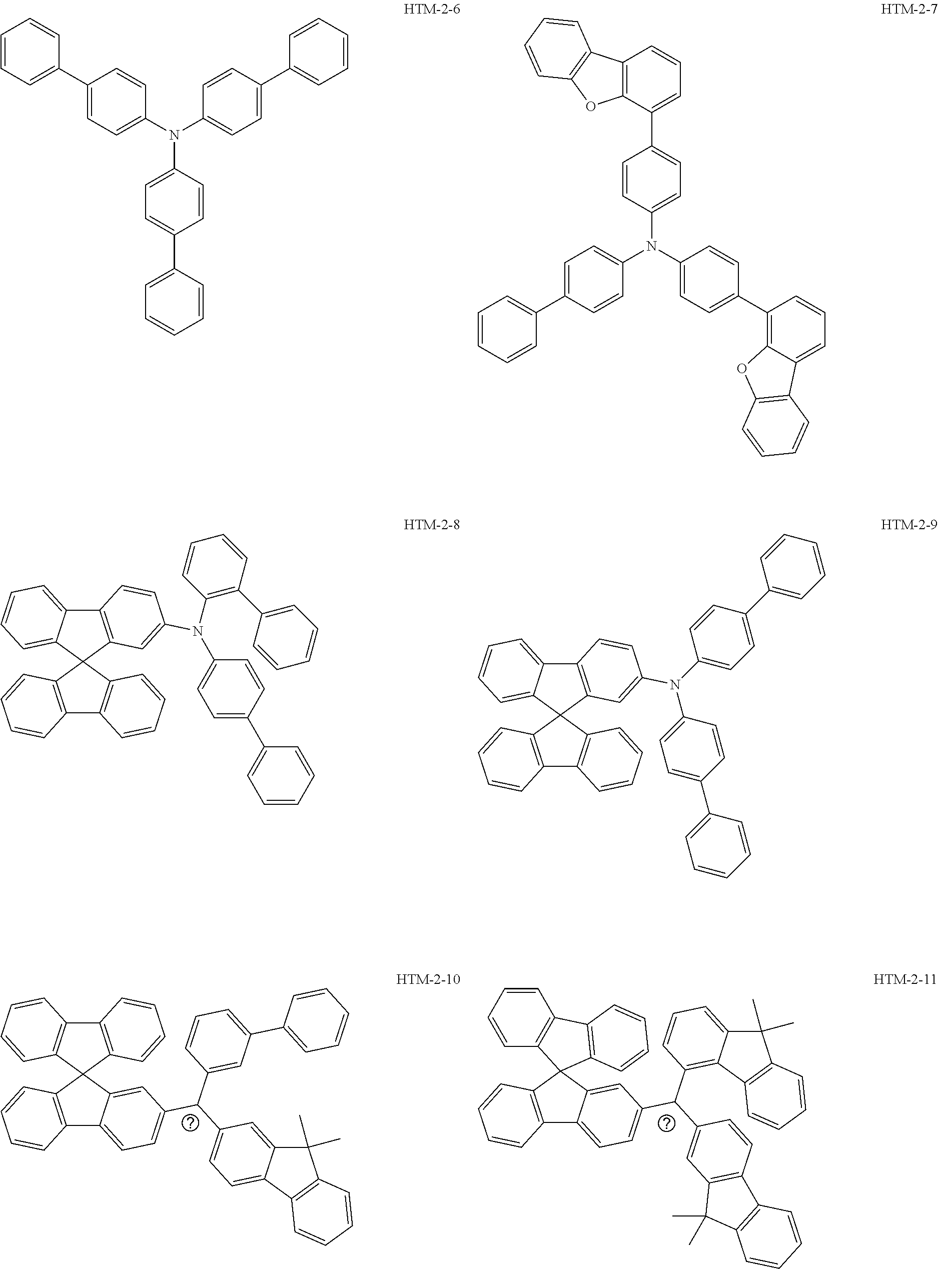

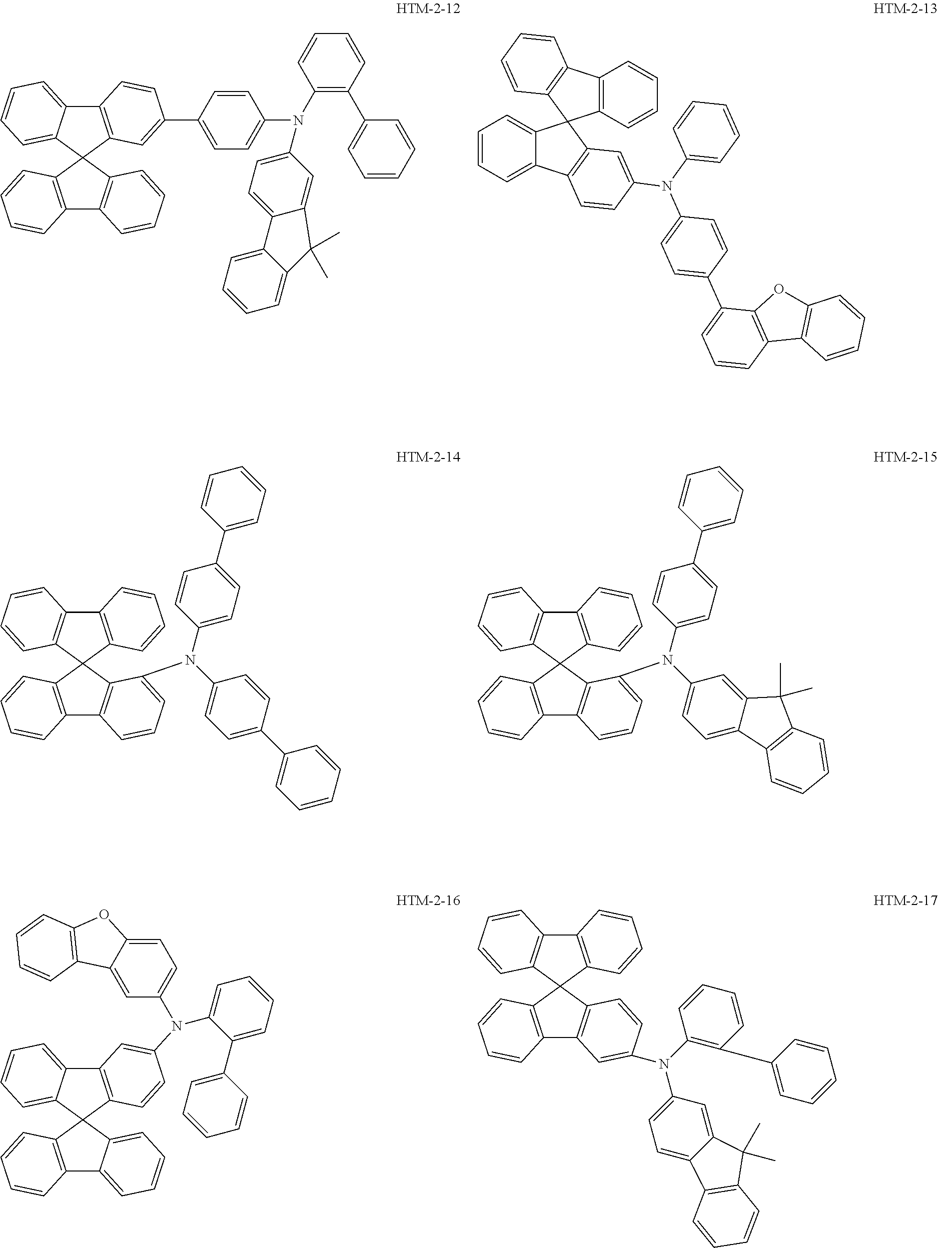

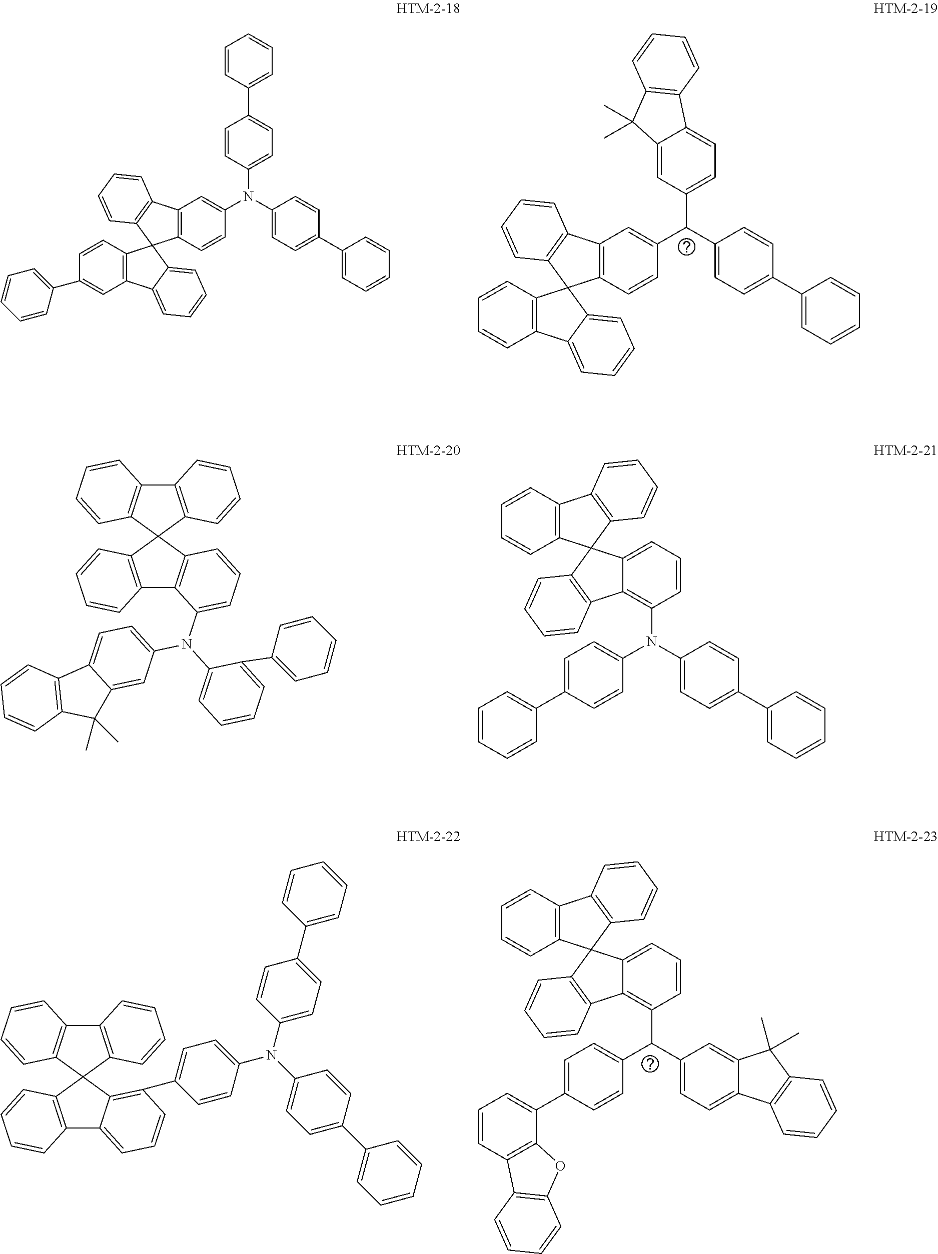

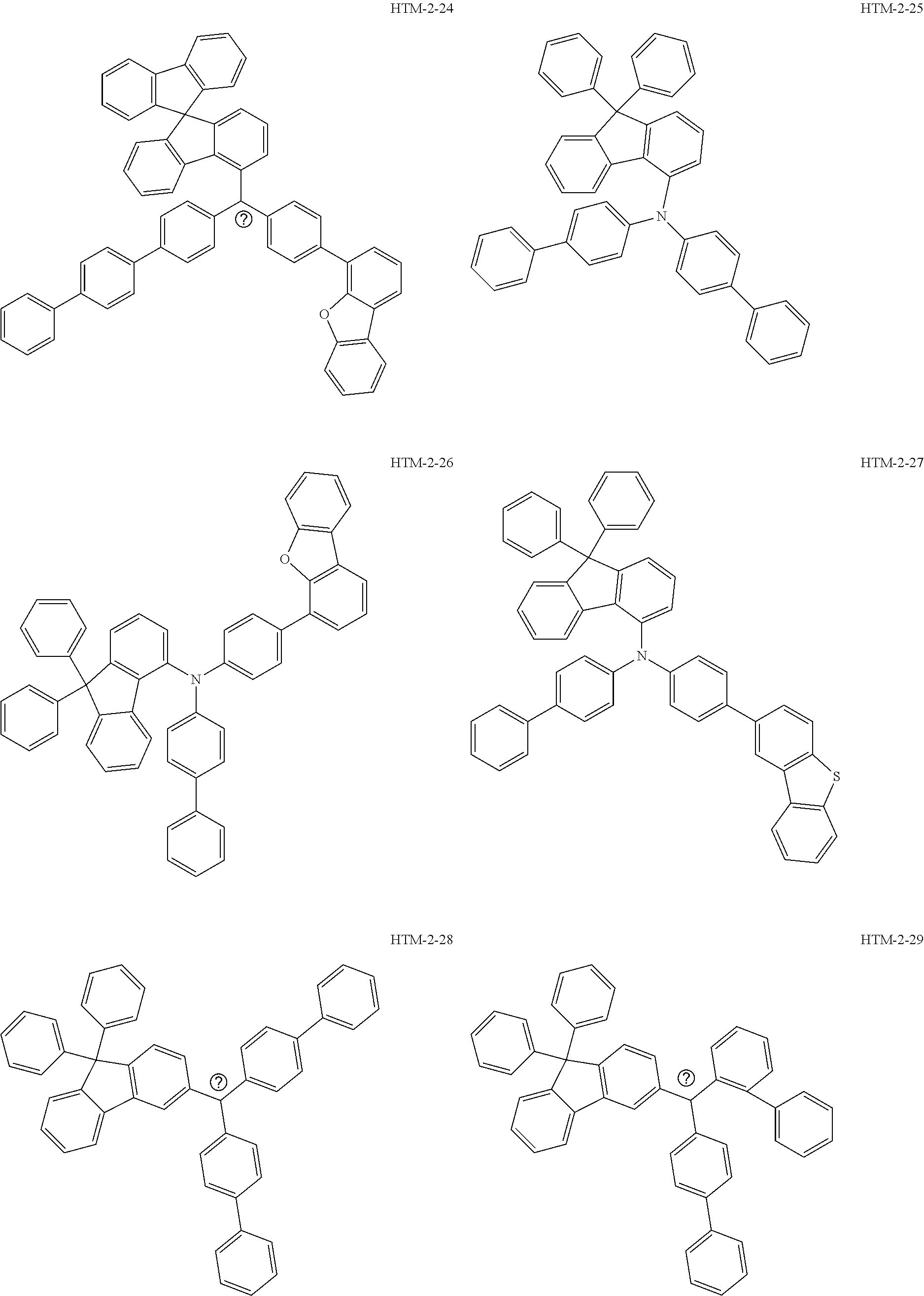

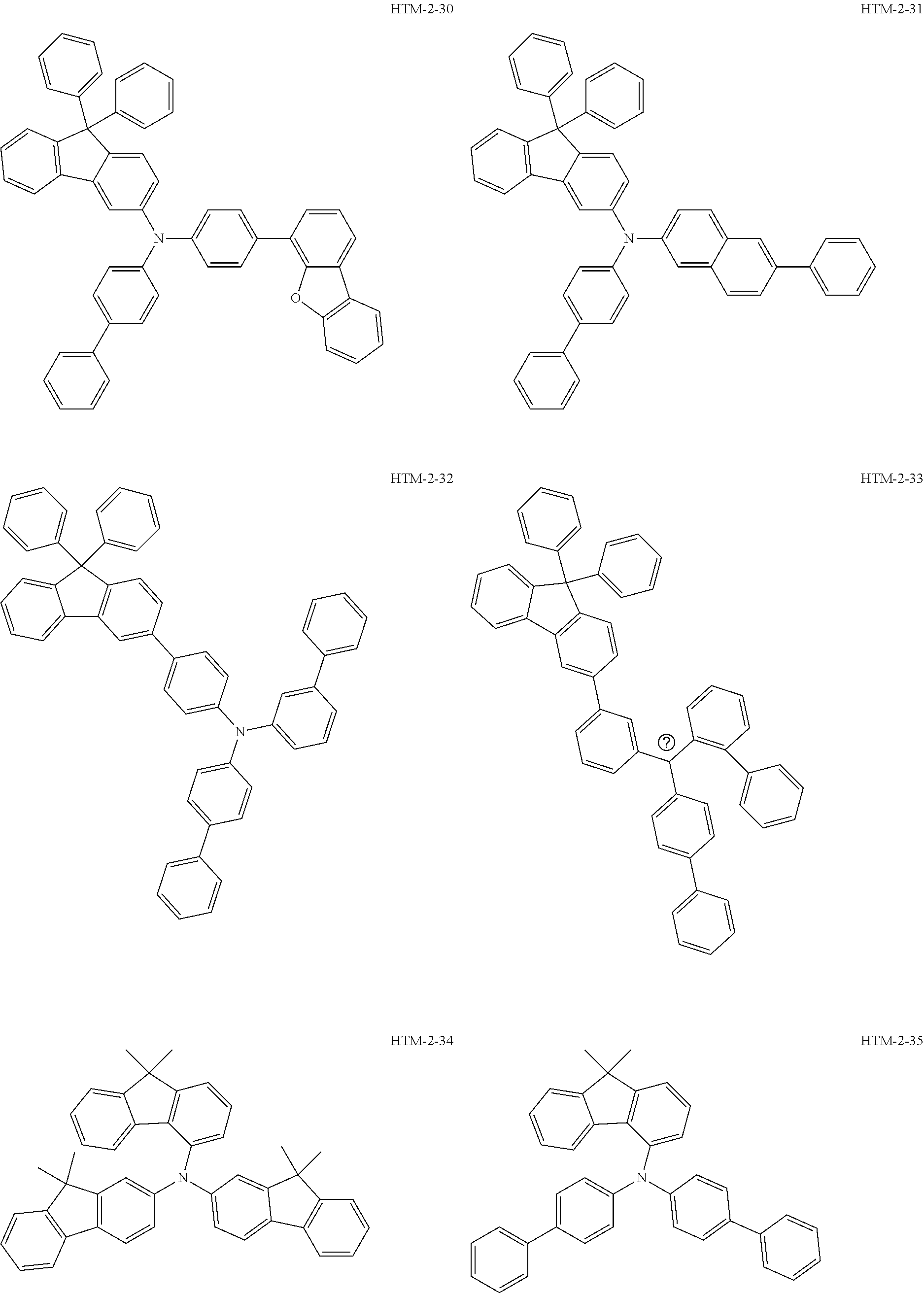

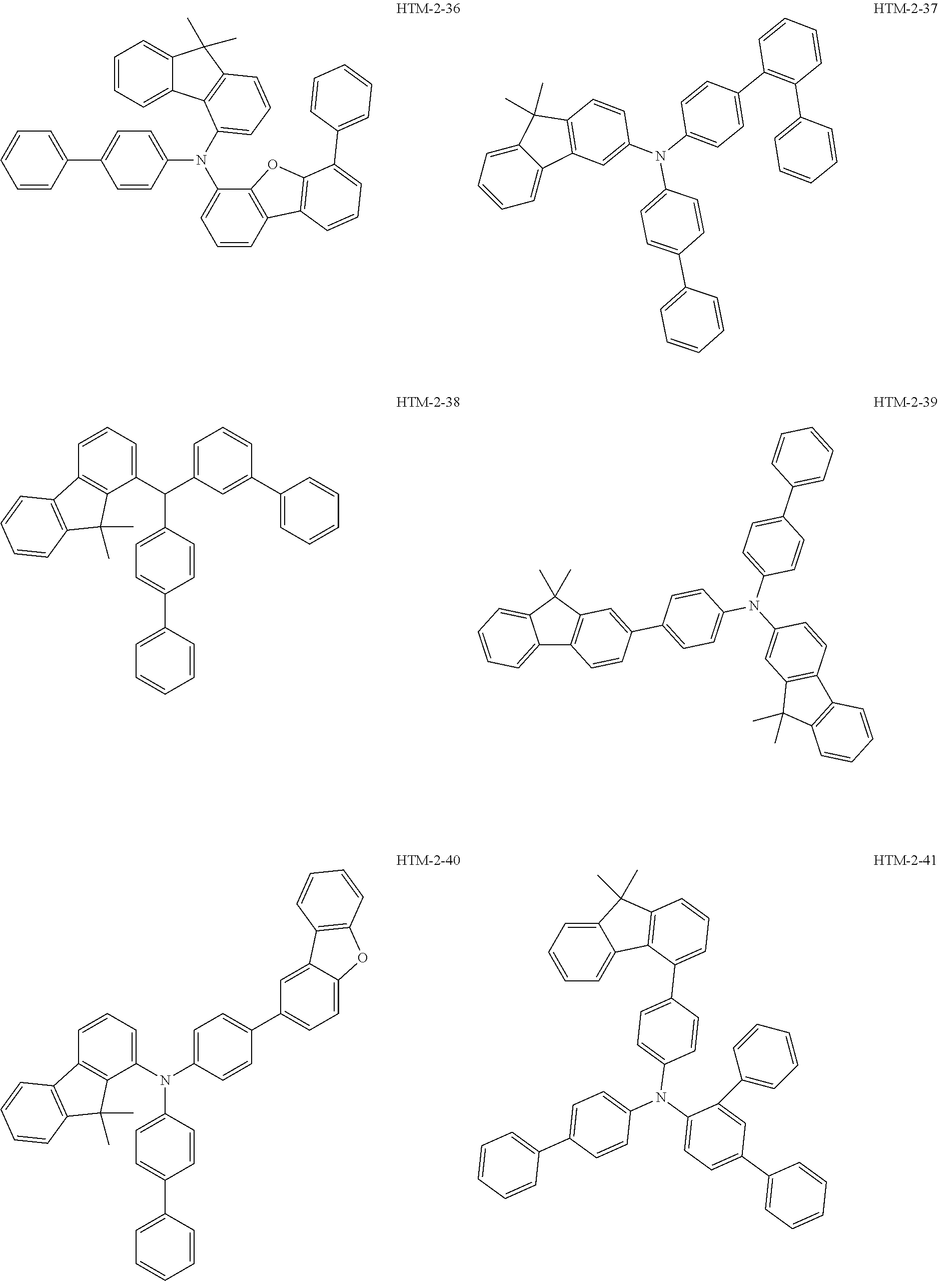

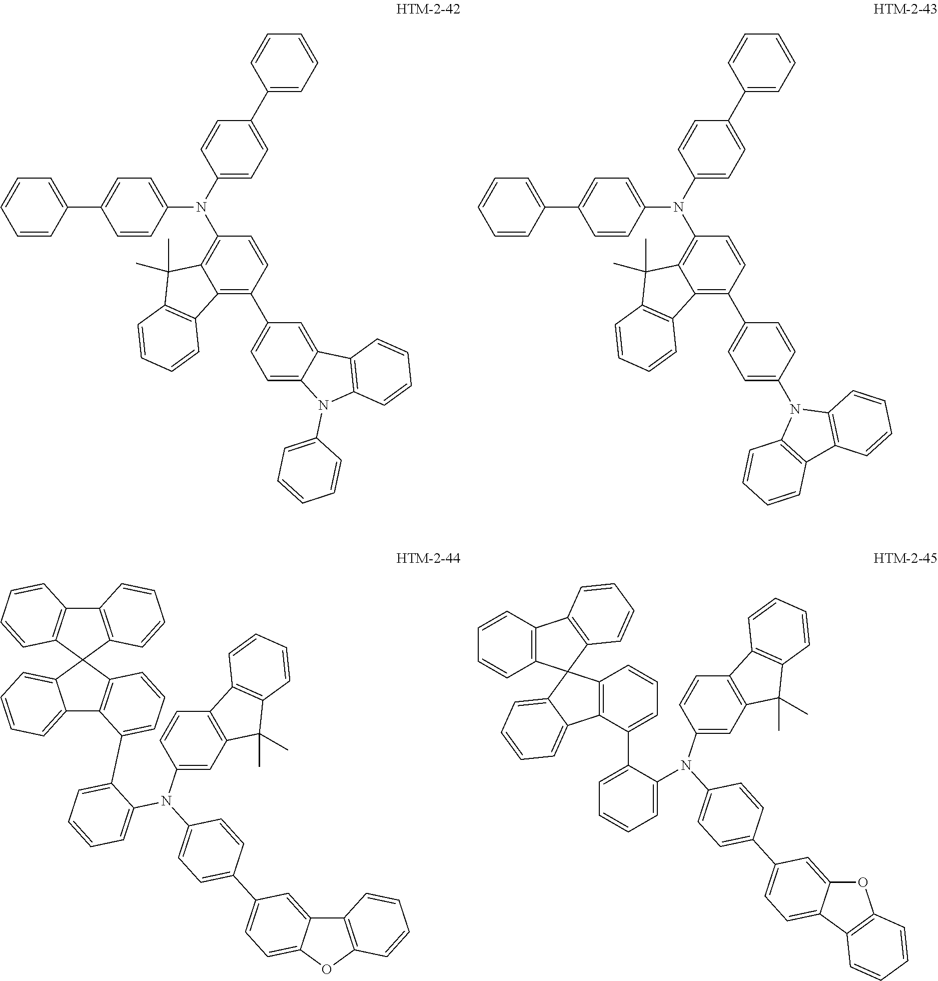 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21 ; CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21 ; CC1(C)c2ccccc2-c2ccc(N(c3ccc(-c4ccc5oc6ccccc6c5c4)cc3)c3ccccc3-c3cccc4c3-c3ccccc3C43c4ccccc4-c4ccccc43)cc21 ; CC1(C)c2ccccc2-c2ccc(N(c3ccc(-c4ccc5c(c4)oc4ccccc45)cc3)c3ccccc3-c3cccc4c3-c3ccccc3C43c4ccccc4-c4ccccc43)cc21]

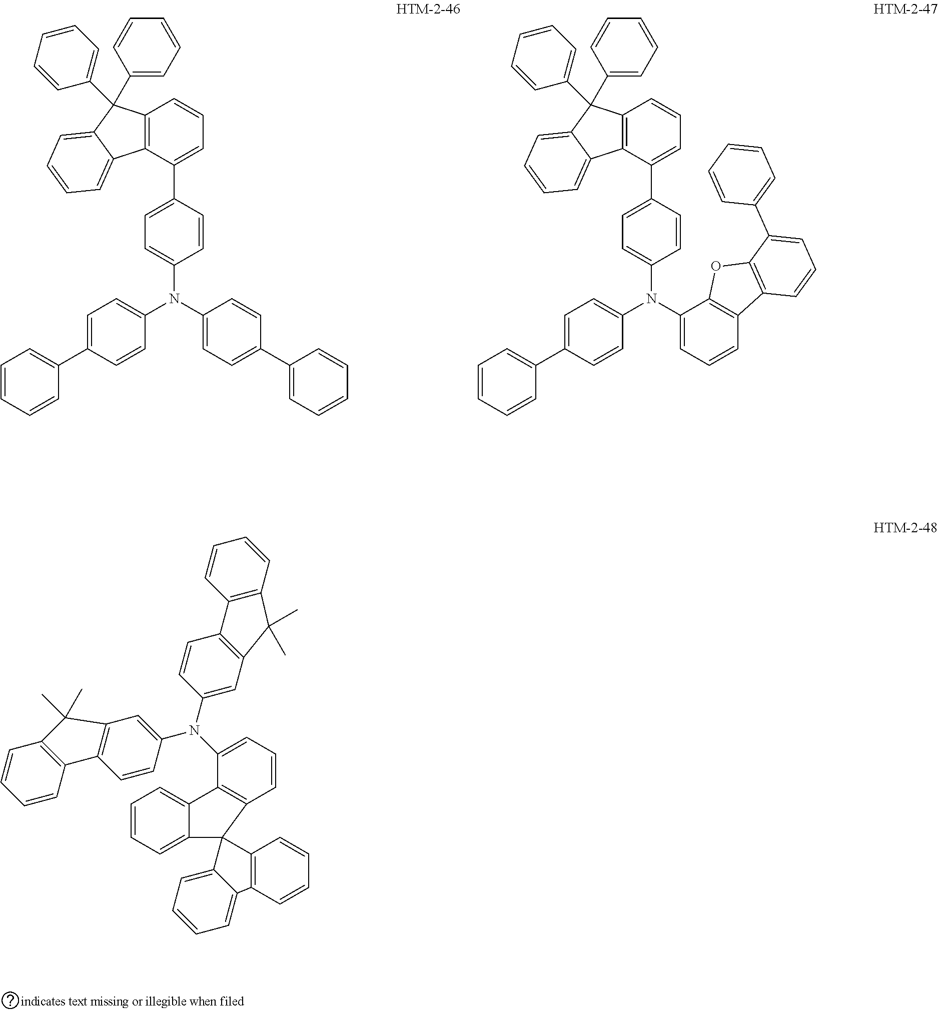 [SMILES: c1ccc(-c2ccc(N(c3ccc(-c4ccccc4)cc3)c3ccc(-c4cccc5c4-c4ccccc4C5(c4ccccc4)c4ccccc4)cc3)cc2)cc1 ; c1ccc(-c2ccc(N(c3ccc(-c4cccc5c4-c4ccccc4C5(c4ccccc4)c4ccccc4)cc3)c3cccc4c3oc3c(-c5ccccc5)cccc34)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21]

The hole injection layer of the electronic device preferably directly adjoins the anode. It is further preferable that it directly adjoins the hole-transporting layer on the anode side. More preferably, the electronic device has the layer sequence anode/hole injection layer/hole-transporting layer/emitting layer, where the layers mentioned directly adjoin one another.

The hole injection layer preferably has a thickness of 2 to 50 nm, more preferably of 2 to 30 nm. It preferably has a thickness of not more than 50 nm, more preferably not more than 30 nm.

In a preferred embodiment, the hole injection layer contains a mixture of a p-dopant, as described above, and a hole transport material. The p-dopant is preferably present here in a proportion of 1% to 10% in the hole injection layer. The hole transport material here is preferably selected from material classes known to the person skilled in the art for hole transport materials for OLEDs, especially triarylamines. Particular preference is given to indenofluoreneamine derivatives, amine derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups.

Preferred specific compounds for use as hole transport material in the hole injection layer are shown in the following table:

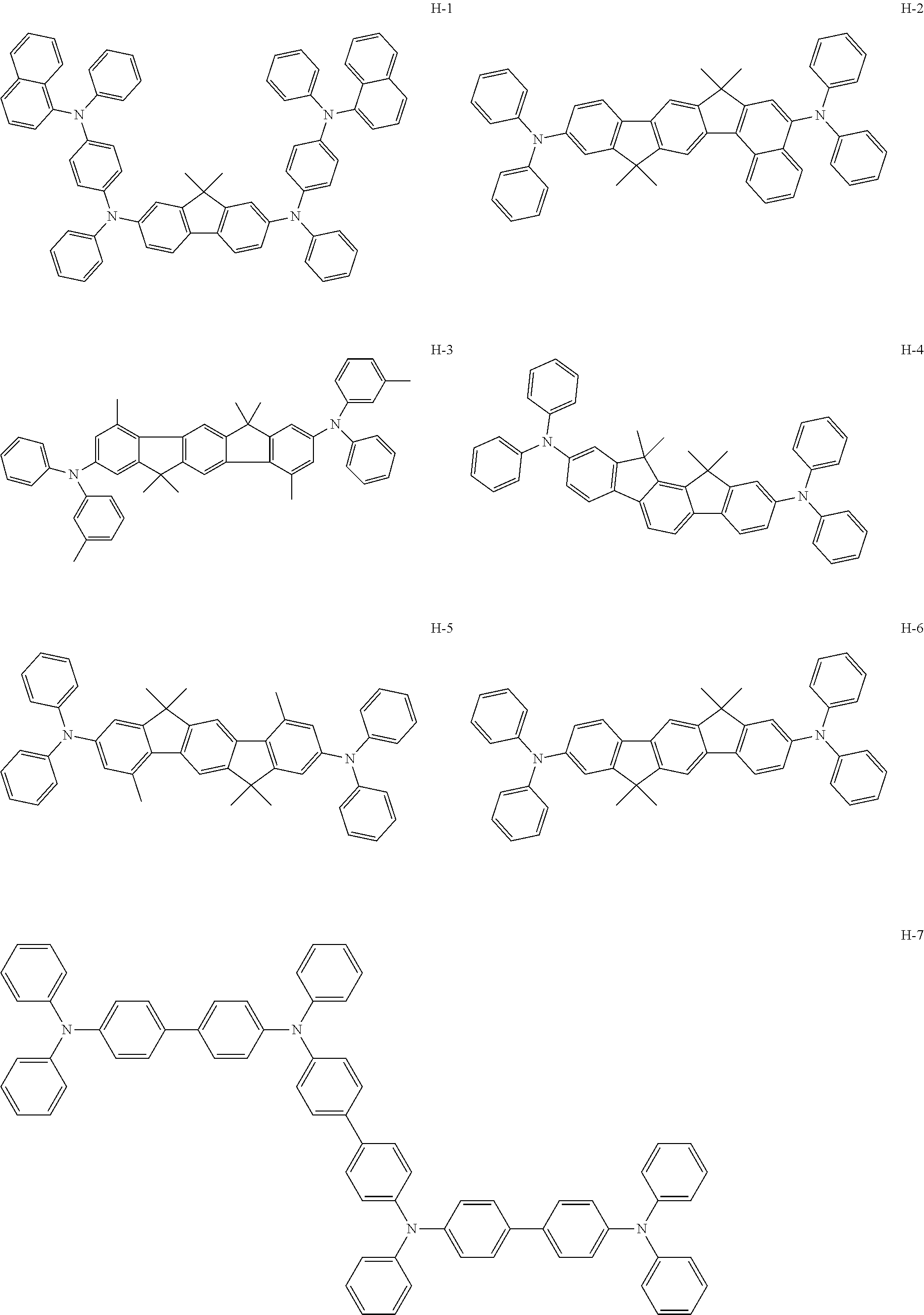

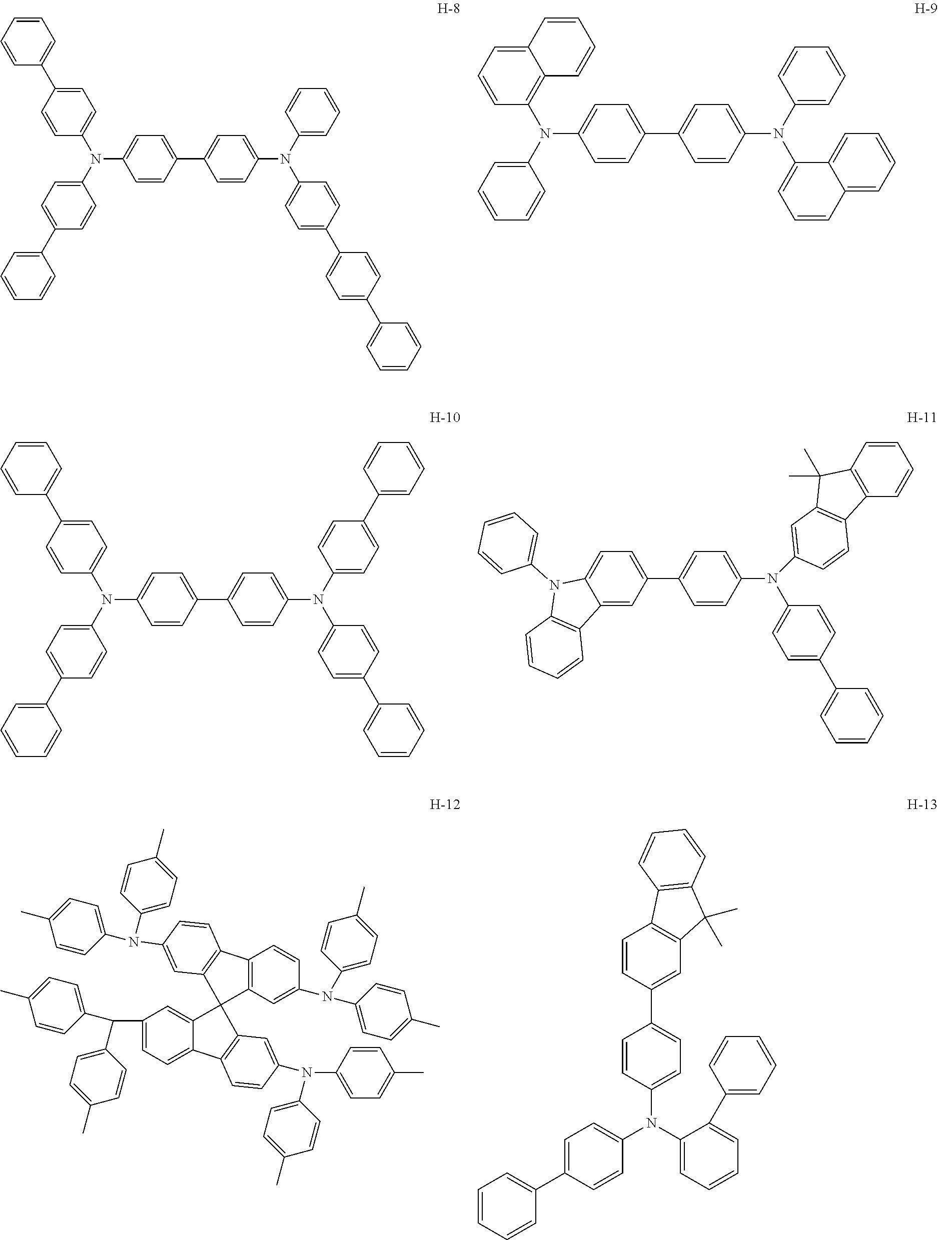

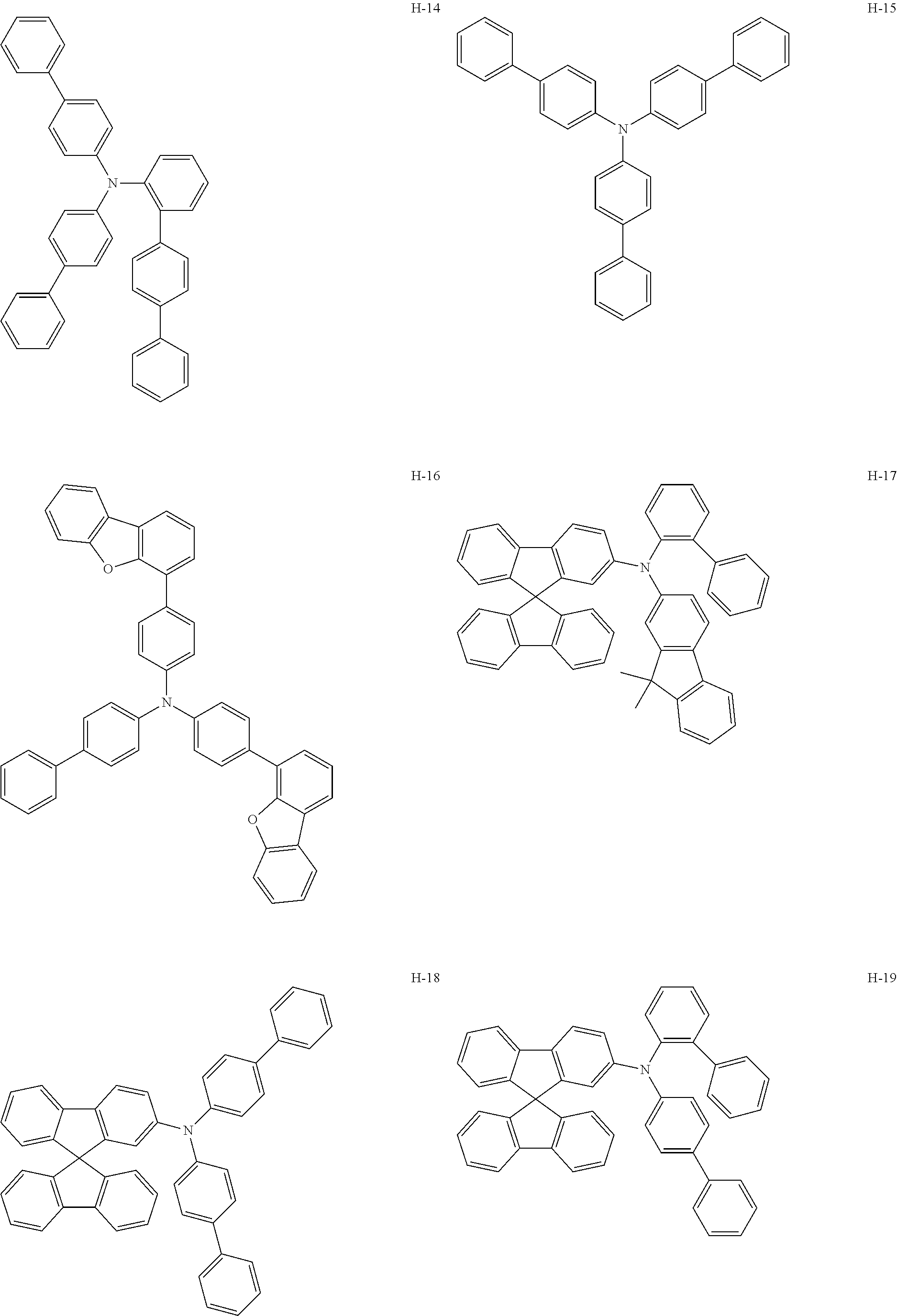 [SMILES: c1ccc(-c2ccc(-c3ccccc3N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4ccccc4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc4c(c3)C3(c5ccccc5-c5ccccc53)c3ccccc3-4)cc2)cc1 ; c1ccc(-c2ccc(N(c3ccc4c(c3)C3(c5ccccc5-c5ccccc53)c3ccccc3-4)c3ccccc3-c3ccccc3)cc2)cc1]

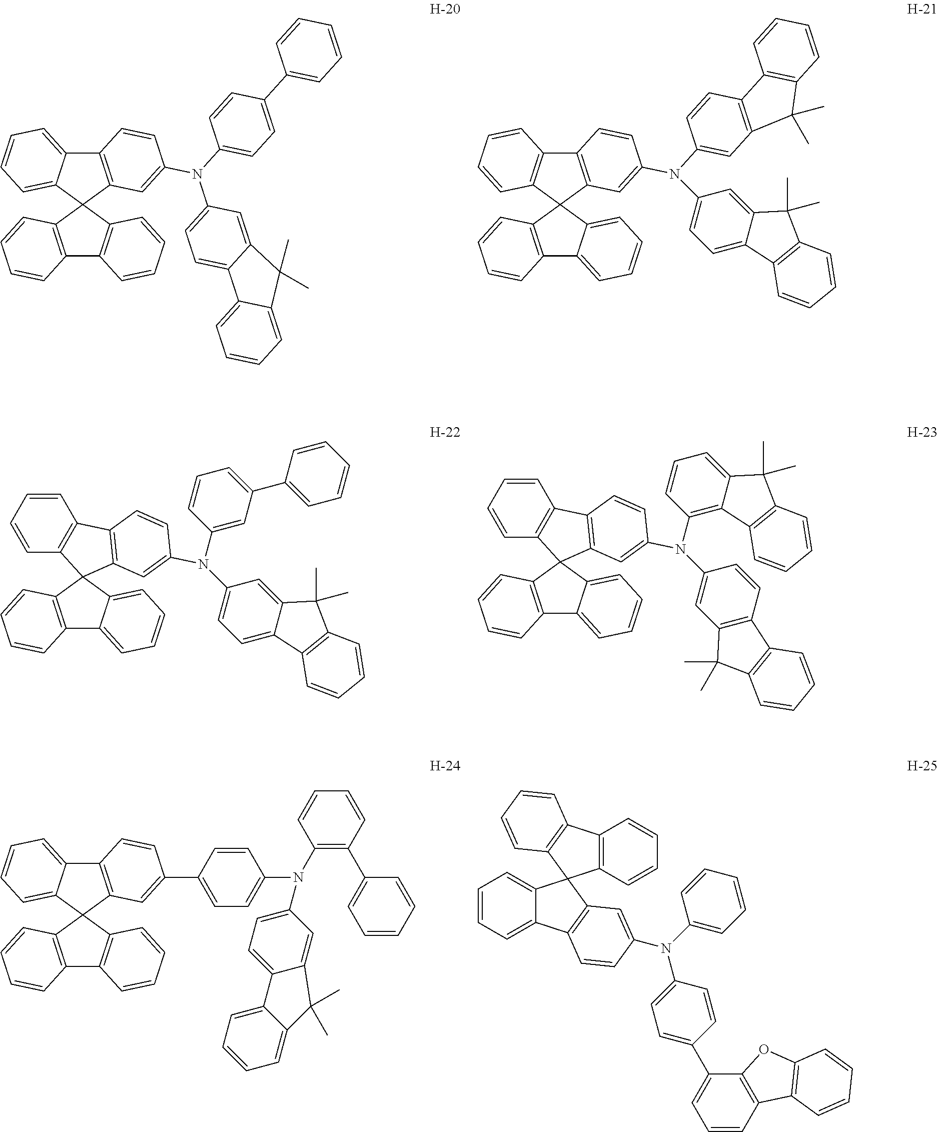

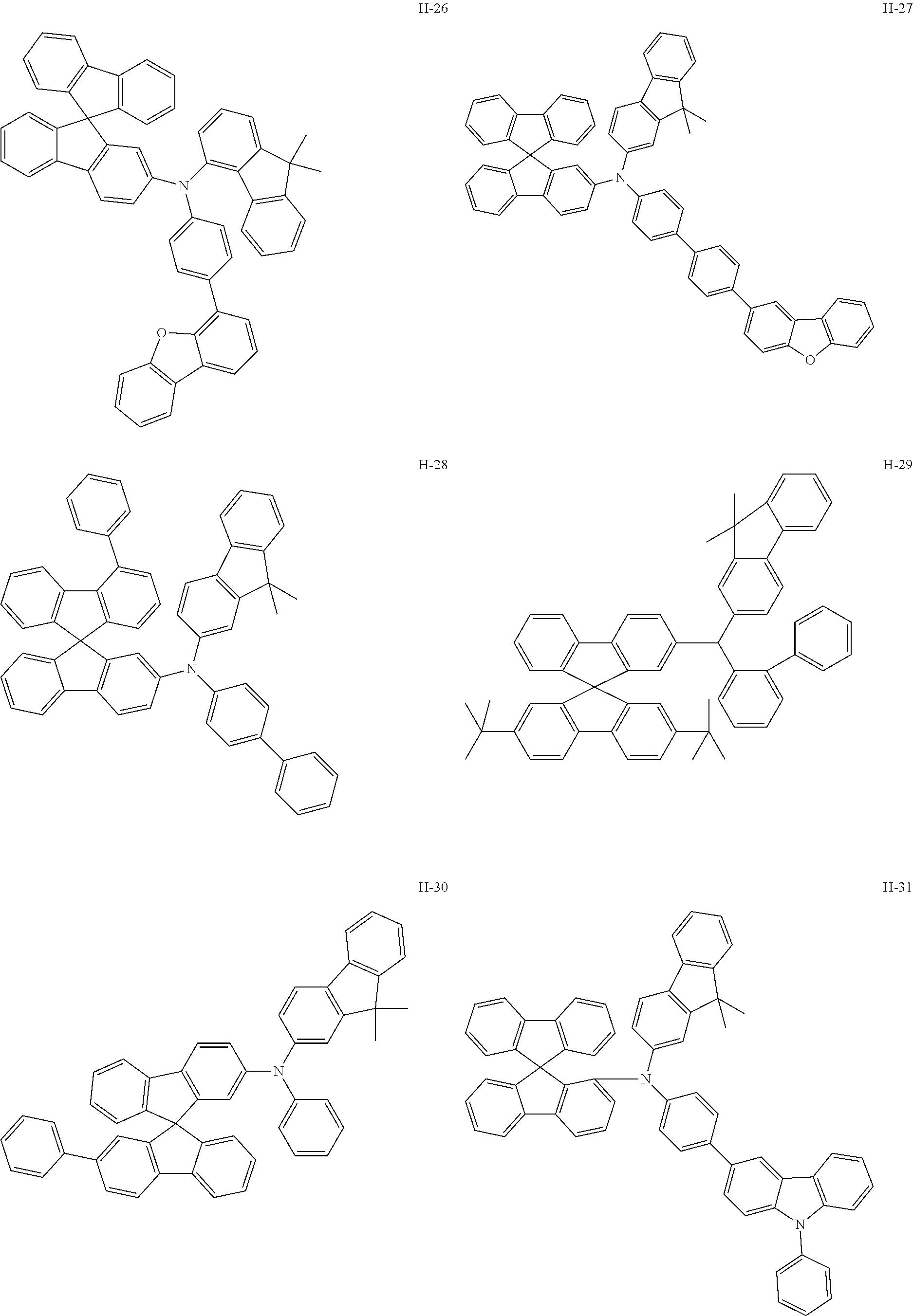

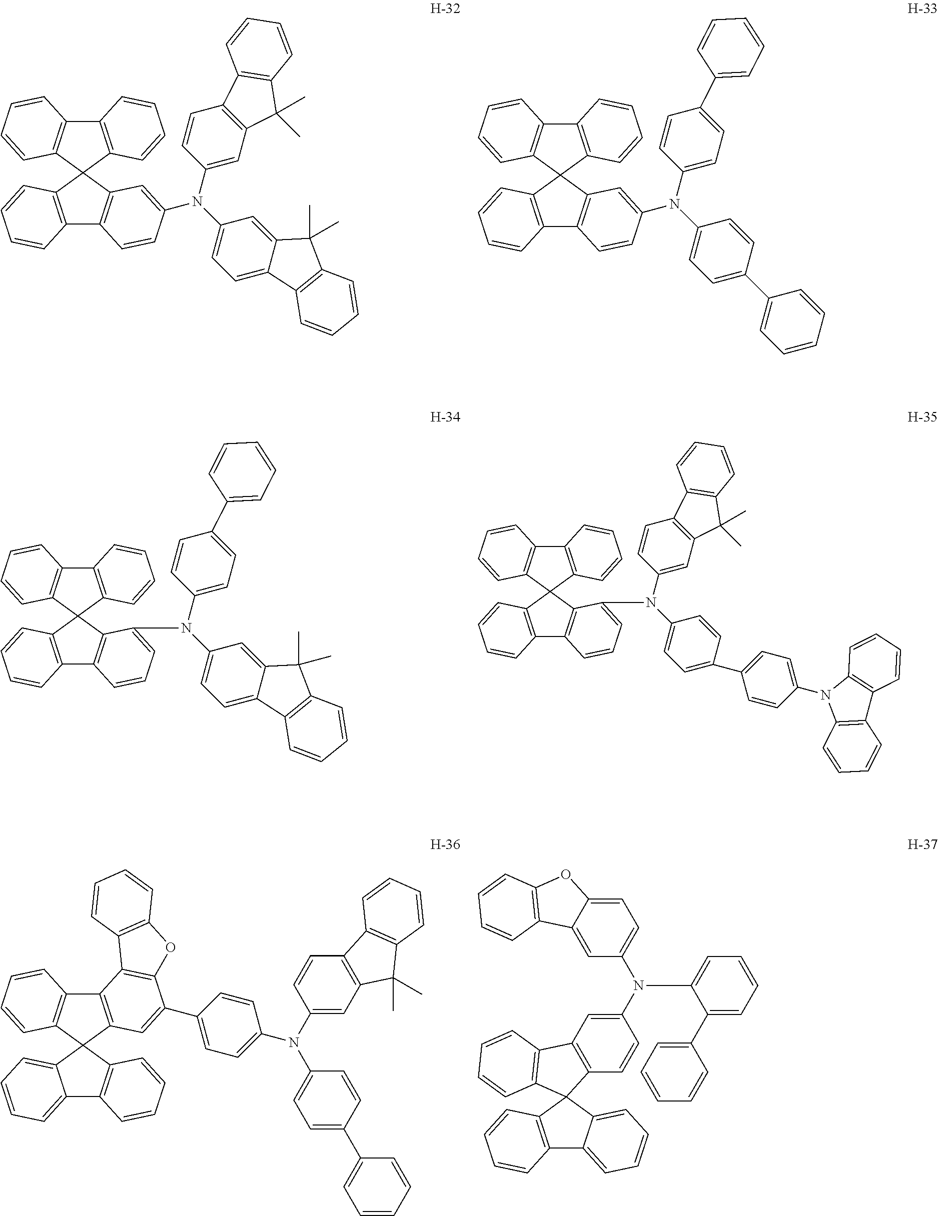

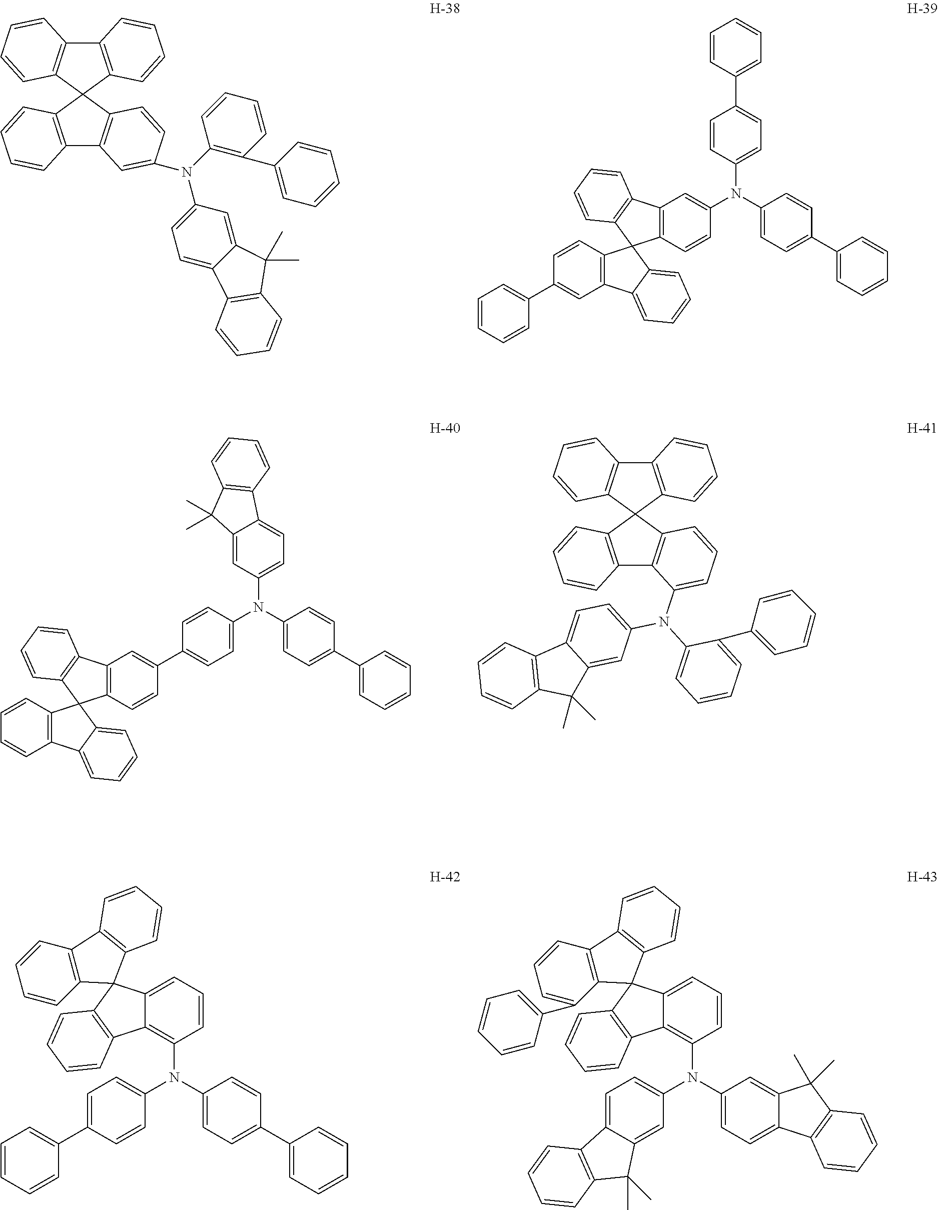

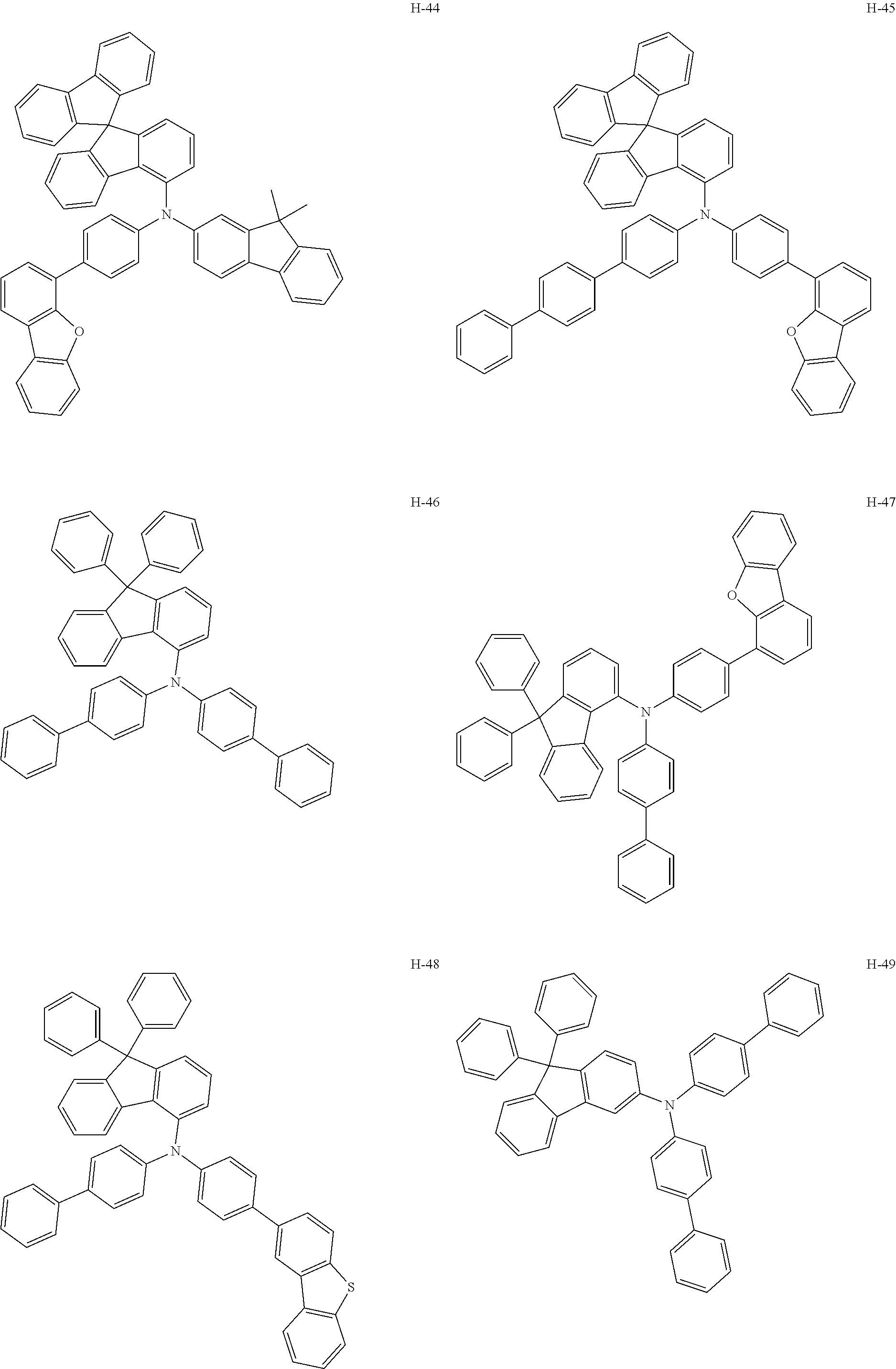

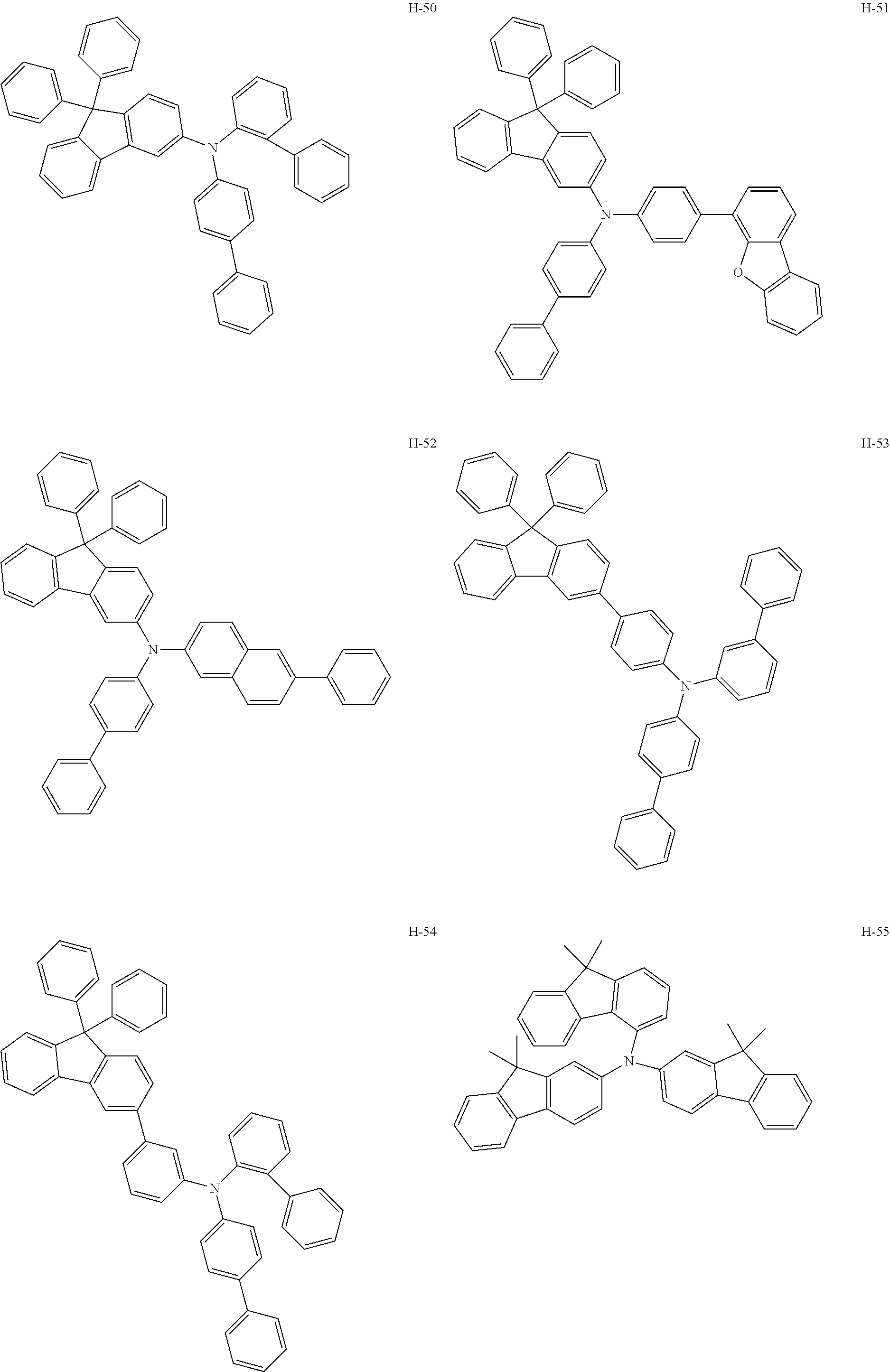

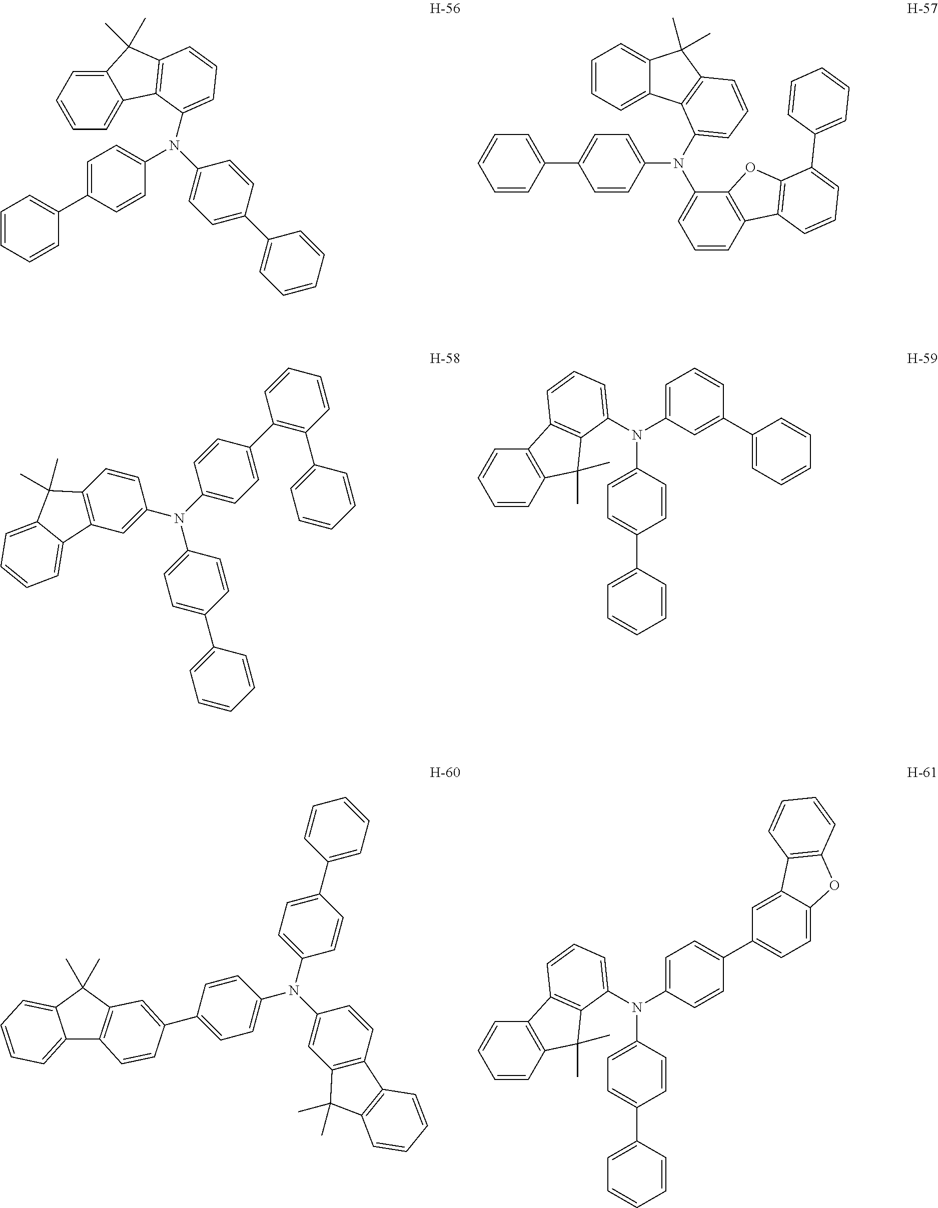 [SMILES: CC1(C)c2ccccc2-c2c(N(c3ccc(-c4ccccc4)cc3)c3ccc(-c4ccccc4)cc3)cccc21 ; CC1(C)c2ccccc2-c2c(N(c3ccc(-c4ccccc4)cc3)c3cccc4c3oc3c(-c5ccccc5)cccc34)cccc21 ; CC1(C)c2ccccc2-c2cc(N(c3ccc(-c4ccccc4)cc3)c3ccc(-c4ccccc4-c4ccccc4)cc3)ccc21 ; CC1(C)c2ccccc2-c2cccc(N(c3ccc(-c4ccccc4)cc3)c3cccc(-c4ccccc4)c3)c21 ; CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2cccc(N(c3ccc(-c4ccccc4)cc3)c3ccc(-c4ccc5oc6ccccc6c5c4)cc3)c21]

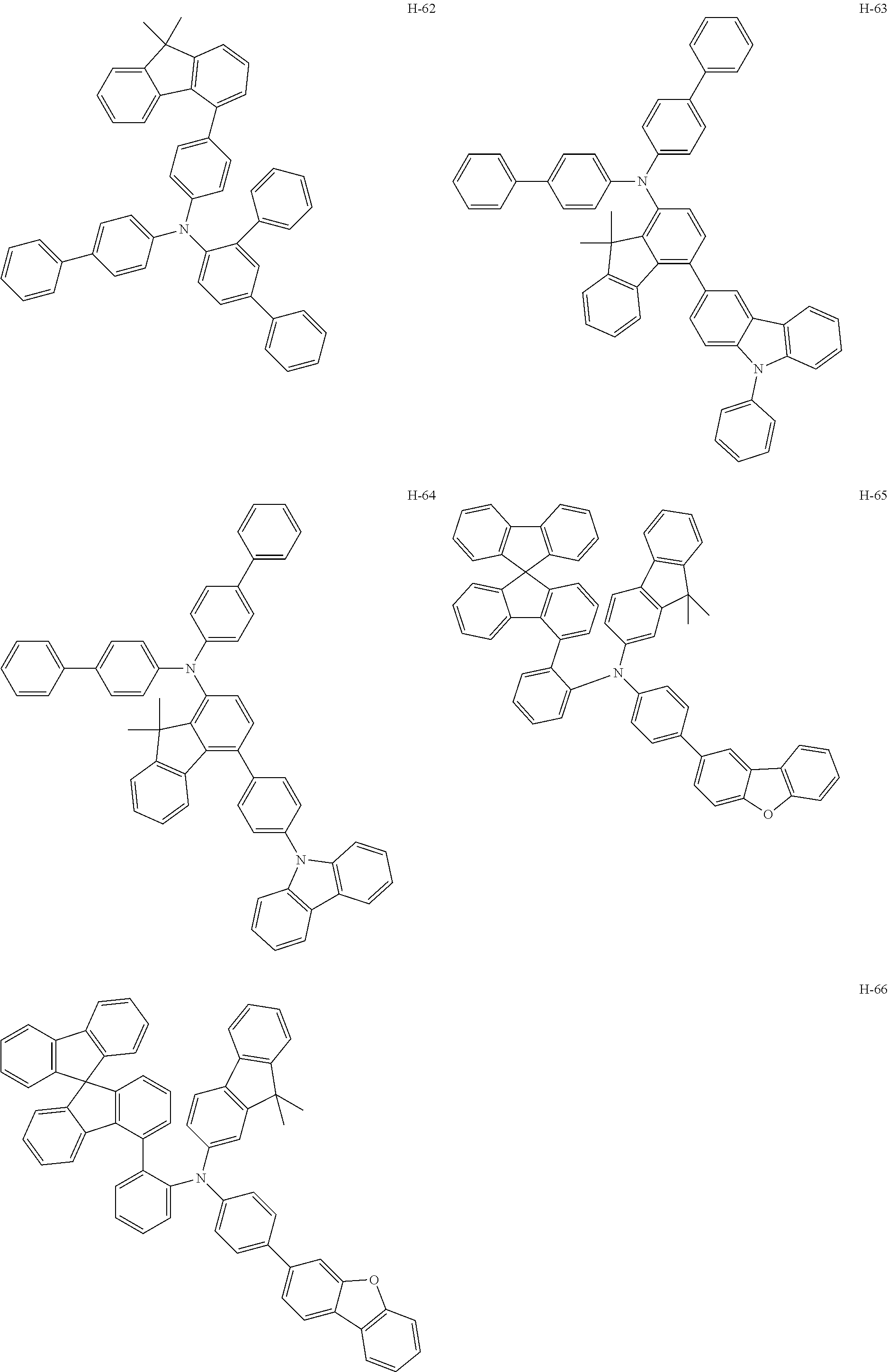

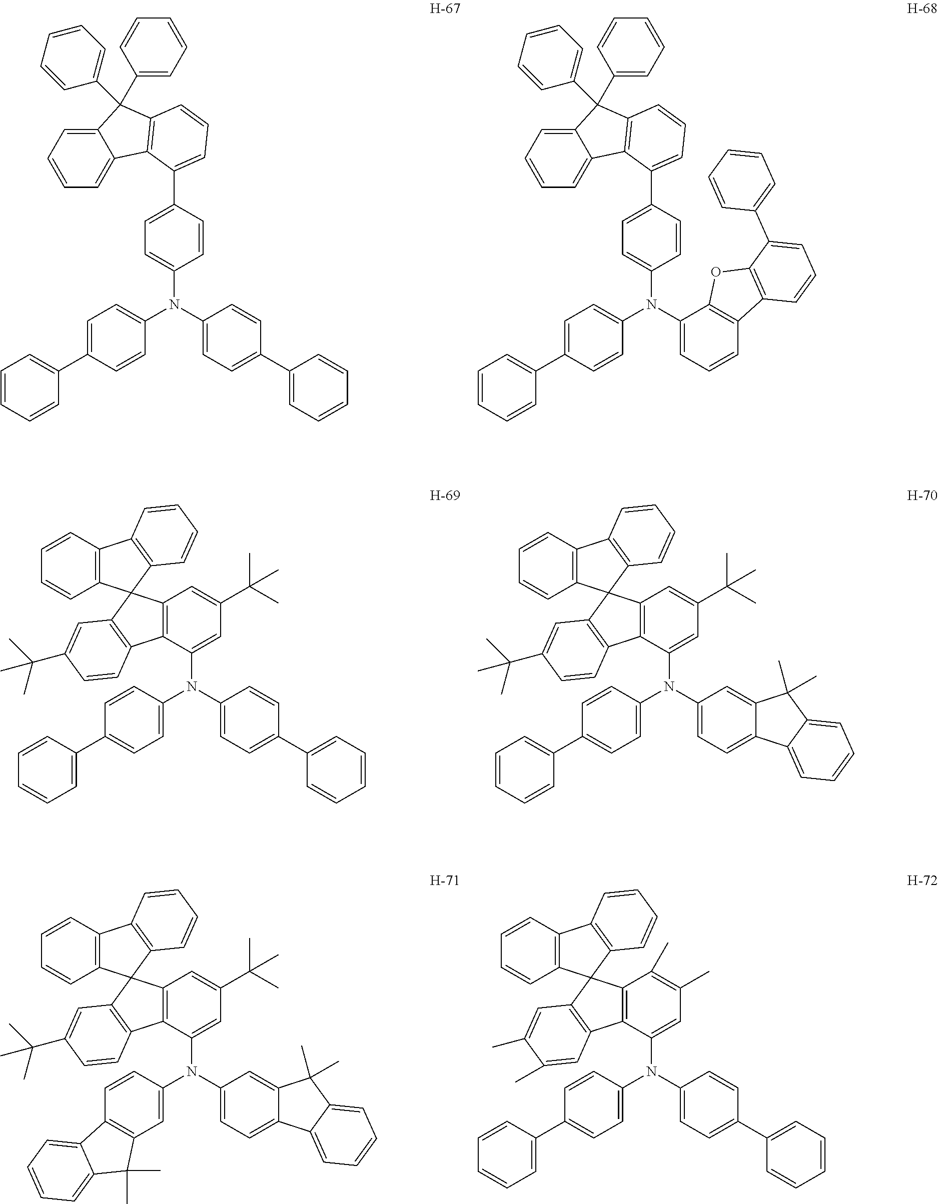

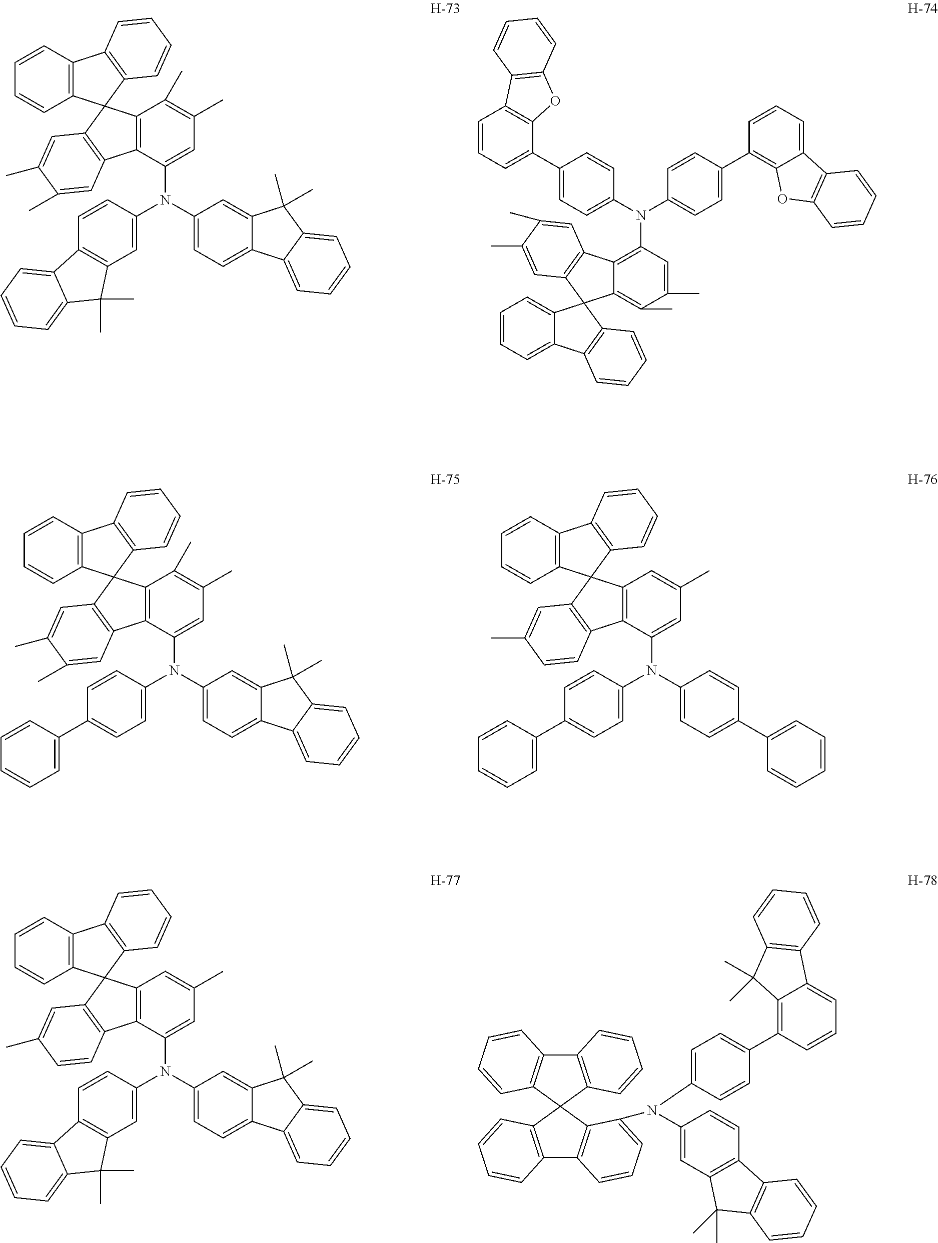

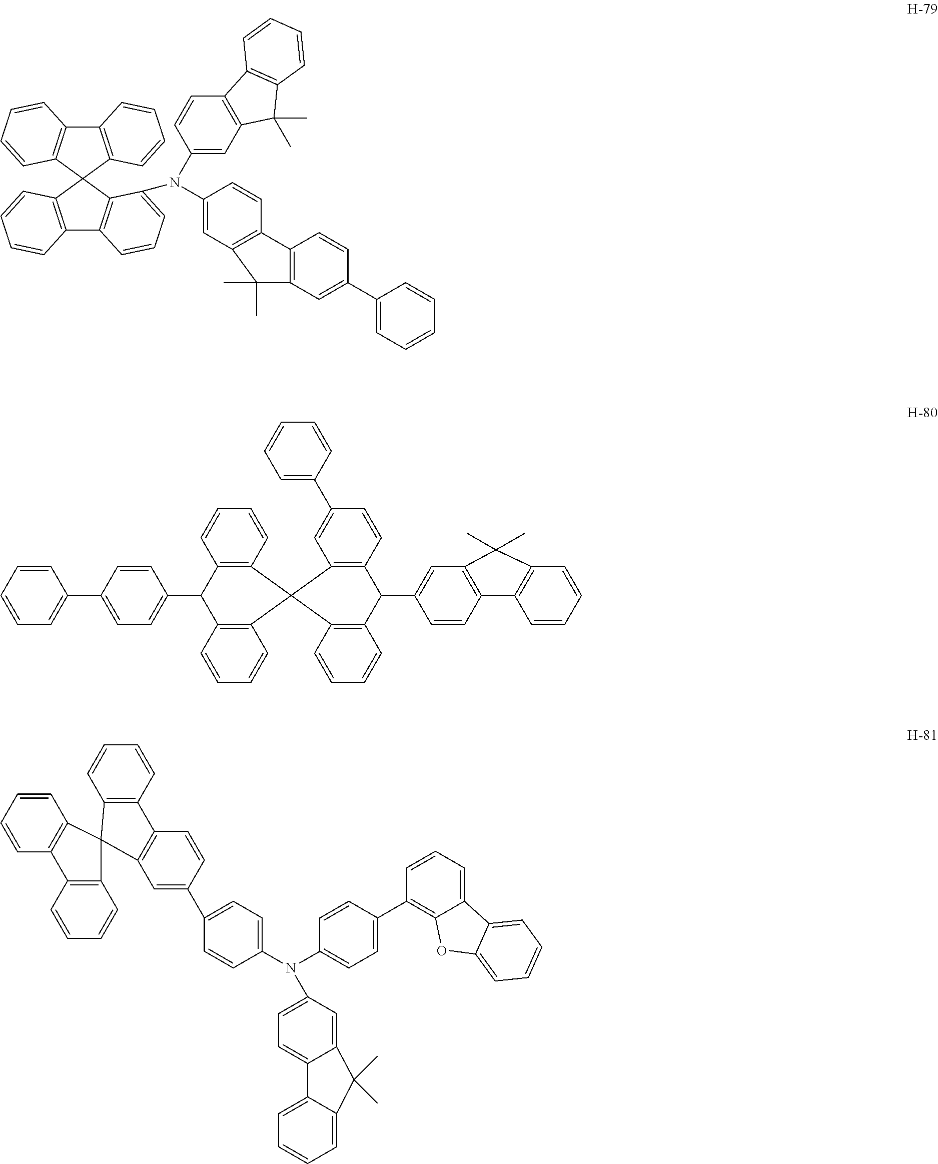

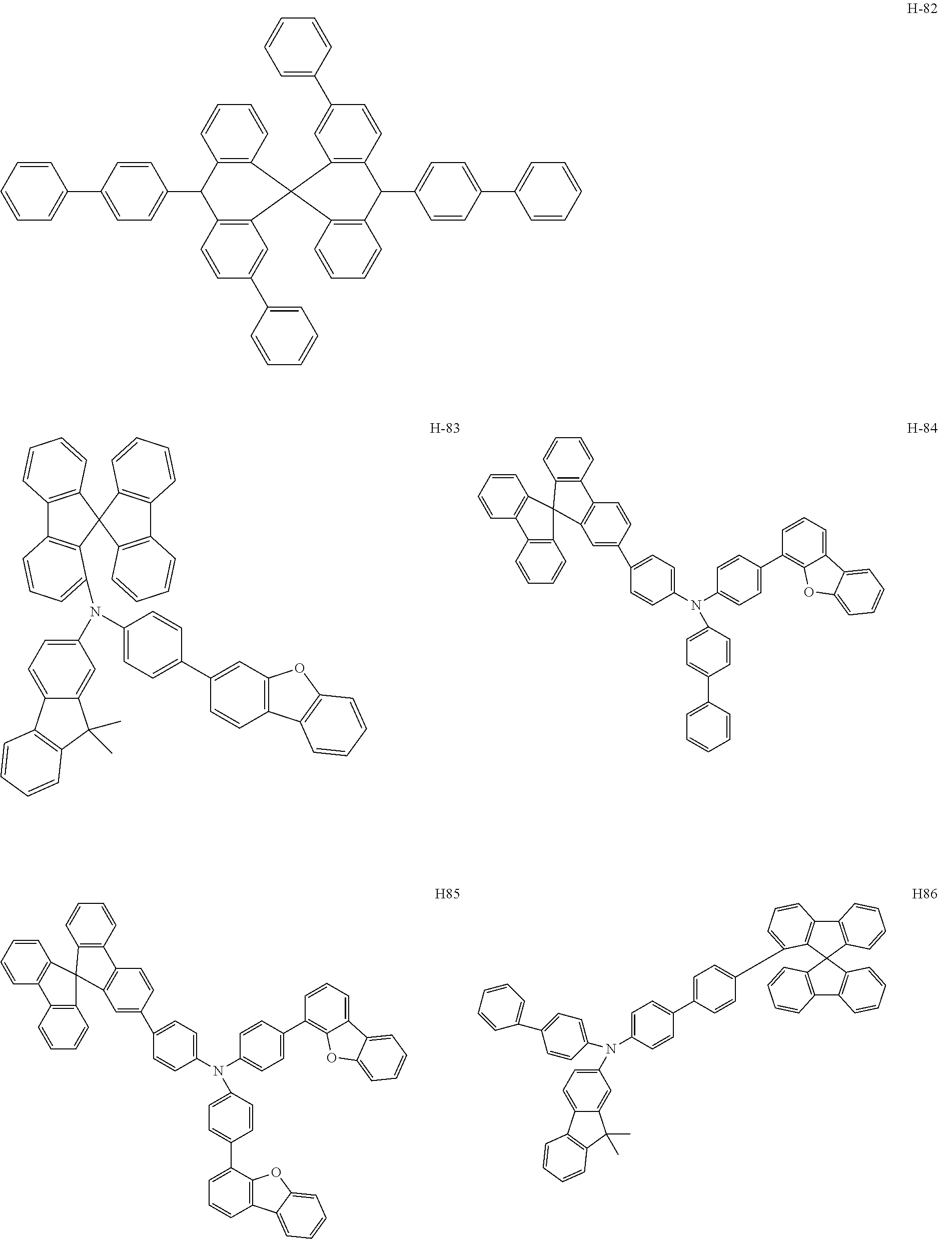

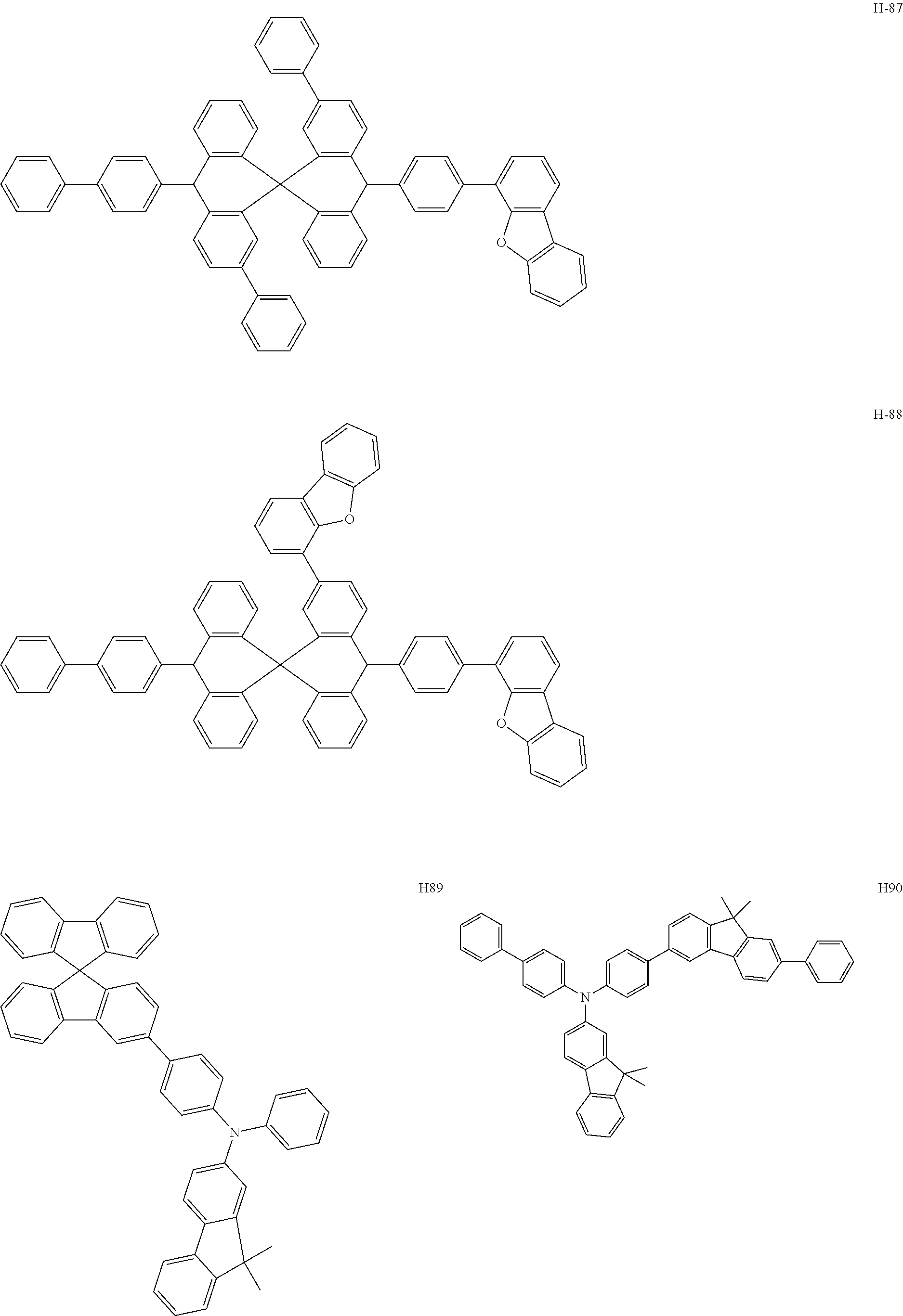 [SMILES: c1ccc(-c2ccc(C3c4ccccc4C4(c5ccccc5C(c5ccc(-c6cccc7c6oc6ccccc67)cc5)c5ccc(-c6ccccc6)cc54)c4cc(-c5ccccc5)ccc43)cc2)cc1 ; c1ccc(-c2ccc(C3c4ccccc4C4(c5ccccc53)c3ccccc3C(c3ccc(-c5cccc6c5oc5ccccc56)cc3)c3ccc(-c5cccc6c5oc5ccccc56)cc34)cc2)cc1 ; CC1(C)c2ccccc2-c2ccc(N(c3ccccc3)c3ccc(-c4ccc5c(c4)-c4ccccc4C54c5ccccc5-c5ccccc54)cc3)cc21 ; CC1(C)c2ccc(-c3ccc(N(c4ccc(-c5ccccc5)cc4)c4ccc5c(c4)C(C)(C)c4ccccc4-5)cc3)cc2-c2ccc(-c3ccccc3)cc21]

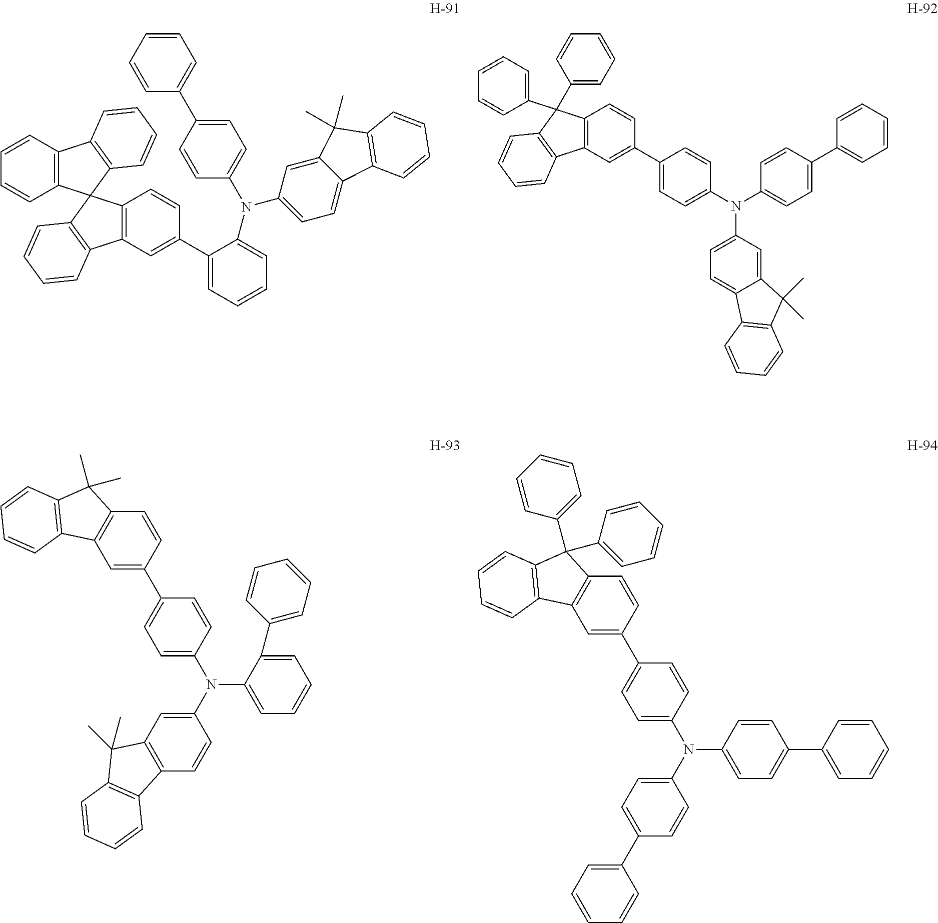 [SMILES: CC1(C)c2ccccc2-c2ccc(N(c3ccc(-c4ccccc4)cc3)c3ccccc3-c3ccc4c(c3)-c3ccccc3C43c4ccccc4-c4ccccc43)cc21 ; CC1(C)c2ccccc2-c2ccc(N(c3ccc(-c4ccccc4)cc3)c3ccc(-c4ccc5c(c4)-c4ccccc4C5(c4ccccc4)c4ccccc4)cc3)cc21 ; CC1(C)c2ccccc2-c2cc(-c3ccc(N(c4ccc5c(c4)C(C)(C)c4ccccc4-5)c4ccccc4-c4ccccc4)cc3)ccc21 ; c1ccc(-c2ccc(N(c3ccc(-c4ccccc4)cc3)c3ccc(-c4ccc5c(c4)-c4ccccc4C5(c4ccccc4)c4ccccc4)cc3)cc2)cc1]

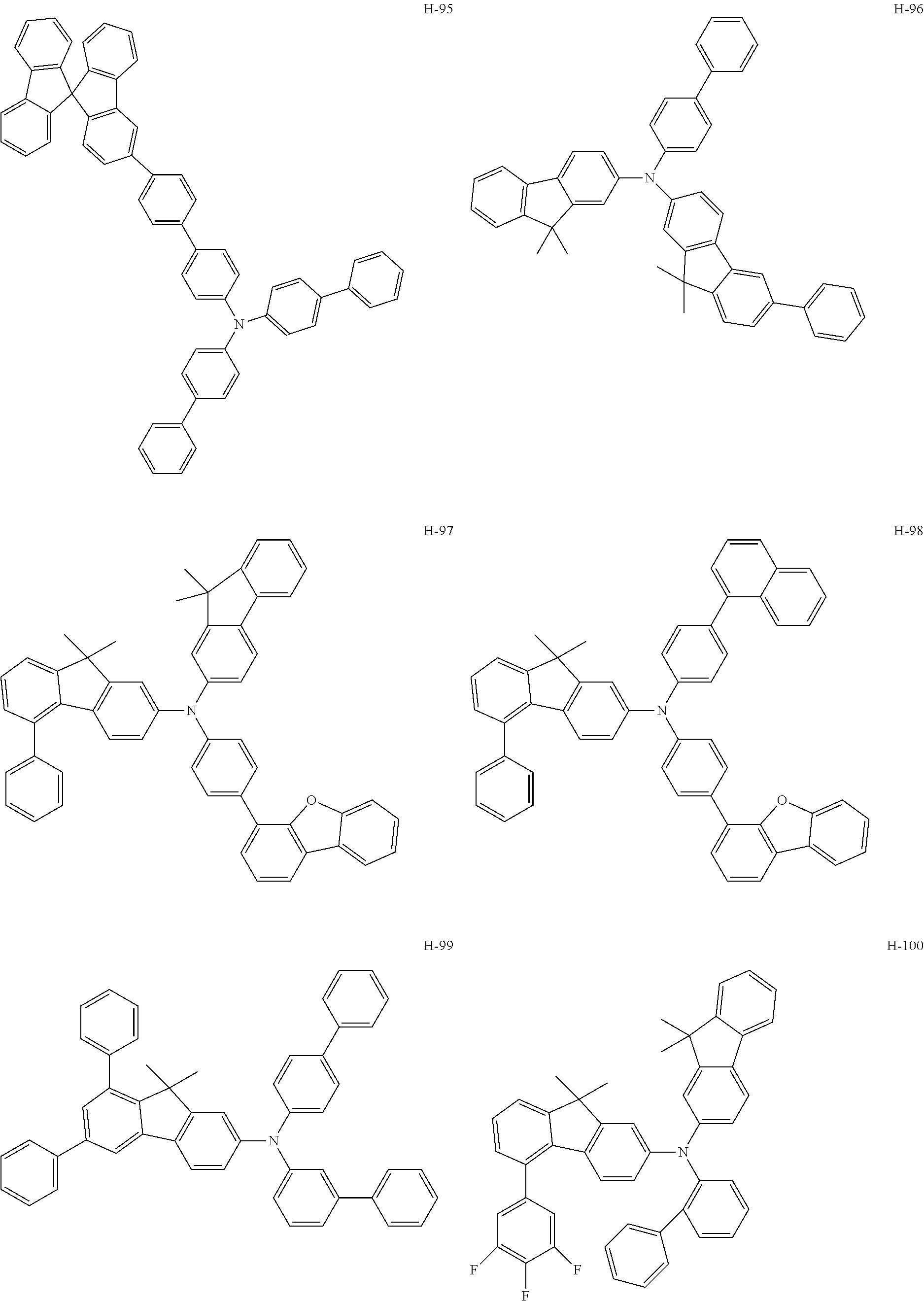

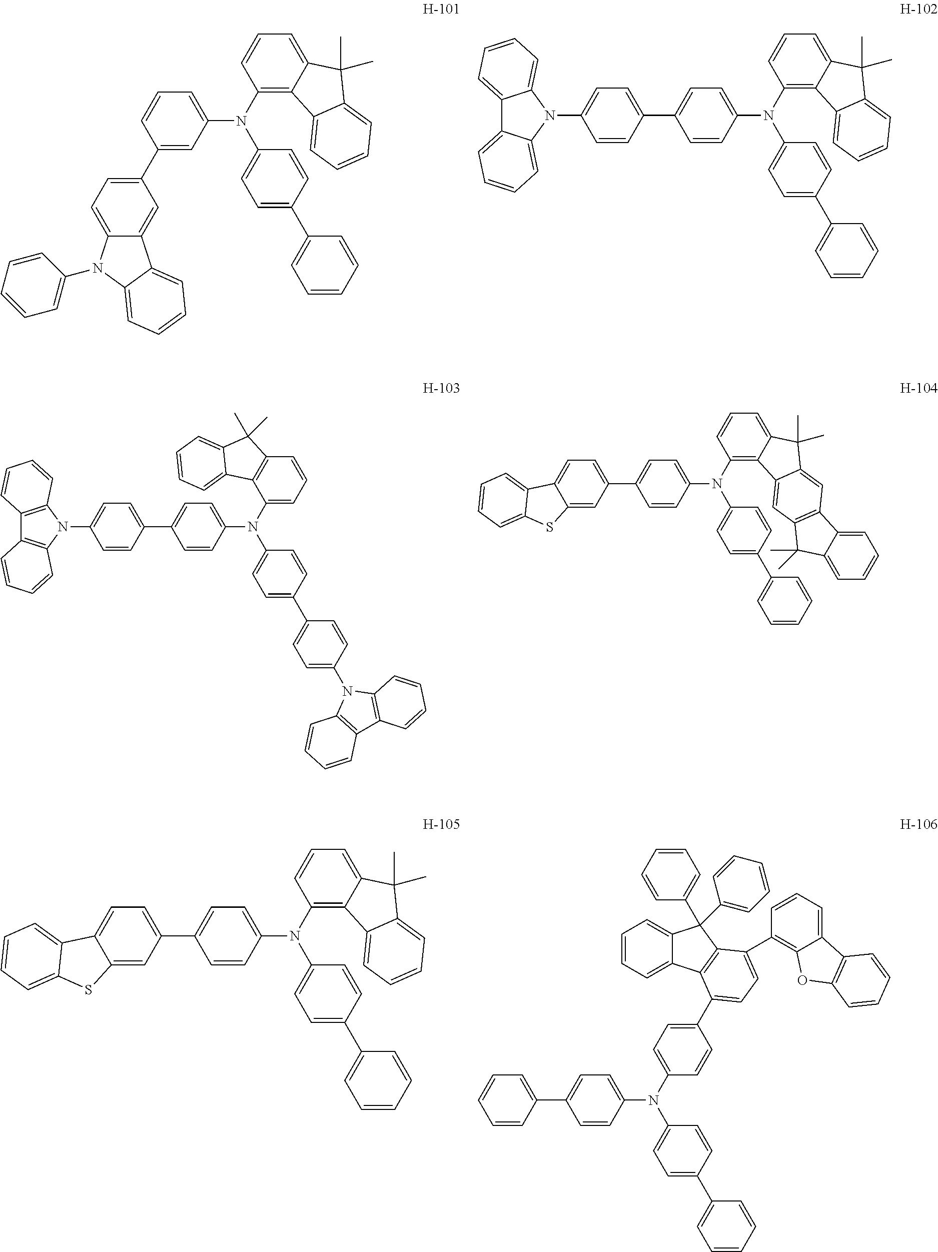

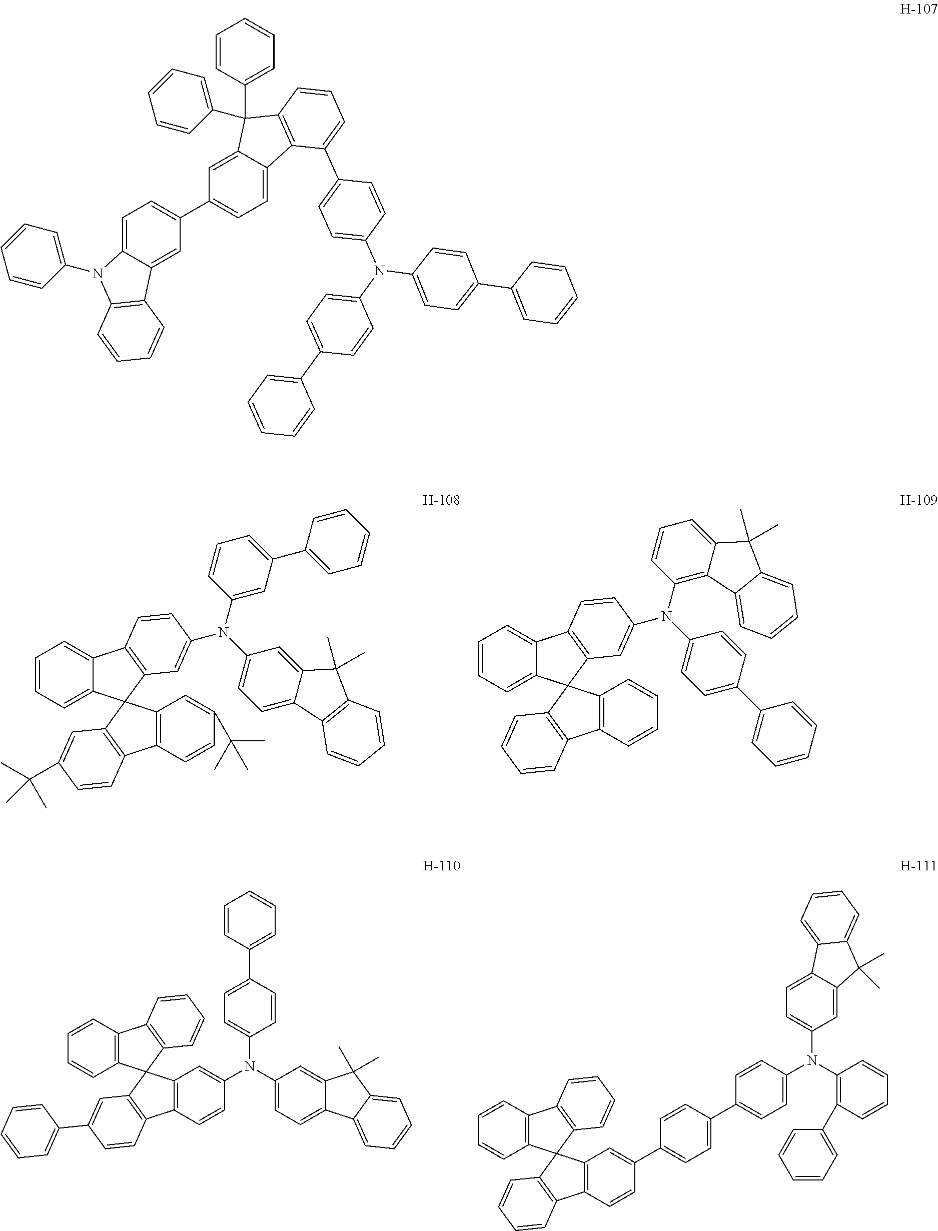

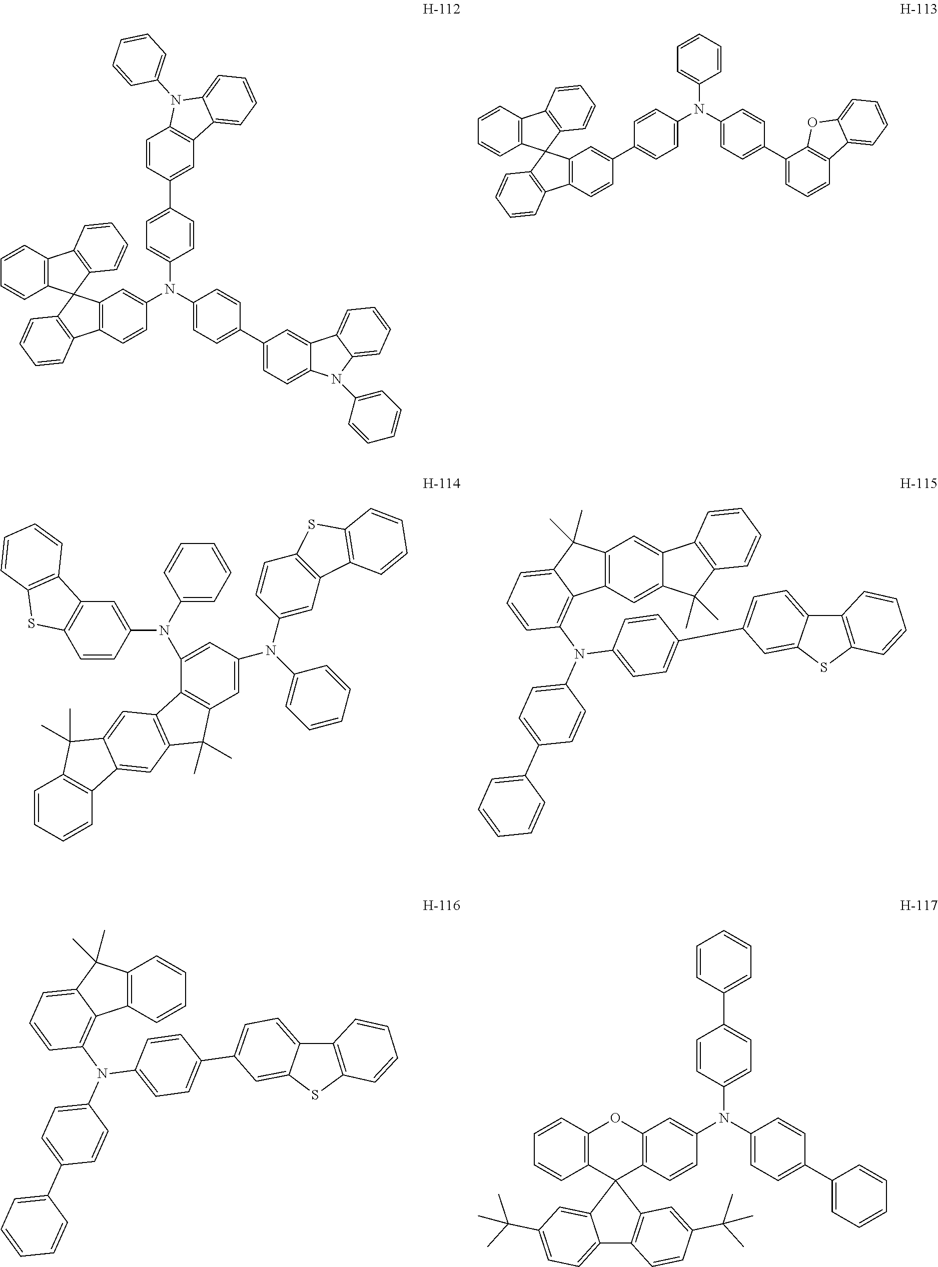

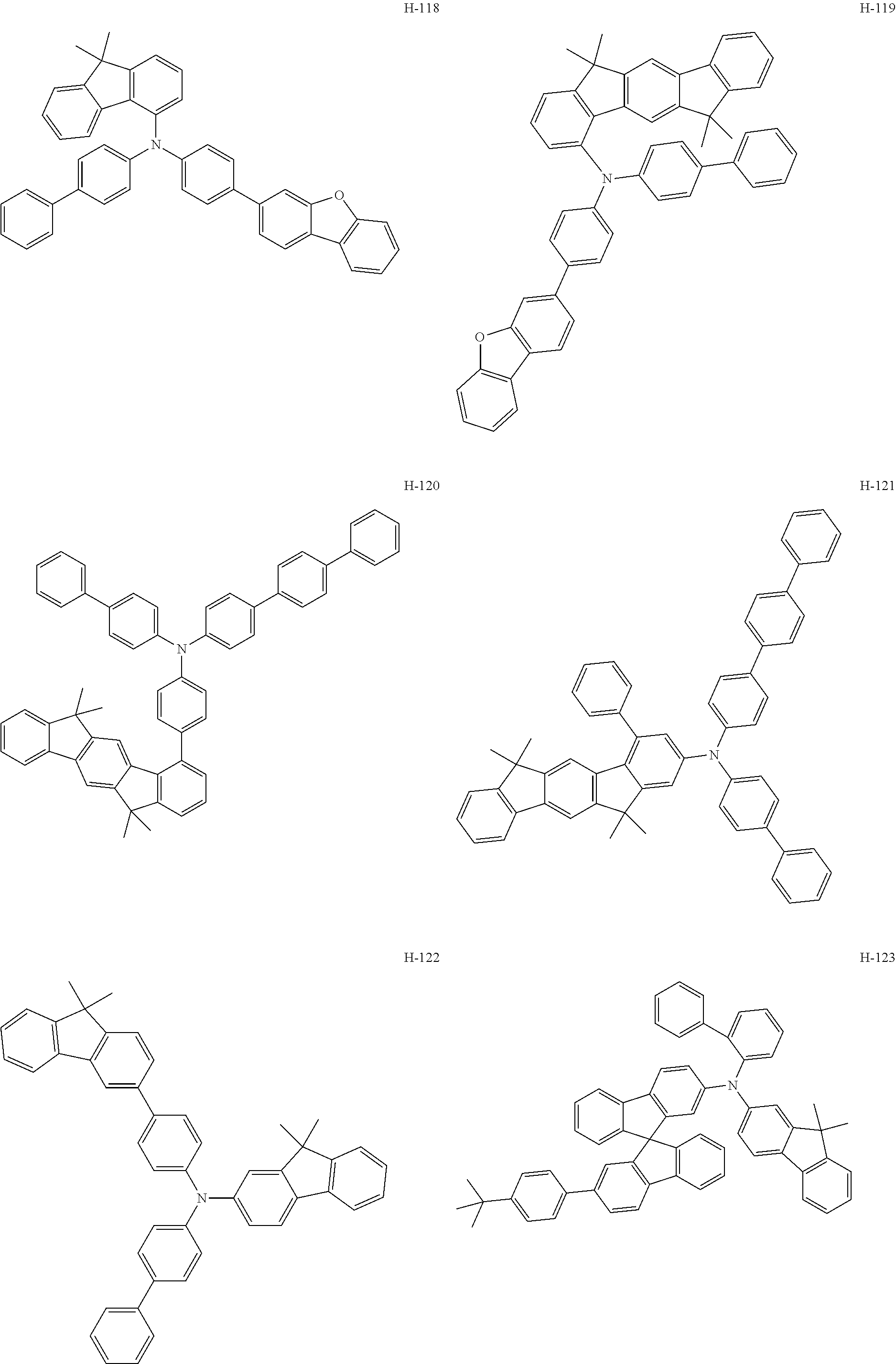

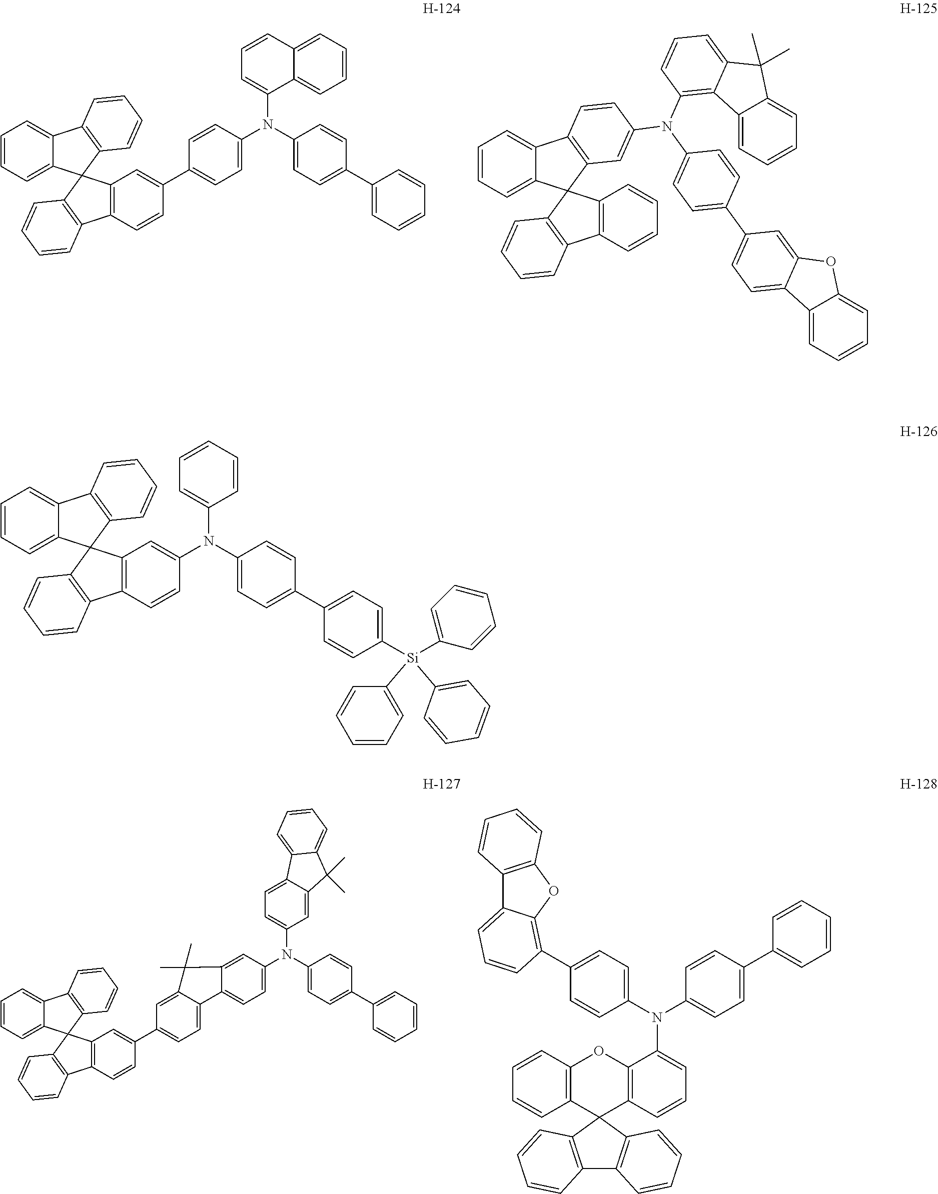

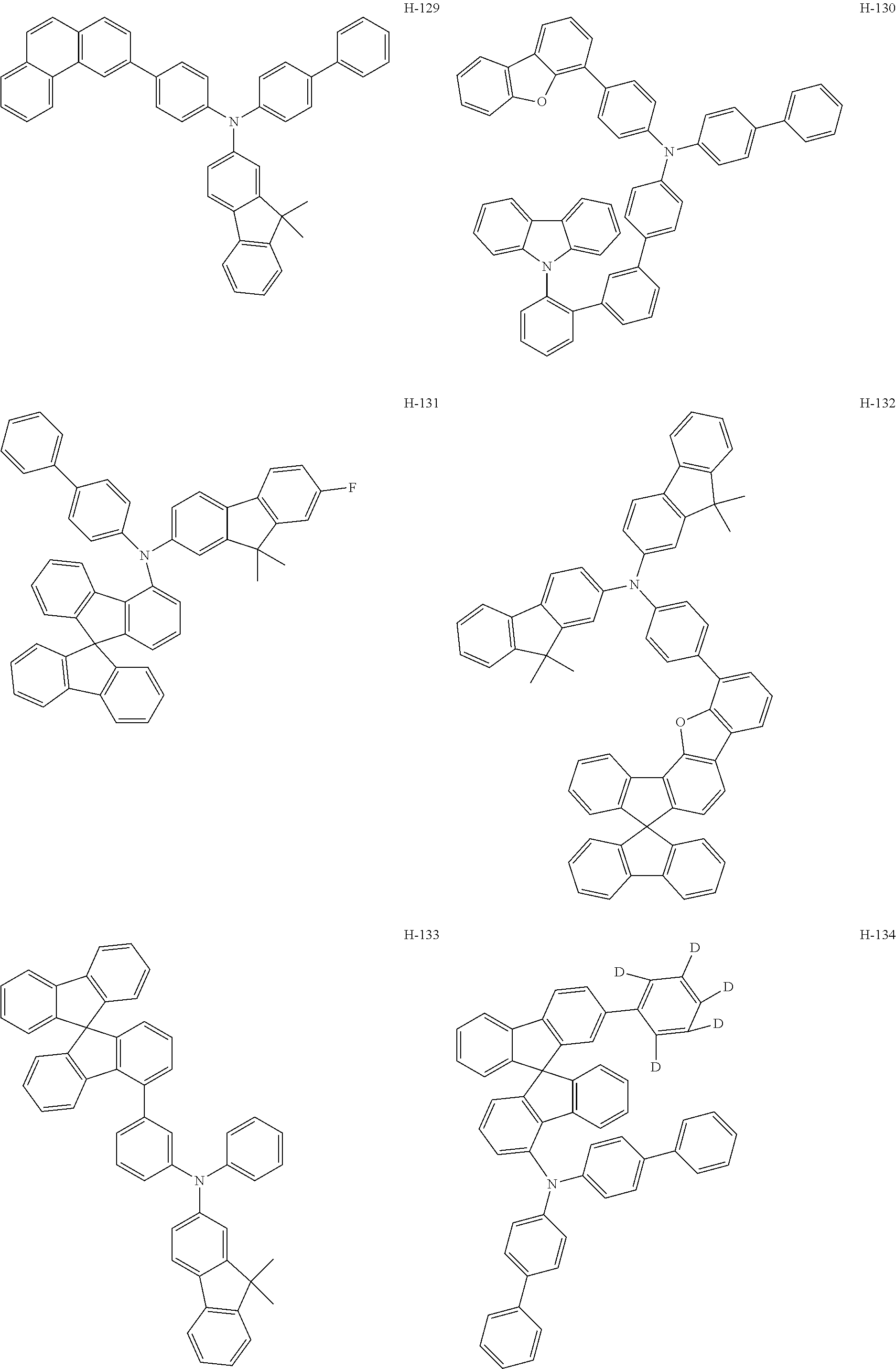

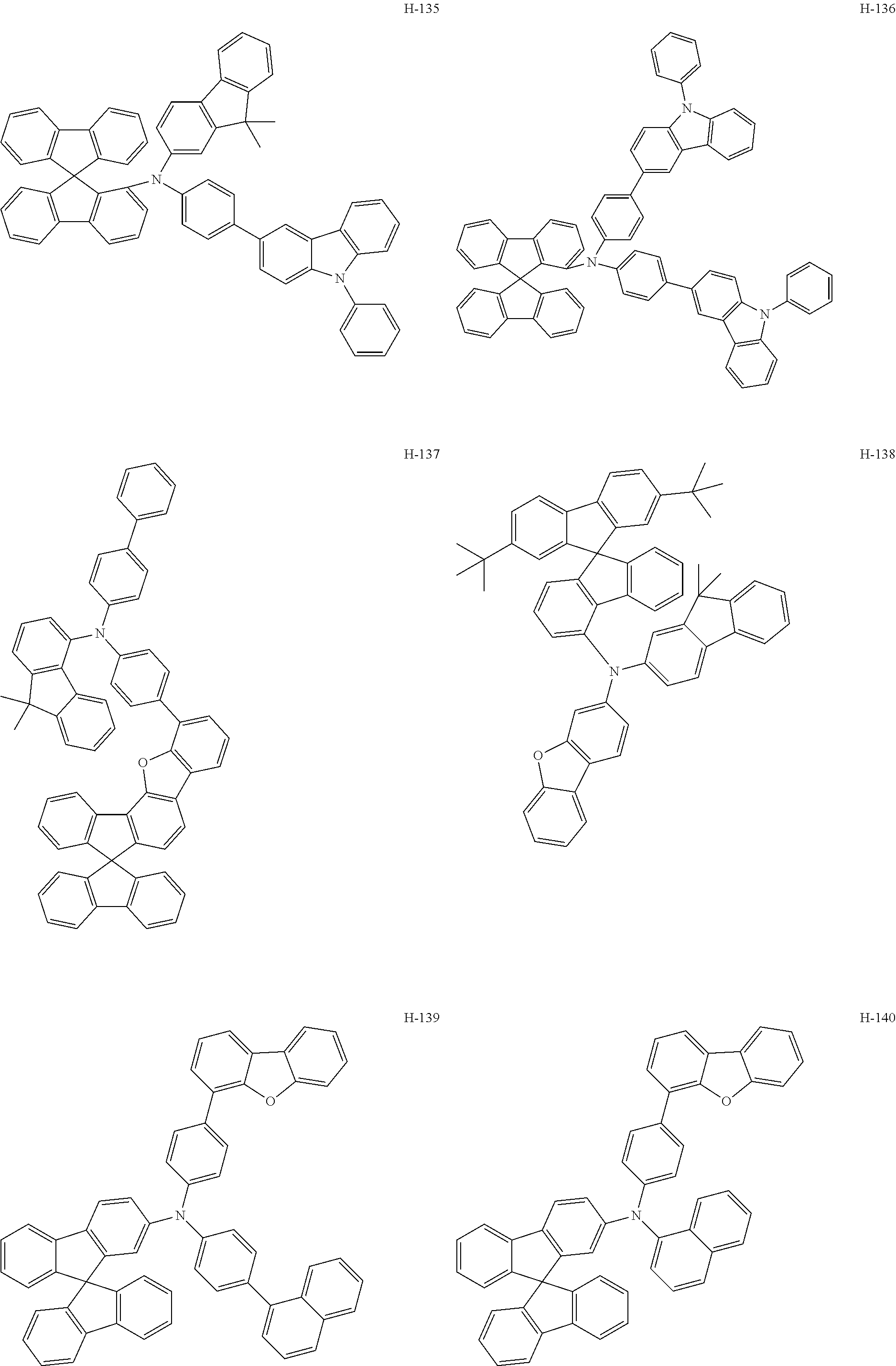

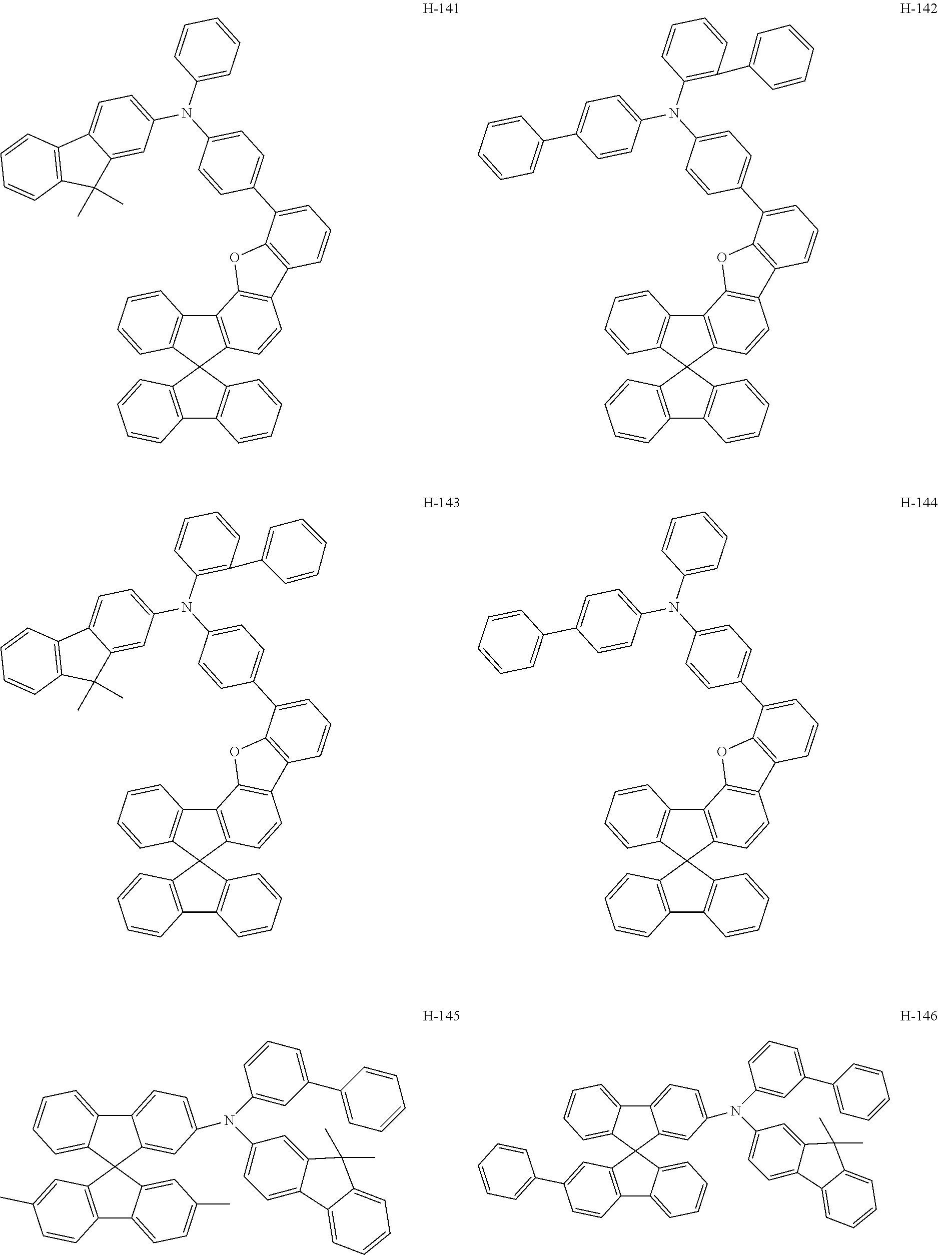

The abovementioned compounds H-1 to H-146 are suitable not just for use in a hole injection layer, but also generally in a layer having a hole-transporting function, for example a hole injection layer, a hole transport layer and/or an electron blocker layer, or suitable for use in an emitting layer as matrix material, especially as matrix material in an emitting layer comprising one or more phosphorescent emitters.

The compounds H-1 to H-146 are generally of good suitability for the abovementioned uses in OLEDs of any design and composition, not just in OLEDs according to the present application. The compounds show good performance data in OLEDs, especially good lifetime and good efficiency.

The hole transport material of the hole injection layer is more preferably selected from spirobifluorenylamines and fluorenylamines, more preferably from spirobifluorenyl monoamines and fluorenyl monoamines. A monoamine is understood here to mean a compound containing a single amine group. Most preferably, the hole transport material of the hole injection layer is selected from the above-defined compounds of the formulae (I-1-A) and (II-1-A), more preferably from compounds of the formula (I-1-A).

In an alternative preferred embodiment, the hole injection layer contains a hexaazatriphenylene derivative, preferably as described in US 2007/0092755, or another highly electron-deficient and/or Lewis-acidic compound, in each case in pure form, i.e. not in a mixture with another compound. Examples of such compounds include bismuth complexes, especially Bi(III) complexes, especially Bi(III) carboxylates such as the abovementioned compound D-13.

Apart from cathode, anode, emitting layer, hole injection layer and hole-transporting layer, the electronic device preferably also contains further layers. These are preferably selected from in each case one or more hole blocker layers, electron transport layers, electron injection layers, exciton blocker layers, interlayers, charge generation layers and/or organic or inorganic p/n junctions. However, it should be pointed out that not necessarily every one of these layers need be present. More particularly, it is preferable that the electronic device contains one or more layers selected from electron transport layers and electron injection layers that are disposed between the emitting layer and the anode. More preferably, the electronic device contains, between the emitting layer and the cathode, in this sequence, one or more electron transport layers, preferably a single electron transport layer, and a single electron injection layer, where the electron injection layer mentioned preferably directly adjoins the cathode.

The sequence of layers in the electronic device is preferably as follows:

—anode—
—hole injection layer—
—hole-transporting layer—
—emitting layer—
—optionally hole blocker layer—
—electron transport layer—
—electron injection layer—
—cathode—.

Suitable materials for hole blocker layers, electron transport layers and electron injection layers of the electronic device are especially aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Examples of specific compounds for use in these layers are shown in the following table:

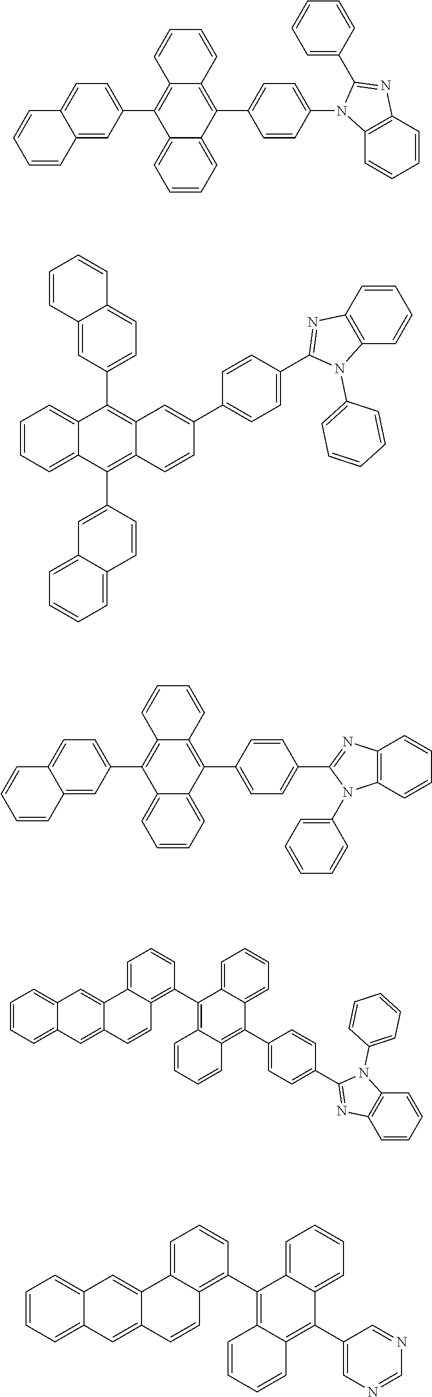 [SMILES: c1ccc(-c2nc3ccccc3n2-c2ccc(-c3c4ccccc4c(-c4ccc5ccccc5c4)c4ccccc34)cc2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-n2c(-c3ccc(-c4c5ccccc5c(-c5ccc6ccccc6c5)c5ccccc45)cc3)nc3ccccc32)cc1 ; c1ccc(-n2c(-c3ccc(-c4c5ccccc5c(-c5cccc6c5ccc5cc7ccccc7cc56)c5ccccc45)cc3)nc3ccccc32)cc1 ; c1ccc2cc3c(ccc4c(-c5c6ccccc6c(-c6cncnc6)c6ccccc56)cccc43)cc2c1]

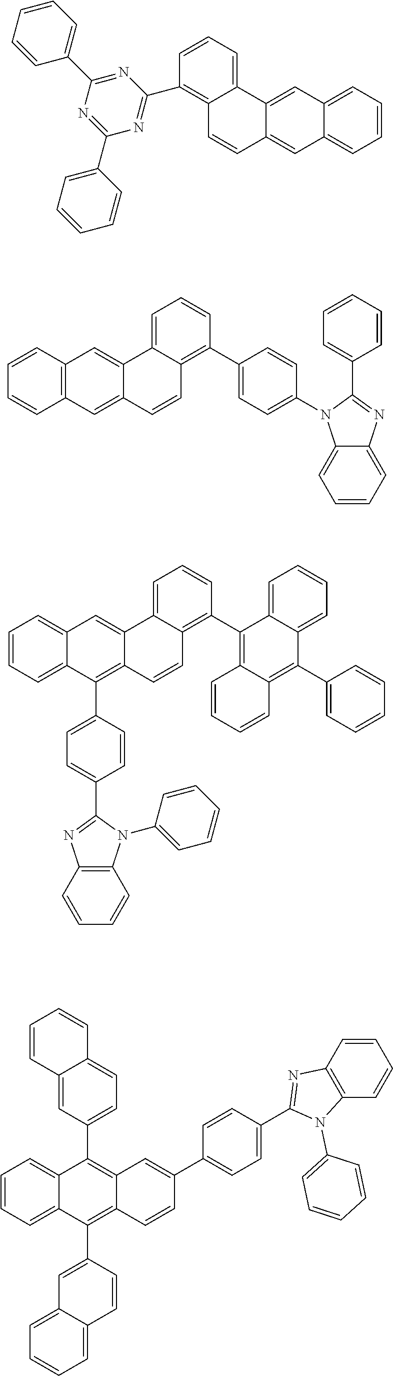 [SMILES: c1ccc(-c2nc(-c3ccccc3)nc(-c3cccc4c3ccc3cc5ccccc5cc34)n2)cc1 ; c1ccc(-c2nc3ccccc3n2-c2ccc(-c3cccc4c3ccc3cc5ccccc5cc34)cc2)cc1 ; c1ccc(-c2c3ccccc3c(-c3cccc4c3ccc3c(-c5ccc(-c6nc7ccccc7n6-c6ccccc6)cc5)c5ccccc5cc34)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

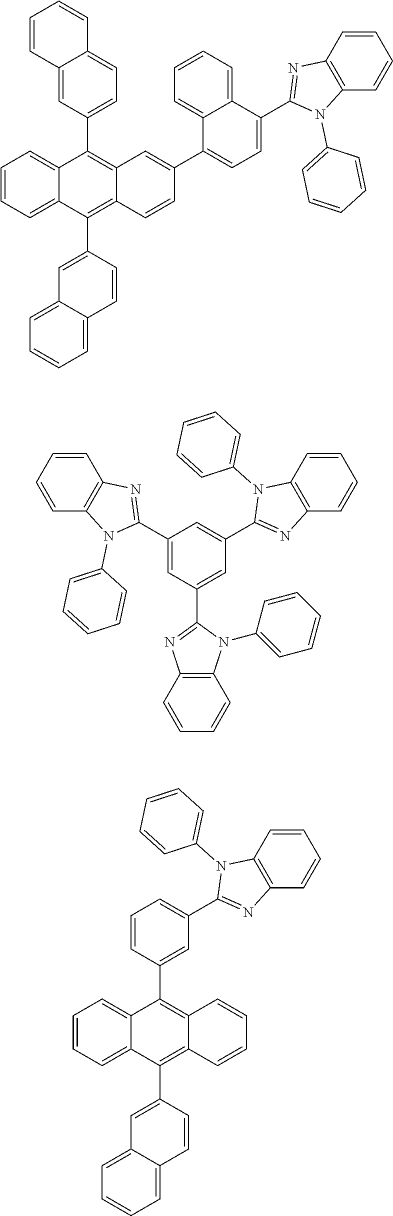 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1 ; c1ccc(-n2c(-c3cc(-c4nc5ccccc5n4-c4ccccc4)cc(-c4nc5ccccc5n4-c4ccccc4)c3)nc3ccccc32)cc1 ; c1ccc(-n2c(-c3cccc(-c4c5ccccc5c(-c5ccc6ccccc6c5)c5ccccc45)c3)nc3ccccc32)cc1]

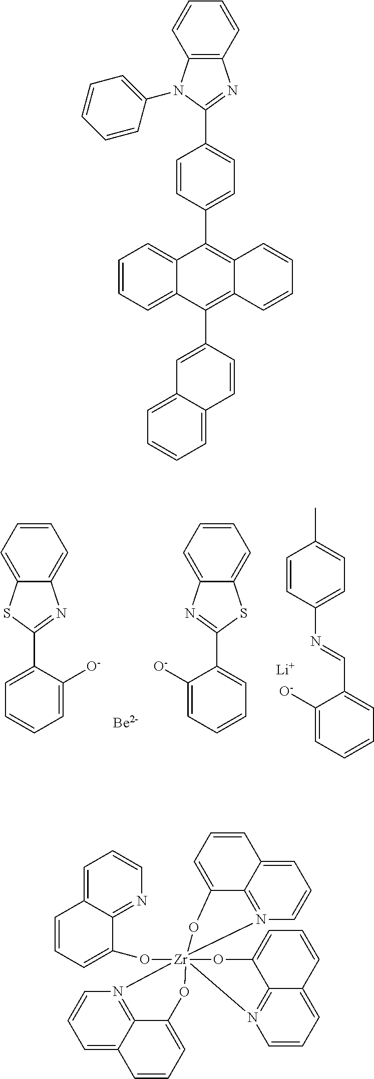

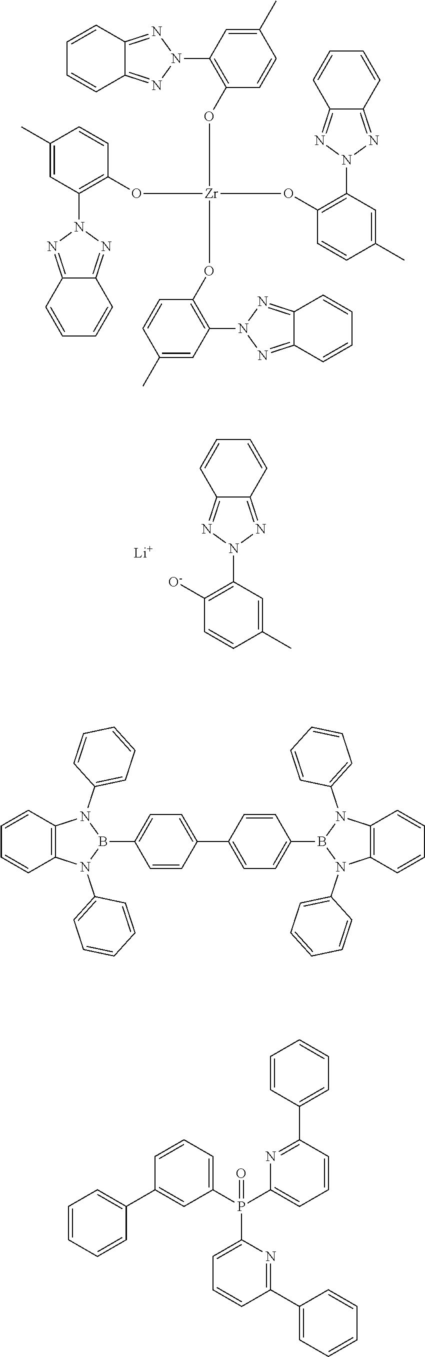

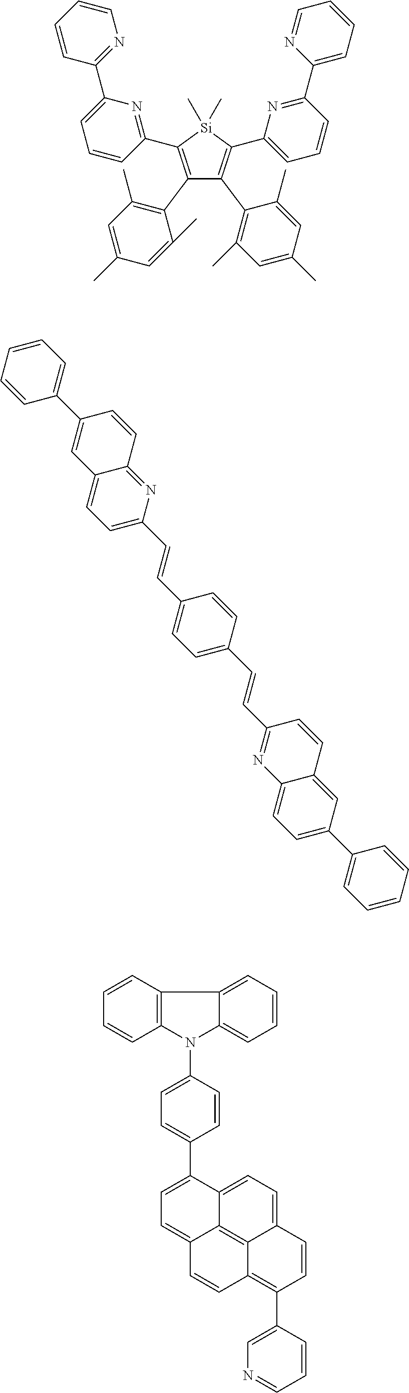 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1 ; C(=C\c1ccc2cc(-c3ccccc3)ccc2n1)/c1ccc(/C=C/c2ccc3cc(-c4ccccc4)ccc3n2)cc1 ; c1cncc(-c2ccc3ccc4c(-c5ccc(-n6c7ccccc7c7ccccc76)cc5)ccc5ccc2c3c54)c1]

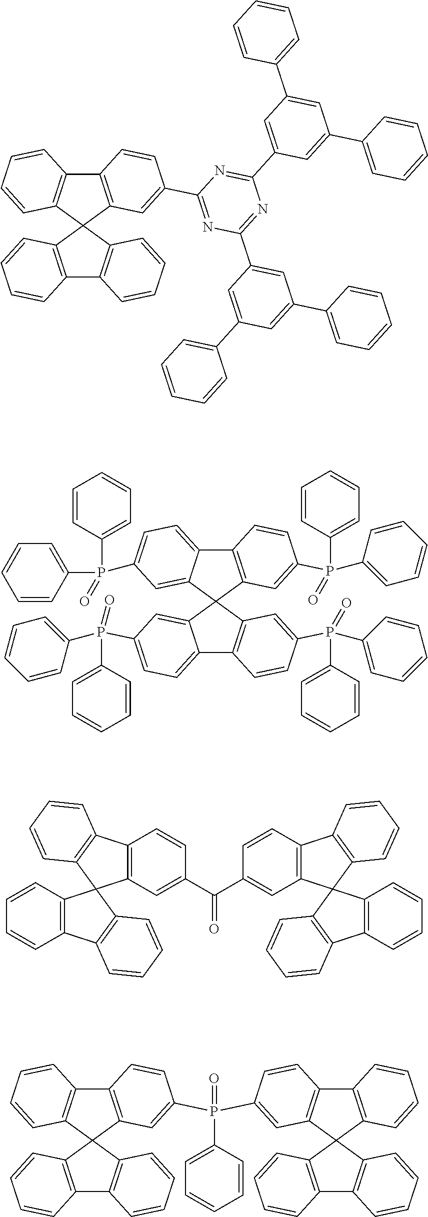 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1 ; O=P(c1ccccc1)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21 ; O=C(c1ccc2c(c1)C1(c3ccccc3-c3ccccc31)c1ccccc1-2)c1ccc2c(c1)C1(c3ccccc3-c3ccccc31)c1ccccc1-2 ; O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

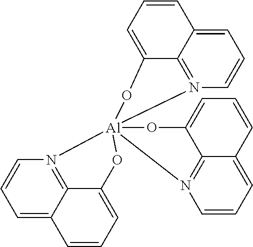

In a preferred embodiment, the electronic device is characterized in that one or more layers are applied by a sublimation process. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are applied by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably

LITI (light-induced thermal imaging, thermal transfer printing) or inkjet printing. For this purpose, soluble compounds are needed. High solubility can be achieved by suitable substitution of the compounds.

It is further preferable that an electronic device of the invention is produced by applying one or more layers from solution and one or more layers by a sublimation method.

After application of the layers (according to the use), the device is structured, contact-connected and finally sealed, in order to rule out damaging effects of water and air.

The electronic devices of the invention are preferably used in displays, as light sources in lighting applications or as light sources in medical and/or cosmetic applications.

### EXAMPLES

### 1) General Production Process for the OLEDs and Characterization of the OLEDs

Glass plaques which have been coated with structured ITO (indium tin oxide) in a thickness of 50 nm are the substrates to which the OLEDs are applied.

The OLEDs basically have the following layer structure: substrate/hole injection layer (HIL)/hole transport layer (HTL)/emission layer (EML)/electron transport layer (ETL)/electron injection layer (EIL) and finally a cathode. The cathode is formed by an aluminium layer of thickness 100 nm. The exact structure of the OLEDs can be found in the Tables 1.

All materials are applied by thermal vapour deposition in a vacuum chamber. The emission layer here, in the present examples, consists of a matrix material (host material) and an emitting dopant (emitter) which is added to the matrix material in a particular proportion by volume by co-evaporation. Details given in such a form as SMB1:SEB1 (5%) mean here that the material SMB1 is present in the layer in a proportion by volume of 95% and the material SEB1 in a proportion by volume of 5%. Analogously, the electron transport layer and, in particular examples, the HIL and/or the HTL as well also consist of a mixture of two materials, where the proportions of the materials are reported as specified above.

The chemical structures of the materials that are used in the OLEDs are shown in Table 2.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @ 10 mA/cm² refers to the external quantum efficiency which is attained at 10 mA/cm². The parameter U @ 10 mA/cm² refers to the operating voltage at 10 mA/cm². The lifetime LT is defined as the time after which the luminance drops from the starting luminance to a certain proportion in the course of operation with constant current density. An LT80 figure means here that the lifetime reported corresponds to the time after which the luminance has dropped to 80% of its starting value. The figure @60 mA/cm² means here that the lifetime in question is measured at 60 mA/cm².

2) OLEDs with a Mixture of Two Different Materials in the HTL and Comparative Examples with a Single Material in HTL, with p-Doped HIL

The following OLEDs are produced:

TABLE 1A

| Ex. | HIL | HTL1 | EML | ETL | EIL |
|---|---|---|---|---|---|
| | Thickness/ | Thickness/nm | Thickness/nm | Thickness/nm | Thickness/ |
| | nm | | | | nm |
| C1 | HTM3: | HTM3 | SMB1:SEB1 (5%) | ETM:LiQ(50%) | LiQ |
| | PDM(5%) | 200 nm | 20 nm | 30 nm | 1 nm |
| | 20 nm | | | | |
| I1 | HTM3: | HTM3:HTM5(20%) | SMB1:SEB1 (5%) | ETM:LiQ(50%) | LiQ |
| | PDM(5%) | 200 nm | 20 nm | 30 nm | 1 nm |
| | 20 nm | | | | |
| C2 | HTM2: | HTM2 | SMB1:SEB1 (5%) | ETM:LiQ(50%) | LiQ |
| | PDM(5%) | 200 nm | 20 nm | 30 nm | 1 nm |
| | 20 nm | | | | |
| I2 | HTM2: | HTM2:HTM6(20%) | SMB1:SEB1 (5%) | ETM:LiQ(50%) | LiQ |
| | PDM(5%) | 200 nm | 20 nm | 30 nm | 1 nm |
| | 20 nm | | | | |

This gives the following measurement data:

| | U | EQE |
|---|---|---|
| | @ 10 mA/cm² | @ 10 mA/cm² |
| | [V] | [%] |
| C1 | 3.8 | 8.8 |
| I1 | 3.8 | 9.2 |
| C2 | 4.3 | 9.7 |
| I2 | 4.5 | 9.9 |

By addition of the compound HTM5 to the HTL containing HTM3, a distinct improvement in efficiency is achieved in OLED 11 at the same voltage. The comparison is made here with the OLED C1 that contains exclusively the compound HTM3 in the HTL, and is otherwise of the same construction.

A distinct improvement in efficiency is also found when the compound HTM6 is added to the HTL containing HTM2 (OLED 12). The comparison is made here with the OLED C2 that contains exclusively the compound HTM2 in the HTL, and is otherwise of the same construction.

Even though the improvements in efficiency are small in percentage terms, they are not negligible since improvements in efficiency are difficult to achieve.

3) OLEDs with a Mixture of Two Different Materials in the HTL and Comparative Examples with a Single Material in HTL, with HIL Composed of a Single Material

The following OLEDs are produced:

TABLE 1B

| Ex. | HIL | HTL1 | EML | ETL | EIL |
|---|---|---|---|---|---|
| | Thickness/ | Thickness/nm | Thickness/nm | Thickness/nm | Thickness/ |
| | nm | | | | nm |
| C3 | HIL1 | HTM1 | SMB1:SEB1(5%) | ETM:LiQ(50%) | LiQ |
| | 5 nm | 200 nm | 20 nm | 30 nm | 1 nm |
| I3 | HIL1 | HTM1:HTM5(20%) | SMB1:SEB1(5%) | ETM:LiQ(50%) | LiQ |
| | 5 nm | 200 nm | 20 nm | 30 nm | 1 nm |
| I4 | HIL1 | HTM1:HTM6(20%) | SMB1:SEB1(5%) | ETM:LiQ(50%) | LiQ |
| | 5 nm | 200 nm | 20 nm | 30 nm | 1 nm |

This gives the following measurement data:

| | U | |
|---|---|---|
| | @ 10 mA/cm² | LT80 @ 60 mA/cm² |
| | [V] | [h] |
| C3 | 3.8 | 285 |
| I3 | 3.8 | 311 |
| I4 | 3.8 | 293 |

By addition of the compounds HTM5 (13) or HTM6 (14) to the HTL containing the compound HTM1, an improvement in lifetime is achieved in each case. The comparison is made here with the OLED C3 that contains exclusively the compound HTM1 in the HTL, and is otherwise of the same construction.

In the case of OLEDs that have a thinner HTL (70 nm) compared to the thicker HTL that is used in the OLEDs C3, 13 and 14, improvements in lifetime likewise occur, as shown by the examples that follow. As before, OLEDs with a mixture of two different materials in the HTL (16, 17 and 18) are compared here with an OLED containing exclusively the compound HTM1 in the HTL (C4).

TABLE 1C

| Ex. | HIL | HTL1 | EML | ETL | EIL |
|---|---|---|---|---|---|
| | Thickness/ | Thickness/nm | Thickness/nm | Thickness/nm | Thickness/ |
| | nm | | | | nm |
| C4 | HIL1 | HTM1 | SMB1:SEB1(5%) | ETM:LiQ(50%) | LiQ |
| | 5 nm | 70 nm | 20 nm | 30 nm | 1 nm |
| I6 | HIL1 | HTM1:HTM7(20%) | SMB1:SEB1(5%) | ETM:LiQ(50%) | LiQ |
| | 5 nm | 70 nm | 20 nm | 30 nm | 1 nm |
| I7 | HIL1 | HTM1:HTM6(20%) | SMB1:SEB1(5%) | ETM:LiQ(50%) | LiQ |
| | 5 nm | 70 nm | 20 nm | 30 nm | 1 nm |
| I8 | HIL1 | HTM1:HTM5(20%) | SMB1:SEB1(5%) | ETM:LiQ(50%) | LiQ |
| | 5 nm | 70 nm | 20 nm | 30 nm | 1 nm |

This gives the following measurement data:

| | U | |
|---|---|---|
| | @ 10 mA/cm² | LT80 @ 60 mA/cm² |
| | [V] | [h] |
| C4 | 3.6 | 285 |
| I6 | 3.5 | 308 |
| I7 | 3.6 | 310 |
| I8 | 3.5 | 306 |

In all cases, addition of a material selected from HTM5, HTM6 and HTM7 improves the lifetime of the OLED.

The second material may also be added in a higher proportion than in the 20% shown above, as shown by the following example:

TABLE 1D

| Ex. | HIL | HTL1 | EML | ETL | EIL |
|---|---|---|---|---|---|
| | Thickness/ | Thickness/nm | Thickness/nm | Thickness/nm | Thickness/ |
| | nm | | | | nm |
| C4 | HIL1 | HTM1 | SMB1:SEB1(5%) | ETM:LiQ(50%) | LiQ |
| | 5 nm | 70 nm | 20 nm | 30 nm | 1 nm |
| I5 | HIL1 | HTM1:HTM5(50%) | SMB1:SEB1(5%) | ETM:LiQ(50%) | LiQ |
| | 5 nm | 70 nm | 20 nm | 30 nm | 1 nm |

The following results are obtained:

| | U | |
|---|---|---|
| | @ 10 mA/cm² | LT80 @ 60 mA/cm² |
| | [V] | [h] |
| C4 | 3.6 | 285 |
| I5 | 3.6 | 353 |

However, the addition of the second material in a high proportion has the disadvantage that losses in efficiency occur. When the second material is used in a proportion of 10-30% by volume, especially 20% by volume, as shown above, these occur to a distinctly lesser degree, if at all.

TABLE 2

| | |
|---|---|
| 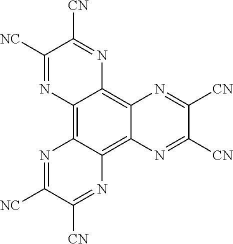 [SMILES: N#Cc1nc2c3nc(C#N)c(C#N)nc3c3nc(C#N)c(C#N)nc3c2nc1C#N] | HIL1 |
| 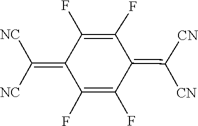 [SMILES: N#CC(C#N)=c1c(F)c(F)c(=C(C#N)C#N)c(F)c1F] | PDM |
| 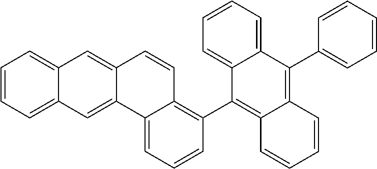 [SMILES: c1ccc(-c2c3ccccc3c(-c3cccc4c3ccc3cc5ccccc5cc34)c3ccccc23)cc1] | SMB1 |
| 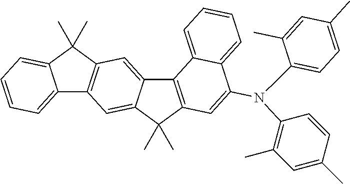 [SMILES: Cc1ccc(N(c2ccc(C)cc2C)c2cc3c(c4ccccc24)-c2cc4c(cc2C3(C)C)-c2ccccc2C4(C)C)c(C)c1] | SEB1 |
| 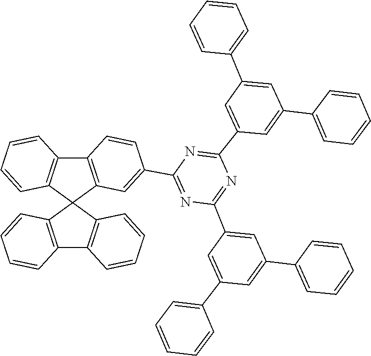 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ETM |
| 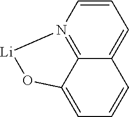 | LiQ |
| 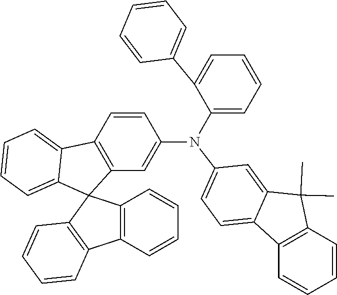 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | HTM1 |
| 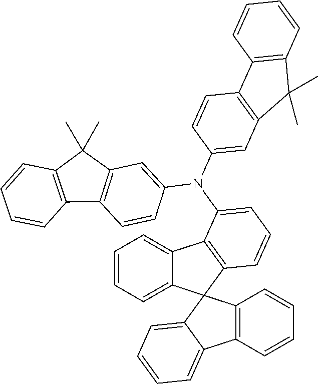 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21] | HTM2 |
| 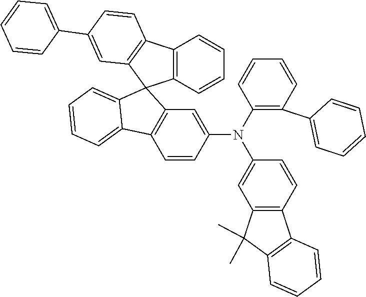 [SMILES: CC1(C)c2ccccc2-c2ccc(N(c3ccc4c(c3)C3(c5ccccc5-c5ccc(-c6ccccc6)cc53)c3ccccc3-4)c3ccccc3-c3ccccc3)cc21] | HTM3 |
| 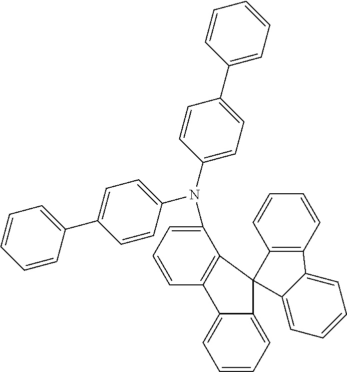 [SMILES: c1ccc(-c2ccc(N(c3ccc(-c4ccccc4)cc3)c3cccc4c3C3(c5ccccc5-c5ccccc53)c3ccccc3-4)cc2)cc1] | HTM5 |
| 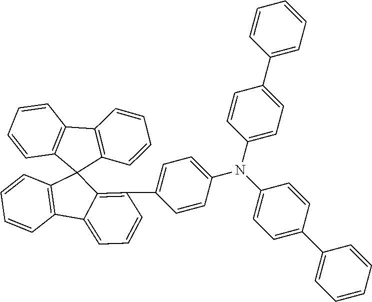 [SMILES: c1ccc(-c2ccc(N(c3ccc(-c4ccccc4)cc3)c3ccc(-c4cccc5c4C4(c6ccccc6-c6ccccc64)c4ccccc4-5)cc3)cc2)cc1] | HTM6 |
| 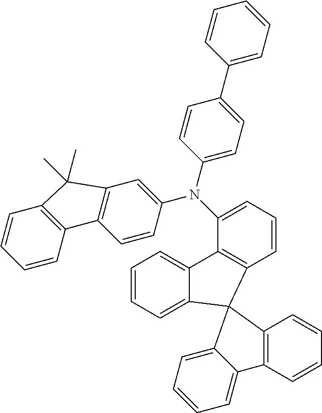 [SMILES: CC1(C)c2ccccc2-c2ccc(N(c3ccc(-c4ccccc4)cc3)c3cccc4c3-c3ccccc3C43c4ccccc4-c4ccccc43)cc21] | HTM7 |

4) Determination of the HOMO of the Compounds that are Used in the Mixed HTL

The method described at page 28 line 1 to page 29 line 21 of published specification WO 2011/032624 gives the following values for the HOMO of the compounds HTM1, HTM2, HTM3, HTM5, HTM6 and HTM7:

| Compound | HOMO (eV) |
|---|---|
| HTM1 | −5.15 |
| HTM2 | −5.18 |
| HTM3 | −5.15 |
| HTM5 | −5.27 |
| HTM6 | −5.23 |
| HTM7 | −5.26 |

1.-22. (canceled)

23. An electronic device comprising
anode,
cathode,
emitting layer disposed between anode and cathode,
a hole injection layer disposed between anode and emitting layer;
a hole-transporting layer disposed between hole injection layer and emitting layer and directly adjoining the emitting layer on the anode side, and containing two different compounds conforming to identical or different formulae selected from formulae (I) and (II) 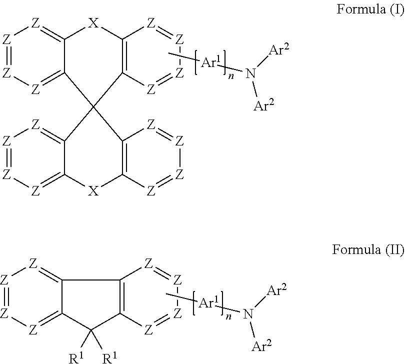
where
Z is the same or different at each instance and is selected from CR¹ and N, where Z is C when a 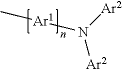
group is bonded thereto;
X is the same or different at each instance and is selected from the group consisting of a single bond, O, S, C(R¹)₂ and NR¹;
Ar¹ and Ar² are the same or different at each instance and are selected from aromatic ring systems which have 6 to 40 aromatic ring atoms and are substituted by one or more R² radicals and heteroaromatic ring systems which have 5 to 40 aromatic ring atoms and are substituted by one or more R² radicals;
R¹ and R² are the same or different at each instance and are selected from H, D, F, Cl, Br, I, C(═O)R³, CN, Si(R³)₃, N(R³)₂, P(═O)(R³)₂, OR³, S(═O)R³, S(═O)₂R³, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ or R² radicals may be joined to one another and may form a ring; where the alkyl, alkoxy, alkenyl and alkynyl groups mentioned and the aromatic ring systems and heteroaromatic ring systems mentioned are each substituted by R³ radicals; and where one or more CH₂ groups in the alkyl, alkoxy, alkenyl and alkynyl groups mentioned may be replaced by —R³C═CR³—, —C≡C—, Si(R³)₂, C═O, C═NR³, —C(═O)O—, —C(═O)NR³—, NR³, P(═O)(R³), —O—, —S—, SO or SO₂;
R³ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R³ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;
n is 0, 1, 2, 3 or 4, where, when n=0, the Ar¹ group is absent and the nitrogen atom is bonded directly to the rest of the formula.

24. The electronic device according to claim 23, wherein the emitting layer is a blue-fluorescing emitting layer or a green-phosphorescing emitting layer.

25. The electronic device according to claim 23, wherein the hole-transporting layer has a layer thickness of 20 nm to 300 nm.

26. The electronic device according to claim 23, wherein the hole-transporting layer has a layer thickness of not more than 250 nm.

27. The electronic device according to claim 23, wherein the hole-transporting layer contains exactly 2 different compounds conforming to identical or different formulae selected from formulae (I) and (II).

28. The electronic device according to claim 23, wherein the hole-transporting layer consists of compounds conforming to identical or different formulae selected from formulae (I) and (II).

29. The electronic device according to claim 23, wherein the hole-transporting layer contains two different compounds conforming to a formula (I).

30. The electronic device according to claim 23, wherein the two different compounds conforming to identical or different formulae selected from formulae (I) and (II) are each present in the hole-transporting layer in a proportion of at least 5%.

31. The electronic device according to claim 23, wherein one of the two different compounds in the hole-transporting layer is a compound HTM-1 selected from formulae (I-1-A) and (II-1-A) 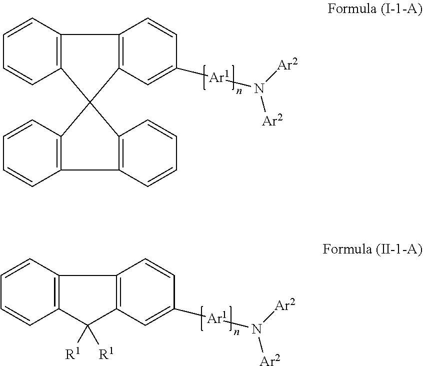
and the other of the two different compounds in the hole-transporting layer is a compound HTM-2 selected from the formulae (I-1-B), (I-1-C), (I-1-D), (II-1-B), (II-1-C), and (II-1-D) 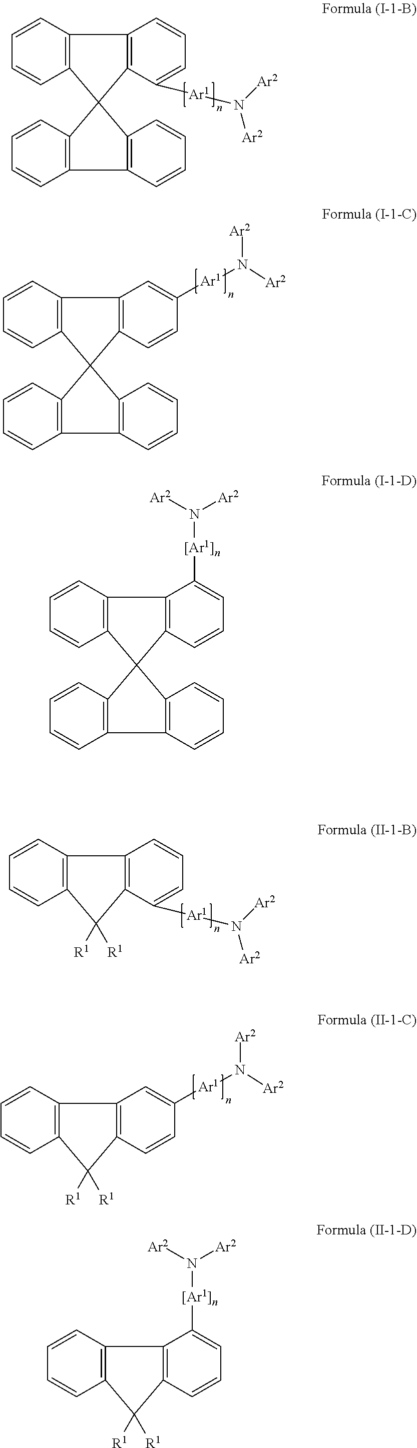
where the groups that occur in the formulae (I-1-A) to (I-1-D) and (II-1-B) to (II-1-D) are as defined in claim 23, and where the unoccupied positions on the spirobifluorene and fluorene are each substituted by R¹ radicals.

32. The electronic device according to claim 31, wherein HTM-1 is present in the hole-transporting layer in a proportion five to two times as high as the proportion of HTM-2 in the hole-transporting layer.

33. The electronic device according to claim 31, wherein HTM-1 is present in the hole-transporting layer in a proportion of 65% to 85%, and HTM-2 in the hole-transporting layer in a proportion of 15% to 35%.

34. The electronic device according to claim 31, wherein HTM-1 has a HOMO of −4.8 eV to −5.2 eV, and HTM-2 a HOMO of −5.1 eV to −5.4 eV.

35. The electronic device according to claim 31, wherein HTM-1 has a HOMO higher than HTM-2 by 0.02 eV to 0.3 eV.

36. The electronic device according to claim 23, wherein the electronic device has the layer sequence anode/hole injection layer/hole-transporting layer/emitting layer, where the layers mentioned directly adjoin one another.

37. The electronic device according to claim 23, wherein the hole injection layer contains a mixture of a p-dopant and a hole transport material.

38. The electronic device according to claim 23, wherein the hole transport material of the hole injection layer is selected from the compounds of the formulae (I-1-A) or (II-1-A), 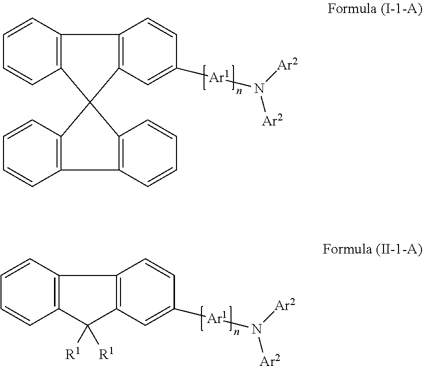
where groups that occur in the formulae (I-1-A) and (II-1-A) are as defined in claim 23, and where the unoccupied positions on the spirobifluorene and fluorene are each substituted by R¹ radicals.

39. The electronic device according to claim 23, wherein the hole injection layer contains a hexaazatriphenylene derivative or another highly electron-deficient and/or Lewis-acidic compound, each in pure form.

40. Process for producing the electronic device according to claim 23, wherein one or more layers of the device are produced from solution or by a sublimation process.

41. A device in displays, as a light source in lighting applications or as a light source in medical and/or cosmetic applications which comprises the electronic device according to claim 23.

42. The compound of one of the following structural formulae H-1 to H-130:

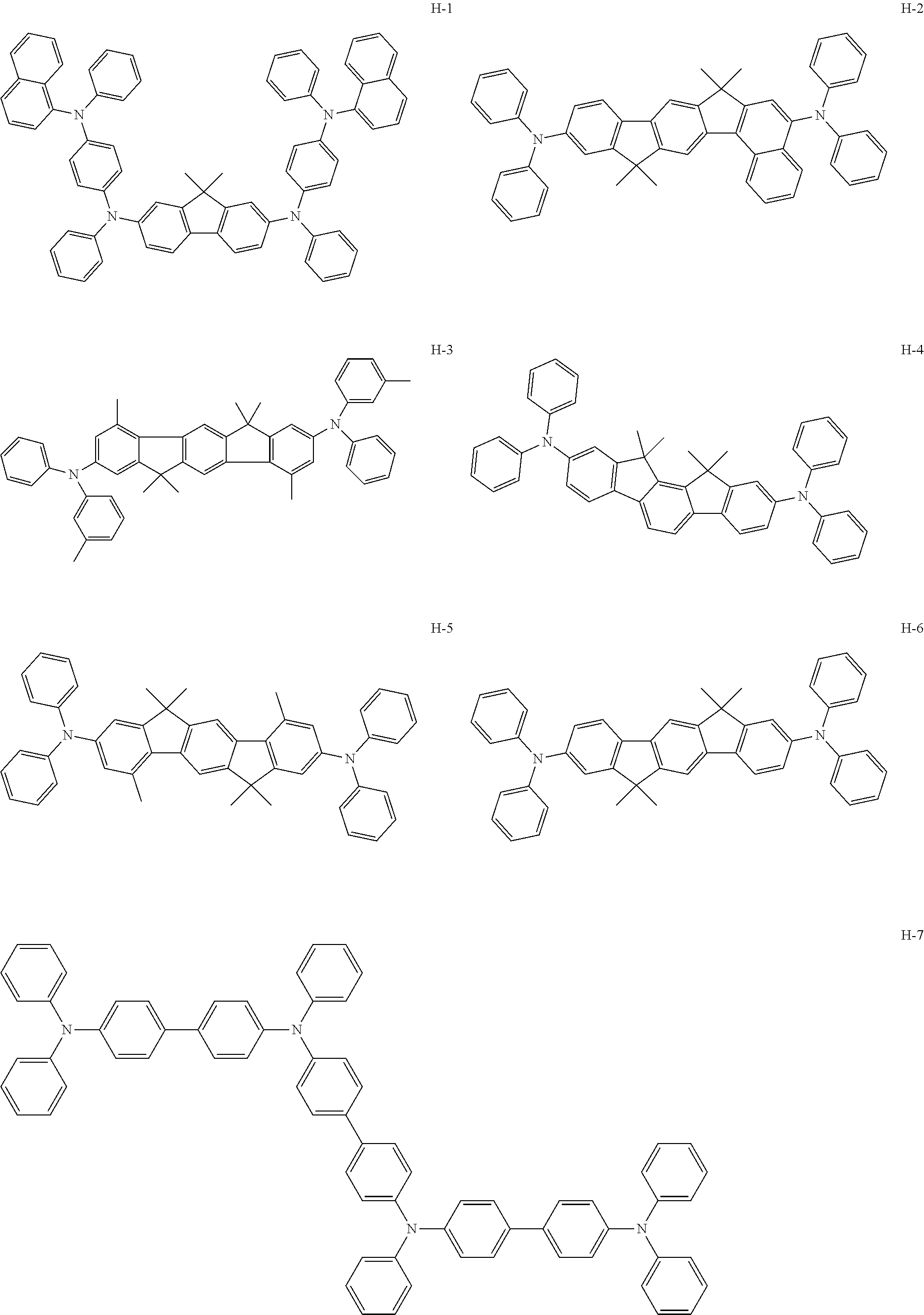

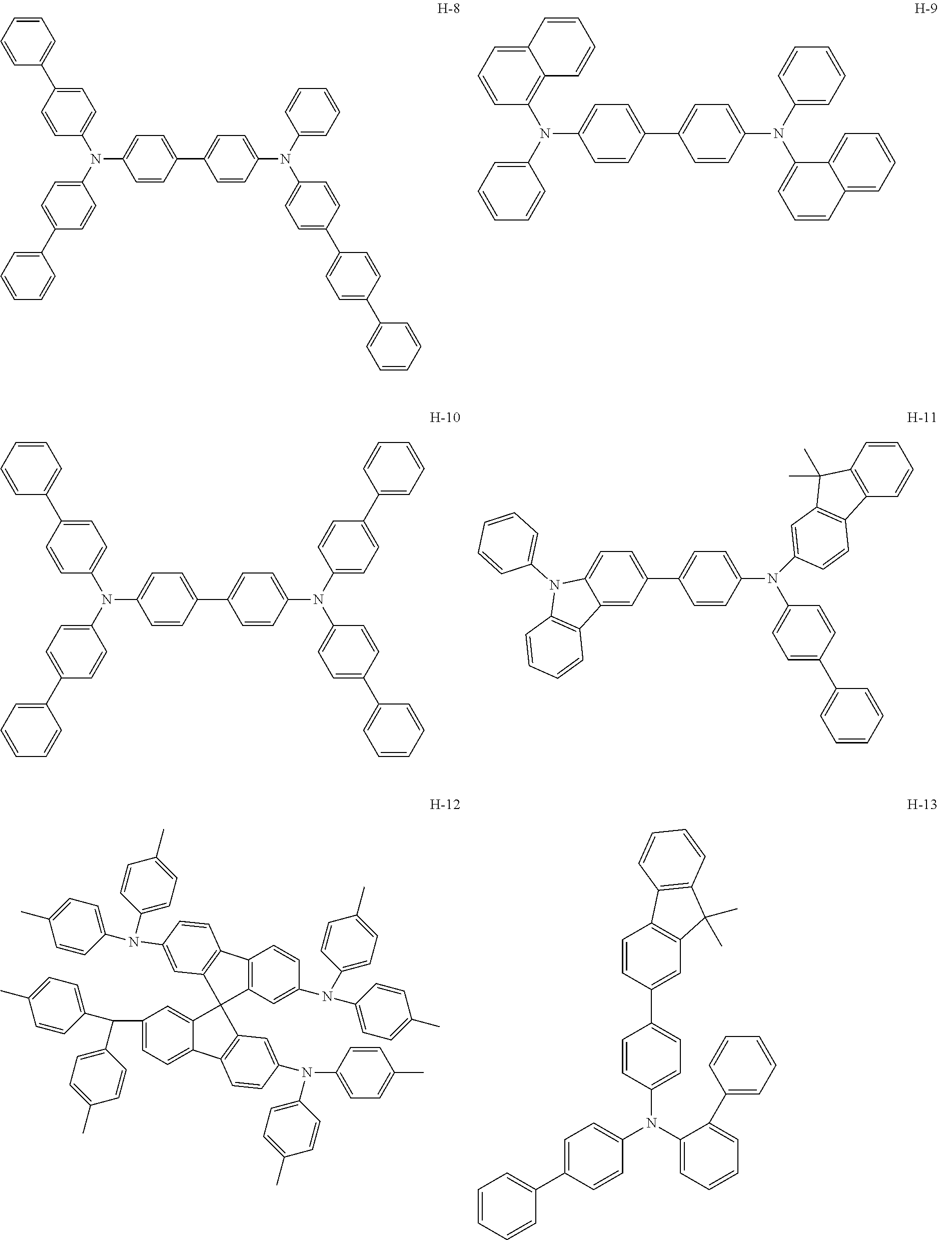

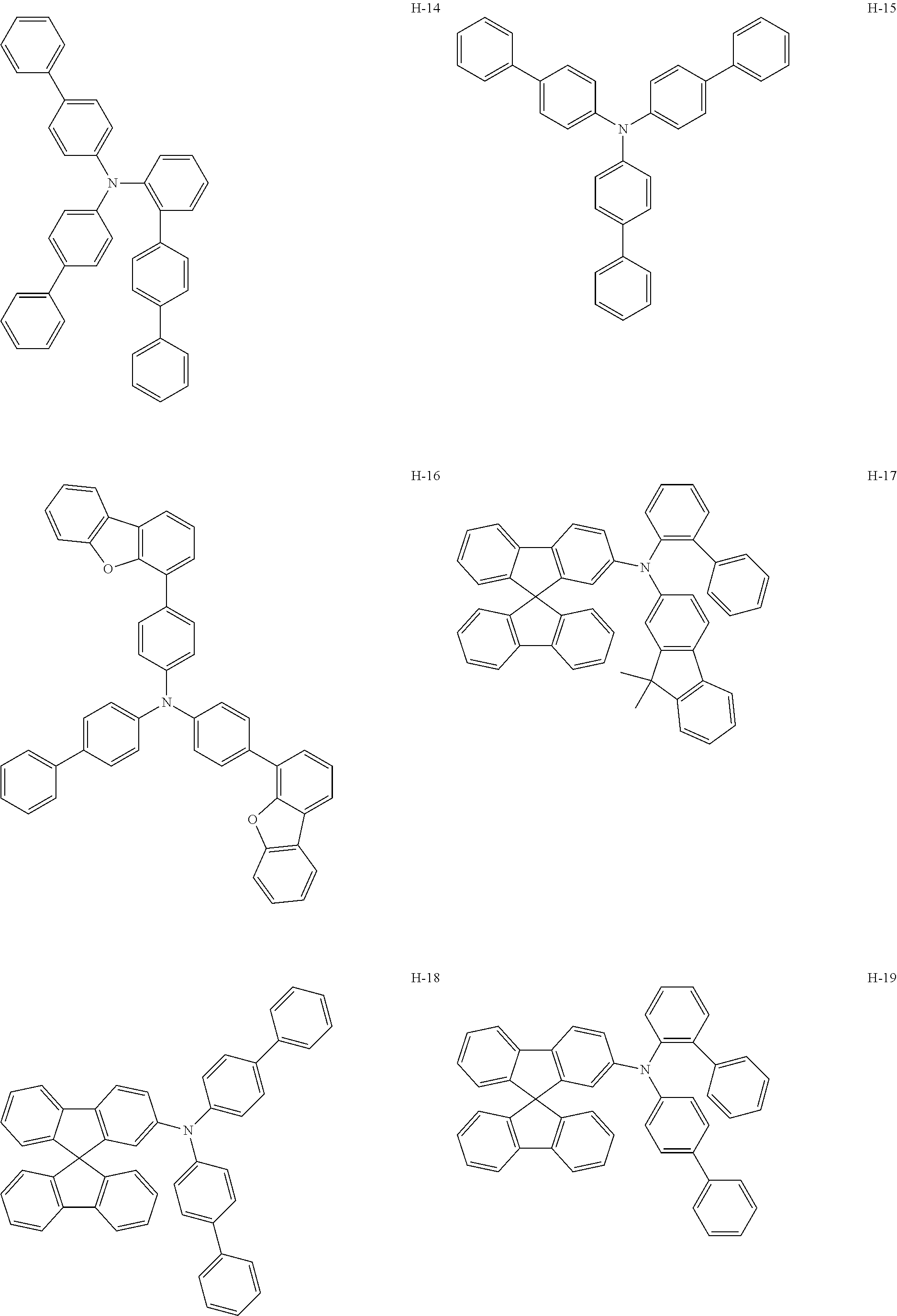 [SMILES: c1ccc(-c2ccc(-c3ccccc3N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4ccccc4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc4c(c3)C3(c5ccccc5-c5ccccc53)c3ccccc3-4)cc2)cc1 ; c1ccc(-c2ccc(N(c3ccc4c(c3)C3(c5ccccc5-c5ccccc53)c3ccccc3-4)c3ccccc3-c3ccccc3)cc2)cc1]

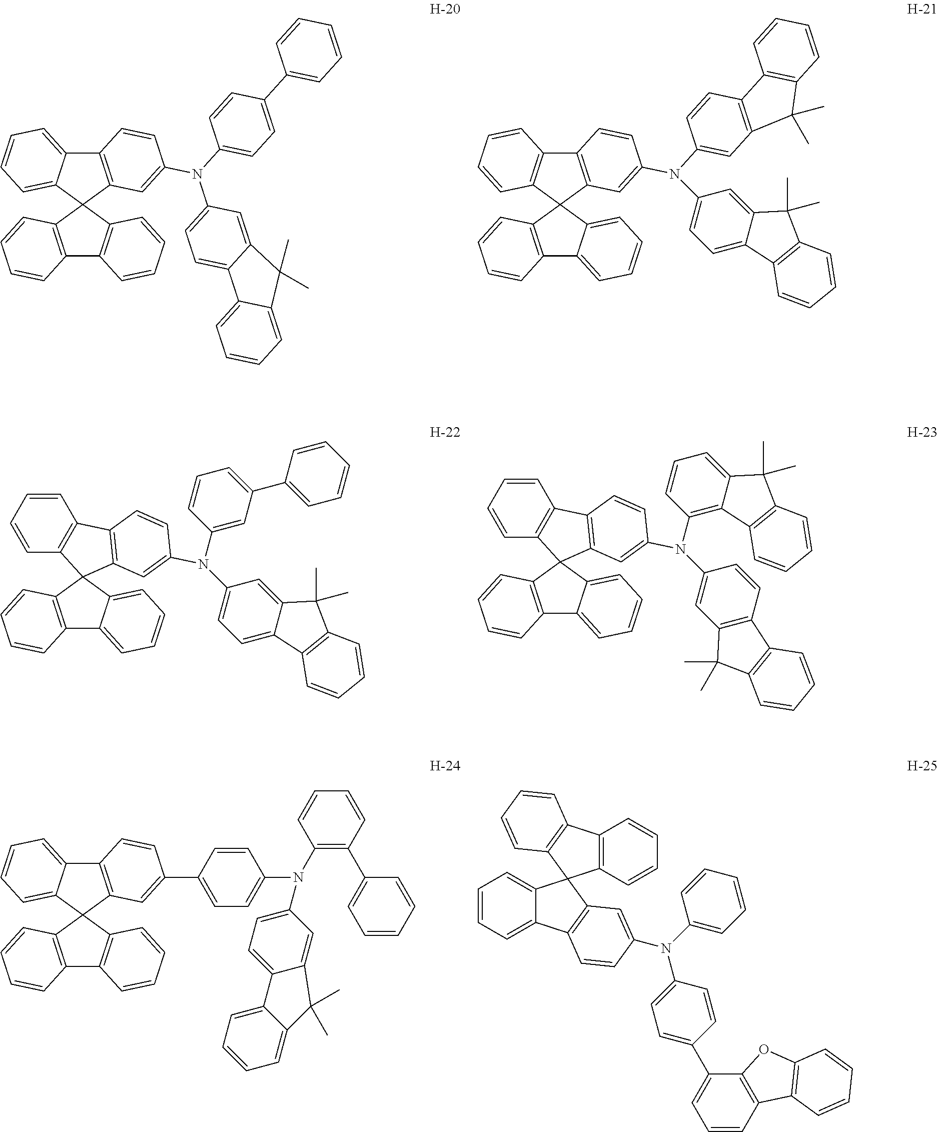

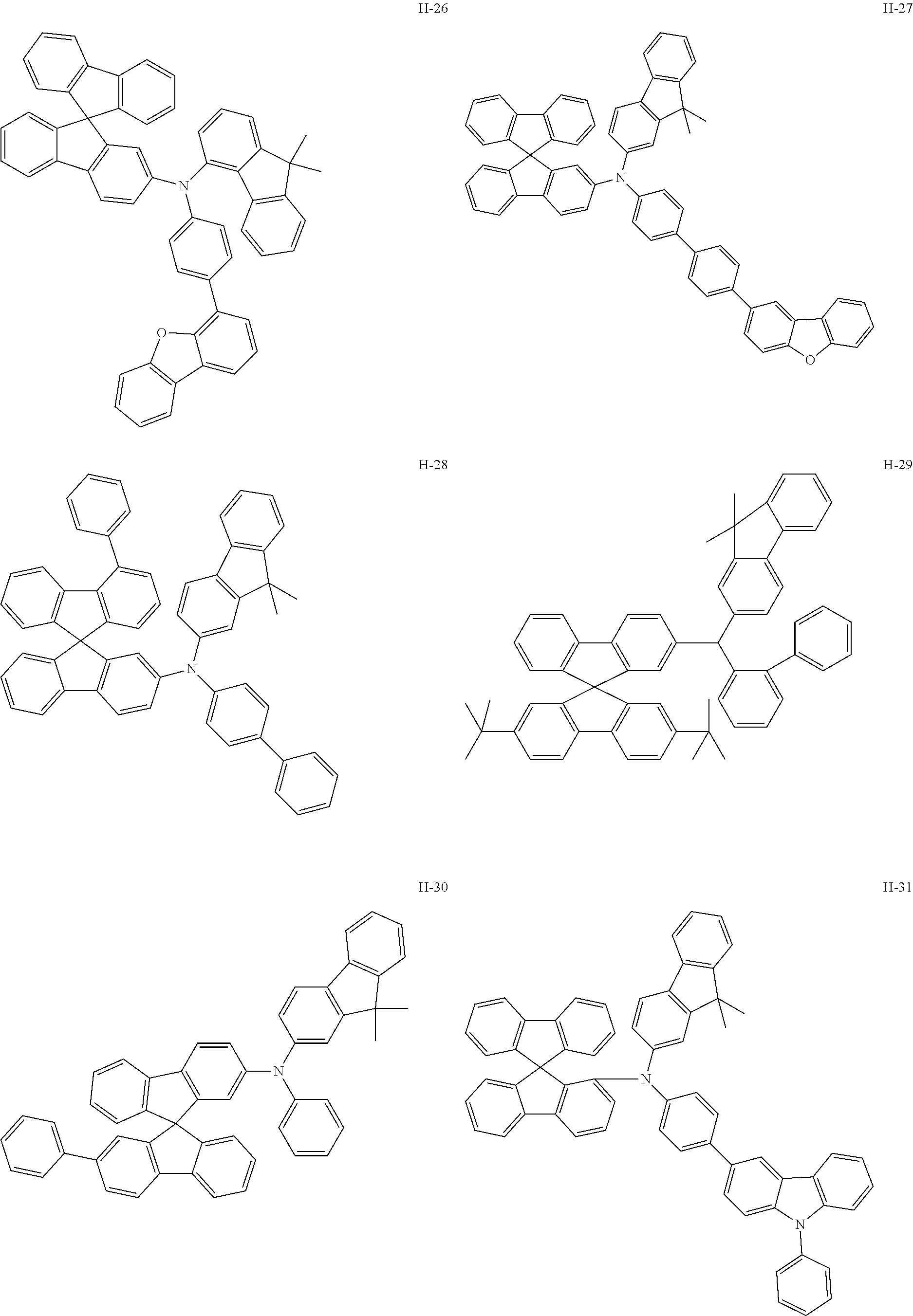

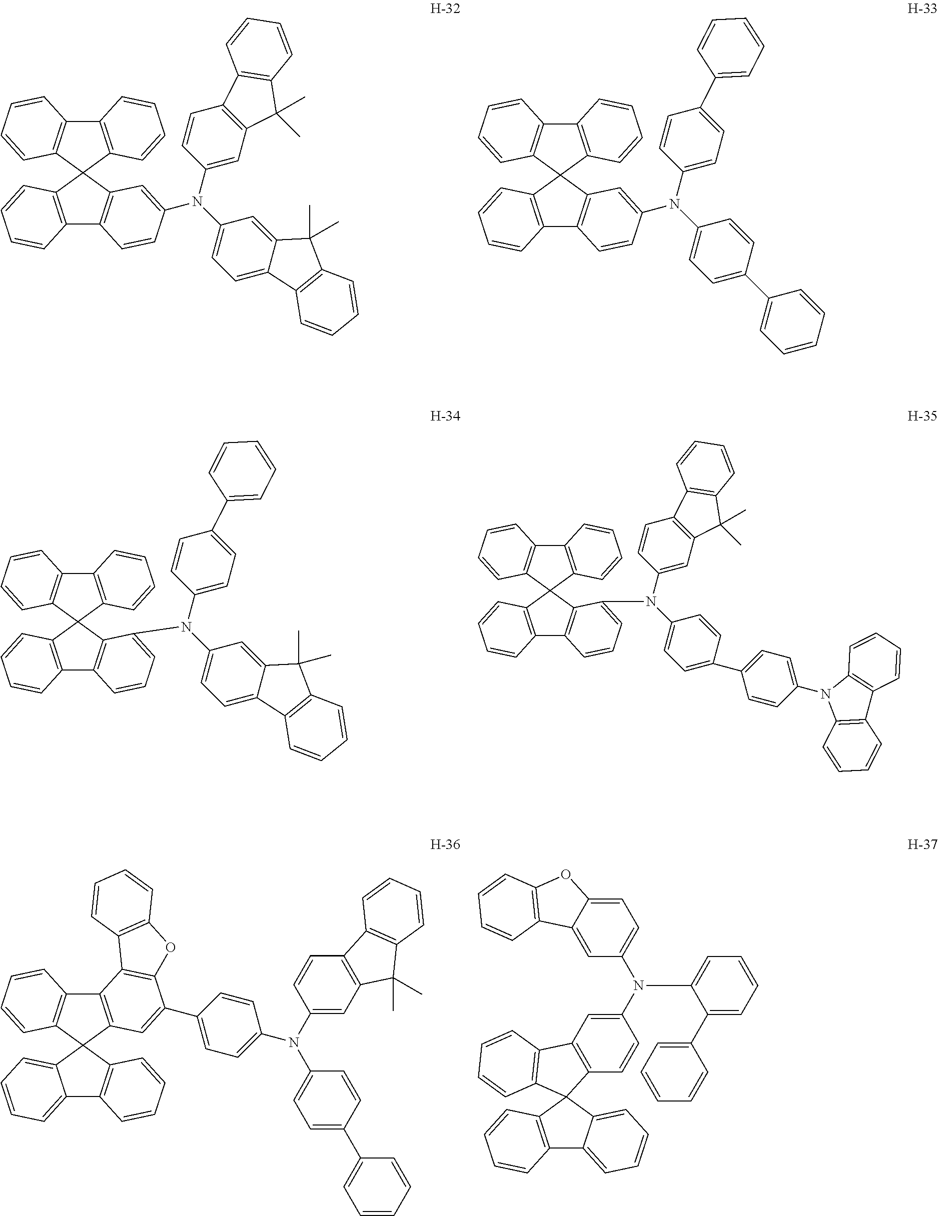

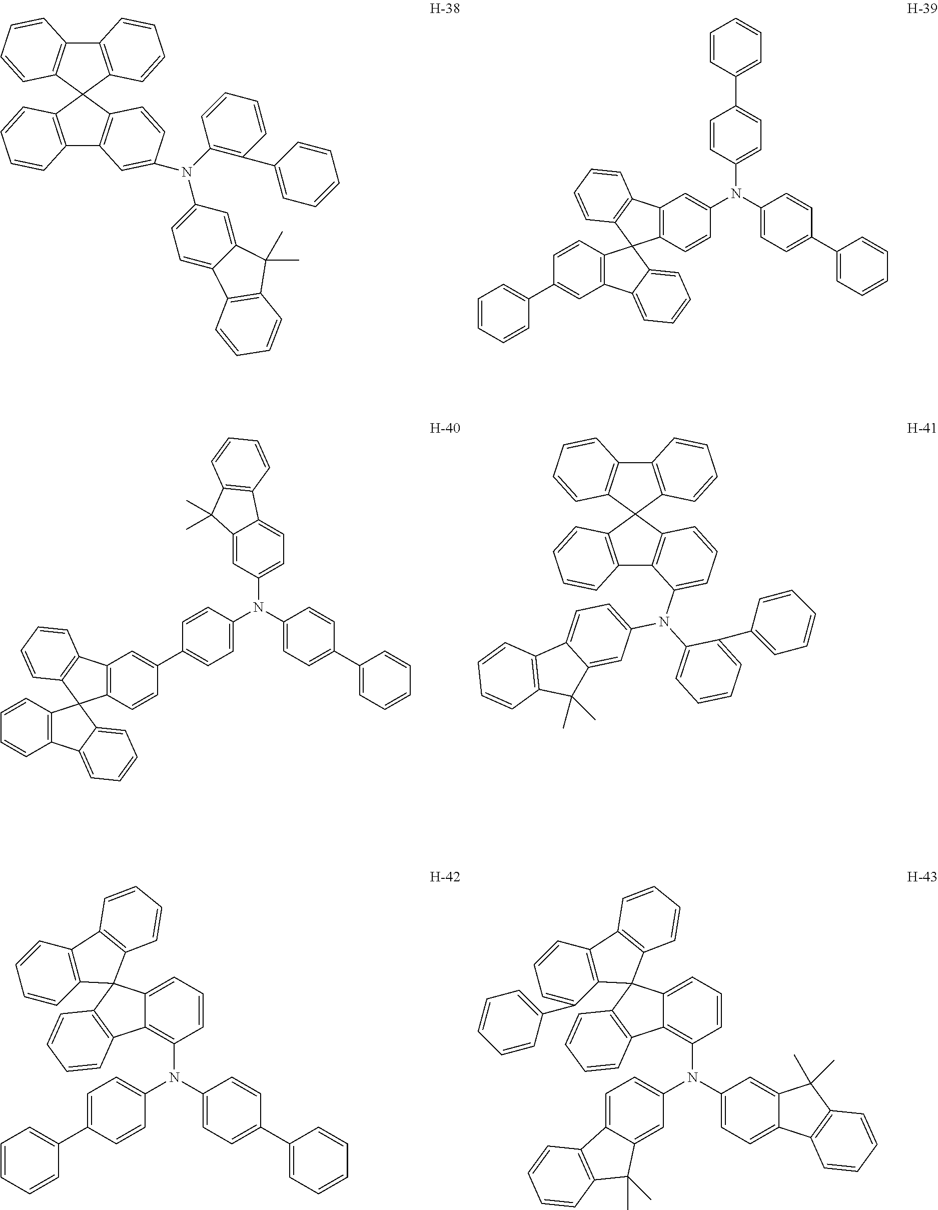

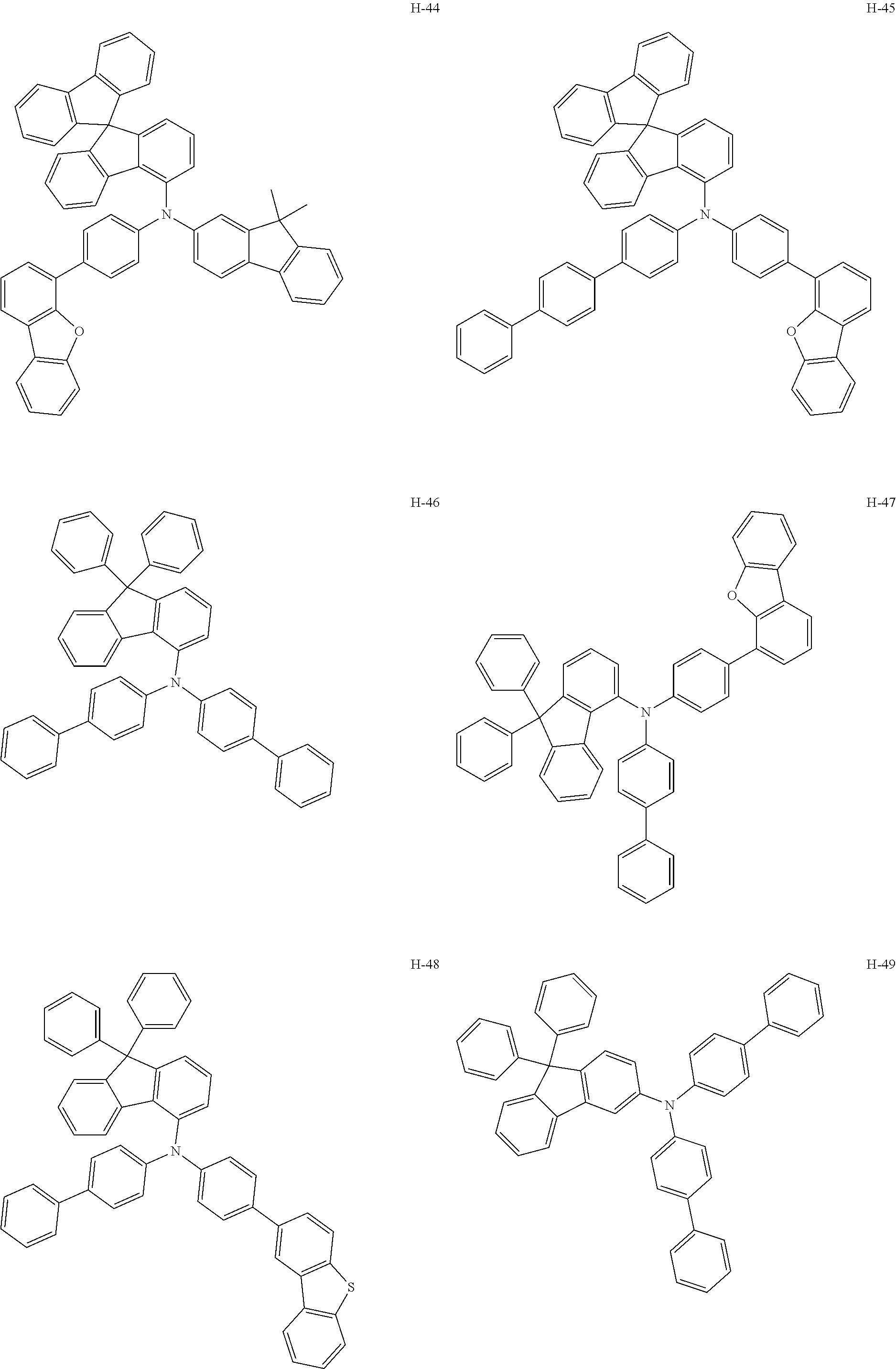

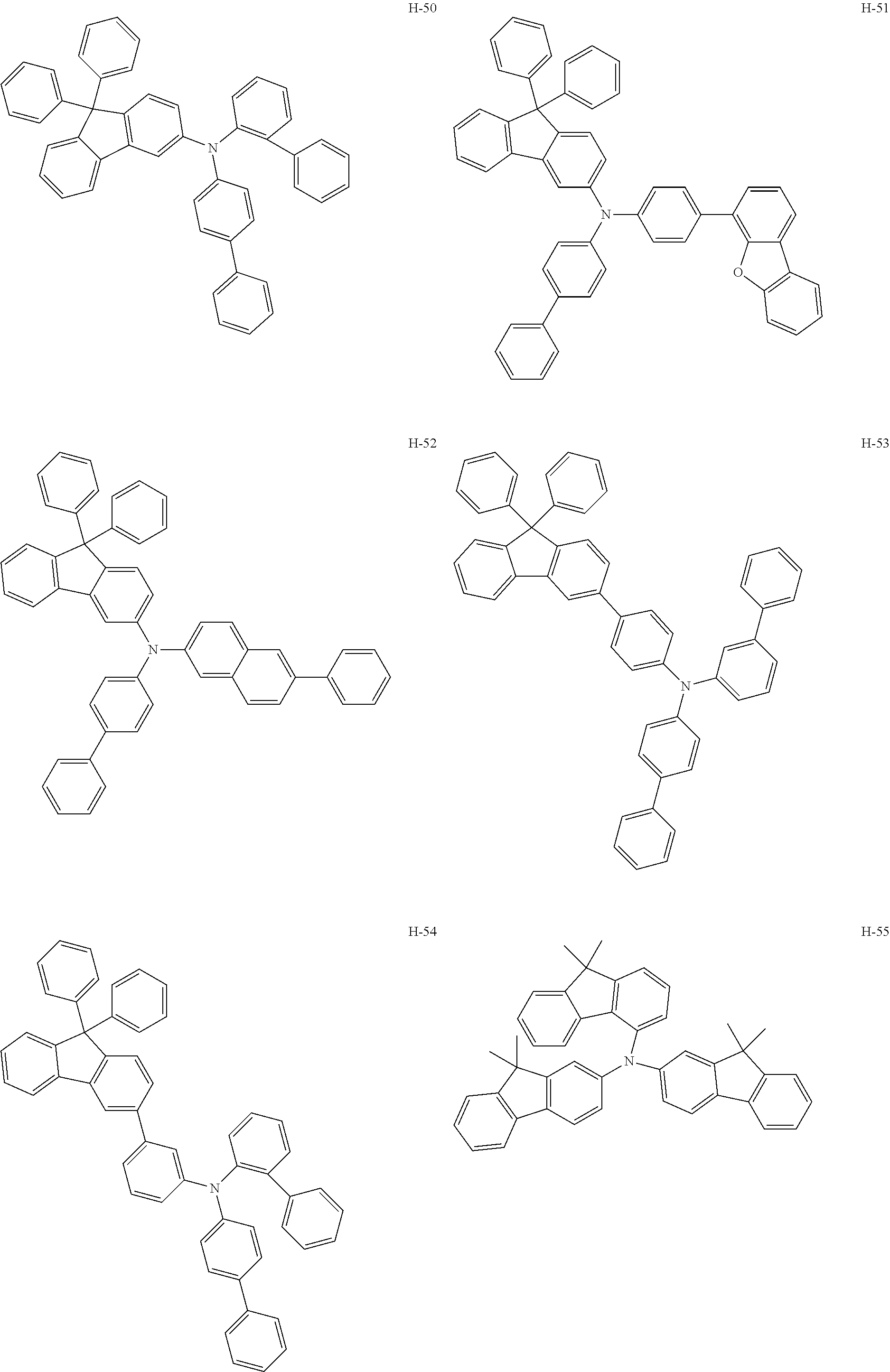

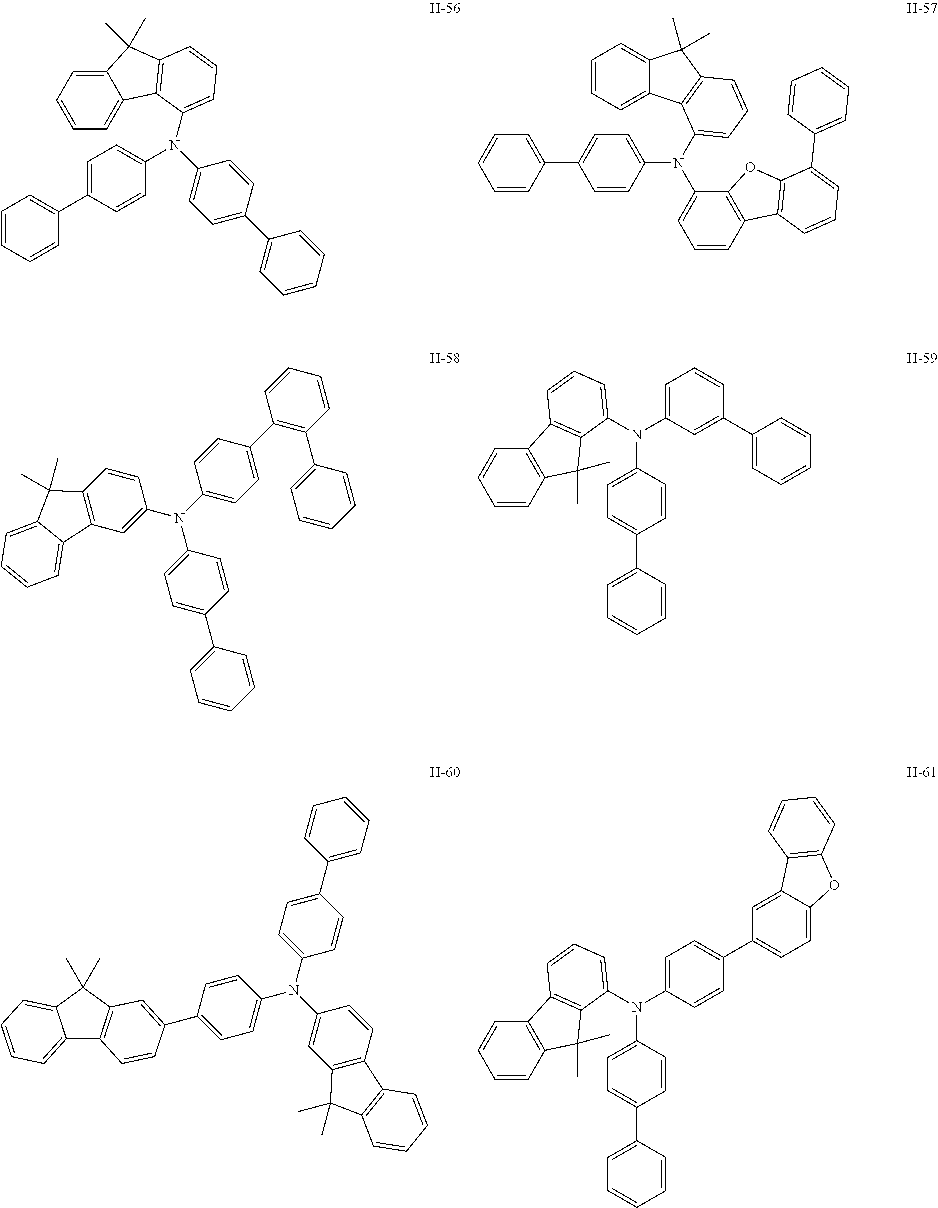 [SMILES: CC1(C)c2ccccc2-c2c(N(c3ccc(-c4ccccc4)cc3)c3ccc(-c4ccccc4)cc3)cccc21 ; CC1(C)c2ccccc2-c2c(N(c3ccc(-c4ccccc4)cc3)c3cccc4c3oc3c(-c5ccccc5)cccc34)cccc21 ; CC1(C)c2ccccc2-c2cc(N(c3ccc(-c4ccccc4)cc3)c3ccc(-c4ccccc4-c4ccccc4)cc3)ccc21 ; CC1(C)c2ccccc2-c2cccc(N(c3ccc(-c4ccccc4)cc3)c3cccc(-c4ccccc4)c3)c21 ; CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2cccc(N(c3ccc(-c4ccccc4)cc3)c3ccc(-c4ccc5oc6ccccc6c5c4)cc3)c21]

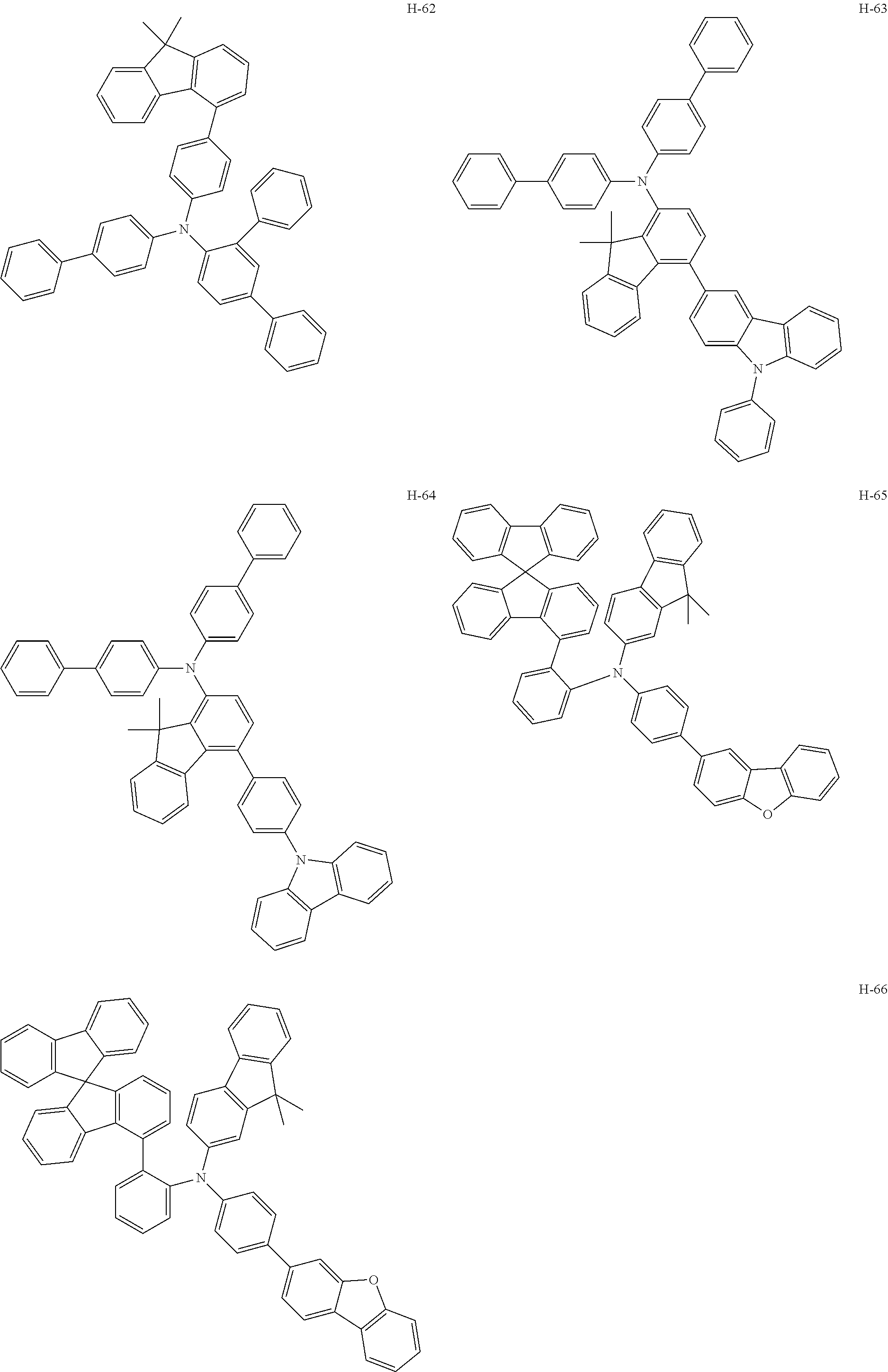

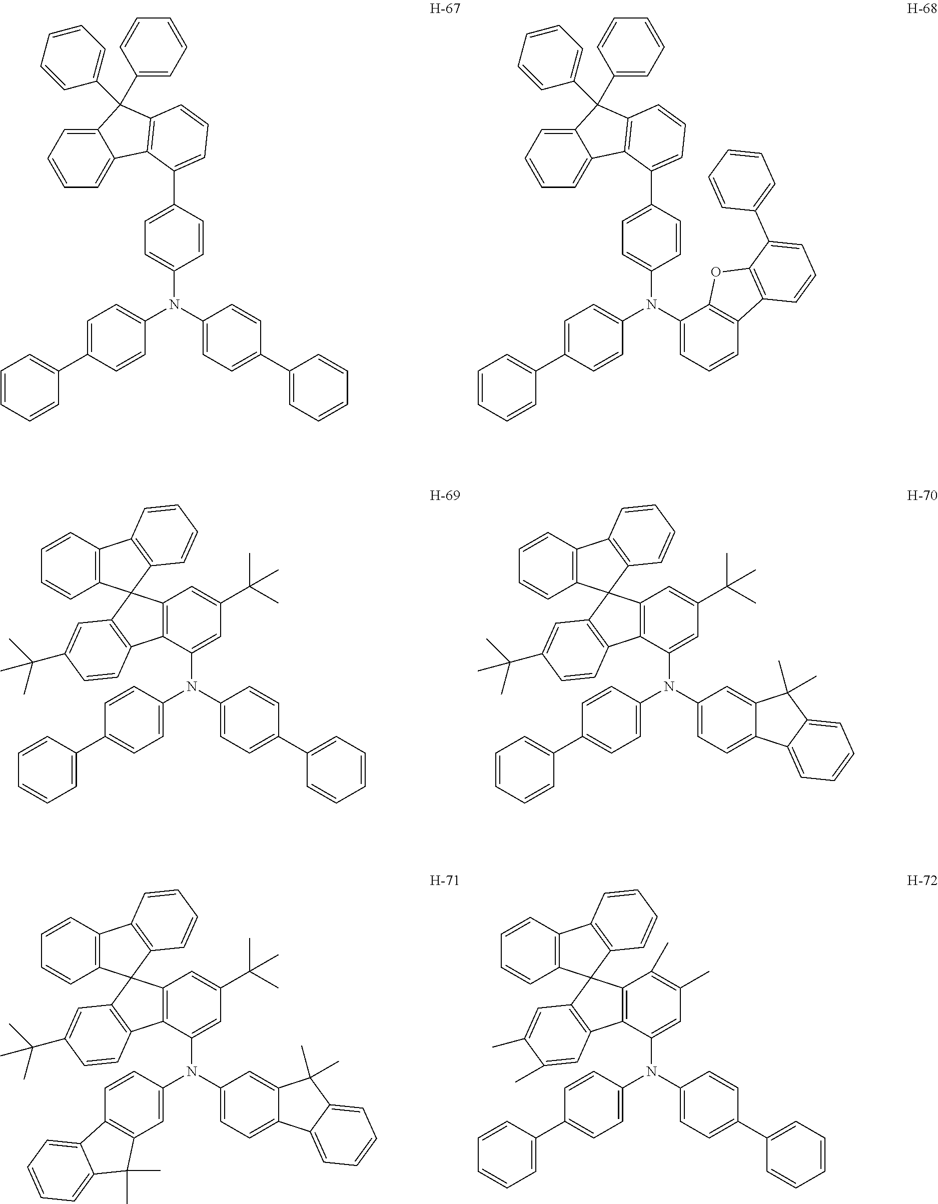

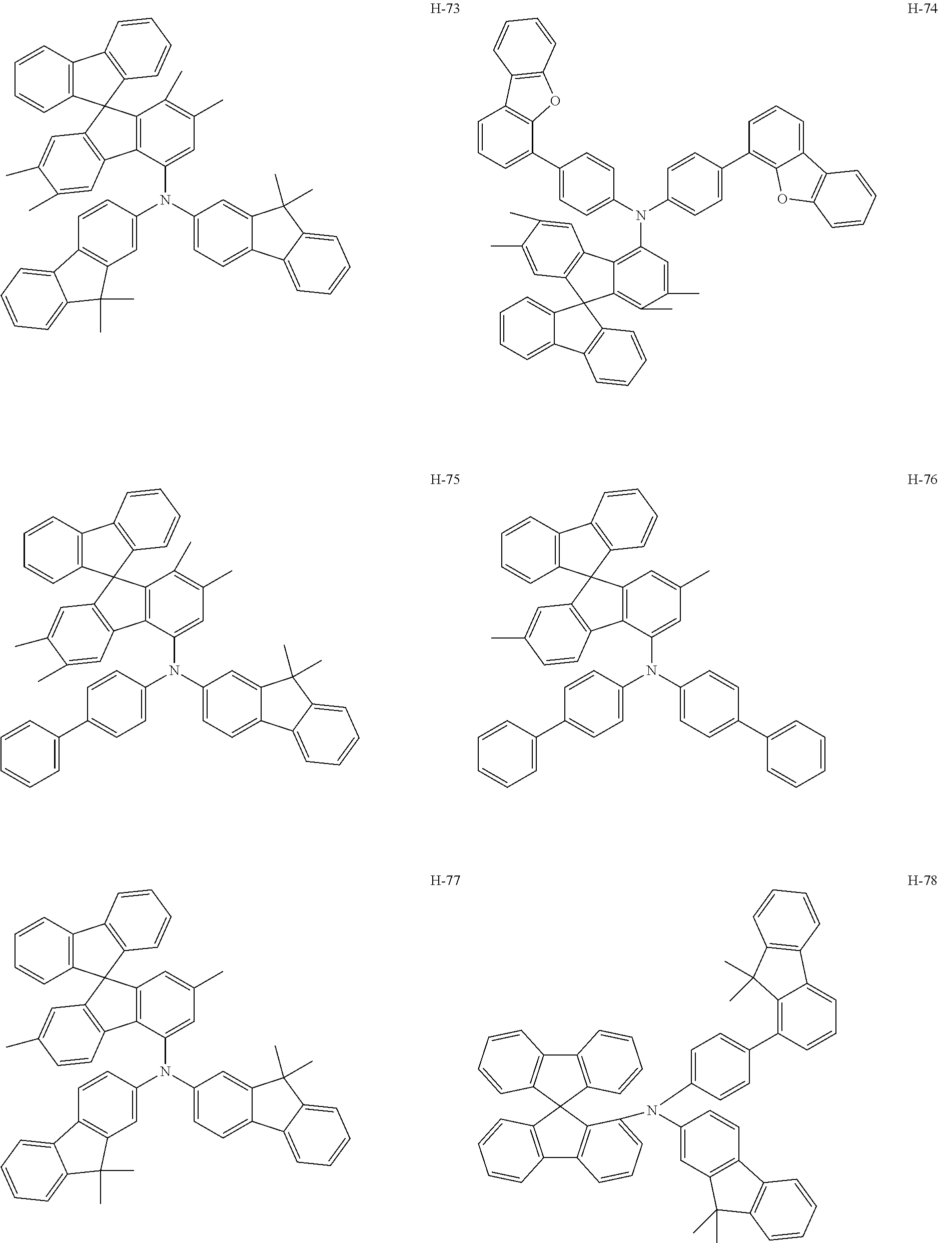

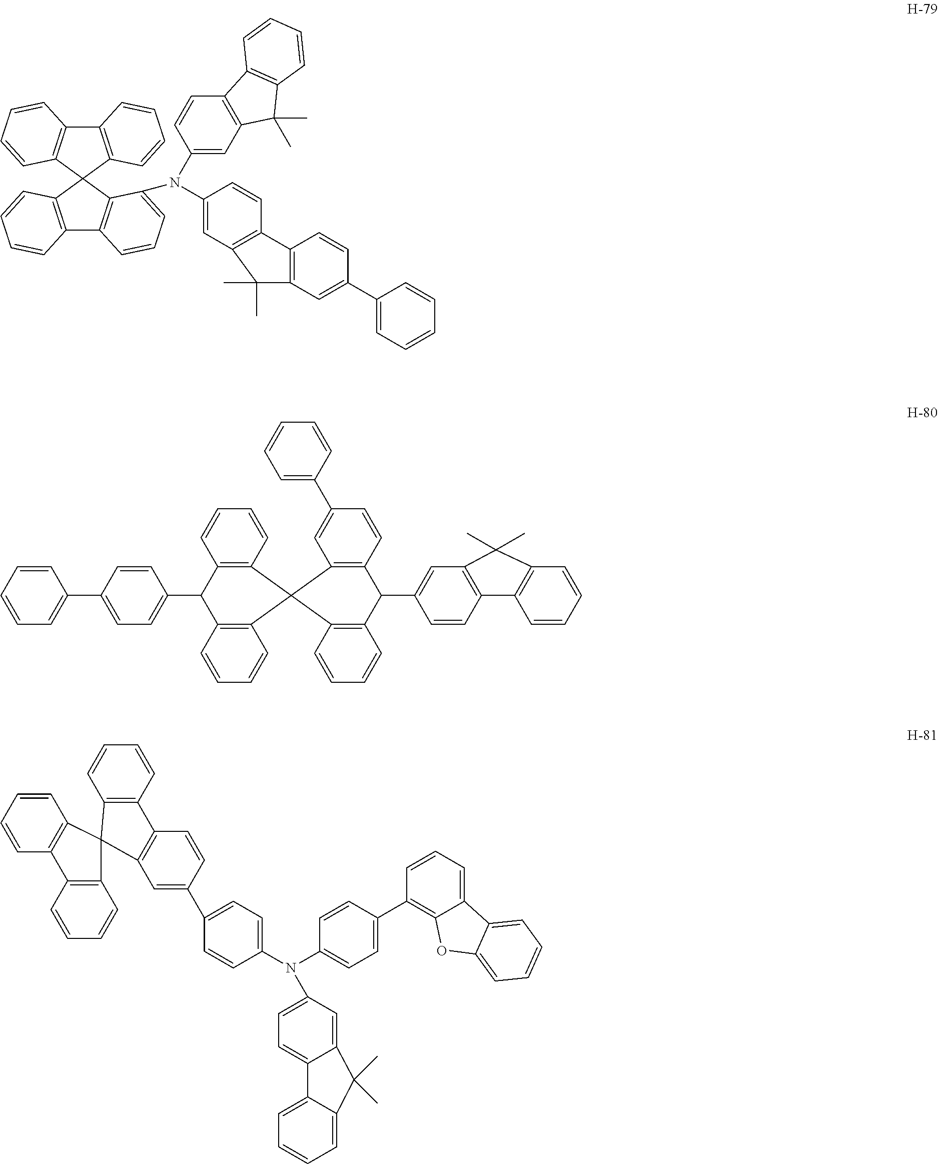

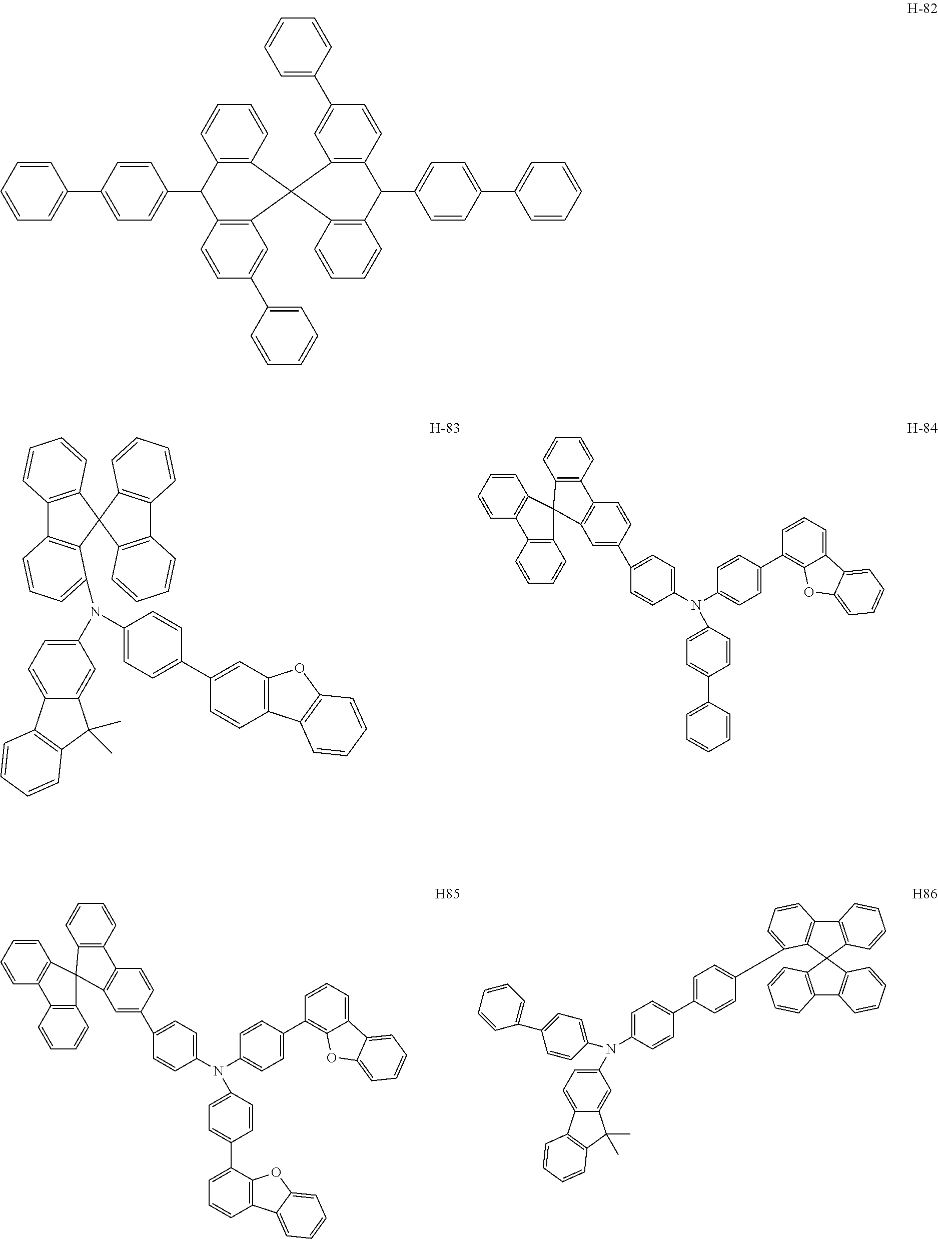

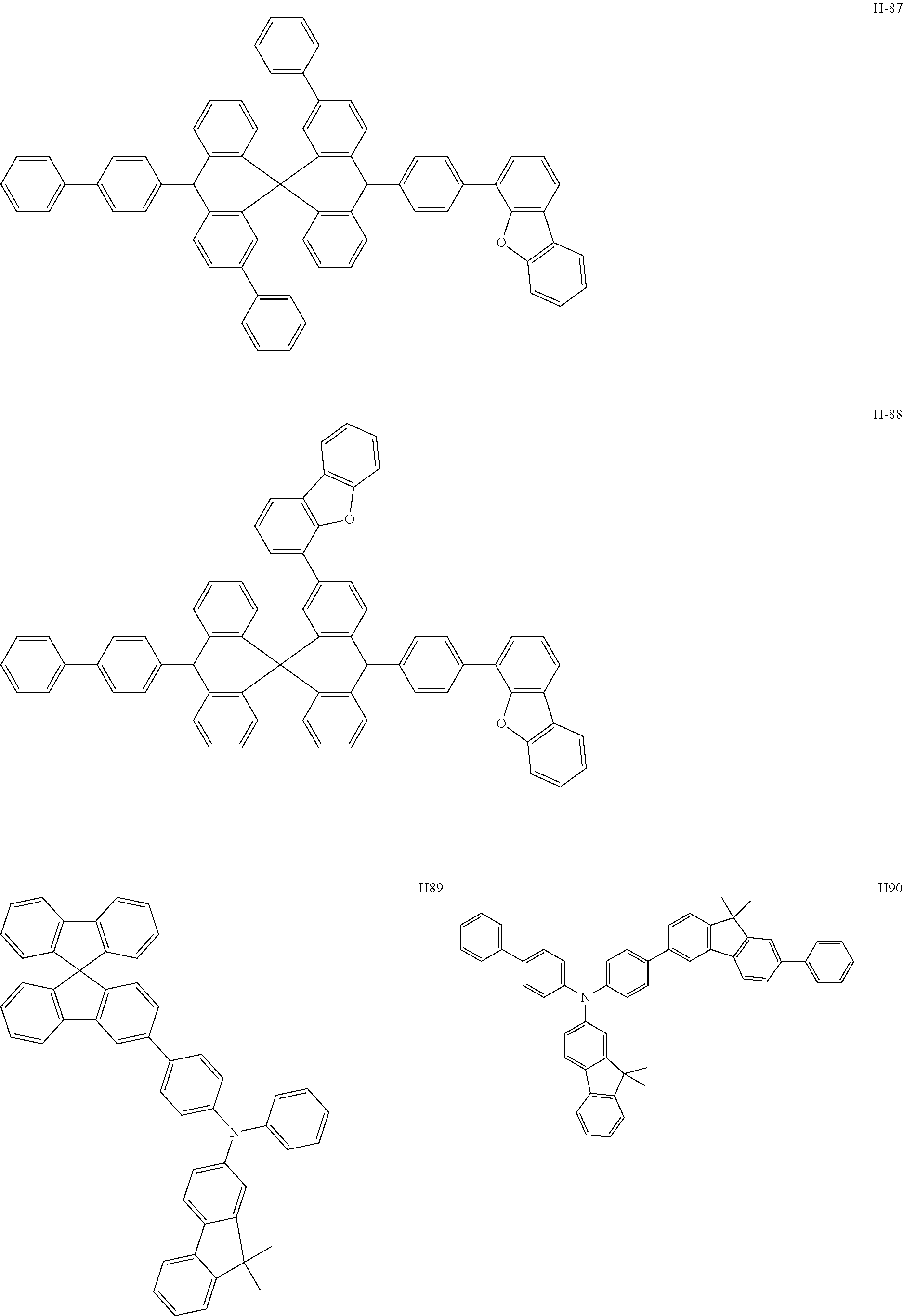 [SMILES: c1ccc(-c2ccc(C3c4ccccc4C4(c5ccccc5C(c5ccc(-c6cccc7c6oc6ccccc67)cc5)c5ccc(-c6ccccc6)cc54)c4cc(-c5ccccc5)ccc43)cc2)cc1 ; c1ccc(-c2ccc(C3c4ccccc4C4(c5ccccc53)c3ccccc3C(c3ccc(-c5cccc6c5oc5ccccc56)cc3)c3ccc(-c5cccc6c5oc5ccccc56)cc34)cc2)cc1 ; CC1(C)c2ccccc2-c2ccc(N(c3ccccc3)c3ccc(-c4ccc5c(c4)-c4ccccc4C54c5ccccc5-c5ccccc54)cc3)cc21 ; CC1(C)c2ccc(-c3ccc(N(c4ccc(-c5ccccc5)cc4)c4ccc5c(c4)C(C)(C)c4ccccc4-5)cc3)cc2-c2ccc(-c3ccccc3)cc21]

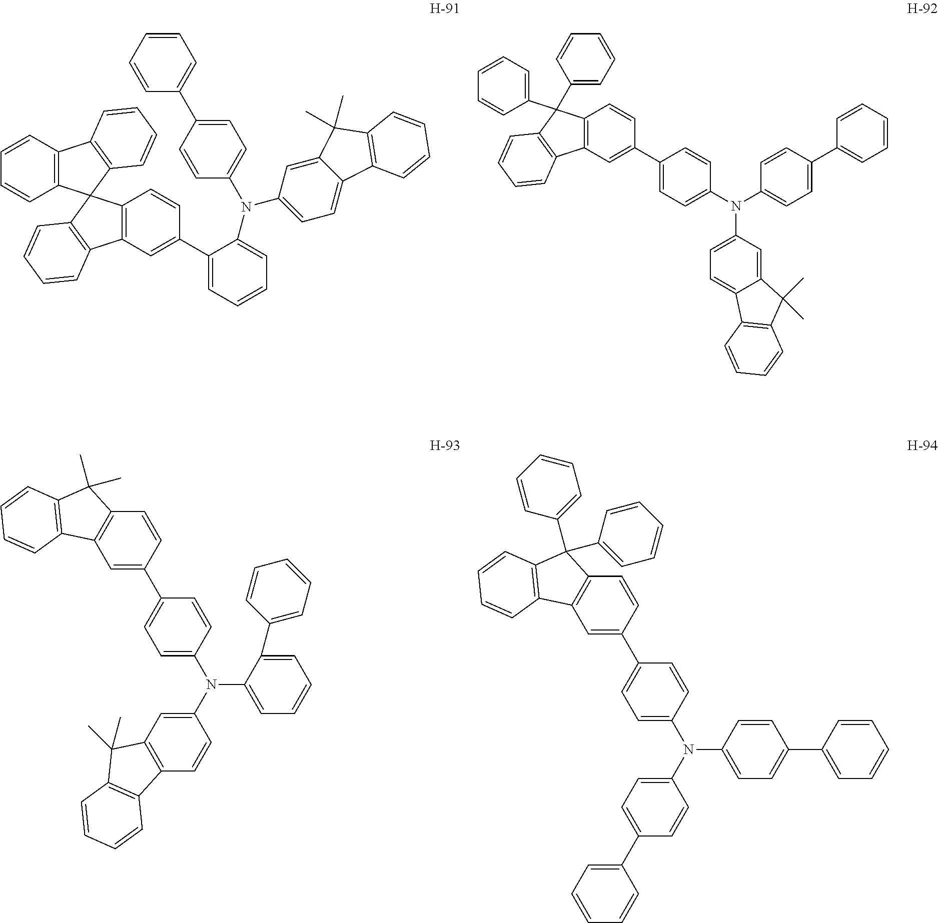 [SMILES: CC1(C)c2ccccc2-c2ccc(N(c3ccc(-c4ccccc4)cc3)c3ccccc3-c3ccc4c(c3)-c3ccccc3C43c4ccccc4-c4ccccc43)cc21 ; CC1(C)c2ccccc2-c2ccc(N(c3ccc(-c4ccccc4)cc3)c3ccc(-c4ccc5c(c4)-c4ccccc4C5(c4ccccc4)c4ccccc4)cc3)cc21 ; CC1(C)c2ccccc2-c2cc(-c3ccc(N(c4ccc5c(c4)C(C)(C)c4ccccc4-5)c4ccccc4-c4ccccc4)cc3)ccc21 ; c1ccc(-c2ccc(N(c3ccc(-c4ccccc4)cc3)c3ccc(-c4ccc5c(c4)-c4ccccc4C5(c4ccccc4)c4ccccc4)cc3)cc2)cc1]

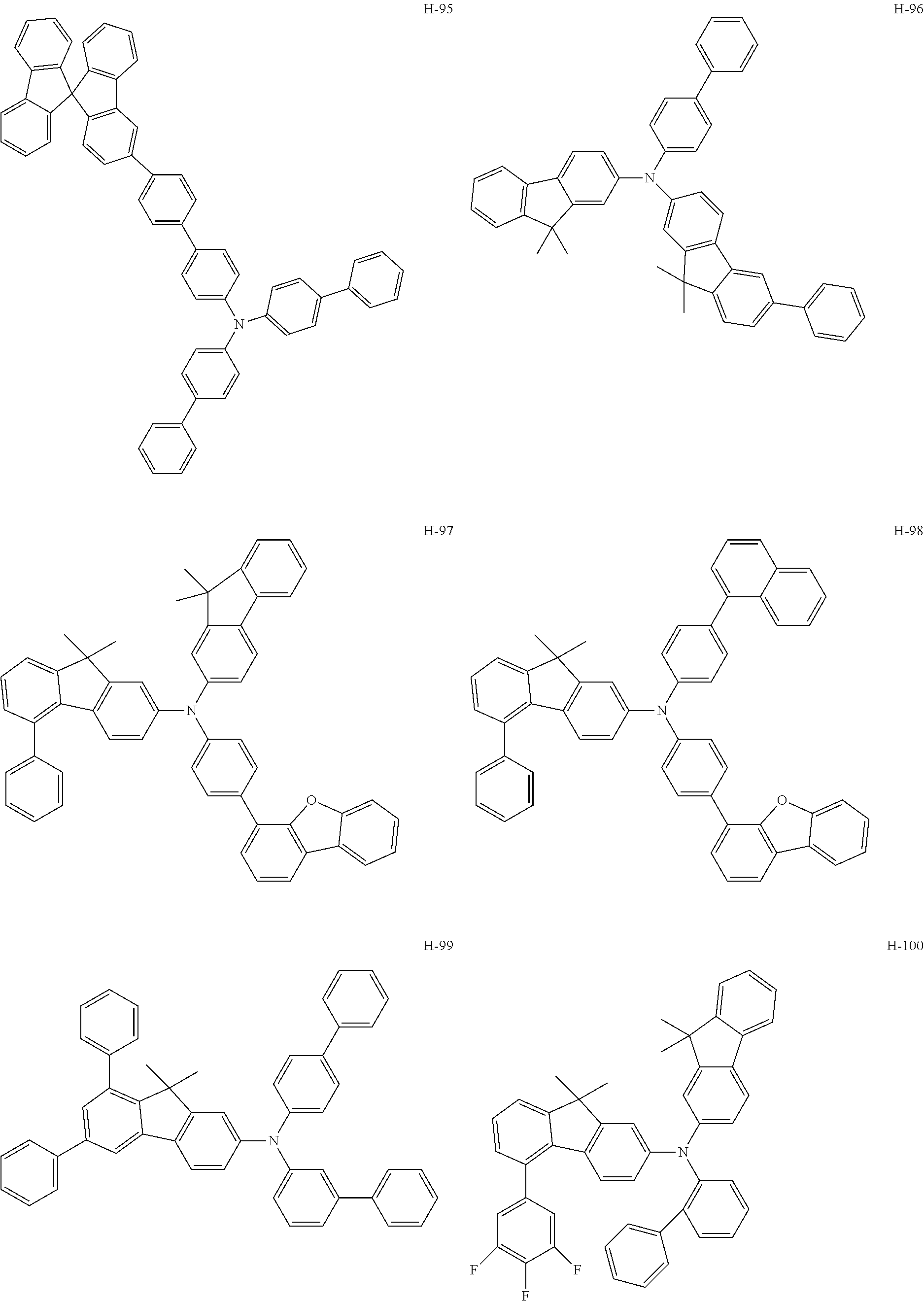

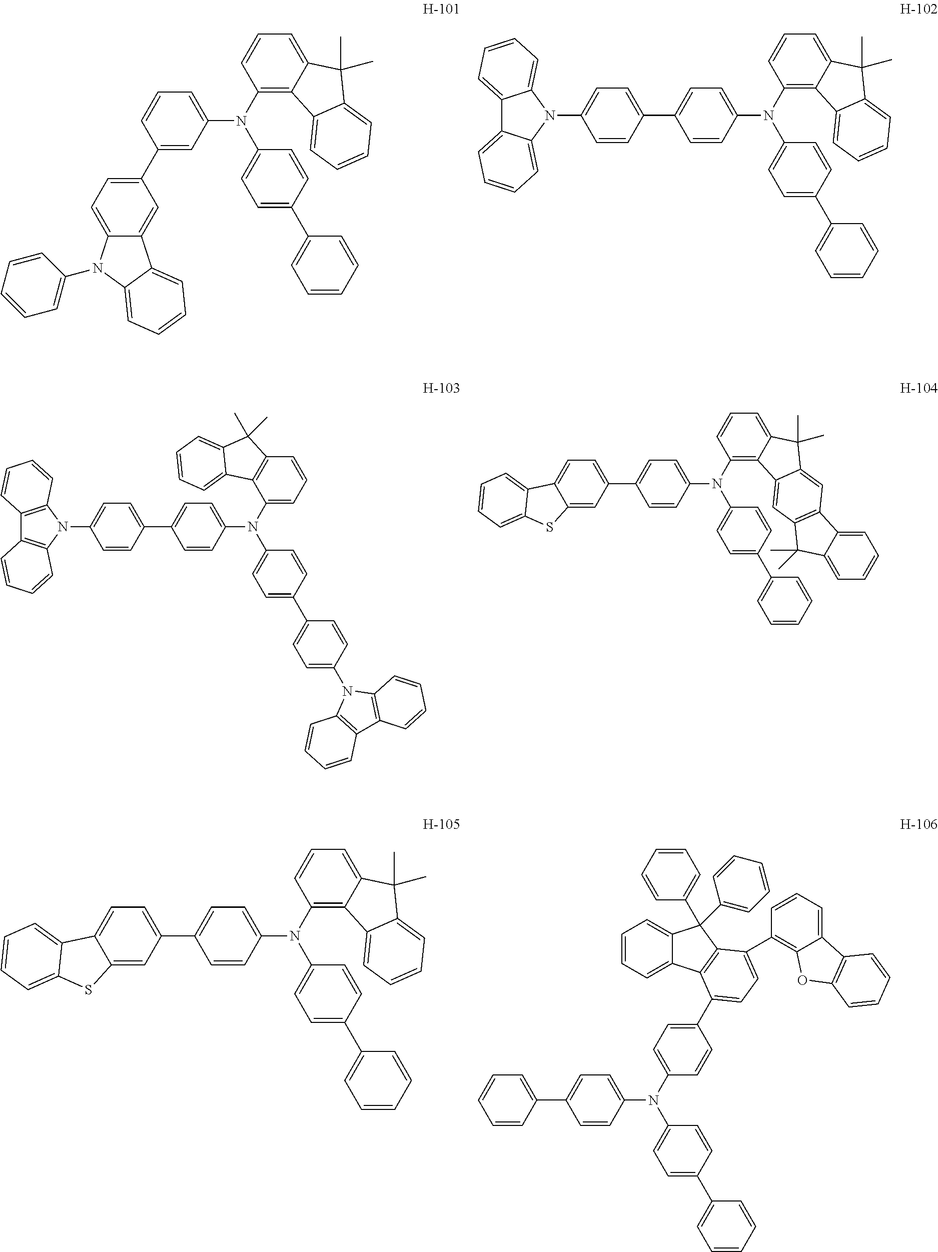

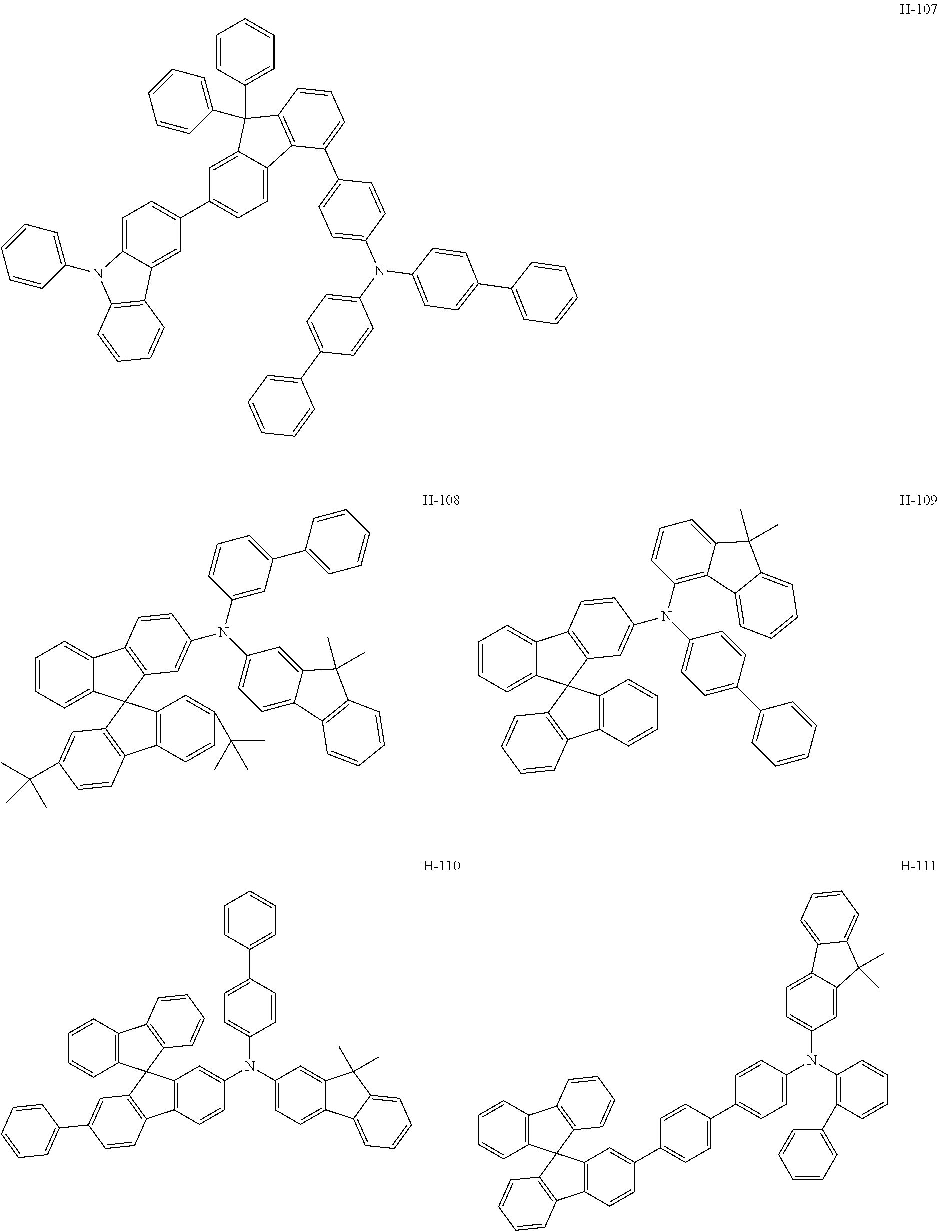

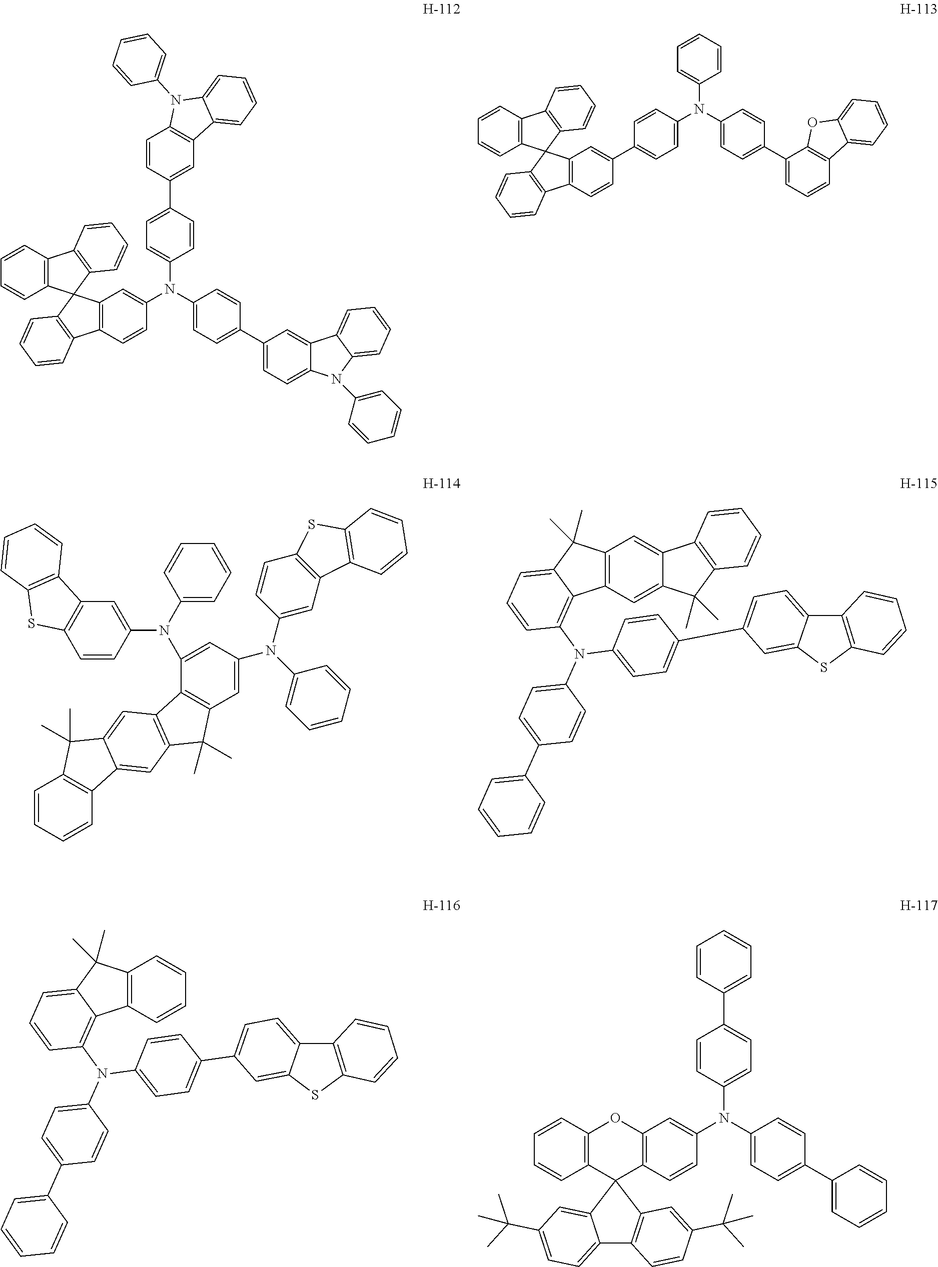

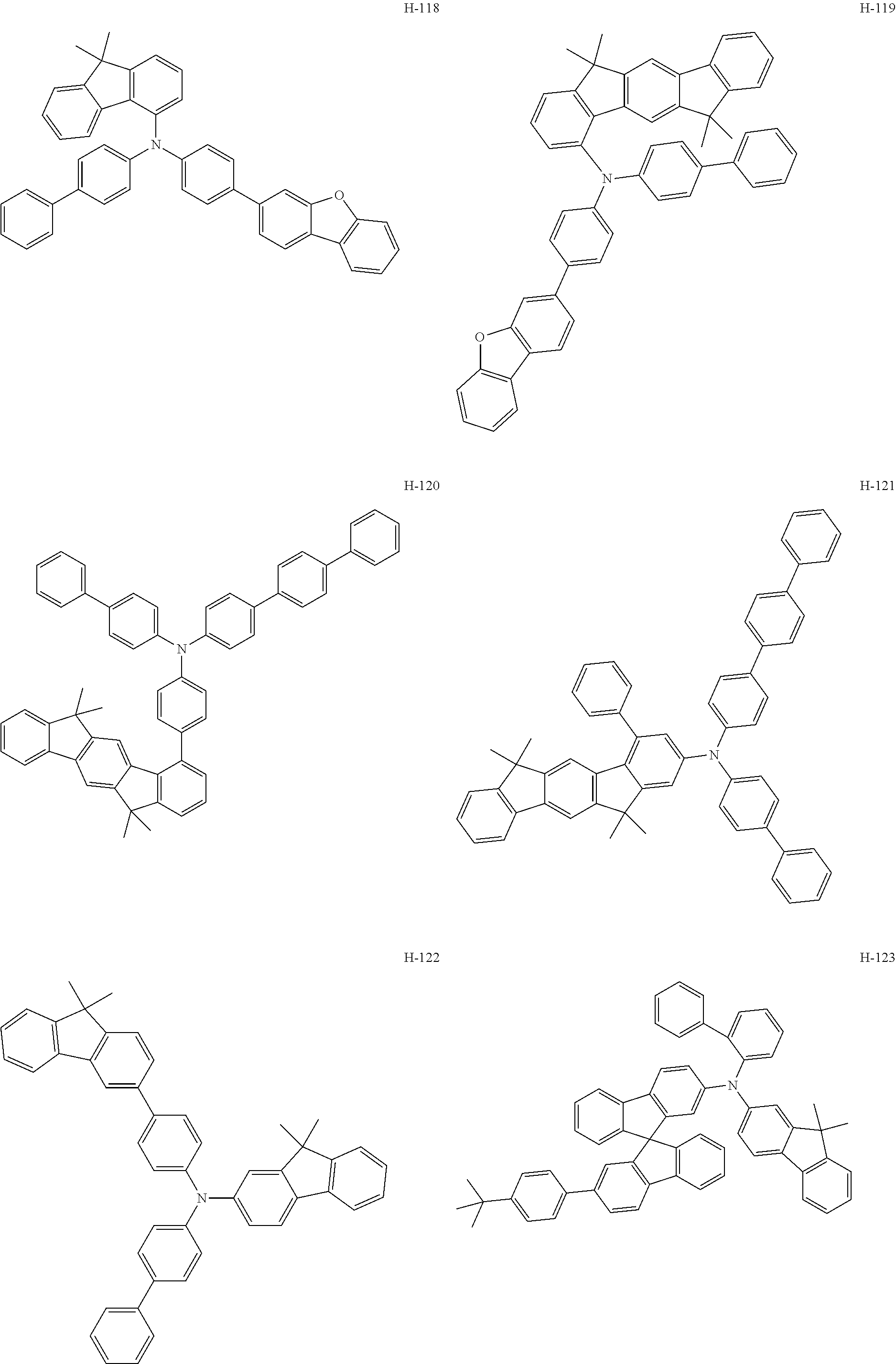

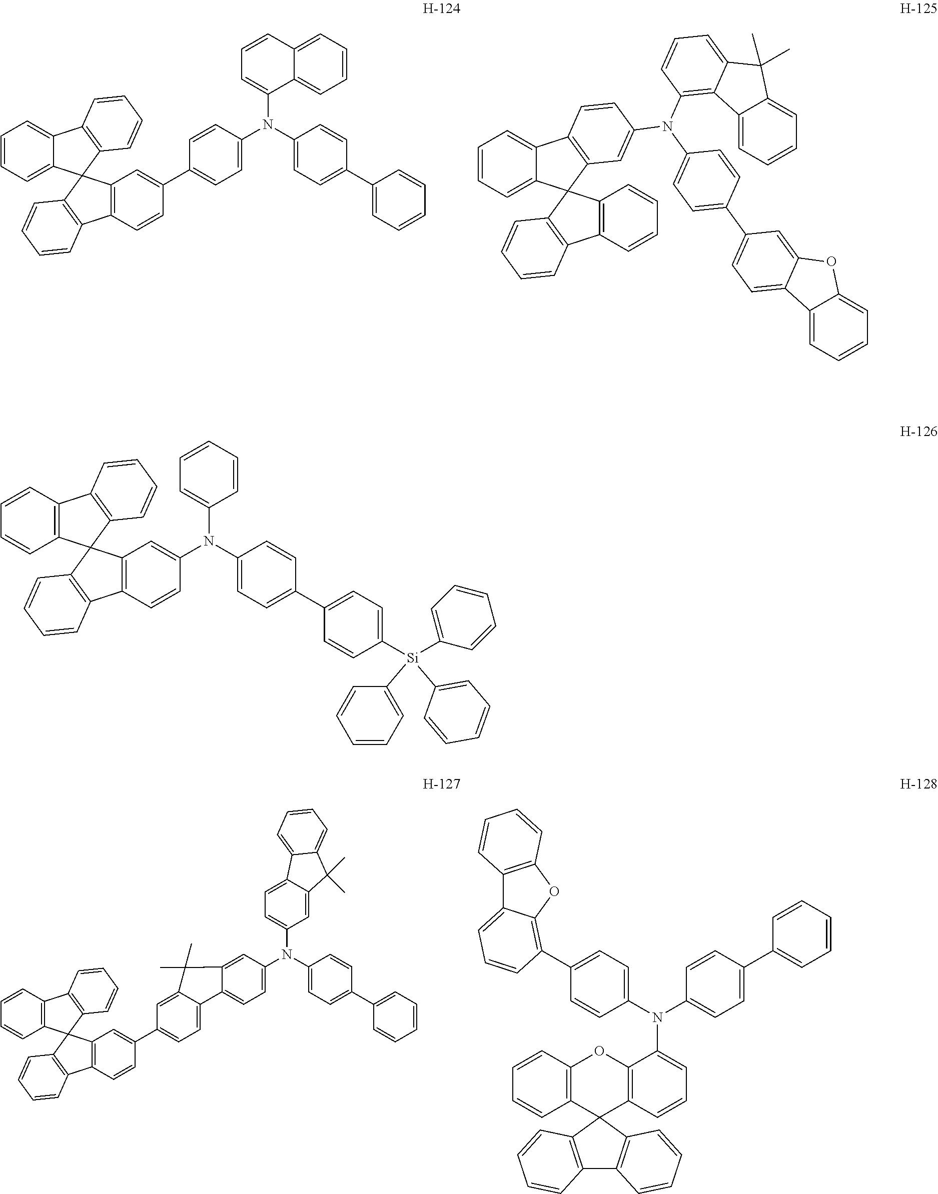

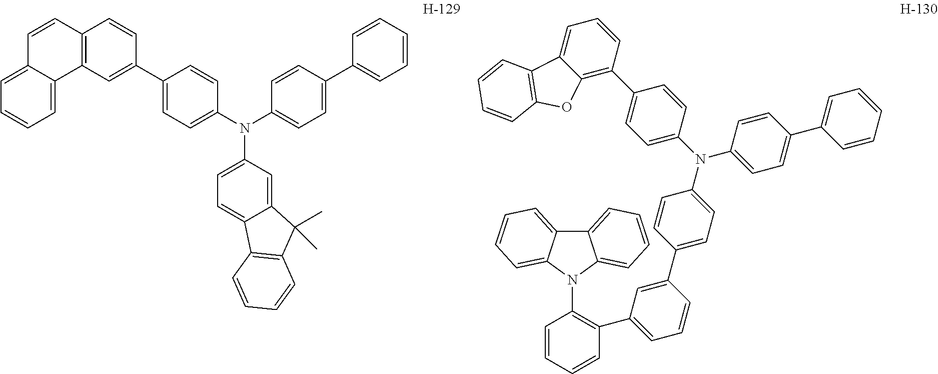 [SMILES: CC1(C)c2ccccc2-c2ccc(N(c3ccc(-c4ccccc4)cc3)c3ccc(-c4ccc5ccc6ccccc6c5c4)cc3)cc21 ; c1ccc(-c2ccc(N(c3ccc(-c4cccc(-c5ccccc5-n5c6ccccc6c6ccccc65)c4)cc3)c3ccc(-c4cccc5c4oc4ccccc45)cc3)cc2)cc1]

43. An organic electroluminescent device comprising the compound according to claim 42.

44. The organic electroluminescent device according to claim 43, wherein the compound is in a hole-transporting layer and/or in an emitting layer as matrix material.

45. An organic electroluminescent device comprising the compound according to claim 42, wherein the compound is in a hole injection layer, a hole transport layer, an electron blocker layer and/or an emitting layer.
